SEQUENCE LISTING (147 sequences)

SEQ ID NO: 1 - protein, 28 aa, Artificial Sequence
  Synthetic peptide
PDFPPEVEEQDASTLPVSCAWESGMKRH

SEQ ID NO: 2 - protein, 40 aa, Mus sp.
SHGFPPEVEEQDDGTLPMSCAQESGMDRHPAACASARINV

SEQ ID NO: 3 - protein, 40 aa, Homo sapiens
NPDFPPEVEEQDASTLPVSCAWESGMKRHRAACASASINV

SEQ ID NO: 4 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 5 - protein, 131 aa, Artificial Sequence
  Synthetic polypeptide
MAEVQLQASGGGFVQPGGSLRLSCAASGFTSWEDTMGWFRQAPGKEREFVSAISFDANDLSDTSVYYADSVKGRFTISRDNSKNTVYLQMNSLRAEDTATYYCAVASFEILLYGESLHIYWGQGTQVTVSS

SEQ ID NO: 6 - protein, 202 aa, Artificial Sequence
  Synthetic polypeptide
MSSQVQLQQSGPGRVKPSQTLSLACVISGDTVSSKTVSWNWIRQSPSRGLEWLGRTYFWPKWYTEYGASVKGRITIHPDTSKNQFSLQLSSVTPEDTAVYYCARSKTKRPPYYYAGMDVWGQGTTVTVSSASTKGPSGILGSGGGGSGILAAAYPYDVPDYANPDFPPEVEEQDASTLPVSCAWESGMKRHRAACASASINV

SEQ ID NO: 7 - protein, 202 aa, Artificial Sequence
  Synthetic polypeptide
MSSQVQLQQSGPGRVKPSQTLSLACVISGDTVSSKTVSWNWIRQSPSRGLEWLGRTYFWPKWYTEYGASVKGRITIHPDTSKNQFSLQLSSVTPEDTAVYYCARSKTKRPPYYYAGMDVWGQGTTVTVSSASTKGPSGILGSGGGGSGILAAAYPYDVPDYANPDFAAEVEEQDASTLPVSCAWESGMKRHRAACASASINV

SEQ ID NO: 8 - protein, 202 aa, Artificial Sequence
  Synthetic polypeptide
MSSQVQLQQSGPGRVKPSQTLSLACVISGDTVSSKTVSWNWIRQSPSRGLEWLGRTYFWPKWYTEYGASVKGRITIHPDTSKNQFSLQLSSVTPEDTAVYYCARSKTKRPPYYYAGMDVWGQGTTVTVSSASTKGPSGILGSGGGGSGILAAAYPYDVPDYANPDFPPEVEEQAASTLPVSCAWESGMKRHRAACASASINV

SEQ ID NO: 9 - protein, 202 aa, Artificial Sequence
  Synthetic polypeptide
MSSQVQLQQSGPGRVKPSQTLSLACVISGDTVSSKTVSWNWIRQSPSRGLEWLGRTYFWPKWYTEYGASVKGRITIHPDTSKNQFSLQLSSVTPEDTAVYYCARSKTKRPPYYYAGMDVWGQGTTVTVSSASTKGPSGILGSGGGGSGILAAAYPYDVPDYANPDFPPEVEEQDASTLPVSAAWESGMKRHRAACASASINV

SEQ ID NO: 10 - protein, 202 aa, Artificial Sequence
  Synthetic polypeptide
MSSQVQLQQSGPGRVKPSQTLSLACVISGDTVSSKTVSWNWIRQSPSRGLEWLGRTYFWPKWYTEYGASVKGRITIHPDTSKNQFSLQLSSVTPEDTAVYYCARSKTKRPPYYYAGMDVWGQGTTVTVSSASTKGPSGILGSGGGGSGILAAAYPYDVPDYANPDFPPEVEEQDASTLPVSCAWEAGMKRHRAACASASINV

SEQ ID NO: 11 - protein, 202 aa, Artificial Sequence
  Synthetic polypeptide
MSSQVQLQQSGPGRVKPSQTLSLACVISGDTVSSKTVSWNWIRQSPSRGLEWLGRTYFWPKWYTEYGASVKGRITIHPDTSKNQFSLQLSSVTPEDTAVYYCARSKTKRPPYYYAGMDVWGQGTTVTVSSASTKGPSGILGSGGGGSGILAAAYPYDVPDYAACCGEHPIRPPVEDFEESRASSTASAWLANMVQNKPVSDA

SEQ ID NO: 12 - protein, 183 aa, Artificial Sequence
  Synthetic polypeptide
MAEVQLQASGGGFVQPGGSLRLSCAASGFTSWEDTMGWFRQAPGKEREFVSAISFDANDLSDTSVYYADSVKGRFTISRDNSKNTVYLQMNSLRAEDTATYYCAVASFEILLYGESLHIYWGQGTQVTVSSAAAYPYDVPDYANPDFPPEVEEQDASTLPVSCAWESGMKRHRAACASASINV

SEQ ID NO: 13 - protein, 186 aa, Artificial Sequence
  Synthetic polypeptide
MSRQVQLVESGGGGLVQPGGSLRLSCVTPGGIFSDYAMGWYRQAPGKQRELVADITVSDTTRYSDPVKGRFTISRDNAKDMVYLQMNSLKPEDAAIYTCNAQKWEDRYGSSERYDYWGQGTQVTVSSAHHSEDPAAAYPYDVPDYANPDFPPEVEEQDASTLPVSCAWESGMKRHRAACASASINV

SEQ ID NO: 14 - protein, 15 aa, Artificial Sequence
  Synthetic peptide
GGGGSGGGGSGGGGS

SEQ ID NO: 15 - protein, 9 aa, Artificial Sequence
  Synthetic peptide
YPYDVPDYA

SEQ ID NO: 16 - protein, 123 aa, Artificial Sequence
  Synthetic polypeptide
VQLVESGGGGLVQPGGSLRLSCVTPGGIFSDYAMGWYRQAPGKQRELVADITVSDTTRYSDPVKGRFTISRDNAKDMVYLQMNSLKPEDAAIYTCNAQKWEDRYGSSERYDYWGQGTQVTVSS

SEQ ID NO: 17 - protein, 129 aa, Artificial Sequence
  Synthetic polypeptide
EVQLQASGGGFVQPGGSLRLSCAASGFTSWEDTMGWFRQAPGKEREFVSAISFDANDLSDTSVYYADSVKGRFTISRDNSKNTVYLQMNSLRAEDTATYYCAVASFEILLYGESLHIYWGQGTQVTVSS

SEQ ID NO: 18 - protein, 127 aa, Artificial Sequence
  Synthetic polypeptide
QVQLQQSGPGRVKPSQTLSLACVISGDTVSSKTVSWNWIRQSPSRGLEWLGRTYFWPKWYTEYGASVKGRITIHPDTSKNQFSLQLSSVTPEDTAVYYCARSKTKRPPYYYAGMDVWGQGTTVTVSS

SEQ ID NO: 19 - DNA, 5427 bp, Artificial Sequence
  Synthetic polynucleotide
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagactcgagcggccgccactgtgctggatatctgcagaattccaccacactggactagtggatccgagctcggtaccaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 20 - DNA, 4650 bp, Artificial Sequence
  Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtggagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgtcaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttgcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacatcgattgaattccccggggatcctctagagtcgacctgcagaagcttgcctcgagcagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 21 - DNA, 8477 bp, Artificial Sequence
  Synthetic polynucleotide
cgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttatcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtacccgggtcgaggtaggcgtgtacggtgggaggcctatataagcagagctcgtttagtgaaccgtcagatcgcctggagacgccatccacgctgttttgacctccatagaagacaccgggaccgatccagcctccgcggccccgaattacaggctagctatcaggatcctacgaggcgcgccagatagaattctcttgggcctcaagggccatgagttaaccagttctagatgtgactcatgtcaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcg

SEQ ID NO: 22 - protein, 441 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 23 - protein, 300 aa, Artificial Sequence
  Synthetic polypeptide
MQVQLVESGGGLVKPGGSLRLSCAASGFTFSSYAMSWVRQAPGKGLEWVAAISGSGDNTYYADSVKGRFTISRDNSENTVHLQMAGLRAEDTALYFCAKDGPEVGNPGGYFDFWGRGTLVTVSSGGGGSGGGGSGGGGSDIVMTKSPDSLAVSLGERATINCKSSQSLLYSSKNKDYLAWYQKKPGQSPRLLISWASTRESGVPDRFSGSGSGTDFTLTINRLQAEDVAVYYCQHYYSYPLTFGQGTKAAAYPYDVPDYANPDFPPEVEEQDASTLPVSCAWESGMKRHRAACASASINV

SEQ ID NO: 24 - protein, 300 aa, Artificial Sequence
  Synthetic polypeptide
MQVQLVESGGGLVKPGGSLRLSCAASGFTFSSYAMSWVRQAPGKGLEWVAAISGSGDNTYYADSVKGRFTISRDNSENTVHLQMAGLRAEDTALYFCAKDGPEVGNPGGYFDFWGRGTLVTVSSGGGGSGGGGSGGGGSDIVMTKSPDSLAVSLGERATINCKSSQSLLYSSKNKDYLAWYQKKPGQSPRLLISWASTRESGVPDRFSGSGSGTDFTLTINRLQAEDVAVYYCQHYYSYPLTFGQGTKAAAYPYDVPDYANPDFPPEVEEQDASTLPVSAAWESGMKRHRAACASASINV

SEQ ID NO: 25 - protein, 300 aa, Artificial Sequence
  Synthetic polypeptide
MQVQLVESGGGLVKPGGSLRLSCAASGFTFSSYAMSWVRQAPGKGLEWVAAISGSGDNTYYADSVKGRFTISRDNSENTVHLQMAGLRAEDTALYFCAKDGPEVGNPGGYFDFWGRGTLVTVSSGGGGSGGGGSGGGGSDIVMTKSPDSLAVSLGERATINCKSSQSLLYSSKNKDYLAWYQKKPGQSPRLLISWASTRESGVPDRFSGSGSGTDFTLTINRLQAEDVAVYYCQHYYSYPLTFGQGTKAAAYPYDVPDYANPDFPPEVEEQAASTLPVSCAWESGMKRHRAACASASINV

SEQ ID NO: 26 - protein, 300 aa, Artificial Sequence
  Synthetic polypeptide
MQVQLVESGGGLVKPGGSLRLSCAASGFTFSSYAMSWVRQAPGKGLEWVAAISGSGDNTYYADSVKGRFTISRDNSENTVHLQMAGLRAEDTALYFCAKDGPEVGNPGGYFDFWGRGTLVTVSSGGGGSGGGGSGGGGSDIVMTKSPDSLAVSLGERATINCKSSQSLLYSSKNKDYLAWYQKKPGQSPRLLISWASTRESGVPDRFSGSGSGTDFTLTINRLQAEDVAVYYCQHYYSYPLTFGQGTKAAAYPYDVPDYANPDFPPAVAAQDASTLPVSCAWESGMKRHRAACASASINV

SEQ ID NO: 27 - protein, 300 aa, Artificial Sequence
  Synthetic polypeptide
MQVQLVESGGGLVKPGGSLRLSCAASGFTFSSYAMSWVRQAPGKGLEWVAAISGSGDNTYYADSVKGRFTISRDNSENTVHLQMAGLRAEDTALYFCAKDGPEVGNPGGYFDFWGRGTLVTVSSGGGGSGGGGSGGGGSDIVMTKSPDSLAVSLGERATINCKSSQSLLYSSKNKDYLAWYQKKPGQSPRLLISWASTRESGVPDRFSGSGSGTDFTLTINRLQAEDVAVYYCQHYYSYPLTFGQGTKAAAYPYDVPDYANPDFPPEVEEQDASTLPVSCAWASGMKRHRAACASASINV

SEQ ID NO: 28 - protein, 300 aa, Artificial Sequence
  Synthetic polypeptide
MQVQLVESGGGLVKPGGSLRLSCAASGFTFSSYAMSWVRQAPGKGLEWVAAISGSGDNTYYADSVKGRFTISRDNSENTVHLQMAGLRAEDTALYFCAKDGPEVGNPGGYFDFWGRGTLVTVSSGGGGSGGGGSGGGGSDIVMTKSPDSLAVSLGERATINCKSSQSLLYSSKNKDYLAWYQKKPGQSPRLLISWASTRESGVPDRFSGSGSGTDFTLTINRLQAEDVAVYYCQHYYSYPLTFGQGTKAAAYPYDVPDYANPDFPPEVEEQDASTLPVSCAWESGMARHRAACASASINV

SEQ ID NO: 29 - protein, 300 aa, Artificial Sequence
  Synthetic polypeptide
MQVQLVESGGGLVKPGGSLRLSCAASGFTFSSYAMSWVRQAPGKGLEWVAAISGSGDNTYYADSVKGRFTISRDNSENTVHLQMAGLRAEDTALYFCAKDGPEVGNPGGYFDFWGRGTLVTVSSGGGGSGGGGSGGGGSDIVMTKSPDSLAVSLGERATINCKSSQSLLYSSKNKDYLAWYQKKPGQSPRLLISWASTRESGVPDRFSGSGSGTDFTLTINRLQAEDVAVYYCQHYYSYPLTFGQGTKAAAYPYDVPDYANPDFAAEVEEQDASTLPVSCAWESGMKRHRAACASASINV

SEQ ID NO: 30 - protein, 300 aa, Artificial Sequence
  Synthetic polypeptide
MQVQLVESGGGLVKPGGSLRLSCAASGFTFSSYAMSWVRQAPGKGLEWVAAISGSGDNTYYADSVKGRFTISRDNSENTVHLQMAGLRAEDTALYFCAKDGPEVGNPGGYFDFWGRGTLVTVSSGGGGSGGGGSGGGGSDIVMTKSPDSLAVSLGERATINCKSSQSLLYSSKNKDYLAWYQKKPGQSPRLLISWASTRESGVPDRFSGSGSGTDFTLTINRLQAEDVAVYYCQHYYSYPLTFGQGTKAAAYPYDVPDYANPDFPPEVEEQDASTLAVSCAWESGMKRHRAACASASINV

SEQ ID NO: 31 - protein, 300 aa, Artificial Sequence
  Synthetic polypeptide
MQVQLVESGGGLVKPGGSLRLSCAASGFTFSSYAMSWVRQAPGKGLEWVAAISGSGDNTYYADSVKGRFTISRDNSENTVHLQMAGLRAEDTALYFCAKDGPEVGNPGGYFDFWGRGTLVTVSSGGGGSGGGGSGGGGSDIVMTKSPDSLAVSLGERATINCKSSQSLLYSSKNKDYLAWYQKKPGQSPRLLISWASTRESGVPDRFSGSGSGTDFTLTINRLQAEDVAVYYCQHYYSYPLTFGQGTKAAAYPYDVPDYANPDFPPEVEEQDAATLPVSCAWESGMKRHRAACASASINV

SEQ ID NO: 32 - protein, 300 aa, Artificial Sequence
  Synthetic polypeptide
MQVQLVESGGGLVKPGGSLRLSCAASGFTFSSYAMSWVRQAPGKGLEWVAAISGSGDNTYYADSVKGRFTISRDNSENTVHLQMAGLRAEDTALYFCAKDGPEVGNPGGYFDFWGRGTLVTVSSGGGGSGGGGSGGGGSDIVMTKSPDSLAVSLGERATINCKSSQSLLYSSKNKDYLAWYQKKPGQSPRLLISWASTRESGVPDRFSGSGSGTDFTLTINRLQAEDVAVYYCQHYYSYPLTFGQGTKAAAYPYDVPDYANPDFPPEVEEQDASTLPVACAWESGMKRHRAACASASINV

SEQ ID NO: 33 - protein, 300 aa, Artificial Sequence
  Synthetic polypeptide
MQVQLVESGGGLVKPGGSLRLSCAASGFTFSSYAMSWVRQAPGKGLEWVAAISGSGDNTYYADSVKGRFTISRDNSENTVHLQMAGLRAEDTALYFCAKDGPEVGNPGGYFDFWGRGTLVTVSSGGGGSGGGGSGGGGSDIVMTKSPDSLAVSLGERATINCKSSQSLLYSSKNKDYLAWYQKKPGQSPRLLISWASTRESGVPDRFSGSGSGTDFTLTINRLQAEDVAVYYCQHYYSYPLTFGQGTKAAAYPYDVPDYANPDFPPEVEEQDASTLPVSCAWEAGMKRHRAACASASINV

SEQ ID NO: 34 - protein, 300 aa, Artificial Sequence
  Synthetic polypeptide
MQVQLVESGGGLVKPGGSLRLSCAASGFTFSSYAMSWVRQAPGKGLEWVAAISGSGDNTYYADSVKGRFTISRDNSENTVHLQMAGLRAEDTALYFCAKDGPEVGNPGGYFDFWGRGTLVTVSSGGGGSGGGGSGGGGSDIVMTKSPDSLAVSLGERATINCKSSQSLLYSSKNKDYLAWYQKKPGQSPRLLISWASTRESGVPDRFSGSGSGTDFTLTINRLQAEDVAVYYCQHYYSYPLTFGQGTKAAAYPYDVPDYAACCGEHPIRPPVEDFEESRASSTASAWLANMVQNKPVSDA

SEQ ID NO: 35 - protein, 299 aa, Artificial Sequence
  Synthetic polypeptide
QVQLVESGGGLVKPGGSLRLSCAASGFTFSSYAMSWVRQAPGKGLEWVAAISGSGDNTYYADSVKGRFTISRDNSENTVHLQMAGLRAEDTALYFCAKDGPAVGNPGGYFDFWGRGTLVTVSSGGGGSGGGGSGGGGSDIVMTQSPDSLAVSLGERATINCKSSQSLLYSSNNKDYLAWYQQKPGQSPRLLIPWASTRESGVPDRFSGSGSGTDFTLTINRLQAEDVAVYYCQHYYSYPLTFGQGTKAAAYPYDVPDYANPDFPPEVEEQDASTLPVSCAWESGMKRHRAACASASINV

SEQ ID NO: 36 - protein, 299 aa, Artificial Sequence
  Synthetic polypeptide
QVQLVESGGGLVKPGGSLRLSCAASGFTFSSYAMSWVRQAPGKGLEWVAAISGSGDNTYYADSVKGRFTISRDNSENTVHLQMAGLRAEDTALYFCAKDGPAVGNPGGYFDFWGRGTLVTVSSGGGGSGGGGSGGGGSDIVMTQSPDSLAVSLGERATINCKSSQSLLYSSNNKDYLAWYQQKPGQSPRLLIPWASTRESGVPDRFSGSGSGTDFTLTINRLQAEDVAVYYCQHYYSYPLTFGQGTKAAAYPYDVPDYANPDFPPEVEEQDASTLPVSAAWESGMKRHRAACASASINV

SEQ ID NO: 37 - protein, 299 aa, Artificial Sequence
  Synthetic polypeptide
QVQLVESGGGLVKPGGSLRLSCAASGFTFSSYAMSWVRQAPGKGLEWVAAISGSGDNTYYADSVKGRFTISRDNSENTVHLQMAGLRAEDTALYFCAKDGPAVGNPGGYFDFWGRGTLVTVSSGGGGSGGGGSGGGGSDIVMTQSPDSLAVSLGERATINCKSSQSLLYSSNNKDYLAWYQQKPGQSPRLLIPWASTRESGVPDRFSGSGSGTDFTLTINRLQAEDVAVYYCQHYYSYPLTFGQGTKAAAYPYDVPDYANPDFPPEVEEQAASTLPVSCAWESGMKRHRAACASASINV

SEQ ID NO: 38 - protein, 300 aa, Artificial Sequence
  Synthetic polypeptide
MQVQLVESGGGLVKPGGSLRLSCAASGFTFSSYAMSWVRQAPGKGLEWVAAISGSGDNTYYADSVKGRFTISRDNSENTVHLQMAGLRAEDTALYFCAKDGPAVGNPGGYFDFWGRGTLVTVSSGGGGSGGGGSGGGGSDIVMTQSPDSLAVSLGERATINCKSSQSLLYSSNNKDYLAWYQQKPGQSPRLLIPWASTRESGVPDRFSGSGSGTDFTLTINRLQAEDVAVYYCQHYYSYPLTFGQGTKAAAYPYDVPDYANPDFPPAVAAQDASTLPVSCAWESGMKRHRAACASASINV

SEQ ID NO: 39 - protein, 300 aa, Artificial Sequence
  Synthetic polypeptide
MQVQLVESGGGLVKPGGSLRLSCAASGFTFSSYAMSWVRQAPGKGLEWVAAISGSGDNTYYADSVKGRFTISRDNSENTVHLQMAGLRAEDTALYFCAKDGPAVGNPGGYFDFWGRGTLVTVSSGGGGSGGGGSGGGGSDIVMTQSPDSLAVSLGERATINCKSSQSLLYSSNNKDYLAWYQQKPGQSPRLLIPWASTRESGVPDRFSGSGSGTDFTLTINRLQAEDVAVYYCQHYYSYPLTFGQGTKAAAYPYDVPDYANPDFPPEVEEQDASTLPVSCAWASGMKRHRAACASASINV

SEQ ID NO: 40 - protein, 300 aa, Artificial Sequence
  Synthetic polypeptide
MQVQLVESGGGLVKPGGSLRLSCAASGFTFSSYAMSWVRQAPGKGLEWVAAISGSGDNTYYADSVKGRFTISRDNSENTVHLQMAGLRAEDTALYFCAKDGPAVGNPGGYFDFWGRGTLVTVSSGGGGSGGGGSGGGGSDIVMTQSPDSLAVSLGERATINCKSSQSLLYSSNNKDYLAWYQQKPGQSPRLLIPWASTRESGVPDRFSGSGSGTDFTLTINRLQAEDVAVYYCQHYYSYPLTFGQGTKAAAYPYDVPDYANPDFPPEVEEQDASTLPVSCAWESGMARHRAACASASINV

SEQ ID NO: 41 - protein, 299 aa, Artificial Sequence
  Synthetic polypeptide
QVQLVESGGGLVKPGGSLRLSCAASGFTFSSYAMSWVRQAPGKGLEWVAAISGSGDNTYYADSVKGRFTISRDNSENTVHLQMAGLRAEDTALYFCAKDGPAVGNPGGYFDFWGRGTLVTVSSGGGGSGGGGSGGGGSDIVMTQSPDSLAVSLGERATINCKSSQSLLYSSNNKDYLAWYQQKPGQSPRLLIPWASTRESGVPDRFSGSGSGTDFTLTINRLQAEDVAVYYCQHYYSYPLTFGQGTKAAAYPYDVPDYANPDFAAEVEEQDASTLPVSCAWESGMKRHRAACASASINV

SEQ ID NO: 42 - protein, 300 aa, Artificial Sequence
  Synthetic polypeptide
MQVQLVESGGGLVKPGGSLRLSCAASGFTFSSYAMSWVRQAPGKGLEWVAAISGSGDNTYYADSVKGRFTISRDNSENTVHLQMAGLRAEDTALYFCAKDGPAVGNPGGYFDFWGRGTLVTVSSGGGGSGGGGSGGGGSDIVMTQSPDSLAVSLGERATINCKSSQSLLYSSNNKDYLAWYQQKPGQSPRLLIPWASTRESGVPDRFSGSGSGTDFTLTINRLQAEDVAVYYCQHYYSYPLTFGQGTKAAAYPYDVPDYANPDFPPEVEEQDASTLAVSCAWESGMKRHRAACASASINV

SEQ ID NO: 43 - protein, 300 aa, Artificial Sequence
  Synthetic polypeptide
MQVQLVESGGGLVKPGGSLRLSCAASGFTFSSYAMSWVRQAPGKGLEWVAAISGSGDNTYYADSVKGRFTISRDNSENTVHLQMAGLRAEDTALYFCAKDGPAVGNPGGYFDFWGRGTLVTVSSGGGGSGGGGSGGGGSDIVMTQSPDSLAVSLGERATINCKSSQSLLYSSNNKDYLAWYQQKPGQSPRLLIPWASTRESGVPDRFSGSGSGTDFTLTINRLQAEDVAVYYCQHYYSYPLTFGQGTKAAAYPYDVPDYANPDFPPEVEEQDAATLPVSCAWESGMKRHRAACASASINV

SEQ ID NO: 44 - protein, 300 aa, Artificial Sequence
  Synthetic polypeptide
MQVQLVESGGGLVKPGGSLRLSCAASGFTFSSYAMSWVRQAPGKGLEWVAAISGSGDNTYYADSVKGRFTISRDNSENTVHLQMAGLRAEDTALYFCAKDGPAVGNPGGYFDFWGRGTLVTVSSGGGGSGGGGSGGGGSDIVMTQSPDSLAVSLGERATINCKSSQSLLYSSNNKDYLAWYQQKPGQSPRLLIPWASTRESGVPDRFSGSGSGTDFTLTINRLQAEDVAVYYCQHYYSYPLTFGQGTKAAAYPYDVPDYANPDFPPEVEEQDASTLPVACAWESGMKRHRAACASASINV

SEQ ID NO: 45 - protein, 299 aa, Artificial Sequence
  Synthetic polypeptide
QVQLVESGGGLVKPGGSLRLSCAASGFTFSSYAMSWVRQAPGKGLEWVAAISGSGDNTYYADSVKGRFTISRDNSENTVHLQMAGLRAEDTALYFCAKDGPAVGNPGGYFDFWGRGTLVTVSSGGGGSGGGGSGGGGSDIVMTQSPDSLAVSLGERATINCKSSQSLLYSSNNKDYLAWYQQKPGQSPRLLIPWASTRESGVPDRFSGSGSGTDFTLTINRLQAEDVAVYYCQHYYSYPLTFGQGTKAAAYPYDVPDYANPDFPPEVEEQDASTLPVSCAWEAGMKRHRAACASASINV

SEQ ID NO: 46 - protein, 299 aa, Artificial Sequence
  Synthetic polypeptide
QVQLVESGGGLVKPGGSLRLSCAASGFTFSSYAMSWVRQAPGKGLEWVAAISGSGDNTYYADSVKGRFTISRDNSENTVHLQMAGLRAEDTALYFCAKDGPAVGNPGGYFDFWGRGTLVTVSSGGGGSGGGGSGGGGSDIVMTQSPDSLAVSLGERATINCKSSQSLLYSSNNKDYLAWYQQKPGQSPRLLIPWASTRESGVPDRFSGSGSGTDFTLTINRLQAEDVAVYYCQHYYSYPLTFGQGTKAAAYPYDVPDYAACCGEHPIRPPVEDFEESRASSTASAWLANMVQNKPVSDA

SEQ ID NO: 47 - protein, 296 aa, Artificial Sequence
  Synthetic polypeptide
MQVQLVESGGGLVKPGGSLRLSCAASGFTFSDYYMSWIRQAPGKGLEWVSSISSSSSYIYYADSVKGRFTISRDNAKNSLYLQMNSLRAEDTAVYYCARDGIAARSGYYGMDVWGQGTLVTVSSGGGGSGGGGSGGGGSEIVLTQSPSTLSASVGDRVTITCRASQSISSWLAWYQQKPGKAPKLLIYAASILQSGVPSRFSGSGSGTDFTLTISSLQPEDFATYYCLQDSNPYPLLTFGGGTKAAAYPYDVPDYANPDFPPEVEEQDASTLPVSCAWESGMKRHRAACASASINV

SEQ ID NO: 48 - protein, 296 aa, Artificial Sequence
  Synthetic polypeptide
MQVQLVESGGGLVKPGGSLRLSCAASGFTFSDYYMSWIRQAPGKGLEWVSSISSSSSYIYYADSVKGRFTISRDNAKNSLYLQMNSLRAEDTAVYYCARDGIAARSGYYGMDVWGQGTLVTVSSGGGGSGGGGSGGGGSEIVLTQSPSTLSASVGDRVTITCRASQSISSWLAWYQQKPGKAPKLLIYAASILQSGVPSRFSGSGSGTDFTLTISSLQPEDFATYYCLQDSNPYPLLTFGGGTKAAAYPYDVPDYANPDFPPEVEEQDASTLPVSAAWESGMKRHRAACASASINV

SEQ ID NO: 49 - protein, 296 aa, Artificial Sequence
  Synthetic polypeptide
MQVQLVESGGGLVKPGGSLRLSCAASGFTFSDYYMSWIRQAPGKGLEWVSSISSSSSYIYYADSVKGRFTISRDNAKNSLYLQMNSLRAEDTAVYYCARDGIAARSGYYGMDVWGQGTLVTVSSGGGGSGGGGSGGGGSEIVLTQSPSTLSASVGDRVTITCRASQSISSWLAWYQQKPGKAPKLLIYAASILQSGVPSRFSGSGSGTDFTLTISSLQPEDFATYYCLQDSNPYPLLTFGGGTKAAAYPYDVPDYANPDFPPEVEEQAASTLPVSCAWESGMKRHRAACASASINV

SEQ ID NO: 50 - protein, 296 aa, Artificial Sequence
  Synthetic polypeptide
MQVQLVESGGGLVKPGGSLRLSCAASGFTFSDYYMSWIRQAPGKGLEWVSSISSSSSYIYYADSVKGRFTISRDNAKNSLYLQMNSLRAEDTAVYYCARDGIAARSGYYGMDVWGQGTLVTVSSGGGGSGGGGSGGGGSEIVLTQSPSTLSASVGDRVTITCRASQSISSWLAWYQQKPGKAPKLLIYAASILQSGVPSRFSGSGSGTDFTLTISSLQPEDFATYYCLQDSNPYPLLTFGGGTKAAAYPYDVPDYANPDFPPAVAAQDASTLPVSCAWESGMKRHRAACASASINV

SEQ ID NO: 51 - protein, 296 aa, Artificial Sequence
  Synthetic polypeptide
MQVQLVESGGGLVKPGGSLRLSCAASGFTFSDYYMSWIRQAPGKGLEWVSSISSSSSYIYYADSVKGRFTISRDNAKNSLYLQMNSLRAEDTAVYYCARDGIAARSGYYGMDVWGQGTLVTVSSGGGGSGGGGSGGGGSEIVLTQSPSTLSASVGDRVTITCRASQSISSWLAWYQQKPGKAPKLLIYAASILQSGVPSRFSGSGSGTDFTLTISSLQPEDFATYYCLQDSNPYPLLTFGGGTKAAAYPYDVPDYANPDFPPEVEEQDASTLPVSCAWASGMKRHRAACASASINV

SEQ ID NO: 52 - protein, 296 aa, Artificial Sequence
  Synthetic polypeptide
MQVQLVESGGGLVKPGGSLRLSCAASGFTFSDYYMSWIRQAPGKGLEWVSSISSSSSYIYYADSVKGRFTISRDNAKNSLYLQMNSLRAEDTAVYYCARDGIAARSGYYGMDVWGQGTLVTVSSGGGGSGGGGSGGGGSEIVLTQSPSTLSASVGDRVTITCRASQSISSWLAWYQQKPGKAPKLLIYAASILQSGVPSRFSGSGSGTDFTLTISSLQPEDFATYYCLQDSNPYPLLTFGGGTKAAAYPYDVPDYANPDFPPEVEEQDASTLPVSCAWESGMARHRAACASASINV

SEQ ID NO: 53 - protein, 296 aa, Artificial Sequence
  Synthetic polypeptide
MQVQLVESGGGLVKPGGSLRLSCAASGFTFSDYYMSWIRQAPGKGLEWVSSISSSSSYIYYADSVKGRFTISRDNAKNSLYLQMNSLRAEDTAVYYCARDGIAARSGYYGMDVWGQGTLVTVSSGGGGSGGGGSGGGGSEIVLTQSPSTLSASVGDRVTITCRASQSISSWLAWYQQKPGKAPKLLIYAASILQSGVPSRFSGSGSGTDFTLTISSLQPEDFATYYCLQDSNPYPLLTFGGGTKAAAYPYDVPDYANPDFAAEVEEQDASTLPVSCAWESGMKRHRAACASASINV

SEQ ID NO: 54 - protein, 296 aa, Artificial Sequence
  Synthetic polypeptide
MQVQLVESGGGLVKPGGSLRLSCAASGFTFSDYYMSWIRQAPGKGLEWVSSISSSSSYIYYADSVKGRFTISRDNAKNSLYLQMNSLRAEDTAVYYCARDGIAARSGYYGMDVWGQGTLVTVSSGGGGSGGGGSGGGGSEIVLTQSPSTLSASVGDRVTITCRASQSISSWLAWYQQKPGKAPKLLIYAASILQSGVPSRFSGSGSGTDFTLTISSLQPEDFATYYCLQDSNPYPLLTFGGGTKAAAYPYDVPDYANPDFPPEVEEQDASTLAVSCAWESGMKRHRAACASASINV

SEQ ID NO: 55 - protein, 296 aa, Artificial Sequence
  Synthetic polypeptide
MQVQLVESGGGLVKPGGSLRLSCAASGFTFSDYYMSWIRQAPGKGLEWVSSISSSSSYIYYADSVKGRFTISRDNAKNSLYLQMNSLRAEDTAVYYCARDGIAARSGYYGMDVWGQGTLVTVSSGGGGSGGGGSGGGGSEIVLTQSPSTLSASVGDRVTITCRASQSISSWLAWYQQKPGKAPKLLIYAASILQSGVPSRFSGSGSGTDFTLTISSLQPEDFATYYCLQDSNPYPLLTFGGGTKAAAYPYDVPDYANPDFPPEVEEQDAATLPVSCAWESGMKRHRAACASASINV

SEQ ID NO: 56 - protein, 296 aa, Artificial Sequence
  Synthetic polypeptide
MQVQLVESGGGLVKPGGSLRLSCAASGFTFSDYYMSWIRQAPGKGLEWVSSISSSSSYIYYADSVKGRFTISRDNAKNSLYLQMNSLRAEDTAVYYCARDGIAARSGYYGMDVWGQGTLVTVSSGGGGSGGGGSGGGGSEIVLTQSPSTLSASVGDRVTITCRASQSISSWLAWYQQKPGKAPKLLIYAASILQSGVPSRFSGSGSGTDFTLTISSLQPEDFATYYCLQDSNPYPLLTFGGGTKAAAYPYDVPDYANPDFPPEVEEQDASTLPVACAWESGMKRHRAACASASINV

SEQ ID NO: 57 - protein, 296 aa, Artificial Sequence
  Synthetic polypeptide
MQVQLVESGGGLVKPGGSLRLSCAASGFTFSDYYMSWIRQAPGKGLEWVSSISSSSSYIYYADSVKGRFTISRDNAKNSLYLQMNSLRAEDTAVYYCARDGIAARSGYYGMDVWGQGTLVTVSSGGGGSGGGGSGGGGSEIVLTQSPSTLSASVGDRVTITCRASQSISSWLAWYQQKPGKAPKLLIYAASILQSGVPSRFSGSGSGTDFTLTISSLQPEDFATYYCLQDSNPYPLLTFGGGTKAAAYPYDVPDYANPDFPPEVEEQDASTLPVSCAWEAGMKRHRAACASASINV

SEQ ID NO: 58 - protein, 288 aa, Artificial Sequence
  Synthetic polypeptide
MQVQLQESGGGLVQPGRSLRLSCAASGFTFDDYAMHWVRQAPGKGLEWVAVISYDGSNKYYADSVKGRFTISRDNSKNTLYLQMNSLRAEDTAVYYCARDFAGAIAYWGQGTLVTVSSGGGGSGGGGSGGGGSEIVLTQSPSFLSASVGDRVTITCRASHGINNYLAWYQQKPGKAPKLLIYAASSLQSGVPSRFSGSGSGTDFTLTISSLQPEDFATYYCQQANSFPLTFGGGTKAAAYPYDVPDYANPDFPPEVEEQDASTLPVSCAWESGMKRHRAACASASINV

SEQ ID NO: 59 - protein, 288 aa, Artificial Sequence
  Synthetic polypeptide
MQVQLQESGGGLVQPGRSLRLSCAASGFTFDDYAMHWVRQAPGKGLEWVAVISYDGSNKYYADSVKGRFTISRDNSKNTLYLQMNSLRAEDTAVYYCARDFAGAIAYWGQGTLVTVSSGGGGSGGGGSGGGGSEIVLTQSPSFLSASVGDRVTITCRASHGINNYLAWYQQKPGKAPKLLIYAASSLQSGVPSRFSGSGSGTDFTLTISSLQPEDFATYYCQQANSFPLTFGGGTKAAAYPYDVPDYANPDFAAEVEEQDASTLPVSCAWESGMKRHRAACASASINV

SEQ ID NO: 60 - protein, 288 aa, Artificial Sequence
  Synthetic polypeptide
MQVQLQESGGGLVQPGRSLRLSCAASGFTFDDYAMHWVRQAPGKGLEWVAVISYDGSNKYYADSVKGRFTISRDNSKNTLYLQMNSLRAEDTAVYYCARDFAGAIAYWGQGTLVTVSSGGGGSGGGGSGGGGSEIVLTQSPSFLSASVGDRVTITCRASHGINNYLAWYQQKPGKAPKLLIYAASSLQSGVPSRFSGSGSGTDFTLTISSLQPEDFATYYCQQANSFPLTFGGGTKAAAYPYDVPDYANPDFPPEVEEQAASTLPVSCAWESGMKRHRAACASASINV

SEQ ID NO: 61 - protein, 288 aa, Artificial Sequence
  Synthetic polypeptide
MQVQLQESGGGLVQPGRSLRLSCAASGFTFDDYAMHWVRQAPGKGLEWVAVISYDGSNKYYADSVKGRFTISRDNSKNTLYLQMNSLRAEDTAVYYCARDFAGAIAYWGQGTLVTVSSGGGGSGGGGSGGGGSEIVLTQSPSFLSASVGDRVTITCRASHGINNYLAWYQQKPGKAPKLLIYAASSLQSGVPSRFSGSGSGTDFTLTISSLQPEDFATYYCQQANSFPLTFGGGTKAAAYPYDVPDYANPDFPPEVEEQDASTLPVSAAWESGMKRHRAACASASINV

SEQ ID NO: 62 - protein, 288 aa, Artificial Sequence
  Synthetic polypeptide
MQVQLQESGGGLVQPGRSLRLSCAASGFTFDDYAMHWVRQAPGKGLEWVAVISYDGSNKYYADSVKGRFTISRDNSKNTLYLQMNSLRAEDTAVYYCARDFAGAIAYWGQGTLVTVSSGGGGSGGGGSGGGGSEIVLTQSPSFLSASVGDRVTITCRASHGINNYLAWYQQKPGKAPKLLIYAASSLQSGVPSRFSGSGSGTDFTLTISSLQPEDFATYYCQQANSFPLTFGGGTKAAAYPYDVPDYANPDFPPEVEEQDASTLPVSCAWEAGMKRHRAACASASINV

SEQ ID NO: 63 - protein, 288 aa, Artificial Sequence
  Synthetic polypeptide
MQVQLQESGGGLVQPGRSLRLSCAASGFTFDDYAMHWVRQAPGKGLEWVAVISYDGSNKYYADSVKGRFTISRDNSKNTLYLQMNSLRAEDTAVYYCARDFAGAIAYWGQGTLVTVSSGGGGSGGGGSGGGGSEIVLTQSPSFLSASVGDRVTITCRASHGINNYLAWYQQKPGKAPKLLIYAASSLQSGVPSRFSGSGSGTDFTLTISSLQPEDFATYYCQQANSFPLTFGGGTKAAAYPYDVPDYAACCGEHPIRPPVEDFEESRASSTASAWLANMVQNKPVSDA

SEQ ID NO: 64 - protein, 117 aa, Artificial Sequence
  Synthetic polypeptide
QVQLQESGGGLVQPGRSLRLSCAASGFTFDDYAMHWVRQAPGKGLEWVAVISYDGSNKYYADSVKGRFTISRDNSKNTLYLQMNSLRAEDTAVYYCARDFAGAIAYWGQGTLVTVSS

SEQ ID NO: 65 - protein, 117 aa, Artificial Sequence
  Synthetic polypeptide
QVQLQQSGGGVVQPGRSLRLSCAASGFTFSSYGMHWVRQAPGKGLEWVAVISYDGSNKYYADSVKGRFTISRDNSKNTLYLQMNSLRAEDTAVYYCAKDLVGAKGNWGQGTLVTVSS

SEQ ID NO: 66 - protein, 117 aa, Artificial Sequence; identical to SEQ ID NO: 64
  Synthetic polypeptide

SEQ ID NO: 67 - protein, 123 aa, Artificial Sequence
  Synthetic polypeptide
QVQLVESGGGLVKPGGSLRLSCAASGFTFSSYAMSWVRQAPGKGLEWVAAISGSGDNTYYADSVKGRFTISRDNSENTVHLQMAGLRAEDTALYFCAKDGPAVGNPQGYFDFWGRGTLVTVSS

SEQ ID NO: 68 - protein, 117 aa, Artificial Sequence
  Synthetic polypeptide
QVQLVQSGGGVVQPGRSLRLSCAASGFTFSSYGMHWVRQAPGKGLEWVASMSYDGNNKYYADSVKGRFTISRDNSKNTLYLQMNSLRAEDTAVYYCARDLRGALDYWGQGTLVTVSS

SEQ ID NO: 69 - protein, 123 aa, Artificial Sequence
  Synthetic polypeptide
QVQLVESGGGLVKPGGSLRLSCAASGFTFSDYYMSWIRQAPGKGLEWVSSISSSSSYIYYADSVKGRFTISRDNAKNSLYLQMNSLRAEDTAVYYCARDGIAARSGYYGMDVWGQGTLVTVSS

SEQ ID NO: 70 - protein, 117 aa, Artificial Sequence; identical to SEQ ID NO: 64
  Synthetic polypeptide

SEQ ID NO: 71 - protein, 123 aa, Artificial Sequence; identical to SEQ ID NO: 67
  Synthetic polypeptide

SEQ ID NO: 72 - protein, 117 aa, Artificial Sequence
  Synthetic polypeptide
QVQLVQSGGGVVQPGRSLRLSCAASGFTFSSYGMHWVRQAPGKGLEWVAVISYDGSNKYYADSVKGRFTISRDNSKNTLYLQMNSLRAEDTAVYYCAKDLPDSNGYWGQGTLVTVSS

SEQ ID NO: 73 - protein, 123 aa, Artificial Sequence
  Synthetic polypeptide
QVQLVESGGGLVKPGGSLRLSCAASGFTFSSYAMSWVRQAPGKGLEWVAAISGSGDNTYYADSVKGRFTISRDNSENTVHLQMAGLRAEDTALYFCAKDGPAVGNPGGYFDFWGRGTLVTVSS

SEQ ID NO: 74 - protein, 117 aa, Artificial Sequence
  Synthetic polypeptide
QVQLVQSGGGVVHPGRSLRLSCAASGFTFSSYGMHWVRQAPGKGLEWVASMSYDGNNKYYADSVKGRFTTPRDNSKNTLYLQMNSLRAEDTAVYYCARDLRGALDYWGQGTLVTVSS

SEQ ID NO: 75 - protein, 117 aa, Artificial Sequence; identical to SEQ ID NO: 64
  Synthetic polypeptide

SEQ ID NO: 76 - protein, 117 aa, Artificial Sequence
  Synthetic polypeptide
QVQLQQSGGGVVQPGRSLRLSCAASGFTFSSYGMHWARQAPGKGLEWVAVISYDGSNKYYADSVKGRFTISRDNSKNTLYLQMNSLRAEDTAVYYCAKDLVGAKGNWAQGTLVTVSS

SEQ ID NO: 77 - protein, 117 aa, Artificial Sequence; identical to SEQ ID NO: 65
  Synthetic polypeptide

SEQ ID NO: 78 - protein, 123 aa, Artificial Sequence
  Synthetic polypeptide
QVQLVESGGGLVKPGGSLRLSCAASGFTFSSYAMSWVRQAPGKGLEWVAAISGSGDNTYYADSVKGRFTISRDNSENTVHLQMAGLRAEDTALYFCAKDGPEVGNPGGYFDFWGRGTLVTVSS

SEQ ID NO: 79 - protein, 117 aa, Artificial Sequence
  Synthetic polypeptide
QVQLQQSGEGVVQPGRSLRLSCAASGFTFSSYGMHWVRQAPGKGLEWVAVISYDGSNKYYADSVKGRFTISRDNSKNTLYLQMNSLRAEDTAVYYCAKDLVGAKGNWGQGTLVTVSS

SEQ ID NO: 80 - protein, 117 aa, Artificial Sequence
  Synthetic polypeptide
QVQLVQSGGGVVQPGRSLRLSCAASGFTFSSYGMHWVRQAPGKGLEWVASMSYDGDNKYYADSVKGRFTISRDNSKNTLYLQMNSLRAEDTAVYYCARDLRGALDYWGQGTLVTVSS

SEQ ID NO: 81 - protein, 103 aa, Artificial Sequence
  Synthetic polypeptide
EIVLTQSPSFLSASVGDRVTITCRASHGINNYLAWYQQKPGKAPKLLIYAASSLQSGVPSRFSGSGSGTDFTLTISSLQPEDFATYYCQQANSFPLTFGGGTK

SEQ ID NO: 82 - protein, 103 aa, Artificial Sequence
  Synthetic polypeptide
EIVLTQSPSTLSASVGERVTITCRASQSISSWLAWYQQKPGKAPKVLIYKASSLESGVPSRFSGSGSGTEFTLTISSLQPDDFATYYCQQYSTYLWTFGQGTK

SEQ ID NO: 83 - protein, 103 aa, Artificial Sequence
  Synthetic polypeptide
EIVLTQSPSILSASVGDRVTITCRASHGINNYLAWYQQKPGKAPKLLIYAASSLQSGVPSRFSGSGSGTDFTLTISSLQPEDFATYYCQQANSFPWTFGQGTK

SEQ ID NO: 84 - protein, 109 aa, Artificial Sequence
  Synthetic polypeptide
DIVMTQSPDSLAVSLGERATINCKSSQSLLYSSNNKDYLAWYQQKPGQSPRLLISWASTRESGVPDRFSGSGSGTDFTLTINRLQAEDVAVYYCQHYYSYPLTFGQGTK

SEQ ID NO: 85 - protein, 103 aa, Artificial Sequence
  Synthetic polypeptide
EIVLTQSPSTLSASIGDRVTITCRASQGISNYLAWYQQKPGKAPKLLIYAASTLQSGVPSRFSGSGSGTEFTLTISGLLPEDFASYFCQQASVFPVTFGGGTK

SEQ ID NO: 86 - protein, 105 aa, Artificial Sequence
  Synthetic polypeptide
EIVLTQSPSTLSASVGDRVTITCRASQSISSWLAWYQQKPGKAPKLLIYAASILQSGVPSRFSGSGSGTDFTLTISSLQPEDFATYYCLQDSNPYPLLTFGGGTK

SEQ ID NO: 87 - protein, 102 aa, Artificial Sequence
  Synthetic polypeptide
EIVLTQSPSFLSASVGDRVTITCRASHGINNYLAWYQQKPGKAPKLLIYAASSLQSGVPSRFSGSGSGTDFTLTISSLQPEDFATYYCQQANSFPLTFARTK

SEQ ID NO: 88 - protein, 109 aa, Artificial Sequence
  Synthetic polypeptide
DIVMTQSPDSLAVSLGERATINCKSSQSLLYSSNNKDYLAWYQQKPGQSPRLLISWASTRESGVPSRFSGSGSGTDFTLTINRLQAEDVAVYYCQHYYSYPLTFGQGTK

SEQ ID NO: 89 - protein, 103 aa, Artificial Sequence
  Synthetic polypeptide
DVVMTQSPSTLSASVGDRVTITCRASENINRWLAWYQQKPGKAPKLLIYKASSLESGVPSRCSGSGSGTEFTLTISSLQPDDFATYYCHQYTTYLWTFGQGTK

SEQ ID NO: 90 - protein, 109 aa, Artificial Sequence
  Synthetic polypeptide
DIVMTQSPDSLAVSLGERATINCKSSQSLLYSSNNKDYLAWYQQKPGQSPRLLIPWASTRESGVPDRFSGSGSGTDFTLTINRLQAEDVAVYYCQHYYSYPLTFGQGTK

SEQ ID NO: 91 - protein, 102 aa, Artificial Sequence
  Synthetic polypeptide
EIVLTQSPSTLSASIGDRVTITCRASQGISNYLAWYQQKPGKAPKLLIYAASTLQSGVPSRFSGSGSGTEFTLTISGLLPEDFASYFCQQASVFPVTFARTK

SEQ ID NO: 92 - protein, 103 aa, Artificial Sequence
  Synthetic polypeptide
EICVTQSPSFLSASVGDRVTITCRASHGINNYLAWYQQKPGKAPKLLIYAASSLQSGVPSRFSGSGSGTDFTLTISSLQPEDFATYYCQQANSFPLTFGGGTK

SEQ ID NO: 93 - protein, 103 aa, Artificial Sequence; identical to SEQ ID NO: 82
  Synthetic polypeptide

SEQ ID NO: 94 - protein, 103 aa, Artificial Sequence
  Synthetic polypeptide
EIVLTQSPSTLSASVGERVTITCRASQSISSWLAWYQQKPGKAPKVLIYKASSLESGVPDRFSGSGSGTEFTLTISSLQPDDFATYYCQQYSTYLWTFGQGTK

SEQ ID NO: 95 - protein, 109 aa, Artificial Sequence
  Synthetic polypeptide
DIVMTKSPDSLAVSLGERATINCKSSQSLLYSSKNKDYLAWYQKKPGQSPRLLISWASTRESGVPDRFSGSGSGTDFTLTINRLQAEDVAVYYCQHYYSYPLTFGQGTK

SEQ ID NO: 96 - protein, 103 aa, Artificial Sequence
  Synthetic polypeptide
EIVLTQSPSTLSASVGERVTITCRASQSISSWLAWYQQKPGKAPKVLIYKASSLESGVPSFRSGSGSGTEFTLTISSLQPDDFATYYCQQYSTYLWTFGQGTK

SEQ ID NO: 97 - protein, 103 aa, Artificial Sequence
  Synthetic polypeptide
EIVLTQSPSTLSASIGDRVTITCRASQGISNYLAWYQQKPGKAPKLLIYAASTLQSGVPSRFSGSGSGTEFTLTISGLLPEDFASYFCLQASVFPVTFGGGTK

SEQ ID NO: 98 - protein, 3144 aa, Homo sapiens
MATLEKLMKAFESLKSFQQQQQQQQQQQQQQQQQQQQQQQPPPPPPPPPPPQLPQPPPQAQPLLPQPQPPPPPPPPPPGPAVAEEPLHRPKKELSATKKDRVNHCLTICENIVAQSVRNSPEFQKLLGIAMELFLLCSDDAESDVRMVADECLNKVIKALMDSNLPRLQLELYKEIKKNGAPRSLRAALWRFAELAHLVRPQKCRPYLVNLLPCLTRTSKRPEESVQETLAAAVPKIMASFGNFANDNEIKVLLKAFIANLKSSSPTIRRTAAGSAVSICQHSRRTQYFYSWLLNVLLGLLVPVEDEHSTLLILGVLLTLRYLVPLLQQQVKDTSLKGSFGVTRKEMEVSPSAEQLVQVYELTLHHTQHQDHNVVTGALELLQQLFRTPPPELLQTLTAVGGIGQLTAAKEESGGRSRSGSIVELIAGGGSSCSPVLSRKQKGKVLLGEEEALEDDSESRSDVSSSALTASVKDEISGELAASSGVSTPGSAGHDIITEQPRSQHTLQADSVDLASCDLTSSATDGDEEDILSHSSSQVSAVPSDPAMDLNDGTQASSPISDSSQTTTEGPDSAVTPSDSSEIVLDGTDNQYLGLQIGQPQDEDEEATGILPDEASEAFRNSSMALQQAHLLKNMSHCRQPSDSSVDKFVLRDEATEPGDQENKPCRIKGDIGQSTDDDSAPLVHCVRLLSASFLLTGGKNVLVPDRDVRVSVKALALSCVGAAVALHPESFFSKLYKVPLDTTEYPEEQYVSDILNYIDHGDPQVRGATAILCGTLICSILSRSRFHVGDWMGTIRTLTGNTFSLADCIPLLRKTLKDESSVTCKLACTAVRNCVMSLCSSSYSELGLQLIIDVLTLRNSSYWLVRTELLETLAEIDFRLVSFLEAKAENLHRGAHHYTGLLKLQERVLNNVVIHLLGDEDPRVRHVAAASLIRLVPKLFYKCDQGQADPVVAVARDQSSVYLKLLMHETQPPSHFSVSTITRIYRGYNLLPSITDVTMENNLSRVIAAVSHELITSTTRALTFGCCEALCLLSTAFPVCIWSLGWHCGVPPLSASDESRKSCTVGMATMILTLLSSAWFPLDLSAHQDALILAGNLLAASAPKSLRSSWASEEEANPAATKQEEVWPALGDRALVPMVEQLFSHLLKVINICAHVLDDVAPGPAIKAALPSLTNPPSLSPIRRKGKEKEPGEQASVPLSPKKGSEASAASRQSDTSGPVTTSKSSSLGSFYHLPSYLKLHDVLKATHANYKVTLDLQNSTEKFGGFLRSALDVLSQILELATLQDIGKCVEEILGYLKSCFSREPMMATVCVQQLLKTLFGTNLASQFDGLSSNPSKSQGRAQRLGSSSVRPGLYHYCFMAPYTHFTQALADASLRNMVQAEQENDTSGWFDVLQKVSTQLKTNLTSVTKNRADKNAIHNHIRLFEPLVIKALKQYTTTTCVQLQKQVLDLLAQLVQLRVNYCLLDSDQVFIGFVLKQFEYIEVGQFRESEAIIPNIFFFLVLLSYERYHSKQIIGIPKIIQLCDGIMASGRKAVTHAIPALQPIVHDLFVLRGTNKADAGKELETQKEVVVSMLLRLIQYHQVLEMFILVLQQCHKENEDKWKRLSRQIADIILPMLAKQQMHIDSHEALGVLNTLFEILAPSSLRPVDMLLRSMFVTPNTMASVSTVQLWISGILAILRVLISQSTEDIVLSRIQELSFSPYLISCTVINRLRDGDSTSTLEEHSEGKQIKNLPEETFSRFLLQLVGILLEDIVTKQLKVEMSEQQHTFYCQELGTLLMCLIHIFKSGMFRRITAAATRLFRSDGCGGSFYTLDSLNLRARSMITTHPALVLLWCQILLLVNHTDYRWWAEVQQTPKRHSLSSTKLLSPQMSGEEEDSDLAAKLGMCNREIVRRGALILFCDYVCQNLHDSEHLTWLIVNHIQDLISLSHEPPVQDFISAVHRNSAASGLFIQAIQSRCENLSTPTMLKKTLQCLEGIHLSQSGAVLTLYVDRLLCTPFRVLARMVDILACRRVEMLLAANLQSSMAQLPMEELNRIQEYLQSSGLAQRHQRLYSLLDRFRLSTMQDSLSPSPPVSSHPLDGDGHVSLETVSPDKDWYVHLVKSQCWTRSDSALLEGAELVNRIPAEDMNAFMMNSEFNLSLLAPCLSLGMSEISGGQKSALFEAAREVTLARVSGTVQQLPAVHHVFQPELPAEPAAYWSKLNDLFGDAALYQSLPTLARALAQYLVVVSKLPSHLHLPPEKEKDIVKFVVATLEALSWHLIHEQIPLSLDLQAGLDCCCLALQLPGLWSVVSSTEFVTHACSLIYCVHFILEAVAVQPGEQLLSPERRTNTPKAISEEEEEVDPNTQNPKYITAACEMVAEMVESLQSVLALGHKRNSGVPAFLTPLLRNIIISLARLPLVNSYTRVPPLVWKLGWSPKPGGDFGTAFPEIPVEFLQEKEVFKEFIYRINTLGWTSRTQFEETWATLLGVLVTQPLVMEQEESPPEEDTERTQINVLAVQAITSLVLSAMTVPVAGNPAVSCLEQQPRNKPLKALDTRFGRKLSIIRGIVEQEIQAMVSKRENIATHHLYQAWDPVPSLSPATTGALISHEKLLLQINPERELGSMSYKLGQVSIHSVWLGNSITPLREEEWDEEEEEEADAPAPSSPPTSPVNSRKHRAGVDIHSCSQFLLELYSRWILPSSSARRTPAILISEVVRSLLVVSDLFTERNQFELMYVTLTELRRVHPSEDEILAQYLVPATCKAAAVLGMDKAVAEPVSRLLESTLRSSHLPSRVGALHGVLYVLECDLLDDTAKQLIPVISDYLLSNLKGIAHCVNIHSQQHVLVMCATAFYLIENYPLDVGPEFSASIIQMCGVMLSGSEESTPSIIYHCALRGLERLLLSEQLSRLDAESLVKLSVDRVNVHSPHRAMAALGLMLTCMYTGKEKVSPGRTSDPNPAAPDSESVIVAMERVSVLFDRIRKGFPCEARVVARILPQFLDDFFPPQDIMNKVIGEFLSNQQPYPQFMATVVYKVFQTLHSTGQSSMVRDWVMLSLSNFTQRAPVAMATWSLSCFFVSASTSPWVAAILPHVISRMGKLEQVDVNLFCLVATDFYRHQIEEELDRRAFQSVLEVVAAPGSPYHRLLTCLRNVHKVTTC

SEQ ID NO: 99 - protein, 294 aa, Artificial Sequence
  Synthetic polypeptide
MAQVQLQESGGGLVQPGGSLRLSCAASGFTFSSYSMSWVRQAPGKGLEWVAVISYDGSNKYYADSVKGRFTISRDNSKNTLYLQMNSLRAEDTAVYYCARDRYFDLWGRGTLVTVSSGGGGSGGGGSGGGGSQSALTQPASVSGSPGQSITISCTGTSSDIGAYNYVSWYQQYPGKAPKLLIYDVSNRPSGISNRFSGSKSGDTASLTISGLQAEDEADYYCSSFANSGPLFGGGTKVTVLGAAAYPYDVPDYANPDFPPEVEEQDASTLPVSCAWESGMKRHRAACASASINV

SEQ ID NO: 100 - protein, 294 aa, Artificial Sequence
  Synthetic polypeptide
MAQVQLQESGGGLVQPGGSLRLSCAASGFTFSSYSMSWVRQAPGKGLEWVAVISYDGSNKYYADSVKGRFTISRDNSKNTLYLQMNSLRAEDTAVYYCARDRYFDLWGRGTLVTVSSGGGGSGGGGSGGGGSQSALTQPASVSGSPGQSITISCTGTSSDIGAYNYVSWYQQYPGKAPKLLIYDVSNRPSGISNRFSGSKSGDTASLTISGLQAEDEADYYCSSFANSGPLFGGGTKVTVLGAAAYPYDVPDYANPDFPPEVEEQDASTLPVACAWESGMKRHRAACASASINV

SEQ ID NO: 101 - protein, 294 aa, Artificial Sequence
  Synthetic polypeptide
MAQVQLQESGGGLVQPGGSLRLSCAASGFTFSSYSMSWVRQAPGKGLEWVAVISYDGSNKYYADSVKGRFTISRDNSKNTLYLQMNSLRAEDTAVYYCARDRYFDLWGRGTLVTVSSGGGGSGGGGSGGGGSQSALTQPASVSGSPGQSITISCTGTSSDIGAYNYVSWYQQYPGKAPKLLIYDVSNRPSGISNRFSGSKSGDTASLTISGLQAEDEADYYCSSFANSGPLFGGGTKVTVLGAAAYPYDVPDYANPDFPPAVAAQDASTLPVSCAWESGMKRHRAACASASINV

SEQ ID NO: 102 - protein, 294 aa, Artificial Sequence
  Synthetic polypeptide
MAQVQLQESGGGLVQPGGSLRLSCAASGFTFSSYSMSWVRQAPGKGLEWVAVISYDGSNKYYADSVKGRFTISRDNSKNTLYLQMNSLRAEDTAVYYCARDRYFDLWGRGTLVTVSSGGGGSGGGGSGGGGSQSALTQPASVSGSPGQSITISCTGTSSDIGAYNYVSWYQQYPGKAPKLLIYDVSNRPSGISNRFSGSKSGDTASLTISGLQAEDEADYYCSSFANSGPLFGGGTKVTVLGAAAYPYDVPDYANPDFPPEVEEQDASTLPVSCAWASGMKRHRAACASASINV

SEQ ID NO: 103 - protein, 294 aa, Artificial Sequence
  Synthetic polypeptide
MAQVQLQESGGGLVQPGGSLRLSCAASGFTFSSYSMSWVRQAPGKGLEWVAVISYDGSNKYYADSVKGRFTISRDNSKNTLYLQMNSLRAEDTAVYYCARDRYFDLWGRGTLVTVSSGGGGSGGGGSGGGGSQSALTQPASVSGSPGQSITISCTGTSSDIGAYNYVSWYQQYPGKAPKLLIYDVSNRPSGISNRFSGSKSGDTASLTISGLQAEDEADYYCSSFANSGPLFGGGTKVTVLGAAAYPYDVPDYANPDFPPEVEEQDASTLAVSCAWESGMKRHRAACASASINV

SEQ ID NO: 104 - protein, 294 aa, Artificial Sequence
  Synthetic polypeptide
MAQVQLQESGGGLVQPGGSLRLSCAASGFTFSSYSMSWVRQAPGKGLEWVAVISYDGSNKYYADSVKGRFTISRDNSKNTLYLQMNSLRAEDTAVYYCARDRYFDLWGRGTLVTVSSGGGGSGGGGSGGGGSQSALTQPASVSGSPGQSITISCTGTSSDIGAYNYVSWYQQYPGKAPKLLIYDVSNRPSGISNRFSGSKSGDTASLTISGLQAEDEADYYCSSFANSGPLFGGGTKVTVLGAAAYPYDVPDYANPDFPPEVEEQDASALPVSCAWESGMKRHRAACASASINV

SEQ ID NO: 105 - protein, 111 aa, Artificial Sequence
  Synthetic polypeptide
QVQLQESGGGLVQPGGSLRLSCAASGFTFSSYSMSWVRQAPGKGLEWVAVISYDGSNKYYADSVKGRFTISRDNSKNTLYLQMNSLRAEDTAVYYCARDRYFDLWGRGTLV

SEQ ID NO: 106 - protein, 109 aa, Artificial Sequence
  Synthetic polypeptide
QSALTQPASVSGSPGQSITISCTGTSSDIGAYNYVSWYQQYPGKAPKLLIYDVSNRPSGISNRFSGSKSGDTASLTISGLQAEDEADYYCSSFANSGPLFGGGTKVTVL

SEQ ID NO: 107 - protein, 109 aa, Artificial Sequence
  Synthetic polypeptide
QPVLTQSPSVSAAPRQRVTISVSGSNSNIGSNTVNWIQQLPGRAPELLMYDDLLAPGVSDRFSGSRSGTSASLTISGLQSEDEADYYAATWDDSLNGWVFGGGTKVTVL

SEQ ID NO: 108 - protein, 40 aa, Artificial Sequence; identical to SEQ ID NO: 3
  Synthetic polypeptide
  SITE (1)..(40): /note=“Variant residues given in the sequence have no preference with respect to those in the annotations for variant positions”
  source (1)..(40): /note=“See specification as filed for detailed description of substitutions and preferred embodiments”
  VARIANT (5)..(5): /replace=“Ala”
  VARIANT (6)..(6): /replace=“Ala”
  VARIANT (7)..(7): /replace=“Ala”
  VARIANT (9)..(9): /replace=“Ala”
  VARIANT (10)..(10): /replace=“Ala”
  VARIANT (12)..(12): /replace=“Ala”
  VARIANT (14)..(14): /replace=“Ala”
  VARIANT (15)..(15): /replace=“Ala”
  VARIANT (17)..(17): /replace=“Ala”
  VARIANT (19)..(19): /replace=“Ala”
  VARIANT (20)..(20): /replace=“Ala”
  VARIANT (23)..(23): /replace=“Ala”
  VARIANT (24)..(24): /replace=“Ala”
  VARIANT (27)..(27): /replace=“Ala”

SEQ ID NO: 109 - protein, 40 aa, Artificial Sequence
  Synthetic polypeptide
NADFPPEVEEQDASTLPVSCAWESGMKRHRAACASASINV

SEQ ID NO: 110 - protein, 40 aa, Artificial Sequence
  Synthetic polypeptide
NPAFPPEVEEQDASTLPVSCAWESGMKRHRAACASASINV

SEQ ID NO: 111 - protein, 40 aa, Artificial Sequence
  Synthetic polypeptide
NPDFAPEVEEQDASTLPVSCAWESGMKRHRAACASASINV

SEQ ID NO: 112 - protein, 40 aa, Artificial Sequence
  Synthetic polypeptide
NPDFPAEVEEQDASTLPVSCAWESGMKRHRAACASASINV

SEQ ID NO: 113 - protein, 40 aa, Artificial Sequence
  Synthetic polypeptide
NPDFPPAVEEQDASTLPVSCAWESGMKRHRAACASASINV

SEQ ID NO: 114 - protein, 40 aa, Artificial Sequence
  Synthetic polypeptide
NPDFPPEVAEQDASTLPVSCAWESGMKRHRAACASASINV

SEQ ID NO: 115 - protein, 40 aa, Artificial Sequence
  Synthetic polypeptide
NPDFPPEVEAQDASTLPVSCAWESGMKRHRAACASASINV

SEQ ID NO: 116 - protein, 40 aa, Artificial Sequence
  Synthetic polypeptide
NPDFPPEVEEQAASTLPVSCAWESGMKRHRAACASASINV

SEQ ID NO: 117 - protein, 40 aa, Artificial Sequence
  Synthetic polypeptide
NPDFPPEVEEQDAATLPVSCAWESGMKRHRAACASASINV

SEQ ID NO: 118 - protein, 40 aa, Artificial Sequence
  Synthetic polypeptide
NPDFPPEVEEQDASALPVSCAWESGMKRHRAACASASINV

SEQ ID NO: 119 - protein, 40 aa, Artificial Sequence
  Synthetic polypeptide
NPDFPPEVEEQDASTLAVSCAWESGMKRHRAACASASINV

SEQ ID NO: 120 - protein, 40 aa, Artificial Sequence
  Synthetic polypeptide
NPDFPPEVEEQDASTLPVACAWESGMKRHRAACASASINV

SEQ ID NO: 121 - protein, 40 aa, Artificial Sequence
  Synthetic polypeptide
NPDFPPEVEEQDASTLPVSAAWESGMKRHRAACASASINV

SEQ ID NO: 122 - protein, 40 aa, Artificial Sequence
  Synthetic polypeptide
NPDFPPEVEEQDASTLPVSCAWASGMKRHRAACASASINV

SEQ ID NO: 123 - protein, 40 aa, Artificial Sequence
  Synthetic polypeptide
NPDFPPEVEEQDASTLPVSCAWEAGMKRHRAACASASINV

SEQ ID NO: 124 - protein, 40 aa, Artificial Sequence
  Synthetic polypeptide
NPDFPPEVEEQDASTAPVSCAWESGMKRHRAACASASINV

SEQ ID NO: 125 - protein, 40 aa, Artificial Sequence
  Synthetic polypeptide
NPDFPPEVEEQDASTLPVSCAWESGMARHRAACASASINV

SEQ ID NO: 126 - protein, 40 aa, Artificial Sequence
  Synthetic polypeptide
NPDFPPEVEEQDASTLPVSCAWESGMKAHRAACASASINV

SEQ ID NO: 127 - protein, 40 aa, Artificial Sequence
  Synthetic polypeptide
NPDFPPEVEEQDASTLPVSCAWESGMKRARAACASASINV

SEQ ID NO: 128 - protein, 40 aa, Artificial Sequence
  Synthetic polypeptide
ACCGEHPIRPPVEDFEESRASSTASAWLANMVQNKPVSDA

SEQ ID NO: 129 - protein, 40 aa, Artificial Sequence
  Synthetic polypeptide
NPDFAAEVEEQDASTLPVSCAWESGMKRHRAACASASINV

SEQ ID NO: 130 - protein, 40 aa, Artificial Sequence
  Synthetic polypeptide
NPDFPPAVAAQDASTLPVSCAWESGMKRHRAACASASINV

SEQ ID NO: 131 - protein, 40 aa, Artificial Sequence
  Synthetic polypeptide
NPDFPPEVEEQDASTLPVSCAWESGMAAARAACASASINV

SEQ ID NO: 132 - protein, 20 aa, Artificial Sequence
  Synthetic peptide
GGGGSGGGGSGGGGSGGGGS

SEQ ID NO: 133 - protein, 6 aa, Artificial Sequence
  Synthetic 6xHis tag
HHHHHH

SEQ ID NO: 134 - DNA, 117 bp, Artificial Sequence
  Synthetic polynucleotide
  misc_feature (1)..(117): /note=“This sequence may encompass 36-39 ′cag′ repeating units”
cagcagcagcagcagcagcagcagcagcagcagcagcagcagcagcagcagcagcagcagcagcagcagcagcagcagcagcagcagcagcagcagcagcagcagcagcagcagcag

SEQ ID NO: 135 - DNA, 105 bp, Artificial Sequence
  Synthetic polynucleotide
  misc_feature (1)..(105): /note=“This sequence may encompass 10-35 ′cag′ repeating units”
cagcagcagcagcagcagcagcagcagcagcagcagcagcagcagcagcagcagcagcagcagcagcagcagcagcagcagcagcagcagcagcagcagcagcag

SEQ ID NO: 136 - DNA, 150 bp, Artificial Sequence
  Synthetic polynucleotide
  misc_feature (1)..(150): /note=“This sequence may encompass 40-50 ′cag′ repeating units”
cagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcag

SEQ ID NO: 137 - DNA, 105 bp, Artificial Sequence; identical to SEQ ID NO: 135
  Synthetic polynucleotide
  misc_feature (1)..(105): /note=“This sequence may encompass 27-35 ′cag′ repeating units”

SEQ ID NO: 138 - protein, 4 aa, Artificial Sequence
  Synthetic peptide
GGGS

SEQ ID NO: 139 - protein, 4 aa, Artificial Sequence
  Synthetic peptide
SGGG

SEQ ID NO: 140 - protein, 5 aa, Artificial Sequence
  Synthetic peptide
GGGGS

SEQ ID NO: 141 - protein, 5 aa, Artificial Sequence
  Synthetic peptide
SGGGG

SEQ ID NO: 142 - protein, 6 aa, Artificial Sequence
  Synthetic peptide
GGGGGS

SEQ ID NO: 143 - protein, 6 aa, Artificial Sequence
  Synthetic peptide
SGGGGG

SEQ ID NO: 144 - protein, 7 aa, Artificial Sequence
  Synthetic peptide
GGGGGGS

SEQ ID NO: 145 - protein, 7 aa, Artificial Sequence
  Synthetic peptide
SGGGGGG

SEQ ID NO: 146 - protein, 5 aa, Artificial Sequence
  Synthetic peptide
  source (1)..(5): /note=“See specification as filed for detailed description of substitutions and preferred embodiments”
GGGGS

SEQ ID NO: 147 - protein, 5 aa, Artificial Sequence
  Synthetic peptide
  source (1)..(5): /note=“See specification as filed for detailed description of substitutions and preferred embodiments”
SGGGG

(19) United States
(12) Patent Application Publication
(10) Pub. No.: US 2024/0009285 A1
(43) Pub. Date: Jan. 11, 2024

(54) Compositions and Methods for Controlled Protein Degradation in Neurodegenerative Disease

(71) Applicant: Regenerative Research Foundation, Rensselaer, NY (US)

(72) Inventors: David Butler, Rensselaer, NY (US); Anne Messer, Rensselaer, NY (US); Sally Temple, Rensselaer, NY (US)

(21) Appl. No.: 18/036,592

(22) PCT Filed: Nov. 11, 2021
(86) PCT No.: PCT/US2021/059001

§ 371 Date: May 11, 2023

Related U.S. Application Data
(60) Provisional application No. 63/112,381, filed on Nov. 11, 2020.
(60) Provisional application No. 63/112,383, filed on Nov. 11, 2020.
(60) Provisional application No. 63/112,385, filed on Nov. 11, 2020.

(51) Int. Cl. A61K 38/51; C07K 16/18; A61P 25/28

(52) U.S. Cl. CPC A61K 38/51; C07K 16/18; A61P 25/28; C12Y 401/01017

(57) ABSTRACT

Disclosed herein are multifunctional polypeptides comprising a first domain comprising an antigen binding domain (e.g., anti-synuclein, anti-tau, anti-huntingtin) and a second domain comprising a programmable proteasome-targeting PEST motif, and methods for using these polypeptides in treatment of protein aggregation diseases, e.g., neurodegenerative diseases.

### CROSS-REFERENCE TO RELATED APPLICATION

This application claims the benefit of priority of U.S. Provisional Appl. No. 63/112,381, filed Nov. 11, 2020, U.S. Provisional Appl. No. 63/112,383, filed Nov. 11, 2020, and U.S. Provisional Appl. No. 63/112,385, filed Nov. 11, 2020, the contents of which are incorporated herein by reference in their entireties.

### SEQUENCE LISTING

The present specification is being filed with a computer readable form (CRF) copy of the Sequence Listing. The CRF entitled 27562-0028WO1_SL.txt, which was created on Nov. 11, 2021 and is 269,442 bytes in size, is incorporated herein by reference in its entirety.

### TECHNICAL FIELD

This disclosure relates to multifunctional polypeptides comprising a first domain comprising an antigen (e.g., anti-α-synuclein, tau, or huntingtin) binding domain and a second domain comprising a programmable proteasome-targeting PEST motif, and methods for using these polypeptides in treatment of protein aggregation diseases. These peptides can be used to treat neurodegenerative diseases (e.g., Parkinson's Disease, Alzheimer's Disease, Huntington's Disease, etc).

### BACKGROUND

Neurodegenerative diseases, such as synucleinopathies and tauopathies, are associated with accumulation of protein aggregates. These include α-synucleinopathies (such as Parkinson's disease, Lewy bodies, multiple system atrophy (MSA), and the like), and tauopathies (such as frontotemporal dementia (FTD), Alzheimer's Disease (AD), progressive supranuclear palsy (PSP), frontotemporal dementia with Parkinsonism on chromosome-17 (FTDP-17), frontotemporal lobar degeneration (FTLD-TAU), corticobasal degeneration (CBD), Alzheimer's disease, primary age-related tauopathy, Pick's disease, chronic traumatic encephalopathy (CTE) including dementia pugilistica, Lytico-bodig disease, ganglioglioma, gangliocytoma, meningioangiomatosis, postencephalitic parkinsonism, subacute sclerosing panencephalitis (SSPE), Hallervorden-Spatz disease, lead encephalopathy, tuberous sclerosis, lipofuscinosis, and the like). Neurodegenerative diseases may also result from repetitions of glutamine, which are associated with accumulation of protein aggregates. For example, Huntington's disease is a disease caused by an expanded, unstable trinucleotide repeat (CAG) in the huntingtin gene (HTT), which translates as a polyglutamine repeat in the protein product. Currently there are no well-established treatments to lower the accumulation of protein aggregates to benefit patients with tauopathies and synucleinopathies, and conditions such as Huntington's Disease, and such treatments are highly desirable.

Neurotrauma such as with traumatic brain injury (TBI) or spinal cord injury (SCI) are associated with accumulation of protein aggregates including α-synuclein and abnormal tau deposition, which can lead to neurodegeneration. Currently, there are no well-established neuroprotective treatments for TBI or SCI, thus treatments that can delay, reduce the impact of, or prevent TBI or SCI induced neurodegeneration are also highly desirable.

### SUMMARY

This disclosure relates to the characterization and use of multifunctional polypeptides that target the degradation of antigens (e.g., α-synuclein, tau, or huntingtin), thereby altering the protein levels of these antigens. This disclosure is based on the finding that certain modifications to the human PEST degron can alter the level of target antigen (e.g., synuclein, tau, huntingtin) degradation. There are various modifications (e.g., substitution of particular amino acids in the PEST degron) that can increase the degradation of a given target antigen or alter the level of degradation to the extent that it is not completely eliminated, but is reduced in pathogenic conditions. Such multifunctional polypeptides can be used to used to prevent the accumulation of disease-causing protein aggregates, thereby treating neurodegenerative conditions associated with such protein aggregation. In some instances, a multifunctional polypeptide of the disclosure comprises a first domain comprising an antigen (e.g., anti-α-synuclein, tau, or huntingtin) binding domain and a second domain comprising a programmable proteasome-targeting PEST motif, and methods for using these polypeptides in treatment of protein aggregation neurodegenerative diseases.

In one aspect, the disclosure relates to a programmable proteasome-targeting human PEST domain comprising a sequence that is at least 85% identical, at least 90% identical, or at least 95% identical to an amino acid sequence as set forth in SEQ ID NO:1 and having at least one amino acid substitution, wherein, when the PEST domain is fused to an antigen binding domain that binds to a protein, the at least one amino acid substitution increases degradation of the protein relative to an empty vector (EV) control, wherein the protein is α-synuclein, tau, or huntingtin.

In some embodiments, at least one amino acid substitution determines the relative increase in degradation in the programmable proteasome-targeting human PEST domain. In some embodiments, the at least one amino acid substitution is selected from alanine, glycine, valine, leucine, or isoleucine.

In another aspect, the programmable proteasome-targeting human PEST domain comprising the sequence NPDFX₁X₂X₃VX₄X₅QX₆AX₇X₈LX₉VX₁₀X₁₁AWX₁₂X₁₃GMX₁₄RHRAACASASINV (SEQ ID NO: 109), wherein X₁ is (P/A), X₂ is (P/A), X₃ (E/A), X₄ is (E/A), X₅ is (E/A), X₆ is (D/A), X₇ is (S/A), X₈ is (T/A), X₉ is (P/A), X₁₀ is (S/A), X₁₁ is (C/A), X₁₂ is (E/A), X₁₃ is (S/A), and X₁₄ is (K/A), wherein the sequence is not NPDFPPEVEEQDASTLPVSCAWESGMKRHR AACASASINV (SEQ ID NO:3).

In some embodiments, the domain comprises the sequence: X₁ is (P), X₂ is (A), X₃ is (E), X₄ is (E), X₅ is (E), X₆ is (D), X₇ is (5), X₈ is (T), X₉ is (P), X₁₀ is (S), X₁₁ is (C), X₁₂ is (E), X₁₃ is (S), and X₁₄ is (K) (amino acids 164-191 in SEQ ID NO:7); or X₁ is (P), X₂ is (P), X₃ is (E), X₄ is (E), X₅ is (E), X₆ is (A), X₇ is (5), X₈ is (T), X₉ is (P), X₁₀ is (S), X₁₁ is (C), X₁₂ is (E), X₁₃ is (S), and X₁₄ is (K) (amino acids 164-191 in SEQ ID NO:8); or X₁ is (P), X₂ is (P), X₃ is (E), X₄ is (E), X₅ is (E), X₆ is (D), X₇ is (5), X₈ is (T), X₉ is (P), X₁₀ is (S), X₁₁ is (C), X₁₂ is (E), X₁₃ is (A), and X₁₄ is (K) (amino acids 164-191 in SEQ ID NO:10).

In some embodiments of the above aspects, the programmable proteasome-targeting PEST domain increases degradation of α-synuclein relative to an empty vector control in an amount of between about 10% to about 30%. In some embodiments, the programmable proteasome-targeting PEST domain increases degradation of α-synuclein relative to an empty vector control in an amount of between about 30% to about 50%. In some embodiments, the programmable proteasome-targeting PEST domain increases degradation of α-synuclein relative to an empty vector control in an amount of between about 50% to about 70%. In some embodiments, the domain comprises amino acids 164-191 of SEQ ID NO:8 or SEQ ID NO:10. In some embodiments, the programmable proteasome-targeting PEST domain increases degradation of α-synuclein relative to empty vector control in an amount of between about 70% to about 99%. In some embodiments, the domain comprises amino acids 164-191 of SEQ ID NO:7.

In another aspect, the disclosure relates to a recombinant polypeptide comprising: an antigen-binding domain that binds α-synuclein; and a programmable proteasome-targeting human PEST domain of any of the above aspects. In some embodiments, the antigen-binding domain is an intrabody. In some embodiments, the intrabody is a single-chain variable fragment (scFv) or a single-domain antibody that binds α-synuclein. In some embodiments, the single-domain antibody comprises an α-synuclein-specific VL domain (VL α-synuclein), an α-synuclein-specific VH domain (VH α-synuclein) or an α-synuclein-specific VHH domain.

In some embodiments, the single-domain antibody comprises a VHH antibody set forth herein as SEQ ID NOs:16-17, or a VH-domain set forth herein as SEQ ID NO:18. In some embodiments, the domains are arranged in the order of VL[α-synuclein]-VH[α-synuclein]-PEST motif. In some embodiments, the domains are arranged in the order of VH[α-synuclein]-VL[α-synuclein]-PEST motif. In some embodiments, the antigen binding domain is selected from an antibody or functional fragment thereof, an antibody heavy-chain, an antibody light-chain, a single-domain antibody, and a scFv. In some embodiments, the α-synuclein-specific VL domain (VL α-synuclein) and an α-synuclein-specific specific VH domain (VH α-synuclein) are connected by a polypeptide linker.

In some embodiments, the linker comprises an amino acid sequence as set forth in SEQ ID NO:14. In some embodiments, the antigen binding domain is a monoclonal antibody, a synthetic antibody, a human antibody, or a humanized antibody.

In yet another aspect, the disclosure relates to a polypeptide that binds α-synuclein, comprising an amino acid sequence that is at least 80%, at least 85%, at least 90%, at least 95%, at least 97%, or 100% identical to any one of SEQ ID NOs:6-10, 12 and 13.

In some embodiments, the disclosure relates to a polynucleotide encoding a recombinant polypeptide of this disclosure. In some embodiments, the disclosure relates to vector comprising the polynucleotide described herein. In some embodiments, the disclosure relates to an isolated host cell transfected with the disclosed polynucleotide. In some embodiments, the disclosure relates to an isolated host cell transfected with the disclosed vector.

In some embodiments, the disclosure relates to pharmaceutical composition comprising a human gene therapy vector that comprises the disclosed polypeptide. In some embodiments, the pharmaceutical composition further comprises a pharmaceutically acceptable carrier.

In some embodiments, the disclosure relates to a method for the preparation of a recombinant polypeptide comprising: cultivating a host cell transfected with, and expressing, the polynucleotide disclosed herein; and isolating the polypeptide from the cell.

In some embodiments, the disclosure relates to a method for the treatment of a protein aggregation disease in a patient in need thereof, the method comprising administering to the patient a therapeutically effective amount of a recombinant polypeptide of the disclosure.

In some embodiments, the protein aggregation disease is selected from the group consisting of Parkinson's Disease (PD), multiple system atrophy (MSA), Lewy Body dementia, Alzheimer's Disease (AD), frontotemporal dementia (FTD), progressive supranuclear palsy (PSP), chronic traumatic encephalopathy (CTE), spinal cord injury (SCI), traumatic brain injury (TBI), and other synucleinopathies.

In some embodiments, the recombinant polypeptide is delivered to, or expressed in, the mid-brain dopaminergic neurons of the patient having PD. In some embodiments, the recombinant polypeptide is delivered to, or expressed in, the oligodendrocytes of the patient having MSA. In some embodiments, the recombinant polypeptide is delivered to, or expressed in, the glutamatergic neurons of the patient having a synucleinopathy such as Lewy body disease. In some embodiments, the method further comprises providing the recombinant polypeptide to the patient by gene therapy.

In some embodiments, the degradation rate of the α-synuclein is changed in a designated neural cell subtype. In some embodiments, the neural cell subtype is selected from neurons including but not limited to dopaminergic neurons, glutamatergic neurons, GABAergic neurons, cholinergic neurons, astrocytes, oligodendrocytes and microglia. In some embodiments, the neural cell subtype is selected from Neuron Specific promoters such as Synapsin I, and cell type specific promoters such as those in VGLUTI or Tyrosine Hydroxylase or Glial specific promotors such as Myelin Basic Protein or GFAP.

In some embodiments, the disclosure relates to method of changing the rate of intracellular degradation of a protein within the cytoplasm of a human cell, the method comprising introducing or expressing in the human cell a recombinant polypeptide comprising the programmable proteasome-targeting PEST domain disclosed herein.

In some embodiments, the programmable proteasome-targeting human PEST domain disclosed herein increases degradation of tau relative to an empty vector control in an amount of between (a) about 10% to about 30%, (b) about 30% to about 50%, (c) about 50% to about 70% or (d) about 70% to about 90%.

In some embodiments, the programmable proteasome-targeting human PEST domain comprises (a) amino acids 163-202 of SEQ ID NO:7; or (b) amino acids 163-202 of SEQ ID NO:8 or SEQ ID NO:10.

In another aspect, the disclosure relates to a recombinant polypeptide comprising an antigen-binding domain that binds tau; and a programmable proteasome-targeting human PEST domain disclosed herein. In some embodiments, the antigen-binding domain is an intrabody. In some embodiments, the intrabody is a single-chain variable fragment (scFv) or a single-domain antibody that binds tau. In some embodiments, the single-domain antibody comprises a tau-specific VL domain (VL tau), a tau-specific VH domain (VH tau) or a tau-specific VHH domain. In some embodiments, the recombinant polypeptide comprises the amino acid sequence set forth in any one of SEQ ID NOs.: 23-33, 35-45, 47-57, and 59-63.

In some embodiments, the single-domain antibody comprises a VH-domain comprising an amino acid sequence set forth in any one of SEQ ID NOs:65-81, or a VL-domain comprising an amino acid sequence set forth in any one of SEQ ID NOs:82-98. In some embodiments, the domains are arranged in the order of VL[tau]-VH[tau]-PEST motif. In other embodiments, the domains are arranged in the order of VH[tau]-VL[tau]-PEST motif. In some embodiments, the antigen binding domain is selected from an antibody or functional fragment thereof, an antibody heavy-chain, an antibody light-chain, a single-domain antibody, and a scFv. In some embodiments, the tau-specific VL domain (VL tau) and a tau-specific specific VH domain (VH tau) are connected by a polypeptide linker. In some embodiments, the linker comprises the amino acid sequence set forth in SEQ ID NO:14. In some embodiments, the antigen binding domain is a monoclonal antibody, a synthetic antibody, a human antibody, or a humanized antibody.

In some embodiments, the disclosure relates to a polynucleotide encoding a recombinant polypeptide of this disclosure. In some embodiments, the disclosure relates to vector comprising the polynucleotide described herein. In some embodiments, the disclosure relates to an isolated host cell transfected with the disclosed polynucleotide. In some embodiments, the disclosure relates to an isolated host cell transfected with the disclosed vector. In some embodiments, the disclosure relates to pharmaceutical composition comprising a human gene therapy vector that comprises the disclosed polypeptide. In some embodiments, the pharmaceutical composition further comprises a pharmaceutically acceptable carrier.

In some embodiments, the disclosure relates to a method for the preparation of a recombinant polypeptide comprising: cultivating a host cell transfected with, and expressing, the disclosed polynucleotide; and isolating the polypeptide from the cell.

In some embodiments, the disclosure relates to method for the treatment of a protein aggregation disease in a patient in need thereof, the method comprising providing to the patient a therapeutically effective amount of a recombinant polypeptide disclosed herein.

In some embodiments, the protein aggregation disease is selected from Frontotemporal dementia (FTD), Alzheimer's Disease (AD) progressive supranuclear palsy (PSP), frontotemporal dementia with Parkinsonism on chromosome-17 (FTDP-17), frontotemporal lobar degeneration (FTLD-TAU), corticobasal degeneration (CBD), primary age-related tauopathy, Pick's disease, chronic traumatic encephalopathy (CTE), Lewy Body dementia, Vascular dementia, tuberous sclerosis, spinal cord injury (SCI), traumatic brain injury (TBI) or other tauopathies. In some embodiments, the recombinant polypeptide is delivered to, or expressed in, the mid-brain dopaminergic neurons of the patient having PD. In some embodiments, the recombinant polypeptide is delivered to, or expressed in, the oligodendrocytes on the patient having MSA. In some embodiments, the recombinant polypeptide is delivered to, or expressed in, the glutamatergic neurons of the patient having a tauopathy. In some embodiments, the method comprises providing the recombinant polypeptide to the patient by gene therapy.

In yet another aspect, the programmable proteasome-targeting human PEST domain disclosed herein increases degradation of Huntingtin relative to an empty vector control in an amount of between about 10% to about 30%. In some embodiments, the programmable proteasome-targeting PEST domain increases degradation of Huntingtin relative to an empty vector control in an amount of between about 30% to about 50%.

In some embodiments, the programmable proteasome-targeting PEST domain increases degradation of Huntingtin relative to empty vector control in an amount of between about 50% to about 70%. In some embodiments, the programmable proteasome-targeting PEST domain increases degradation of Huntingtin relative to empty vector control in an amount of between about 70% to about 99%. In some embodiments, the domain comprises amino acids 259-283 of SEQ ID NO:100, SEQ ID NO:101, SEQ ID NO:102, SEQ ID NO:103, or SEQ ID NO:105.

In some aspects the disclosure relates to a recombinant polypeptide that binds huntingtin, comprising an amino acid sequence that is at least 80%, at least 85%, at least 90%, at least 95%, at least 97%, or 100% identical to any one of SEQ ID NOs:100-108.

In some embodiments, the recombinant polypeptide comprises: an antigen-binding domain that binds Huntingtin; and a programmable proteasome-targeting human PEST domain of disclosed herein. In some embodiments, the antigen-binding domain is an intrabody. In some embodiments, the intrabody is a single-chain variable fragment (scFv) or a single-domain antibody that binds Huntingtin.

In some embodiments, the single-domain antibody comprises a Huntingtin-specific VL domain (VL Huntingtin), a Huntingtin-specific VH domain (VH Huntingtin) or a Huntingtin-specific VHH domain. In some embodiments, the scFv comprises a VH domain set forth herein as SEQ ID NO:106, and a VL-domain set forth herein as SEQ ID NO:107. In some embodiments, the domains are arranged in the order of VL[Huntingtin]-VH[Huntingtin]-PEST motif or VH[Huntingtin]-VL[Huntingtin]-PEST motif. In some embodiments, the Huntingtin-specific VL domain comprises the sequence set forth herein as SEQ ID NO:108. In some embodiments, the antigen binding domain is selected from an antibody or functional fragment thereof, an antibody heavy-chain, an antibody light-chain, a single-domain antibody, and a scFv. In some embodiments, the Huntingtin-specific VL domain (VL Huntingtin) and the Huntingtin-specific specific VH domain (VH Huntingtin) are connected by a polypeptide linker. In some embodiments, the linker comprises an amino acid sequence as set forth in SEQ ID NO:14. In some embodiments, the antigen binding domain is a monoclonal antibody, a synthetic antibody, a human antibody, or a humanized antibody.

In some embodiments, the disclosure relates to a polynucleotide encoding a recombinant polypeptide of this disclosure. In some embodiments, the disclosure relates to vector comprising the polynucleotide described herein. In some embodiments, the disclosure relates to an isolated host cell transfected with the disclosed polynucleotide. In some embodiments, the disclosure relates to an isolated host cell transfected with the disclosed vector. In some embodiments, the disclosure relates to pharmaceutical composition comprising a human gene therapy vector that comprises the disclosed polypeptide. In some embodiments, the pharmaceutical composition further comprises a pharmaceutically acceptable carrier.

In some embodiments, the disclosure relates to a method for the preparation of a recombinant polypeptide comprising: cultivating a host cell transfected with, and expressing, the polynucleotide disclosed herein; and isolating the polypeptide from the cell.

In some embodiments, the disclosure relates to a method for the treatment of a protein aggregation disease in a patient in need thereof, the method comprising providing to the patient a therapeutically effective amount of the disclosed recombinant polypeptide. In some embodiments, the protein aggregation disease is selected from Huntington's disease, or other protein aggregation neurodegeneration diseases including Parkinson's Disease (PD), multiple system atrophy (MSA), and Lewy Body dementia, Alzheimer's Disease (AD), frontotemporal dementia (FTD), progressive supranuclear palsy (PSP), chronic traumatic encephalopathy (CTE), and spinal cord injury (SCI), and traumatic brain injury (TBI).

In some embodiments, the protein aggregation disease is Huntington's Disease. In some embodiments, the recombinant polypeptide is delivered to, or expressed in, the mid-brain dopaminergic neurons of the patient having PD. In some embodiments, the recombinant polypeptide is delivered to, or expressed in, the oligodendrocytes on the patient having MSA. In some embodiments, the recombinant polypeptide is delivered to, or expressed in, the glutamatergic neurons of the patient having Huntington's Disease. In some embodiments, the recombinant polypeptide to the patient by gene therapy.

In another aspect, the disclosure relates to a programmable proteasome-targeting human PEST domain comprising a sequence that is at least 85% identical, at least 90% identical, or at least 95% identical to an amino acid sequence as set forth in SEQ ID NO:1 and having at least one amino acid substitution, wherein, when the PEST domain is fused to an antigen binding domain that binds to a protein, the at least one amino acid substitution decreases degradation of the protein relative to an empty vector (EV) control, wherein the protein is α-synuclein, tau, or huntingtin. In some embodiments, the at least one amino acid substitution determines the relative decrease in degradation. In some embodiments, the at least one amino acid substitution is selected from alanine, glycine, valine, leucine, or isoleucine.

In some embodiments, the programmable proteasome-targeting PEST domain decreases degradation of α-synuclein relative to an empty vector control in an amount of between (a) about 10% to about 30%; (b) about 30% to about 50%; (c) about 50% to about 70% or (d) about 70% to about 99%. In some embodiments, the domain comprises amino acids 164-191 of SEQ ID NO:9.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

For the avoidance of any doubt it is emphasized that the expressions “in some embodiments”, “in a certain embodiments”, “in certain instances”, “in some instances”, “in a further embodiment”, “in one embodiment” and “in a further embodiment” and the like are used and meant such that any of the embodiments described therein are to be read with a mind to combine each of the features of those embodiments and that the disclosure has to be treated in the same way as if the combination of the features of those embodiments would be spelled out in one embodiment. The same is true for any combination of embodiments and features of the appended claims and illustrated in the Examples, which are also intended to be combined with features from corresponding embodiments disclosed in the description, wherein only for the sake of consistency and conciseness the embodiments are characterized by dependencies while in fact each embodiment and combination of features, which could be construed due to the (multiple) dependencies must be seen to be literally disclosed and not considered as a selection among different choices. In this context, the person skilled in the art will appreciate that the embodiments and features disclosed in the Examples are intended to be generalized to equivalents having the same function as those exemplified therein.

Other features and advantages of the disclosure will be apparent from the following detailed description and from the claims.

### BRIEF DESCRIPTION OF THE DRAWINGS

FIGS. 1A-C—Show a schematic of a development strategy for a bi-functional scFv intrabody from a conventional IgG antibody and how it is delivered into a target cell by transfection. (A) Illustration of a conventional antibody (left), a camelid antibody (middle), and a single-domain antibody (right). An scFv intrabody is assembled by linking the shortest variable-region fragment (Fv) genes that encode the variable heavy (VH) and variable light (VL) domains of a conventional antibody together with a flexible peptide linker. Camelid antibodies are composed of two heavy chains, with a single variable domain (VHH). Single domain antibodies are composed of either a VH domain, a VL domain, or a VHH domain. (B) A bi-functional intrabody is composed of an antigen binding domain, i.e., a scFv or single-domain antibody comprised of either a VH, VL, or VHH, fused to a human ornithine decarboxylase (ODC) PEST degron (a PEST sequence is one that is rich in proline (P), glutamic acid (E), serine (S), and threonine (T)). In some embodiments, an epitope tag such as Human influenza hemagglutinin (HA) can be used to identify the intracellular expression of the bi-functional intrabody. (C) Experimental design of bi-functional intrabodies in cells. Bi-functional intrabody-mediated targeted degradation occurs through the proteasome. The PEST degron binds to the 19S lid of the proteasome, where it facilitates degradation of the intrabody and its bound cargo by the 26S proteasome. (1) The bi-functional intrabody is delivered into the cell by vector-based or protein-based delivery. (2) The cell will then make an mRNA encoding the intrabody that is translated into a protein at the (3) Ribosome. (4) The bi-functional intrabody prevents aggregation of its target protein. (5) The bi-functional intrabody and its target protein are degraded by the proteasome. FIGS. 1A-C disclose SEQ ID NOs.:133, 14, 15, and 15, respectively, in order of appearance.

FIG. 2—Demonstrates that VH14-hPEST lowers steady state levels of human α-synuclein in ST14A neuronal cells. α-synuclein-GFP was co-transfected with either VH14-mouse PEST (VH14-mPEST) or VH14-human PEST (VH14-hPEST). 72 h after transfection, cells were live imaged. Western blot shows the reduction of the α-synuclein-GFP monomer and higher molecular weight species in VH14-mPEST and VH14-hPEST transfected cells compared to empty vector (EV) control. α-synuclein-GFP was detected using a pan-synuclein antibody that recognizes all forms of the synucleins (1:500; Abcam #6176). Actin was probed as a loading control. Graphs show densitometric analysis of western blot signals. Each bar represents pan-synuclein/actin loading control expressed as a percentage of empty vector control. Pan-synuclein refers to Rabbit polyclonal antibody (ab6176) that recognizes multiple forms of the synucleins.

FIG. 3—Shows verification of endogenous synuclein expression in human iPSC-derived 3D cortical organoids and increased expression in mutant organoids having a SNCA gene triplication (3×SNCA). 3×-SNCA and WT iPSCs were differentiated into 3D forebrain organoids. Following 60 days in vitro (DIV), organoids were harvested for western blotting. 20 ug (left 4 lanes) or 10 μg (right two lanes 5 and 6) of total protein was separated by gel electrophoresis and transferred onto PVDF membranes. Endogenous synuclein was detected using an MJFR1 anti-α-synuclein antibody (1:1000; Abcam #ab138501). GAPDH was used as a loading control (1:10,000; Abcam, #ab181602).

FIGS. 4A-B—Show that VH14-hPEST significantly reduced endogenous α-synuclein in human cortical neurons. (A) Design of Tet-On inducible anti-synuclein lentiviral constructs. Anti-synuclein mPEST and hPEST intrabodies were subcloned into pTet-O-Ngn2-puro (Addgene plasmid #52047). The Ngn2 insert was replaced with VH14-hPEST and VH14-hPEST-Scramble-control. The 5′ cloning site was EcoRI and the 3′ cloning site was XbaI. (B) VH14-hPEST reduced levels of endogenous human α-synuclein in 3×SNCA forebrain organoids. Immunofluorescence shows reduction in α-synuclein (MJFR1; Abcam, lowest panel) levels in organoids transduced (n=3) with lentivirus carrying VH14-hPEST compared to empty vector (EV)-treated organoids (control) and organoids treated with VH14 fused to a scrambled (Scr) PEST. Densitometric quantification of α-synuclein signal in organoids confirm statistically significant reduction of target protein in both VH14-treated cohorts. (all ****=p<0.0001).

FIG. 5—Shows that a bi-functional intrabody targeting botulinum toxin (B8) with hPEST does not alter the steady-state protein levels of lamprey DY-synuclein˜GFP (DY-syn˜GFP). ST14A neuronal cells were co-transfected with DY-syn˜GFP and either B8-hPEST or Empty Vector control. 48 hours after transfection, cells were live cell imaged and then harvested for western blotting. Right two panels: Live Cell Imaging for DY-syn˜GFP (Scale bar 200 μm). Left panel: Representative western blotting is as described above. The B8 construct did not degrade the DY-syn˜GFP, demonstrating specificity of the synuclein-targeting constructs.

FIG. 6—Diagram of human α-synuclein protein. The proposed intrabody binding site locations of VH14, VHH-4C, VHH-4C-N77D, and DB1 are to the non-amyloid component (NAC) hydrophobic domain of α-synuclein that is prone to aggregation and has been shown to be critical for misfolding.

FIG. 7—Shows expression of lamprey synuclein. DY-syn˜GFP, FD-syn˜GFP, and Syn3˜GFP were separately transfected into ST14A neuronal cells. 48 hours after transfection, cells were live cell imaged. Inset: DY-synuclein puncta are visible (arrowhead).

FIGS. 8A-B—Show that VHH-4C-N77D-PEST (significantly (p<0.05) reduced steady-state levels of synuclein relative to EV-control. ST14A neuronal cells were co-transfected with (A) lamprey DY-synuclein˜GFP or (B) human α-synuclein˜GFP and either VHH-4C-PEST (4C-PEST), VHH-4C-N77D-PEST (N77D-PEST) or Empty Vector control (EV-CON). 48 hours after transfection, cells were live imaged and then harvested for western blotting. Scale bar=50 μm. 10 μg of total protein was separated by gel electrophoresis and transferred onto PVDF membranes. (A) DY-synuclein˜GFP was detected using a pan-synuclein antibody (1:500; Abcam #6176). (B) α-synuclein was detected using purified mouse anti-human α-synuclein (1:1000; BD Biosciences #610787). GAPDH was used as a loading control (1:10,000; Abcam, #ab181602). The secondary antibody used was a goat anti-rabbit HRP conjugated IgG (H+L) (1:2000; Thermo Scientific). Protein bands were detected using Western Lightning™ Chemiluminescence Reagent Plus western blotting substrate (PerkinElmer®). Synuclein densitometry was conducted using imageJ software from three independent experiments. Graphs show relative densitometric quantification of synuclein western blot band intensities. Densitometry bars represent synuclein mean optical density values relative to GAPDH loading control. The data are presented relative to EV-CON. A Brown-Forsythe ANOVA followed by Dunnett's post-hoc test for multiple comparisons; * denotes p<0.05.

FIG. 9—Shows that VHH-4C-N77D-hPEST (N77D-hPEST) with the human PEST significantly (p<0.05) reduces DY-syn-GFP expression. ST14A neuronal cells were co-transfected with DY-synuclein˜GFP and either VHH-4C-N77D-hPEST, hPEST-Scr, or Empty Vector control (EV-CON). Cells were live imaged, 48 hours after transfection, and harvested for western blotting. (A) Live Cell Imaging (Scale bar 200 μm). (B) Representative western blot of DY-synuclein˜GFP. (C) Densitometry bars represent DY-syn˜GFP mean optical density values relative to GAPDH loading control. Data represent mean±SEM (n=3 per group). One-way ANOVA was performed followed by Tukey's multiple comparisons post-hoc tests. * denotes p<0.05. compared with EV-Con treatment. Densitometry bars represent DY-syn˜GFP mean optical density values relative to GAPDH loading control.

FIGS. 10A-B—Show that VHH-4C-N77D-hPEST (N77D-hPEST) targeting α-synuclein does not significantly reduce β-synuclein˜GFP (A) or γ-synuclein˜GFP (B) levels. ST14A neuronal cells were co-transfected with either (A) β-synuclein˜GFP or (B) γ-synuclein˜GFP and either Empty Vector Control (CON), B8-PEST (targeting botulinim toxin), N77D-hPEST, or N77D-hPEST-Scr. 72 hours after transfection, cells were harvested for western blotting. 10 μg of total protein was separated by gel electrophoresis and transferred onto PVDF membranes. β-synuclein˜GFP and γ-synuclein˜GFP was detected using GFP antibodies (1:1000; Abcam #6556). GAPDH was used as a loading control (1:10,000; Abcam, #ab181602). The secondary antibody used was a goat anti-rabbit HRP conjugated IgG (H+L) (1:2000; Thermo Scientific). Protein bands were detected using Western Lightning™ Chemiluminescence Reagent Plus western blotting substrate (PerkinElmer). Densitometry bars represent GFP mean optical density values relative to GAPDH loading control. Data represent mean±SEM (n=3 per group). One-way ANOVA was performed followed by Tukey's multiple comparisons post-hoc tests; * denotes p<0.05.

FIGS. 11A-B—Show controlled degradation of human α-synuclein by varying the hPEST construct sequence. ST14A cells were transfected with human α-synuclein-GFP and either empty vector control (EV CON), VH14-hPEST, and VH14-hPEST variants (D433A, S445A), compound mutation variant (P426A/P427A), or inactive degron control (C441A). 72 hours after being transfected, the cells were collected for western blotting: (A) Western blot. (B) Quantification of the western blot by densitometry. The relative protein expression was determined by the ratio of total α-synuclein to an internal standard control (GAPDH). Samples were then normalized to EV-CON. Human PEST degron variants D433A and S445A result in altered protein degradation levels compared to inactive control (CON) or empty vector control (EV CON). Compound mutation variant P426/P427A resulted in altered synuclein expression compared to all groups. C441A is a mutation that renders the hPEST inactive.

FIGS. 12A-C—Show controlled degradation of human α-synuclein by varying the hPEST construct sequence. ST14A cells were transfected with human α-synuclein-GFP and either empty vector control (EV CON), VH14-hPEST, and VH14-hPEST variants (D433A, S445A, C441A), compound mutation variant (P426A/P427A), inactive scrambled PEST degron control (SCR) or B8-PEST (targeting botulinim toxin) irrelevant antigen control. 72 hours after being transfected, the cells were live imaged for α-synuclein-GFP fluorescence and then collected for western blotting: (A) Live Cell Imaging (Scale bar 50 μm). (B) Western blot. (C) Quantification of the western blot by densitometry. Data represent mean±SEM. One-way ANOVA was performed followed by Tukey's multiple comparisons post-hoc tests. * Indicates p<0.05 and *** p<0.001 compared with EV-CON treatment. The relative protein expression was determined by the ratio of total α-synuclein to an internal standard control (GAPDH). Samples were then normalized to EV-CON. VH14-hPEST and VH14-PEST degron variant D433A resulted in significant (p<0.05) protein degradation levels compared to empty vector control (EV CON). Compound mutation variant P426A/P427A resulted in altered synuclein expression compared to VH14-hPEST and a significant (p<0.001) protein degradation level compared to empty vector control (EV CON).

FIG. 13—Shows that VH14-hPEST and hPEST degron variants reduce endogenous α-synuclein in human midbrain neuronal cultures compared to EV CON and VH14-hPEST-SCR (SCR) control. 3×-SNCA iPSCs were differentiated into 3D midbrain organoids. Following 30 days in vitro (DIV), organoids were transduced with lentivirus carrying VH14-hPEST, VH14-hPEST degron variants P426A/P427A, D433A, S445A, or an inactive scrambled PEST degron control and compared to empty vector Empty Vector Controls (EV CON). After 30 days of treatment, organoids were processed for immunofluorescent staining. Endogenous synuclein was detected using an MJFR1 anti-α-synuclein antibody (1:1000; Abcam #ab138501). Intrabody expression was determine using an anti-HA antibody (1:1000; Abcam #ab117515). Because inclusions of anti-α-synuclein can occur in neurons and glia, astrocytes were identified using anti-S100(3, a marker for mature astrocytes (1:100; Abcam #ab52642). Immunofluorescence shows reduction in α-synuclein (MJFR1; Abcam, Green) levels in organoids transduced (n=3) with lentivirus carrying VH14-hPEST and hPEST degron variants compared to empty vector (EV)-treated organoids (control) and organoids treated with VH14 fused to a scrambled (Scr) PEST.

FIG. 14—Shows that VH14-hPEST and hPEST degron variants reduce DNA fragmentation generated during apoptosis. WT and 3×-SNCA iPSCs were differentiated into 3D midbrain organoids. Following 30 days in vitro (DIV), organoids were transduced with lentivirus carrying VH14-hPEST, VH14-hPEST degron variants P426A/P427A, D433A, S445A, or an inactive scrambled PEST degron control and compared to empty vector Empty Vector Controls (EV CON). After 30 days of treatment, organoids were processed for immunofluorescent staining using a terminal deoxynucleotidyl transferase dUTP nick end labeling staining, also called the TUNEL assay (DeadEnd Fluorometric TUNEL system; Promega #G3250). 3×SNCA midbrain organoids displayed increased TUNEL reactivity (bright dots) compared to WT midbrain organoids. There is minimal TUNEL reactivity in WT or 3×SNCA midbrain organoids treated with VH14-hPEST and VH14-hPEST degron variants P426A/P427A, D433A, S445A.

FIG. 15—Shows human Ornithine Decarboxylase PEST (hPEST) degron variants targeting α-synuclein to the proteasome for degradation via a human PEST degron fusion. Certain single (A) and compound (B) mutations within the hPEST degron (highlighted in grey) are predicted to alter the targeted degradation of the intrabody and its bound antigen. This has been demonstrated for ones highlighted in bold, i.e., D433A, C441A, S445A and P426A/P427A. The PEST degron is shaded in the top row (ODC amino acids 423-450). FIG. 15 discloses SEQ ID NOs.:3 and 110-132, respectively, in order of appearance.

FIG. 16—Shows anti-α-synuclein VHH and VH sequences: VHH-4C-N77D (top), VHH-DB1 (middle), and VH14 (bottom), corresponding to SEQ ID NO:16, SEQ ID NO:17, and SEQ ID NO:18, respectively. CDR=complementarity determining region.

FIG. 17—Shows a comparison of mouse and human ornithine decarboxylase (ODC) PEST degron (amino acids 422-461). PEST region is indicated in bold. Conserved areas are shaded grey. Non-conserved regions have no marking. The C-terminal 40 amino acids of mODC degron share 77.5% (31/40) homology with hODC, with the C-terminal 37 amino acids sharing 83.7% (31/37) homology. Consensus PEST sequence is shown in underlining/italics. FIG. 17 discloses SEQ ID NOs:2 and 3, respectively, in order of appearance.

FIGS. 18A-C—Show a comparison of tau reduction with mPEST and hPEST degron. ST14A cells were transfected with GFP-Tau and either empty vector control (EV CON), V-mPEST, V-hPEST, N-mPEST, N-hPEST, F-mPEST, and F-hPEST. 72 h after transfection, samples were collected for: (A) Live cell imaging. (B) Western blot. (C) The relative protein expression was determined by the ratio of total tau to an internal standard control (GAPDH). Samples were then normalized to EV-control.

FIGS. 19A-B—Show controlled degradation of tau by varying the hPEST construct sequence. ST14A cells were transfected with GFP-Tau and either empty vector control (EV CON), V-hPEST or N-hPEST degron variants P426A/P427A, D433A, S445A, or C441A. (A) Western blot. (B) The relative protein expression was determined by the ratio of total tau to an internal standard control (GAPDH). Samples were then normalized to EV-control. Human PEST degron variants (P426A/P427A, D433A, and S445A) resulted in altered protein degradation levels compared to inactive hPEST degron (C441A) or empty vector control (EV CON).

FIGS. 20A-C—Show controlled degradation of tau by varying the hPEST construct sequence with V-hPEST degron variants. ST14A cells were transfected with GFP-Tau and either empty vector control (EV CON), V-hPEST, V-hPEST degron variants E428A/E430A/E431A, D433A, S435A, P438A, S440A, C441A, E444A, S445A, or inactive scrambled PEST degron control (SCR). 72 h after transfection, samples were collected for: (A) Live cell imaging. Scale bar=50 μm. (B) Western blot. (C) The relative protein expression was determined by the ratio of total tau to an internal standard control (GAPDH). Samples were then normalized to EV-control. Human PEST degron variants resulted in altered protein degradation levels compared empty vector control (EV CON). V-PEST variants E428A/E430A/E431A, D433A, S435A, P438A, S440A, C441A and E444A reduced GFP-Tau to 75-100% relative to control. V-PEST and V-PEST variant S445A reduced GFP-Tau to 50-75% of control.

FIGS. 21A-C—Show controlled degradation of tau by varying the hPEST construct sequence with N-hPEST degron variants. ST14A cells were transfected with GFP-Tau and either empty vector control (EV CON), N-hPEST, N-hPEST degron variants E428A/E430A/E431A, D433A, S435A, P438A, S440A, C441A, E444A, S445A, or inactive scrambled PEST degron control (SCR). 72 h after transfection, samples were collected for: (A) Live cell imaging. Scale bar=50 μm. (B) Western blot. (C) The relative protein expression was determined by the ratio of total tau to an internal standard control (GAPDH). Samples were then normalized to EV-control. Human PEST degron variants resulted in altered protein degradation levels compared empty vector control (EV CON). N-PEST reduced GFP-Tau 50-75% relative to control and N-PEST variants P426A/P427A, P438A, E444A, and K448A/R449A/H450A reduced GFP-Tau 25-50% relative to control. N-PEST variants E428A/E430A/E431A, S435A, S440A, and S445A reduced GFP-Tau 0-25% relative to control. B8-PEST, N-PEST-SCR, and N-PEST variants D433A and C441A increased the level of GFP-Tau compared to EV CON.

FIGS. 22A-C—Show controlled degradation of tau by varying the hPEST construct sequence with F-hPEST degron variants. ST14A cells were transfected with GFP-Tau and either empty vector control (EV CON), F-hPEST, F-hPEST degron variants P246A/P427A, E428A/E430A/E431A, D433A, S435A, P438A, S440A, C441A, E444A, S445A, K448A/R449A/H450A, or inactive scrambled PEST degron control (SCR). 72 h after transfection, samples were collected for: (A) Live cell imaging. Scale bar=50 μm. (B) Western blot. (C) The relative protein expression was determined by the ratio of total tau to an internal standard control (GAPDH). Samples were then normalized to EV-control. Human PEST degron variants resulted in altered protein degradation levels compared empty vector control (EV CON). F-PEST and F-PEST variants E428A/E430A/E431A, D433A, P438A, E444A, S445A, and K448A/R449A/H450A reduced GFP-Tau 50-75% relative to control. F-PEST variant P426A/P427A reduced GFP-Tau 25-50% relative to control. F-PEST-SCR expressed similar levels of GFP-Tau compared to EV CON.

FIGS. 23A-E—Show elevated cell death in induced pluripotent stem cell (iPSC) derived cortical neurons with a MAPT V337M mutation compared to isogenic V337V controls. (A) Forebrain specification at 20 days. Neural Precursor cells stained positive for forebrain identity markers PAX6 (dorsal forebrain progenitors), SOX2 (neural ectoderm marker), and general neuronal marker TUJI (neuron-specific class III β-tubulin). (B) Forebrain specification. FOXG1 (forebrain marker), TUJI, and were negative for SOX10 (Neural crest). Scale bar=50 μm. (C) Cell viability staing with Ethidium homodimer (EthD). Viable cells, Hoechst 33342 positive cell (light gray circles). Dead cells EthD (bright puncta, some shown with arrows). Scale bar=100 μm (D) V337M mutant neuronal cultures have significantly elevated cell death (** P=***P=0.0007) compared to isogenic V337V control. Cortical cultures (110 days in vitro) were treated with either Rotenone (0.75 μM) or DMSO vehicle control. 24 h after treatment cell death was detected by ethidium homodimers staining. Cell Death was quantified with CellProfiler™. Scale bar=100 μm. Data represent mean±SEM (n=3 per group). One-way ANOVA was performed followed by Tukey's multiple comparisons post-hoc tests. * Indicates p<0.05 and ** p<0.01 compared with V337V (WT). (E) Lactate dehydrogenase (LDH) levels are significantly elevated in conditioned media isolated from V337M cultures compared to isogenic V337V control (* P=0.0240).

FIG. 24—Shows proteasome impairment in induced pluripotent stem cell (iPSC) derived cortical neurons with a MAPT V337M mutation compared to isogenic V337V controls. Mutant (V337M) and isogenic control (V337V) cortical cultures transduced with a Ubiquitin^{G76V}GFP (Ub^{G76V}GFP) reporter after 90 days in culture to monitor proteasome function in live cells. At 110 days in culture, a timepoint where we observed cell death in MAPT V377M mutant cultures compared to isogenic V337V control, cells were live imaged. Scale bar=100 μm. In healthy, V337V control cultures, the Ub^{G76V}GFP (depicted by a lack of arrows in the top middle and merged panels) reporter is rapidly degraded; however, in MAPT V337M mutant cortical neurons, there is accumulation of UB G76V GFP reporter (arrows) which implies that ubiquitin proteasome system is impaired.

FIG. 25—Shows bifunctional anti-tau-PEST intrabodies can alleviate proteasome impairment in induced pluripotent stem cell (iPSC) derived cortical neurons with a MAPT V337M mutation compared to isogenic V337V controls. Mutant (V337M) and isogenic control (V337V) cortical cultures were transduced with Ubiquitin^{G76V}GFP (Ub^{G76V}GFP) reporter and either empty vector control (EV), V-hPEST, N-hPEST, or F-hPEST after 90 days in culture to determine if targeted degradation of tau via ubiquitin independent proteolysis with bifunctional anti-tau-hPEST intrabodies can counteract proteasome impairment caused by mutant V337M tau. After 20 days of treatment, the cells were live imaged. Scale bar=100 μm. In V337M mutant cultures, there is an accumulation of the Ub^{G76V}GFP (arrows) reporter, indicating proteasome impairment. In healthy V337V controls and V337M mutant cultures treated with anti-tau-hPEST intrabodies, the Ub^{G76V}GFP (arrowheads in 2^{nd} and 4^{th} panels) reporter is rapidly degraded.

FIG. 26—Shows that anti-Tau-hPEST intrabodies, V-hPEST and N-hPEST, reduced cell death in human iPSC derived cortical neurons with a MAPT V337M mutation. V337M cortical cultures were transduced at 90 days with either empty vector control (EV-CON), V-hPEST, N-hPEST, or B8-hPEST control intrabody to an irrelevant antigen, botulinum toxin. At 110 days, an ethidium homodimer (EtHD) assay was used to detect dead and/or dying cells. Cell death was then quantified with CellProfiler™. Data represent mean±SEM. A Two-way ANOVA was performed followed by Tukey's multiple comparisons post-hoc tests. V337M mutant neuronal cultures have significantly elevated cell death (***, p=0.0004) compared to isogenic V337V control. Cell death levels were significantly reduced by V-hPEST (*, p=0.0271). N-hPEST approached significance (p=0.0517) in V337M cultures compared to isogenic V337V control.

FIG. 27—Shows that with programmable target antigen proteolysis (P-TAP) technology, the lowest effective level of tau degradation to achieve neuroprotection can be determined in human iPSC derived cortical neurons. Mutant MAPT V337M cortical cultures were transduced with either empty vector control (EV CON), N-hPEST, N-hPEST variant S445A, or N-hPEST with an inactive scrambled hPEST degron (SCR) at 60 days. At 90 days, an ethidium homodimer (EtHD) assay was used to detect dead and/or dying cells. Cell death was quantified with CellProfiler™. Data represent mean±SEM. A One-way ANOVA was performed followed by Tukey's multiple comparisons post-hoc tests. Cell death levels were significantly (*, p<0.05) reduced by N-hPEST, a strong reducer of Tau (50-75%). N-hPEST degron variant S445A, a low reducer of Tau (0-25%), significantly (*, p<0.05) reduced cell death in V337M cortical cultures compared to EV CON.

FIG. 28—Shows a map of the MAPT (tau) gene, along with mutations in particular exons and introns.

FIG. 29—Shows human Ornithine Decarboxylase PEST (hPEST) degron variants targeting tau to the proteasome for degradation via a human PEST degron fusion. Certain single (A) and compound (B) mutations within the hPEST degron (highlighted in grey) are predicted to alter the targeted degradation of the intrabody and its bound antigen. This has been demonstrated by ones highlighted in bold, i.e., D433A, S435A, P438A, S440A, C441A, E444A, S445A, P426A/P427A, E428A-E430A-E431A, and K448A, R449A, and H450A. The PEST degron is shaded in the top row (ODC amino acids 423-450). FIG. 29 discloses SEQ ID NOs.:3 and 110-132, respectively, in order of appearance.

FIG. 30—Shows anti-tau scFv sequences. Top shows VH sequences, corresponding to SEQ ID NOs:65-81, respectively; bottom shows VL sequences, corresponding to SEQ ID NOs:82-98, respectively.

FIGS. 31A-B—Demonstrate that bifunctional anti-mutant HTT (mHTT) intrabodies, C4 and VL12.3, with a human PEST degron counteract mHTT aggregation and promote clearance of mHTT fragments in ST14A neuronal cells. mHTTex1-72Q-eGFP was co-transfected with either Empty Vector Control (Control), C4-PEST, VL12.3-PEST, or C4 and VL12.3 with an inactive scrambled PEST degron that does not promote protein degradation (C4-PEST-SCR), or (VL12.3-PEST-SCR). 72 h after transfection, cells were live imaged and harvested for western blotting. mHTTex1-72Q-GFP was detected using a monoclonal antibody EM48 (Millipore, Cat #MAB5374). Actin was probed as a loading control. The intrabodies were detected by probing for their HA-tag with anti-HA. (A) C4-PEST and VL12.3-PEST degrade mHTTex1-72Q-GFP compared to control cells which contain mHTTex1-72Q-GFP aggregates (arrows). C4-PEST and VL12.3-PEST prevent mHTTex1-72Q-GFP aggregation compared to control. (B). Western blot shows the reduction of the soluble mHTTex1-72Q-GFP monomer and higher molecular weight species in C4-PEST and VL12.3-PEST transfected cells compared to empty vector control and C4-PEST-SCR and VL12.3-PEST-SCR controls. Aggregated mHTTex1-72Q-GFP is trapped in the stacking gel. Graphs show densitometric analysis of western blot signals. Each bar represents mHTTex1-72Q-GFP/Actin loading control expressed as a percentage of empty vector control.

FIGS. 32A-B—Show that mHTT exon 1 protein fragments impair the Ubiquitin Proteasome System. ST14A cells were co-transfected with a Ubiquitin^{G76V}GFP (Ubiquitin^{G76V}GFP) reporter and either empty vector control (EV CON), mHTTex1-25Q-RFP (46Q-RFP), mHTTex1-46Q-RFP (46Q-RFP), or mHTTex1-72Q-RFP (72Q-RFP). 72 hours after transfection, the cells are live imaged for mHTT aggregation (arrows) and the accumulation of UB G76V GFP (asterisks). Scale bars 50 μm. (A) In healthy EV CON cells, the Ubiquitin^{G76V}GFP is quickly degraded by the proteasome. In cells transfected with a non-pathogenic polyglutamine repeat length, 25Q-RFP, the UB G76V GFP reported is efficiently degraded. In cells treated with pathogenic polyglutamine repeat lengths, 46Q-RFP and 72-RFP, there is accumulation of UB G76V GFP which implies that ubiquitin proteasome system is impaired. Additionally, ubiquitin (asterisks) is being incorporated into mHTT 46Q and 72Q aggregates (arrows), merged image (arrowheads). (B) A zoomed section of merged images.

FIG. 33—Shows that C4-PEST degradation counteracts proteasome impairment caused by mHTT exon 1 protein fragments. ST14A cells were co-transfected with Ubiquitin^{G76V}GFP (Ubiquitin^{G76V}GFP) reporter, mHTTex1-72Q-RFP (72Q-RFP), and either Empty Vector Control (EV CON), C4 with a human PEST degron (C4-PEST), or C4 with an inactive scrambled human PEST degron (C4-PEST-SCR). 72 hours after transfection, the cells were live imaged for mHTT aggregation (arrows) and the accumulation of UB G76V GFP (asterisk). Scale bars 50 μm. In EV CON cells, the proteasome is impaired by Q72-RFP aggregation. The UB G76V GFP reporter (asterisks) is accumulating in these cells and incorporated into 72Q-RFP aggregates (arrows), merged image (arrowheads). In cells treated with C4-PEST-SCR, aggregation of 72Q-RFP is prevented as 72Q-RFP is diffuse within the cell. The Ubiquitin^{G76V}GFP reporter quickly degraded by the proteasome. In cells treated with C4-PEST, aggregation of 72Q-RFP is also prevented as 72Q-RFP is diffuse within the cell. The PEST degron results in efficient degradation of mHTT as 72Q-RFP (arrows) expression is barely detectable. The UB G76V GFP reporter quickly degraded by the proteasome.

FIGS. 34A-B—Show controlled degradation of mHTT by varying the human PEST degron construct sequence. ST14A neuronal cells were transfected with human mHTTex1-72Q-GFP and either empty vector control (EV CON), C4, C4-PEST, and C4-PEST variants (T436A, P438A, S440A, E444A), compound mutation variant (E428A/E430A/E431A), or inactive scrambled PEST degron control (SCR). 72 hours after being transfected, the cells were collected for western blotting: mHTTex1-72Q-GFP was detected using a monoclonal antibody EM48 (Millipore, Cat #MAB5374). The intrabodies were detected by probing for their HA-tag with anti-HA, and GAPDH was probed as a loading control. (A) Live cell imaging. C4-PEST degron variants reduce the amount mHTTex1-72Q-eGFP to various levels compared to C4-PEST. C4 and C4-PEST-SCR prevent the aggregation of mHTT as demonstrated by presence of diffuse mHTTex1-72Q-GFP (arrows) compared to aggregated mHTTex1-72Q-GFP (asterisks) in EV CON cells. (B) Western blot. Graph shows quantification of the western blot by densitometry. The relative protein expression was determined by the ratio of soluble mHTTex1-72Q-GFP (EM48) to an internal standard control (GAPDH). Samples were then normalized to EV-CON. Human PEST degron variants T436A, S440A, E444A, and compound mutation variant E428/430/431A result in altered protein degradation levels compared to EV CON. C4-PEST and C4-PEST variant T436A reduced mHTT to 75-100% relative to control. C4-PEST variants E428A/E430/E431A and E444A reduced mHTT to 50-75% of control. The C4-PEST variant S440A reduced mHTT to 25-0% of control, whereas the C4, C4-PEST-SCR, and C4-PEST variant P438A all increased mHTT relative to control. FIG. 35—Shows human Ornithine Decarboxylase PEST (hPEST) degron variants targeting mHTT to the proteasome for degradation via a human PEST degron fusion. Certain single (A) and compound (B) mutations within the hPEST degron (highlighted in grey) are predicted to alter the targeted degradation of the intrabody and its bound antigen. This has been demonstrated for ones highlighted in bold, i.e., T436A, P438A, S440A, E444A, and E428A-E430A-E431A. The PEST degron is shaded in the top row (ODC amino acids 423-450). FIG. 35 discloses SEQ ID NOs.:3 and 110-132, respectively, in order of appearance.

FIG. 36—Shows anti-α-huntingtin scFv C4 and VL12.3 sequences: C4 variable heavy VH sequence (top), C4 variable light VL sequence (middle), and VL12.3 (bottom), corresponding to SEQ ID NO:106, SEQ ID NO:107, and SEQ ID NO:108, respectively. CDR=complementarity determining region.

FIGS. 37A-C—Show bifunctional anti-tau intrabodies significantly (p<0.05) reduce endogenous tau protein levels in human iPSC derived organoids. (A) Inducible lentiviral construct design. (B) organoid size after 1 day in vitro and 180 (DIV). (C) Quantitative western blot analysis of cell extracts from individual organoids WT at the MAPT gene transduced with bifunctional anti-tau-PEST (V-PEST, N-PEST, or F-PEST) intrabodies or empty virus control at 45DIV (n=3 per group). Organoids were then treated with 211 g/mL of doxycycline every other day to induce transgene expression for a duration of 21 days. V-PEST, N-PEST, and F-PEST significantly (p<0.05) reduced endogenous tau protein levels compared to untreated control (CON) and empty vector (EV) control.

### DETAILED DESCRIPTION

### Overview

This disclosure is based on the finding that modifications to the human PEST degron can alter the level of target antigen (synuclein, tau, huntingtin) degradation. Provided herein are polypeptides, e.g., bi-functional polypeptides, comprising an antigen binding domain of an antibody or functional fragment thereof, which binds to an epitope of an antigen (e.g., α-synuclein, tau, or huntingtin), and a programmable proteasome-targeting PEST motif. The bi-functional polypeptides are useful in the treatment and prevention of protein aggregation diseases, such as synucleinopathies and taupathies, Huntington's disease, and also spinal cord injury (SCI) and traumatic brain injury (TBI).

### α-Synuclein Protein

α-synuclein is a critical molecule for nervous system function. It builds up to toxic levels in a number of neurodegenerative diseases, as well as traumatic injury, and therefore, reducing the intracellular levels of the protein is a beneficial therapeutic approach. Because α-synuclein is an essential protein, it would be detrimental to cells to remove it all. Thus, provided herein are methods in which the therapeutic goal is to achieve a level of degradation of α-synuclein that reduces the amount of intracellular α-synuclein to levels that are not toxic to cells but not completely eliminate the protein. Rather, the level of α-synuclein is reduced to a desired level.

As described herein, an intrabody targeting α-synuclein provides both specificity to the protein and the PEST degron provides the target to the proteasome. Modification of the PEST degron by specific changes in the protein sequence provides the ability to regulate the level of degradation.

In synucleinopathies, α-synuclein undergoes an intracellular cascade of pathogenic misfolding, abnormal accumulation, and trans-cellular propagation. This process induces synuclein aggregation and neurotoxicity, as observed in vertebrate animal models, implicating this process as novel therapeutic target. However, none of these events proceed in the absence of the primary intracellular α-synuclein misfolding event. Therefore, targeting α-synuclein to prevent this pathological cascade is important. This was addressed by developing bi-functional intrabodies with the potential to eliminate synuclein accumulation using the cell's normal protein clearing process. Anti-α-synuclein intrabodies targeting synuclein to the proteasome for degradation were identified. To avoid a potential immunogenic response, the proteasomal targeting signal was optimized for human use by substitution of the mouse PEST degron with the human PEST (hPEST) degron from ornithine decarboxylase (ODC). One particular intrabody, referred to herein as VH14-hPEST, resulted in efficient degradation of endogenous α-synuclein in human induced pluripotent stem cell (iPSC)-derived neurons. Additionally, a novel anti-synuclein bi-functional intrabodies, N77D and DB1, can efficiently degrade both human and lamprey synuclein.

### Tau Protein

Tau is a critical molecule for nervous system function. It builds up to toxic levels in a number of neurodegenerative diseases, as well as traumatic injury, and therefore reducing the intracellular levels of the protein is a beneficial therapeutic approach. Because tau has important functions in the nervous system, it would be detrimental to remove it all. Thus, provided herein are methods in which the therapeutic goal is to achieve a level of degradation of tau that reduces the amount of intracellular tau to levels that are not toxic to cells but not completely eliminate the protein. Rather, the level of tau is reduced to a desired level.

As described herein, an intrabody targeting tau provides both specificity to the protein and the PEST degron provides the target to the proteasome. Modification of the PEST degron by specific changes in the protein sequence provides the ability to regulate the level of degradation.

Tau is a microtubule-associated phosphoprotein expressed in the central and peripheral nervous system. Tau plays a role in many biological processes such as microtubule stabilization, neurite outgrowth, neuronal migration, signal transduction, and organelle transport. Under normal conditions, tau expression is abundant within the axons of neurons. The misfolding and aggregation of tau within neurons are defining pathological hallmarks in a variety of tauopathies. The incidence of tauopathies represent an urgent and unmet medical need.

In tauopathies, tau protein may lose its ability to bind to microtubules, and as a result tau is mis-localized to the soma-dendritic compartment of the neuron. During this process, tau is hyperphosphorylated and misfolds into insoluble aggregates of straight filaments and paired helical filaments (PHF) which comprise neurofibrillary tangles and threads (NFTs). Tau hyperphosphorylation is presumed to occur prior to NFT formation. Furthermore, abnormal tau can recruit the properly folded isoform into misfolded complexes and, the abnormal form can be secreted from one cell to be taken up by other cells, which can trigger a cascade of misfolded tau complexes and disease spreading through the central nervous system.

Immunotherapy for the reduction in the intracellular levels of tau available for misfolding and/or aggregation represents a potential therapeutic approach for the treatment of tauopathies. Full-length antibodies that bind tau, however, have limited penetration into brain cells where tau protein aggregates reside.

### Huntingtin Protein

Huntingtin is a critical molecule for nervous system function. It builds up to toxic levels in a number of neurodegenerative conditions, most notably in Huntington's disease, and therefore reducing the intracellular levels of the protein is a beneficial therapeutic approach. Because Huntingtin has important functions in the nervous system, it would be detrimental to remove it all. Thus, provided herein are methods in which the therapeutic goal is to achieve a level of degradation of huntingtin that reduces the amount of intracellular huntingtin to levels that are not toxic to cells but not completely eliminate the protein. Rather, the level of huntingtin is reduced to a desired level.

Huntingtin is a protein present in many of the body's tissues and is the causal gene/protein (HTT) in Huntington's disease. The inherited mutation that causes Huntington's disease is known as a CAG trinucleotide repeat expansion. This mutation increases the size of the CAG segment in the HTT gene. People with Huntington's disease have 36 to more than 120 CAG repeats. People with 36 to 39 CAG repeats (SEQ ID NO: 135) may or may not develop the signs and symptoms of Huntington's disease, while people with 40 or more repeats almost always develop the disorder.

Although the exact function of the huntingtin protein is unknown, it appears to play an important role in nerve cells (neurons) in the brain and is essential for normal development before birth. Huntingtin is found in many of the body's tissues, with the highest levels of activity in the brain. Within cells, this protein may be involved in chemical signaling, transporting materials, attaching (binding) to proteins and other structures, and protecting the cell from self-destruction (apoptosis). Some studies suggest it plays a role in repairing damaged DNA.

One region of the HTT gene contains a particular DNA segment known as a CAG trinucleotide repeat. This segment is made up of a series of three DNA building blocks (cytosine, adenine, and guanine) that appear multiple times in a row. Normally, the CAG segment is repeated 10 to 35 times (SEQ ID NO: 136) within the gene.

The expanded CAG segment leads to the production of an abnormally long version of the huntingtin protein. The elongated protein is cut into smaller, toxic fragments that bind together and accumulate in neurons, disrupting the normal functions of these cells. It has also been suggested that loss of the huntingtin protein's DNA repair function may result in the accumulation of DNA damage in neurons, particularly as damaging molecules increase during aging. Regions of the brain that help coordinate movement and control thinking and emotions (the striatum and cerebral cortex) are particularly affected. The dysfunction and eventual death of neurons in these areas of the brain underlie the signs and symptoms of Huntington's disease.

As the altered HTT gene is passed from one generation to the next, the size of the CAG trinucleotide repeat often increases in size. A larger number of repeats is usually associated with an earlier onset of signs and symptoms. This phenomenon is called anticipation. People with the adult-onset form of Huntington's disease (which appears in mid-adulthood) typically have 40 to 50 CAG repeats (SEQ ID NO: 137) in the HTT gene, while people with the less common, juvenile form of the disorder (which appears in childhood or adolescence) tend to have more than 60 CAG repeats.

Individuals who have 27 to 35 CAG repeats (SEQ ID NO: 138) in the HTT gene do not develop Huntington's disease, but they are at risk of having children who will develop the disorder. As the gene is passed from parent to child, the size of the CAG trinucleotide repeat may lengthen into the range associated with Huntington's disease (36 repeats or more).

### Intrabodies Targeting α-Synuclein, Tau, and Huntingtin

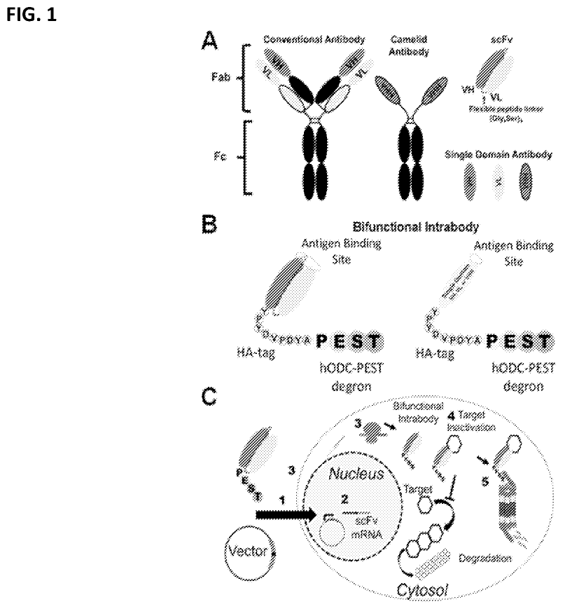

In some embodiments, an intrabody useful for achieving increased degradation of α-synuclein as described herein may have a structure as described herein (see FIG. 1A and FIG. 1B for schematic). In some embodiments, an intrabody may have a first domain having an antigen binding domain of an antibody or functional fragment thereof which binds to an epitope of an antigen of the disclosure (e.g., α-synuclein, tau, or huntingtin), and a second domain having a programmable proteasome-targeting PEST motif.

In some embodiments, an intrabody useful for increasing degradation of α-synuclein comprises a sequence that is at least 80%, at least 85%, at least 90%, at least 95%, at least 96%, at least 97%, at least 98%, at least 99%, or 100% identical to an amino acid as set forth in SEQ ID NO:5-18. In some embodiments, an intrabody useful for increasing degradation of α-synuclein consists of a sequence that is at least 80%, at least 85%, at least 90%, at least 95%, at least 96%, at least 97%, at least 98%, at least 99%, or 100% identical to an amino acid as set forth in SEQ ID NO:5-18.

In some embodiments, an intrabody useful for increasing degradation of α-tau comprises a sequence that is at least 80%, at least 85%, at least 90%, at least 95%, at least 96%, at least 97%, at least 98%, at least 99%, or 100% identical to an amino acid as set forth in SEQ ID NO:23-98. In some embodiments, an intrabody useful for increasing degradation of α-tau consists of a sequence that is at least 80%, at least 85%, at least 90%, at least 95%, at least 96%, at least 97%, at least 98%, at least 99%, or 100% identical to an amino acid as set forth in SEQ ID NO:23-98.

In some embodiments, an intrabody useful for increasing degradation of huntingtin comprises a sequence that is at least 80%, at least 85%, at least 90%, at least 95%, at least 96%, at least 97%, at least 98%, at least 99%, or 100% identical to an amino acid as set forth in SEQ ID NO:100-108. In some embodiments, an intrabody useful for increasing degradation of huntingtin consists of a sequence that is at least 80%, at least 85%, at least 90%, at least 95%, at least 96%, at least 97%, at least 98%, at least 99%, or 100% identical to an amino acid as set forth in SEQ ID NO:100-108.

In some embodiments, an intrabody useful for increasing degradation of α-synuclein, tau, or huntingtin comprises a flexible linker having a sequence that is at least 80%, at least 85%, at least 90%, at least 95%, at least 96%, at least 97%, at least 98%, at least 99%, or 100% identical to the amino acid as set forth in SEQ ID NO:14. In some embodiments, an intrabody useful for increasing degradation of α-synuclein comprises a flexible linker having a sequence that is at least 80%, at least 85%, at least 90%, at least 95%, at least 96%, at least 97%, at least 98%, at least 99%, or 100% identical to the amino acid as set forth in any one of SEQ ID NOs.:139-148.

In some embodiments, an epitope tag can be used to identify expression of the intrabody. Examples of epitope tags are known in the art and can include, but are not limited to, FLAG, 6× His (SEQ ID NO: 134), HA, c-myc, GST, Protein A, CD, Strep-tag, maltose-binding peptide (MBP), chitin-binding domain (CBD), S-tag, Avitag, CBP, TAP, SF-TAP. In one embodiment, an intrabody as described herein may have an HA tag for identification of expression of the intrabody under experimental conditions. In some embodiments, the HA tag does not affect the function of the intrabody. In some embodiments, an intrabody useful for increasing degradation of α-synuclein, tau, or huntingtin comprises an HA tag having a sequence that is at least 80%, at least 85%, at least 90%, at least 95%, at least 96%, at least 97%, at least 98%, at least 99%, or 100% identical to the amino acid as set forth in SEQ ID NO:15.

In some embodiments, an intrabody as described above comprises a single-chain antibody that comprises an α-synuclein-specific VH domain (VH-synuclein), or an α-synuclein-specific VHH antibody (i.e., nanobody), or antigen binding fragment thereof having an amino acid sequence that is at least 80%, at least 85%, at least 90%, at least 95%, at least 96%, at least 97%, at least 98%, at least 99% or 100% identical to an amino acid as set forth in SEQ ID NO:1, or set forth in SEQ ID NOs:4-13, or SEQ ID NOs:16-18.

The antigen binding domain of an antibody or functional fragment thereof can bind to unmodified or modified α-synuclein, and/or aggregated α-synuclein with high specificity and/or high affinity. The amino acid sequence of the human α-synuclein protein (Genbank® Accession No. CR541653) is provided as SEQ ID NO:4. In some embodiments, a specific number of amino acids at either the carboxy or the amino terminus can be targeted by an intrabody as described herein. For example, an intrabody may target a region or portion of the α-synuclein protein, such as including, but not limited to, a particular region or group of amino acids. In some embodiments, amino acids 53-95 of α-synuclein are targeted by an intrabody as described herein, to result in reduced phosphorylation of the protein.

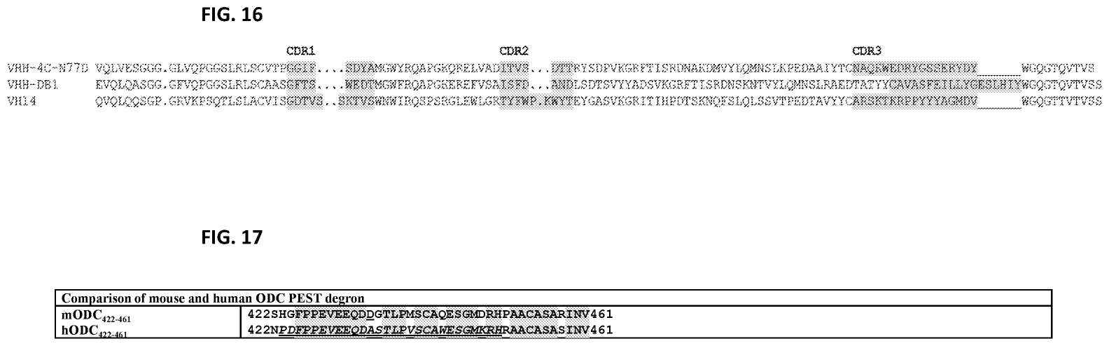

In some embodiments, an intrabody as described above comprises an scFv that comprises a tau-specific VH domain (VH-tau), or antigen binding fragments thereof having an amino acid sequence that is: at least 80%, at least 85%, at least 90%, at least 95%, at least 96%, at least 97%, at least 98%, at least 99% or 100% identical to an amino acid as set forth in SEQ ID NOs:65-81. In some embodiments, a VH domain (VH-tau) may comprise a CDR set forth in FIG. 16 (top).

In some embodiments, an intrabody as described above comprises an scFv that comprises a tau-specific VL domain (VL-tau), or antigen binding fragments thereof having an amino acid sequence that is: at least 80%, at least 85%, at least 90%, at least 95%, at least 96%, at least 97%, at least 98%, at least 99% or 100% identical to an amino acid as set forth in SEQ ID NOs:82-98. In some embodiments, a VL domain (VL-tau) may comprise a CDR set forth in FIG. 16 (bottom).

The antigen binding domain of an intrabody or antibody or functional fragment thereof can bind to phosphorylated tau, hyperphosphorylated tau, and/or aggregated tau with high specificity and/or high affinity. The amino acid sequence of the human tau protein (Genbank® Accession No. NP_005901) is provided as SEQ ID NO:22.

The antigen binding domain of an antibody or functional fragment thereof can bind to huntingtin and/or aggregated huntingtin with high specificity and/or high affinity. The amino acid sequence of the human huntingtin protein (Genbank® Accession No. NM 002111) is provided as SEQ ID NO:99. In some embodiments, a specific number of amino acids at either the carboxy or the amino terminus can be targeted by an intrabody as described herein. For example, an intrabody disclosed herein may target a region or portion of the huntingtin protein, such as including, but not limited to, a particular exon or intron of interest. In some embodiments, seventeen (17) amino acids at the amino terminus of the huntingtin gene may be targeted by an intrabody described herein. In some embodiments, exon 1, which includes the CAG trinucleotide repeat causative of Huntington's Disease, as well as a proline-rich region (PRR), is targeted by an intrabody described herein.

The antigen binding domain of an intrabody or antibody or functional fragment thereof may include, but is not limited to, single chain (scFv), single-chain (Fv)2 (sc(Fv)2), single domain antibodies (dAb; VH; VL), and diabodies. scFV and single domain antibodies retain the binding specificity of full-length antibodies, but they can be expressed as single genes. scFV and single domain VH or VL antibodies may be applied both extracellularly and intracellularly (intrabodies). In some embodiments, an intrabody can be a single-chain variable fragment (scFv), a variable heavy region (VH), a hypervariable region, a variable light region (VL), a VHH antibody (i.e., nanobody), a single-chain antigen-binding domain, or the like. In some embodiments, an intrabody (e.g., an α-synuclein, or tau intrabody) comprises a single-chain antigen-binding domain, referred to herein as a nanobody.

An scFv is a single-chain polypeptide antibody obtained by linking the VH and VL of an antibody with a linker. The order of VH's and VL's to be linked is not particularly limited, and they may be arranged in any order. Examples of arrangements include: [VH]-linker-[VL]; or [VL]-linker-[VH]. The heavy chain variable region (VH) and light chain variable region (VL) in an scFv may be derived from any antibody of the disclosure (e.g., anti-α-synuclein antibody, anti-tau antibody, or anti-huntingtin antibody), or antigen-binding fragment thereof described herein.

An sc(Fv)₂ contains two VH's and two VL's which are linked by a linker to form a single chain. An sc(Fv)₂ can be prepared, for example, by connecting scFvs with a linker. sc(Fv)₂'s may include two VH's and two VL's arranged in the order of: VH, VL, VH, and VL ([VH]-linker-[VL]-linker-[VH]-linker-[VL]), beginning from the N terminus of a single-chain polypeptide; however, the order of the two VH's and two VL's is not limited to the above arrangement, and they may be arranged in any order. Examples of arrangements include the following:
[VL]-linker-[VH]-linker-[VH]-linker-[VL]
[VH]-linker-[VL]-linker-[VL]-linker-[VH]
[VH]-linker-[VH]-linker-[VL]-linker-[VL]
[VL]-linker-[VL]-linker-[VH]-linker-[VH]
[VL]-linker-[VH]-linker-[VL]-linker-[VH]

In some embodiments, three linkers are required when four antibody variable regions are linked; the linkers used may be identical or different. In some embodiments, a linker as described herein may be a glycine-serine linker that connects the VH to the VL. In some embodiments, the linker length may be optimized to allow proper folding between the VH and VL in the intracellular compartment of cells. An exemplary linker that may be used in accordance with the present disclosure is set forth herein as SEQ ID NO:14. There is no particular limitation on the linkers that link the VH and VL regions of the scFvs or sc(FV)₂'s. In some embodiments, the linker is a peptide linker. Any arbitrary single-chain peptide comprising about three to about 25 residues (e.g., 5, 6, 7, 8, 9, 10, 11, 12, 13, 14, 15, 16, 17, 18, 19, 20, 21, 22, 23, 24, or 25) can be used as a linker.

In other embodiments, the linker is 10 to 20, 10 to 30, 10 to 40, 10 to 50, 10 to 60, 10 to 70, 10 to 80, 10 to 90, 10 to 100, 10 to 144, or 10 to 150 amino acids in length. In certain instances, the linker contains only glycine and/or serine residues. Examples of such peptide linkers include: Gly, Ser; Gly Ser; Gly Gly Ser; Ser Gly Gly; Gly Gly Gly Ser (SEQ ID NO:139); Ser Gly Gly Gly (SEQ ID NO:140); Gly Gly Gly Gly Ser (SEQ ID NO:141); Ser Gly Gly Gly Gly (SEQ ID NO:142); Gly Gly Gly Gly Gly Ser (SEQ ID NO:143); Ser Gly Gly Gly Gly Gly (SEQ ID NO:144); Gly Gly Gly Gly Gly Gly Ser (SEQ ID NO:145); Ser Gly Gly Gly Gly Gly Gly (SEQ ID NO:146); (Gly Gly Gly Gly Ser)n (SEQ ID NO:147)n, wherein n is an integer of one or more; and (Ser Gly Gly Gly Gly)n (SEQ ID NO:148)n, wherein n is an integer of one or more. In some instances, the linker has multiple copies (e.g., 1, 2, 3, 4, 5, 6, 7, 8, 9, 10) of the amino acid sequence of SEQ ID NO:139 with the exception that the serine residue in each copy of the linker is replaced with another amino acid.

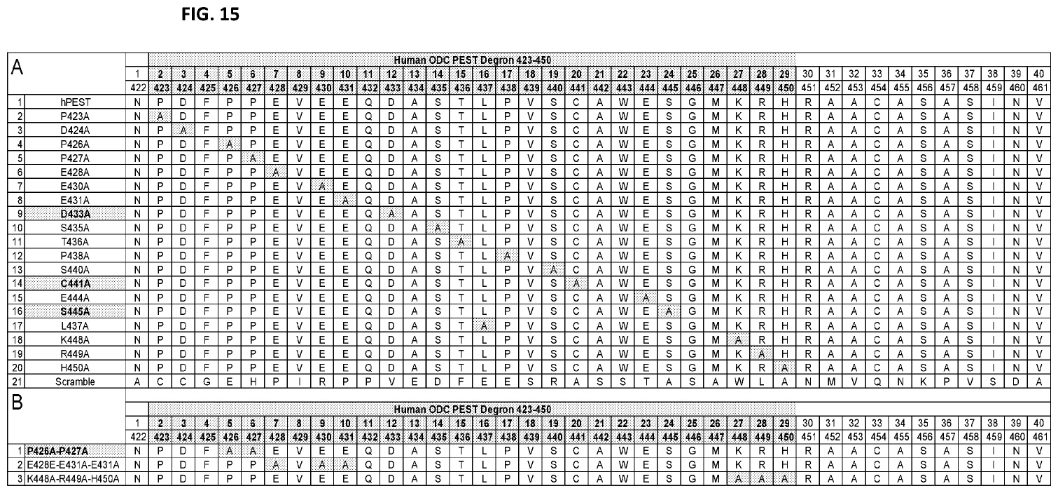

The amino acid sequence of the VH or VL in the antigen binding domain of an antibody or functional fragment thereof may include modifications such as substitutions, deletions, additions, and/or insertions. For example, modifications, such as substitutions, deletions, additions, and/or insertions, made within the amino acid sequence of the VH or VL may be in one or more of the CDRs. In certain embodiments, the modification involves one, two, or three amino acid substitutions in one or more CDRs and/or framework regions of the VH and/or VL domain of the anti-α-synuclein antigen binding domain of an antibody or functional fragment thereof. Such substitutions are made to improve the binding, functional activity and/or reduce immunogenicity of the antigen (e.g., α-synuclein, tau, huntingtin) binding domain of an antibody or functional fragment thereof. In certain embodiments, the substitutions are conservative amino acid substitutions. In some embodiments, one, two, or three amino acids of the CDRs of the antigen (e.g., α-synuclein, tau, huntingtin) binding domain of an antibody or functional fragment thereof may be deleted or added, so as long as there is antigen (e.g., α-synuclein, tau, huntingtin) binding and/or functional activity when VH and VL are associated. In some embodiments, a CDR may be a CDR provided in FIG. 15 and within SEQ ID NOs:16-18.

The proteasome-targeting PEST motif is a peptide sequence containing regions enriched in prolyl (P), glutamyl (E), aspartyl (D), seryl (S) and threonyl (T) residues (PEST regions) and are targeted for accelerated proteasomal degradation. This sequence is associated with proteins that have a short intracellular half-life. Mouse Ornithine Decarboxylase (MODC) is one of the shortest half-lived proteins in mammals. The constitutive degradation of MODC by the proteasome is controlled by PEST sequences in its carboxy terminus (amino acids 422-461).

Exemplary murine-derived PEST motif sequences include, for example, an amino acid sequence at least 80%, at least 85%, at least 90%, at least 95%, at least 96%, at least 97%, at least 98%, at least 99%, or 100% identical to an amino acid sequence as set forth in SEQ ID NO:2 (SHGFPPEVEEQDDGTLPMSCAQESGMDRHPAACASARINV) and corresponding to ornithine decarboxylase (ODC) amino acids 422-461.

Exemplary human-derived PEST motif sequences (hPEST) include, for example, an amino acid sequence at least 80%, at least 85%, at least 90%, at least 95%, at least 96%, at least 97%, at least 98%, at least 99% or 100% identical to an amino acid sequence as set forth in SEQ ID NO:3 (NPDFPPEVEEQDASTLPVSCAWESGMKRHRAACASASINV) and corresponding to human ornithine decarboxylase (ODC) amino acids 422-461.

A comparison of mouse PEST (mPEST; SEQ ID NO:2) and human PEST (hPEST; SEQ ID NO:3) sequences is provided in Table 1, demonstrating 82.5% sequence homology between mouse mPEST and human hPEST.

TABLE 1
Comparison of mouse PEST and human PEST sequences

| | |
|---|---|
| mPEST | SHGFPPEVEEQDDGTLPMSCAQESGMDRHPAACASARINV |
| (ODC) | (SEQ ID NO: 2) |
| hPEST | NPDFPPEVEEQDASTLPVSCAWESGMKRHRAACASASINV |
| (ODC) | (SEQ ID NO: 3) |

In some embodiments, the PEST degron is any one of the sequences disclosed in International Patent Publication WO2018049219 (PCT/US2017/050764), which is incorporated herein in its entirety.

The term “% identical” between two polypeptide (or polynucleotide) sequences refers to the number of identical matched positions shared by the sequences over a comparison window, taking into account additions or deletions (i.e., gaps) that must be introduced for optimal alignment of the two sequences. A matched position is any position where an identical nucleotide or amino acid is presented in both the target and reference sequence. Gaps presented in the target sequence are not counted since gaps are not nucleotides or amino acids. Likewise, gaps presented in the reference sequence are not counted since target sequence nucleotides or amino acids are counted, not nucleotides or amino acids from the reference sequence. The percentage of sequence identity is calculated by determining the number of positions at which the identical amino acid residue or nucleic acid base occurs in both sequences to yield the number of matched positions, dividing the number of matched positions by the total number of positions in the window of comparison and multiplying the result by 100 to yield the percentage of sequence identity. The comparison of sequences and determination of percent sequence identity between two sequences can be accomplished using readily available software both for online use and for download. Suitable software programs are available from various sources, and for alignment of both protein and nucleotide sequences. One suitable program to determine percent sequence identity is bl2seq, part of the BLAST suite of program available from the U.S. government's National Center for Biotechnology Information BLAST web site (blast.ncbi.nlm.nih.gov). Bl2seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at ebi.ac.uk/Tools/psa. In certain embodiments, the percentage identity “X” of a first amino acid sequence to a second sequence amino acid is calculated as 100×(Y/Z), where Y is the number of amino acid residues scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence. One skilled in the art will appreciate that the generation of a sequence alignment for the calculation of a percent sequence identity is not limited to binary sequence-sequence comparisons exclusively driven by primary sequence data. Sequence alignments can be derived from multiple sequence alignments. One suitable program to generate multiple sequence alignments is Clustal Omega, available from clustal.org. Another suitable program is MUSCLE, available from drive5.com/muscle. ClustalW2 and MUSCLE are alternatively available, e.g., from the EBI. The most preferred program of use is Clustal Omega.

The terms “linked” or “fused” refers to linkage via a peptide bonds (e.g., genetic fusion), chemical conjugation, or other means known in the art. For example, one way in which molecules or moieties can be linked employs peptide linkers that link the molecules or moieties via peptide bonds.

The term “associated with” refers to a covalent or non-covalent bond formed between a first amino acid chain and a second amino acid chain. In one embodiment, the term “associated with” means a covalent, non-peptide bond or a non-covalent bond. In another embodiment, the term “associated with” refers to a covalent, non-peptide bond or a non-covalent bond that is not chemically crosslinked. In another embodiment, it means a covalent bond except a peptide bond. In some embodiments this association is indicated by a colon, i.e., (:).

### Method of Producing Polypeptides

The bi-functional polypeptides (or antigen binding domain of an antibody or functional fragment thereof) described herein may be produced in bacterial or eukaryotic cells. To produce the polypeptide, a polynucleotide encoding the polypeptide is constructed, introduced into an expression vector, and then expressed in suitable host cells. Standard molecular biology techniques are used to prepare the recombinant expression vector, transfect the host cells, select for transformants, culture the host cells and recover the antibody.

If the polypeptide is to be expressed in bacterial cells (e.g., E. coli), the expression vector should have characteristics that permit amplification of the vector in the bacterial cells. Additionally, when E. coli, such as JM109, DH5a, HB101, or XL1-Blue is used as a host, the vector must have a promoter, for example, a lacZ promoter, araB promoter, or T7 promoter that can allow efficient expression in E. coli. Examples of such vectors include, for example, M13-series vectors, pUC-series vectors, pBR322, pBluescript, pCR-Script, pGEX-5X-1 (Pharmacia), “QIAexpress system” (QIAGEN), pEGFP, and pET (when this expression vector is used, the host is, in some embodiments, BL21 expressing T7 RNA polymerase).

The expression vector may contain a signal sequence for antibody secretion. For production into the periplasm of E. coli, the pelB signal sequence may be used as the signal sequence for antibody secretion. For bacterial expression, calcium chloride methods or electroporation methods may be used to introduce the expression vector into the bacterial cell.

In one embodiment, the polypeptides are produced in mammalian cells. Exemplary mammalian host cells for expressing a polypeptide include Chinese Hamster Ovary (CHO cells) (including dhfr CHO cells, used with a DHFR selectable marker, human embryonic kidney 293 cells (e.g., 293, 293E, 293T), COS cells, NIH3T3 cells, lymphocytic cell lines, e.g., NS0 myeloma cells and SP2 cells, and a cell from a transgenic animal, e.g., a transgenic mammal.

If the polypeptide is to be expressed in mammalian cells such as CHO, COS, 293, 293T, and NIH3T3 cells, the expression vector includes a promoter necessary for expression in these cells, for example, an SV40 promoter, MMLV-LTR promoter, EFla promoter, or CMV promoter. In addition to the nucleic acid sequence encoding the immunoglobulin or domain thereof, the recombinant expression vectors may carry additional sequences, such as sequences that regulate replication of the vector in host cells (e.g., origins of replication) and selectable marker genes. The selectable marker gene facilitates selection of host cells into which the vector has been introduced. For example, typically the selectable marker gene confers resistance to drugs, such as G418, hygromycin, or methotrexate, on a host cell into which the vector has been introduced. Examples of vectors with selectable markers include pMAM, pDR2, pBK-RSV, pBK-CMV, pOPRSV, and pOP13.

The polypeptides described herein can be isolated from inside or outside (such as medium) of the host cell and purified as substantially pure and homogenous antibodies. Methods for isolation and purification commonly used for polypeptides purification may be used for the isolation and purification of polypeptides, and are not limited to any particular method. Polypeptides may be isolated and purified by appropriately selecting and combining, for example, column chromatography, filtration, ultrafiltration, salting out, solvent precipitation, solvent extraction, distillation, immunoprecipitation, SDS-polyacrylamide gel electrophoresis, isoelectric focusing, dialysis, and recrystallization. Chromatography includes, for example, affinity chromatography, ion exchange chromatography, hydrophobic chromatography, gel filtration, reverse-phase chromatography, and adsorption chromatography. Chromatography can be carried out using liquid phase chromatography such as HPLC and FPLC. Columns used for affinity chromatography include protein A column and protein G column. Examples of columns using protein A column include Hyper D, POROS, and Sepharose FF (GE Healthcare Biosciences). Disclosed also are polypeptides that are highly purified using these purification methods.

### Characterization of the Antigen Binding Domain of an Antibody or Antigen Binding Functional Fragment Thereof

The antigen-binding properties of a polypeptide (e.g., α-synuclein-binding, tau-binding, or huntingtin-binding) described herein may be measured by any standard method, e.g., one or more of the following methods: OCTET®, Surface Plasmon Resonance (SPR), BIACORE™ analysis, Enzyme Linked Immunosorbent Assay (ELISA), EIA (enzyme immunoassay), RIA (radioimmunoassay), and Fluorescence Resonance Energy Transfer (FRET).

The binding interaction of a protein of interest (anti-synuclein, anti-tau, or anti-huntingtin antibody-binding domain or functional fragment thereof) and a target (e.g., α-synuclein, tau, or huntingtin) can be analyzed using the OCTET ° systems. In this method, one of several variations of instruments (e.g., OCTET ° QKe and QK), made by the ForteBio company are used to determine protein interactions, binding specificity, and epitope mapping. The OCTET ° systems provide an easy way to monitor real-time binding by measuring the changes in polarized light that travels down a custom tip and then back to a sensor.

The binding interaction of a protein of interest (an anti-synuclein, anti-tau, or anti-huntingtin antibody-binding domain or functional fragment thereof) and a target (e.g., α-synuclein, tau, or huntingtin) can be analyzed using Surface Plasmon Resonance (SPR). SPR or Biomolecular Interaction Analysis (BIA) detects biospecific interactions in real time, without labeling any of the interactants. Changes in the mass at the binding surface (indicative of a binding event) of the BIA chip result in alterations of the refractive index of light near the surface (the optical phenomenon of surface plasmon resonance (SPR)). The changes in the refractivity generate a detectable signal, which is measured as an indication of real-time reactions between biological molecules. Methods for using SPR are known and described in the art. Information from SPR can be used to provide an accurate and quantitative measure of the equilibrium dissociation constant (K_{d}), and kinetic parameters, including Kₒₙ and K_{off} for the binding of a biomolecule to a target.

Epitopes can also be directly mapped by assessing the ability of different anti-α-synuclein antibody binding domains or functional fragment thereof to compete with each other for binding to human α-synuclein, tau, or synuclein using BIACORE chromatographic techniques.

When employing an enzyme immunoassay, a sample containing an antibody, for example, a culture supernatant of antibody-producing cells or a purified antibody is added to an antigen-coated plate. A secondary antibody labeled with an enzyme such as alkaline phosphatase is added, the plate is incubated, and after washing, an enzyme substrate such as p-nitrophenylphosphate is added, and the absorbance is measured to evaluate the antigen binding activity.

Additional general guidance for evaluating antibodies, e.g., western blots and immunoprecipitation assays, can be found in Antibodies: A Laboratory Manual, ed. by Harlow and Lane, Cold Spring Harbor press (1988)).

### Methods of Treatment

Provided is a method for treatment or prevention of protein aggregation caused by diseases or trauma that result in aggregation of α-synuclein, such as Parkinson's Disease, Multiple System Atrophy, spinal cord injury (SCI) or traumatic brain injury (TBI), comprising administration of a therapeutically effective amount of a gene therapy encoding an anti-α-synuclein bi-functional intrabody as described herein to a patient in need thereof.

In some embodiments, the methods described herein may treat or prevent protein aggregation resulting from a spinal cord injury or traumatic brain injury, as described herein in a subject or patient as described herein. Administration of a composition comprising a gene therapy encoding an anti-α-synuclein intrabody as described herein may be in a clinical setting as described herein, or may be in an alternate setting as deemed appropriate by a clinician or practitioner.

In some embodiments, such a composition comprising a gene therapy encoding an anti-α-synuclein intrabody as described herein may be combined with other therapies or treatments for treatment of the brain injury or spinal cord injury in a patient. Other drug treatments may be used as deemed appropriate by a clinician.

The bi-functional polypeptides described herein also can be used, either alone or in combination with other therapies, in the treatment, including prevention, of synucleinopathies, such as, but not limited to, Parkinson's Disease (PD), Multiple System Atrophy (MSA), Alzheimer's disease (AD), Frontotemporal Dementia (FTD), including Fronto-temporal Dementia with Parkinsonism on chromosome-17 (FTDP-17), Pick's disease, Corticobasal Degeneration (CBD), Progressive Supranuclear Palsy (PSP), Chronic Traumatic Encephalopathy (CTE), Lytico-Bodig disease, Ganglioglioma and gangliocytoma, Meningioangiomatosis, Subacute sclerosing panencephalitis, lead encephalopathy, Tuberous sclerosis, and Hallervorden-Spatz disease and treatment of traumatic damage such as traumatic brain injury (TBI) or spinal cord injury (SCI). Such methods comprise administering to a subject in need thereof (e.g., a subject suffering from or at risk of having a synucleinopathy) a therapeutically effective amount of a bi-functional polypeptide, which comprises a first domain comprising an antigen binding domain of an antibody or fragment thereof which binds to an epitope of α-synuclein; and a second domain comprising a programmable proteasome-targeting PEST motif. As would be understood by one of skill in the art, a bi-functional polypeptide as described herein is administered in a form necessary or useful to the subject for treatment of, e.g., a synucleinopathy, such as a gene therapy encoding the bi-functional polypeptide.

Provided also is a method for treatment or prevention of protein aggregation caused by diseases of trauma that result in aggregation of tau, such as, but not limited to Alzheimer's disease (AD), Frontotemporal dementia (FTD), Fronto-temporal Dementia with Parkinsonism on chromosome-17 (FTDP-17), Pick's disease, Corticobasal Degeneration (CBD), Progressive Supranuclear Palsy (PSP), Chronic traumatic encephalopathy (CTE), Lytico-Bodig disease, Ganglioglioma and gangliocytoma, Meningioangiomatosis, Subacute sclerosing panencephalitis, lead encephalopathy, Tuberous sclerosis and Hallervorden-Spatz disease. Such methods comprise administering to a subject in need thereof (e.g., a subject suffering from or at risk of having a tauopathy) a therapeutically effective amount of a bi-functional intrabody polypeptide, which comprises a first domain comprising an antigen binding domain of an antibody or fragment thereof which binds to an epitope of tau; and a second domain comprising a programmable proteasome-targeting PEST motif. As would be understood by one of skill in the art, a bi-functional polypeptide as described herein is administered in a form necessary or useful to the subject for treatment of, e.g., a tauopathy, such as a gene therapy encoding the bi-functional polypeptide.

The bi-functional polypeptides described herein can be used in the treatment, including prevention, of diseases associated with huntingtin, such as Huntington's disease. Such methods comprise administering to a subject in need thereof (e.g., a subject suffering from or at risk of having Huntington's disease) a therapeutically effective amount of a bi-functional polypeptide, which comprises a first domain comprising an antigen binding domain of an antibody or fragment thereof which binds to an epitope of huntingtin; and a second domain comprising a programmable proteasome-targeting PEST motif. As would be understood by one of skill in the art, a bi-functional polypeptide as described herein is administered in a form necessary or useful to the subject for treatment of Huntington's Disease, such as a gene therapy encoding the bi-functional polypeptide.

Gene therapies and their uses are known in the art and can include, in some embodiments, administration of a nucleic acid, such as a DNA or RNA construct, for example a stabilized RNA construct, or of a bi-functional polypeptide in a form that enables its biological function in the cytoplasm of the cell. In some embodiments, a nucleic acid may be administered in a vector, such as a gene therapy vector encoding a bi-functional polypeptide as described herein. In other embodiments, a gene therapy useful for a bi-functional polypeptide may be administered in a formulation or composition that is optimized for uptake or delivery into a particular cell type, such as through the use of cell-specific receptors or genetic promoters. For example, in some embodiments, a genetic promoter may be useful for targeting a bi-functional polypeptide as described herein to oligodendrocytes for specific treatment of some diseases. In some embodiments, use of a specific genetic promoter may allow targeted expression of a bi-functional polypeptide as described herein restricted to certain cell types, such as neurons, astrocytes, and/or oligodendrocytes. In other embodiments, expression within a certain sub-population of a cell type, such as dopaminergic neurons or glutamatergic neurons, may be accomplished with the use of a tyrosine hydroxylase promoter or a VGLUT1 promoter, respectively. In some embodiments, expression within a certain sub-population of a cell type, such as excitatory neurons, may be accomplished with the use of, for example, a VGLUT1 promoter.

Also provided is a method for treatment or prevention of protein aggregation caused by spinal cord injury (SCI) or traumatic brain injury (TBI), or a disease such as a tauopathy comprising administration of a therapeutically effective amount of a gene therapy encoding an anti-tau intrabody as described herein to a patient in need thereof.

In some embodiments, the methods described herein may treat or prevent protein aggregation resulting from a spinal cord injury or traumatic brain injury, or a tauopathy as described herein in a subject or patient as described herein. Administration of a composition comprising a gene therapy encoding an anti-tau intrabody as described herein may be in a clinical setting as described herein, or may be in an alternate setting as deemed appropriate by a clinician or practitioner. Further embodiments for administration of such an anti-tau intrabody are described herein elsewhere.

In some embodiments, such a composition comprising a gene therapy encoding an anti-tau intrabody as described herein may be combined with other therapies or treatments for treatment of tauopathies or TBI or spinal cord injury (SCI) in a patient.

The term “subject” refers to an animal or human, or to one or more cells derived from an animal or human. Preferably, the subject is a human. Subjects can also include non-human primates.

In some embodiments, provided herein is a method for treatment or prevention of protein aggregation caused by a disease such as Huntington's disease or TBI or SCI comprising administration of a therapeutically effective amount of an anti-huntingtin intrabody as described herein to a patient in need thereof.

A method disclosed herein may treat or prevent protein aggregation resulting from a spinal cord injury or traumatic brain injury, or Huntington's disease as described herein in a subject or patient as described herein. Administration of a composition comprising an anti-huntingtin intrabody as described herein may be in a clinical setting as described herein, or may be in an alternate setting as deemed appropriate by a clinician or practitioner. Further embodiments for administration of such an anti-huntingtin intrabody are described herein elsewhere.

In some embodiments, such a composition comprising an anti-huntingtin intrabody as described herein may be combined with other therapies or treatments for treatment of Huntington's disease or a related neurodegenerative or neurotraumatic condition in a patient. Other drug treatments may be used as deemed appropriate by a clinician.

Unless otherwise specified herein, the methods described herein can be performed in accordance with the procedures exemplified herein or routinely practiced methods well known in the art. The following sections provide additional guidance for practicing the methods described herein.

### Pharmaceutical Compositions

As described herein, a bi-functional polypeptide as described herein can be formulated as a pharmaceutical composition, such as a gene therapy encoding a bi-functional polypeptide suitable for administration to a subject, e.g., to treat a disorder described herein. Typically, a pharmaceutical composition includes a pharmaceutically acceptable carrier. Pharmaceutical formulation is well established and known in the art.

The pharmaceutical compositions described herein may be in a variety of forms. These include, for example, liquid, semi-solid and solid dosage forms, such as liquid solutions (e.g., injectable and infusible solutions), dispersions or suspensions, tablets, pills, powders, liposomes and suppositories. The form can depend on the intended mode of administration and therapeutic application. Typically, compositions for the agents described herein are in the form of injectable or infusible solutions.

In one embodiment, a gene therapy encoding a bi-functional polypeptide described herein is formulated with excipient materials, such as sodium citrate, sodium dibasic phosphate heptahydrate, sodium monobasic phosphate, Tween-80, and a stabilizer. It can be provided, for example, in a buffered solution at a suitable concentration and can be stored at 2-8° C. In some other embodiments, the pH of the composition is between about 5.5 and about 7.5 (e.g., 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, or 7.5).

The pharmaceutical compositions can also include agents that reduce aggregation of the bi-functional polypeptide when formulated. Examples of aggregation reducing agents include one or more amino acids selected from methionine, arginine, lysine, aspartic acid, glycine, and glutamic acid. The pharmaceutical compositions can also include a sugar (e.g., sucrose, trehalose, mannitol, sorbitol, or xylitol) and/or a tonicity modifier (e.g., sodium chloride, mannitol, or sorbitol) and/or a surfactant (e.g., polysorbate-20 or polysorbate-80).

Such compositions can be administered by a parenteral mode (e.g., intravenous, subcutaneous, intraperitoneal, or intramuscular injection). In one embodiment, the bi-functional polypeptide compositions are administered subcutaneously. In one embodiment, the bi-functional polypeptide compositions are administered intravenously. The phrases “parenteral administration” and “administered parenterally” as used herein mean modes of administration other than enteral and topical administration, usually by injection, and include, without limitation, intravenous, intramuscular, intra-arterial, intrathecal, intracapsular, intraocular, intracardiac, intradermal, intraperitoneal, transtracheal, subcutaneous, subcuticular, intraarticular, subcapsular, subarachnoid, intraspinal, epidural and intrasternal injection, and infusion.

The composition can be formulated as a solution, microemulsion, dispersion, liposome, or other ordered structure suitable for stable storage at high concentration. Sterile injectable solutions can be prepared by incorporating an agent described herein in the required amount in an appropriate solvent with one or a combination of ingredients enumerated above, as required, followed by filtered sterilization. Generally, dispersions are prepared by incorporating an agent described herein into a sterile vehicle that contains a basic dispersion medium and the required other ingredients from those enumerated above. In the case of sterile powders for the preparation of sterile injectable solutions, the methods of preparation are vacuum drying and freeze drying that yield a powder of an agent described herein plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

In certain embodiments, a composition or gene therapy encoding a bi-functional polypeptide as described herein may be prepared with a carrier that will protect the compound against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known.

In some embodiments, a composition comprising a gene therapy encoding the bi-functional polypeptide is formulated in sterile distilled water or phosphate buffered saline. The pH of the pharmaceutical formulation may be between about 5.5 and about 7.5 (e.g., 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, or 7.5).

### Administration of Polypeptides

A composition comprising a gene therapy encoding a polypeptide (e.g., a bi-functional polypeptide) as described herein can be administered to a subject, e.g., a subject in need thereof, for example, a human or animal subject, by a variety of methods. For many applications, the route of administration is one of: intravenous injection or infusion (IV), subcutaneous injection (SC), intraperitoneally (IP), or intramuscular injection. Other modes of parenteral administration can also be used. Examples of such modes include: intraarterial, intrathecal, intracapsular, intraocular, intracardiac, intradermal, transtracheal, subcuticular, intraarticular, subcapsular, subarachnoid, intraspinal, and epidural and intrasternal injection.

The route and/or mode of administration of the bi-functional polypeptide can also be tailored for the individual case, e.g., by monitoring the subject.

The composition comprising a gene therapy encoding a bi-functional polypeptide can be administered as a fixed dose, or in a mg/kg dose. The dose can also be chosen to reduce or avoid production of antibodies against the bi-functional polypeptide. Dosage regimens are adjusted to provide the desired response, e.g., a therapeutic response or a combinatorial therapeutic effect. Generally, doses of the bi-functional polypeptide (and optionally a second agent) can be used in order to provide a subject with the agent in bioavailable quantities.

Dosage unit form or “fixed dose” as used herein refers to physically discrete units suited as unitary dosages for the subjects to be treated; each unit contains a predetermined quantity of bi-functional polypeptide calculated to produce the desired therapeutic effect in association with the required pharmaceutical carrier and optionally in association with the other agent. Single or multiple dosages may be given. Alternatively, or in addition, the composition comprising the gene therapy encoding a bi-functional polypeptide may be administered via continuous infusion.

A composition comprising a gene therapy encoding a bi-functional polypeptide dose can be administered in one dose or multiple times, e.g., at a periodic interval over a period of time (a course of treatment) sufficient to encompass at least 2 doses, 3 doses, 5 doses, 10 doses, or more, e.g., once or twice daily, or about one to four times per week, or such as weekly, biweekly (every two weeks), every three weeks, monthly, e.g., for between about 1 to 12 weeks, such as between 2 to 8 weeks, such as between about 3 to 7 weeks, and such as for about 4, 5, or 6 weeks. Factors that may influence the dosage and timing required to effectively treat a subject, include, e.g., the stage or severity of the disease or disorder, formulation, route of delivery, previous treatments, the general health and/or age of the subject, and other diseases present. Moreover, treatment of a subject with a therapeutically effective amount of a compound can include a single treatment or can include a series of treatments.

If a subject is at risk for developing a disorder described herein, the bi-functional polypeptide can be administered before the full onset of the disorder, e.g., as a preventative measure. The duration of such preventative treatment can be a single dosage of the composition or the treatment may continue (e.g., multiple dosages). For example, a subject at risk for the disorder or who has a predisposition for the disorder may be treated with a composition as described herein for days, weeks, months, or even years so as to prevent the disorder from occurring or fulminating.

The composition comprising a gene therapy encoding a bi-functional polypeptide can be administered to a patient in need thereof (e.g., a patient that has had or is at risk of having a protein aggregation disease, such as a synucleinopathy, a tauopathy or Huntington's Disease) alone or in combination with (i.e., by co-administration or sequential administration) other therapeutic proteins (e.g., antibodies, intrabodies, polypeptides) useful for treating a synucleinopathy, a tauopathy or Huntington's Disease may be desirable. In one embodiment, the additional therapeutic proteins are included in the pharmaceutical composition described herein. Examples of therapeutic proteins which can be used to treat a subject include, but are not limited to, therapeutic proteins targeting β-amyloid, α-synuclein, huntingtin, TDP-43, and/or SOD-1.

The composition can be administered to a patient in need thereof (e.g., a patient that has or is at risk of having a protein aggregation disease, such as a synucleinopathy, tauopathy, or Huntington's Disease) in combination with (i.e., by co-administration or sequential administration) other neuroprotective agents useful for treating a protein aggregation disease, such as a synucleinopathy or a tauopathy. In one embodiment, the additional agent is comprised of the pharmaceutical composition described herein. Examples of neuroprotective agents include, but are not limited to, an acetylcholinesterase inhibitor, a glutamatergic receptor antagonist, kinase inhibitors, HDAC inhibitors, anti-inflammatory agents, divalproex sodium, dopamine or a dopamine receptor agonist, or any combination thereof.

In some aspects, the composition comprising a gene therapy encoding a bi-functional polypeptide described herein can be used in methods designed to express the bi-functional polypeptide intracellularly so as to bind intracellular α-synuclein, tau, or huntingtin. Such methods comprise delivering to a cell a bi-functional polypeptide which may be in any form used by one skilled in the art, for example, a protein, an RNA molecule which is translated, or a DNA vector which is transcribed and translated.

In instances where a polynucleotide molecule encoding a bi-functional polypeptide is used, the polynucleotide may be recombinantly engineered into a variety of host vector systems that can be introduced in vivo such that it is taken up by a cell and directs the transcription of the bi-functional polypeptide molecule. Such a vector can remain episomal or become chromosomally integrated, as long as it can be expressed to produce the desired polypeptide. Such vectors can be constructed by recombinant DNA technology methods that are well known and standard in the art. Vectors encoding the domain intrabody of interest can be plasmid, viral, or others known in the art, used for replication and expression in mammalian cells.

A wide variety of viral and non-viral vectors for delivery of a polynucleotide encoding a bi-functional polypeptide described herein are known in the art and may be employed in making the products and practicing the methods described herein. Vectors include, for example, eukaryotic expression vectors, including but not limited to viral expression vectors such as those derived from the class of retroviruses, adenoviruses or adeno-associated viruses.

Some examples of suitable viral vectors include retrovirus-based vectors (e.g., lentiviruses), adenoviruses, adeno-associated viruses (AAV), Herpes vectors, and vaccinia vectors. In some embodiments, the structure of the vector may be modified as necessary for optimization of expression or to achieve a desired cellular level, of the recombinant polypeptide, such as including expression controlling elements (e.g., promoter or enhancer sequences). In some embodiments, expression of a programmable PEST degron sequence as described herein may be accomplished with the use of a strong promoter that produces high rates of gene transcription in a cell. Various vector systems are known to those skilled in the art and can be used to transfer the compositions described herein into cells, e.g., encapsulation in liposomes, microparticles, microcapsules, recombinant cells capable of expressing the composition, construction of a nucleic acid as part of a retroviral, adenoviral, adeno-associated viral or other vector, injection of DNA, electroporation, calcium phosphate-mediated transfection, etc.

### Devices and Kits for Therapy

Pharmaceutical compositions that include a gene therapy encoding the bi-functional polypeptide described herein can be administered with a medical device. The device can be designed with features such as portability, room temperature storage, and ease of use so that it can be used in emergency situations, e.g., by an untrained subject or by emergency personnel in the field, removed from medical facilities and other medical equipment. The device can include, e.g., one or more housings for storing pharmaceutical preparations that include a gene therapy encoding a bi-functional polypeptide, and can be configured to deliver one or more unit doses of the antibody. The device can be further configured to administer a second agent, e.g., a neuroprotective agent, either as a single pharmaceutical composition that also includes the gene therapy encoding the bi-functional polypeptide or as two separate pharmaceutical compositions.

A gene therapy encoding a bi-functional polypeptide can be provided in a kit. In one embodiment, the kit includes (a) a container that contains a composition that includes a gene therapy encoding a bi-functional polypeptide as described herein, and optionally (b) informational material. The informational material can be descriptive, instructional, marketing or other material that relates to the methods described herein and/or the use of the agents for therapeutic benefit.

In an embodiment, the kit also includes a second agent for treating a disorder described herein. For example, the kit includes a first container that contains a composition that includes the gene therapy encoding the bi-functional polypeptide, and a second container that includes the second agent.

The informational material of the kits is not limited in its form. In one embodiment, the informational material can include information about production of the compound, molecular weight of the compound, concentration, date of expiration, batch or production site information, and so forth. In one embodiment, the informational material relates to methods of administering the gene therapy encoding the bi-functional polypeptide, e.g., in a suitable dose, dosage form, or mode of administration (e.g., a dose, dosage form, or mode of administration described herein), to treat a subject who has had or who is at risk for a protein aggregation disease, such as a synucleinopathy, tauopathy, or Huntington's Disease described herein. The information can be provided in a variety of formats, include printed text, computer readable material, video recording, or audio recording, or information that provides a link or address to substantive material, e.g., on the internet.

In addition to the gene therapy encoding the bi-functional polypeptide, the composition in the kit can include other ingredients, such as a solvent or buffer, a stabilizer, or a preservative. The gene therapy encoding the bi-functional polypeptide can be provided in any form, e.g., liquid, dried or lyophilized form, substantially pure and/or sterile. When the agents are provided in a liquid solution, the liquid solution is an aqueous solution. When the agents are provided as a lyophilized product, the lyophilized powder is generally reconstituted by the addition of a suitable solvent. The solvent, e.g., sterile water or buffer (e.g., PBS), can optionally be provided in the kit.

The kit can include one or more containers for the composition or compositions containing the agents. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the agents. The containers can include a combination unit dosage, e.g., a unit that includes both the gene therapy encoding the bi-functional polypeptide and the second agent, e.g., in a desired ratio. For example, the kit includes a plurality of syringes, ampules, foil packets, blister packs, or medical devices, e.g., each containing a single combination unit dose. The containers of the kits can be air-tight, waterproof (e.g., impermeable to changes in moisture or evaporation), and/or light-tight.

The kit optionally includes a device suitable for administration of the composition, e.g., a syringe or other suitable delivery device. The device can be provided pre-loaded with one or both of the agents or can be empty, but suitable for loading.

### Definitions

Where a range of values is provided, it is understood that each intervening value, to the tenth of the unit of the lower limit unless the context clearly dictates otherwise, between the upper and lower limit of that range and any other stated or intervening value in that stated range, is provided herein. The upper and lower limits of these smaller ranges may independently be included in the smaller ranges, and are also provided herein, subject to any specifically excluded limit in the stated range. Where the stated range includes one or both of the limits, ranges excluding either or both of those included limits are also provided.

Unless defined otherwise, all technical and scientific terms used herein have the same meaning as commonly understood by one of ordinary skill in the relevant art. Although any methods and materials similar or equivalent to those described herein can also be used in the practice or testing of the embodiments herein, the preferred methods and materials are now described. All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

Unless defined otherwise, all technical and scientific terms used herein have the meaning commonly understood by one of ordinary skill in the relevant art. Specific terminology of particular is defined below.

As used in this specification and the appended claims, the singular forms “a,” “an,” and “the,” along with similar references used in the context of describing a particular embodiment (especially in the context of certain of the following claims), can be construed to cover both the singular and the plural, unless specifically noted otherwise. Thus, for example, “an active agent” refers not only to a single active agent, but also to a combination of two or more different active agents, “a dosage form” refers to a combination of dosage forms, as well as to a single dosage form, and the like. In some embodiments, the term “or” as used herein, including the claims, is used to mean “and/or” unless explicitly indicated to refer to alternatives only or the alternatives are mutually exclusive.

In some embodiments, numbers expressing quantities of ingredients, properties such as molecular weight, reaction conditions, and so forth, used to describe and claim certain embodiments described herein are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments described herein are approximations, the numerical values set forth in the specific examples are reported as precisely as practicable. The numerical values presented in some embodiments described herein may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. In some embodiments, “about” refers to a specified value +/−10%.

The terms “comprise,” “have,” and “include” are open-ended linking verbs. Any forms or tenses of one or more of these verbs, such as “comprises,” “comprising,” “has,” “having,” “includes,” and “including,” are also open-ended. For example, any method that “comprises,” “has,” or “includes” one or more steps is not limited to possessing only those one or more steps and can also cover other unlisted steps. Similarly, any composition or device that “comprises,” “has,” or “includes” one or more features is not limited to possessing only those one or more features and can cover other unlisted features.

As used herein, “protein” refers to a molecule consisting of amino acid residues joined by peptide bonds. In the context of this disclosure, a protein is one that is implicated in a protein aggregation diseases, e.g., α-synuclein, tau, and huntingtin. As described herein, a protein is the target of an intrabody as described herein, which is degraded to a desired level due to the addition of a human programmable PEST sequence.

As defined herein the term “rate of delivery” of a protein to a proteasome refers to the rate at which the protein of interest is degraded in a cell over time in the presence of the recombinant polypeptide of this disclosure containing a PEST domain relative to a control (e.g., an empty vector control).

It will be appreciated that throughout this disclosure reference is made to amino acids according to the single letter or three letter codes. The single and three letter amino acid codes are provided as follows: A=Ala=Alanine; C=Cys=Cysteine; D=Asp=Aspartate; E=Glu=Glutamate; F=Phe=Phenylalanine; G=Gly=Glycine; H=His=Histidine; I=Ile=Isoleucine; K=Lys=Lysine; L=Leu=Leucine; M=Met=Methionine; N=Asn=Asparagine; P=Pro=Proline; Q=Gln=Glutamine; R=Arg=Arginine; S=Ser=Serine; T=Thr=Threonine; V=Val=Valine; W=Trp=Tryptophan; X=Xaa=unknown [non standard—Unk]; and Y=Tyr=Tyrosine.

As used herein, “α-synuclein” refers to human α-synuclein, and is a protein implicated in a number of neurological diseases. As described herein, α-synuclein is the target of an intrabody as described herein, which is degraded to a desired level due to the addition of a human programmable PEST sequence. In some embodiments, the term “synuclein” may refer generally to proteins of the synuclein family, e.g., α-synuclein, β-synuclein, or γ-synuclein. For example, an anti-synuclein antibody may bind to any member of the synuclein family, while an anti-α-synuclein antibody binds only to α-synuclein.

As used herein, the term “antibody” includes intact immunoglobulins derived from natural sources or from recombinant sources, as well as immunoreactive portions (i.e., ‘antigen binding domains’ or ‘antigen binding portions’) of intact immunoglobulins. The antibodies described herein may exist in a variety of forms including, for example, polyclonal antibodies, monoclonal antibodies, intracellular antibodies (“intrabodies”), antibody fragments (e.g., Fv, Fab, Fab′, and F(ab′)₂), as well as single chain antibodies (scFv), single domain VH or VL antibodies, chimeric antibodies, human antibodies and humanized antibodies.

Antibody fragments (e.g., Fv, Fab, Fab′, and F(ab′)₂), such as antibody fragments of an anti-α-synuclein-binding antibody may be prepared by proteolytic digestion of an intact antibody (e.g., an anti-α-synuclein antibody, an anti-tau antibody, or an anti-huntingtin antibody). For example, antibody fragments can be obtained by treating a whole antibody with an enzyme such as papain, pepsin, or plasmin. Other enzymes appropriate for preparation of antibody fragments are known in the art.

Papain digestion of whole antibodies produces F(ab)₂ or Fab fragments; pepsin digestion of whole antibodies yields F(ab′)₂ or Fab′; and plasmin digestion of whole antibodies yields Fab fragments.

Alternatively, antibody fragments, such as antibody fragments of an anti-α-synuclein-binding antibody, can be produced recombinantly. For example, nucleic acids encoding the antibody fragments of interest can be constructed, introduced into an expression vector, and expressed in suitable host cells. For example, antibody fragments can be expressed in and secreted from E. coli, thus allowing the facile production of large amounts of these fragments. According to another approach, antibody fragments can be isolated directly from recombinant host cell culture.

As used herein, the term “epitope” designates a specific amino acid sequence, modified amino acid sequence, or protein secondary or tertiary structure which is specifically recognized by an antibody. The terms “specifically recognizing,” “specifically recognizes,” and any grammatical variants mean that the antibody or antigen-binding molecule thereof is capable of specifically interacting with and/or binding to at least two, at least three, or at least four amino acids of an epitope, e.g., an α-synuclein, tau, or huntingtin epitope. Such binding can be exemplified by the specificity of a “lock-and-key principle.” Thus, specific motifs in the amino acid sequence of the antigen-binding domain of the α-synuclein, tau, or huntingtin antibody, or antigen-binding molecules thereof, and the epitopes bind to each other as a result of their primary, secondary, or tertiary structure, as well as the result of secondary modifications of the structure.

As used herein “intrabody” refers to an antibody fragment, or antigen binding domain, that is active intracellularly. Intracellular antibody fragments can be, for example, single-chain variable fragments (scFvs) or single-domain antibodies (also known as nanobodies; an antibody fragment consisting of a single monomeric variable antibody domain). Intrabodies act as a neutralizing agent by direct binding to the intracellular target antigen, thereby altering protein folding, protein-protein, protein-DNA, protein-RNA interactions, and protein modification intracellularly. In some embodiments, intrabodies may also include camelid nanobodies, which are small heavy-chain-only antibody fragments (VHH) from naturally occurring heavy-chain only antibodies made in alpacas, llamas, camels, and guanacos.

As used herein, the terms “antigen” or “immunogen” are used interchangeably to refer to a substance, typically a protein, which is capable of inducing an immune response in a subject. For example, an α-synuclein tau, or huntingtin protein may be an antigen. The term also refers to proteins that are immunologically active in the sense that once administered to a subject (either directly or by administering to the subject a nucleotide sequence or vector that encodes the protein) is able to evoke an immune response of the humoral and/or cellular type directed against that protein.

As used herein, “co-administration” refers to the simultaneous administration of one or more drugs with another. In other embodiments, both drugs are administered at the same time. As described herein elsewhere, co-administration may also refer to any particular time period of administration of either drug, or both drugs. For example, as described herein, a drug may be administered hours or days before administration of another drug and still be considered to have been co-administered. In some embodiments, co-administration may refer to any time of administration of either drug such that both drugs are present in the body of a patient at the same. In some embodiments, either drug may be administered before or after the other, so long as they are both present within the patient for a sufficient amount of time that the patient received the intended clinical or pharmacological benefits.

Conservative amino acid substitutions providing functionally similar amino acids are well known in the art. The following six groups each contain amino acids that are conservative substitutions for one another: 1) Alanine (A), Serine (S), Threonine (T); 2) Aspartic acid (D), Glutamic acid (E); 3) Asparagine (N), Glutamine (Q); Arginine (R), Lysine (K); 5) Isoleucine (I), Leucine (L), Methionine (M), Valine (V); and 6) Phenylalanine (F), Tyrosine (Y), Tryptophan (W). Not all residue positions within a protein will tolerate an otherwise “conservative” substitution. For instance, if an amino acid residue is essential for a function of the protein, even an otherwise conservative substitution may disrupt that activity, for example the specific binding of an antibody to a target epitope may be disrupted by a conservative mutation in the target epitope.

As described herein, a substitution mutation that may be present in a PEST degron relative to SEQ ID NO:1 as described herein for targeting a protein to the proteasome to achieve increased degradation of the protein may be a mutation of a proline (P) residue to an alanine (A) residue, such as P426A/P427A (i.e., mutation of 2 consecutive P residues to 2 consecutive A residues), a mutation of an aspartic acid (D) residue to an A residue, such as D433A, a mutation of a serine (S) residue to an A residue, such as S445A, and/or a mutation of a lysine (K) residue to an A residue, such as K448A.

In other embodiments, a mutation that may be present in a PEST degron relative to SEQ ID NO:1 as described herein for targeting a protein to the proteasome to achieve reduced degradation of the protein may be a mutation of a P residue to an A residue, such as P438A, a mutation of a glutamic acid (E) residue to an A residue, such as E444A, a mutation of an S residue to an A residue, such as S440A, and/or a mutation of a threonine (T) residue to an A residue, such as T436A. In some embodiments, the mutation is one of those shown in FIG. 15.

In some embodiments, conservative amino acid substitutions, e.g., substituting one acidic or basic amino acid for another, can often be made without affecting the biological activity of a recombinant polypeptide as described herein. Minor variations in sequence of this nature may be made in any of the peptides disclosed herein, provided that these changes do not substantially alter (e.g., by 15% or more) the desired activity of the protein.

As used herein, a “degron” refers to a portion of a protein that is important in regulation of protein degradation rates. Known degrons include short amino acid sequences, structural motifs and exposed amino acids (e.g., lysine or arginine) located anywhere in the protein. In some embodiments, some proteins contain multiple degrons. As used herein, a PEST degron refers to a sequence useful for targeting a particular protein(s) to the proteasome for degradation. As described herein, a useful PEST degron may be from a mouse or a human, and may have a consensus sequence set forth herein as SEQ ID NO:1.

As used herein, a “diabody” refers to a noncovalent dimer of single-chain Fv (scFv) fragments that consists of heavy chain variable (VH) and light chain variable (VL) regions connected by a small peptide linker. In some embodiments, a diabody is two intrabodies tied or joined together with a linker, e.g., Vh-linker-Vh. In other embodiments, a diabody is a single-chain (Fv)2 in which two scFv fragments are covalently linked to each other.

A pharmaceutical composition described herein comprising a gene therapy encoding a bi-functional polypeptide may include a “therapeutically effective amount” of a bi-functional polypeptide as described herein. The term “therapeutically effective amount,” “pharmacologically effective dose,” “pharmacologically effective amount,” or simply “effective amount” may be used interchangeably and refers to that amount of an agent effective to produce the intended pharmacological, therapeutic or preventive result, e.g., an amount necessary to achieve a desired level of a protein, such as α-synuclein, tau, or huntingtin. The pharmacologically effective amount results in the amelioration of one or more symptoms of a disorder, or prevents the advancement of a disorder, or causes the regression of the disorder, or prevents the disorder. Such effective amounts can be determined based on the effect of the administered agent, or the combinatorial effect of agents if more than one agent is used. A therapeutically effective amount of an agent may also vary according to factors such as the disease stage, state, age, sex, and weight of the individual, and the ability of the compound to elicit a desired response in the individual, e.g., amelioration of at least one disorder parameter or amelioration of at least one symptom of the disorder. A therapeutically effective amount is also one in which any toxic or detrimental effects of the composition are outweighed by the therapeutically beneficial effects. In general, this amount will be sufficient to measurably target the protein target to the proteasome for degradation. When administered to a subject, a dosage will generally be used that will achieve target tissue concentrations (for example, in neurons) that has been shown to achieve a desired level of protein degradation (i.e., a desired level of protein being targeted/sent to the proteasome). In some examples, an “effective amount” is one that treats (including prophylaxis) one or more symptoms and/or underlying causes of any of a protein aggregation disorder or disease. In one example, an effective amount is a therapeutically effective amount. In one example, an effective amount is an amount that prevents one or more signs or symptoms of a particular disease or condition from developing.

As used herein, “epitope” refers to an antigenic determinant. Epitopes are particular chemical groups or peptide sequences on a molecule that are antigenic, such that they elicit a specific immune response, for example, an epitope is the region of an antigen to which B and/or T cells respond. Epitopes may be formed both from contiguous amino acids or noncontiguous amino acids juxtaposed by tertiary folding of a protein.

As used herein, “exogenous sequence” refers to a nucleic acid sequence that originates outside the host cell. An exogenous sequence may be a DNA sequence, an RNA sequence, or a combination thereof. Any type of nucleic acid available in the art may be used, as would be understood by one of skill in the art. Such a nucleic acid sequence can be obtained from a different species, e.g., mouse, or the same species, as that of the cell into which it is being delivered. In some embodiments, an exogenous nucleic acid sequence may encode a PEST degron sequence for targeting a desired protein to the proteasome for degradation as described herein, suitable for administration to a subject or patient. Such a recombinant polypeptide may be administered to a subject or patient in order to treat or prevent protein aggregation diseases or protein accumulation in a particular cell, tissue, organ, or the like.

As used herein, “gene delivery” refers to the introduction of an exogenous polynucleotide into a cell for gene transfer, and may encompass targeting, binding, uptake, transport, localization, replicon integration and expression. In some embodiments, gene delivery may refer to the introduction of an encoded product of a gene, i.e., a polypeptide or protein, such as a bi-functional polypeptide described herein.

As used herein, “gene transfer” refers to the introduction of an exogenous polynucleotide into a cell which may encompass targeting, binding, uptake, transport, localization and replicon integration, but is distinct from and does not imply subsequent expression of the gene.

As used herein, “gene expression” or “expression” refers to the process of gene transcription, translation, and post-translational modification.

By “pharmaceutically acceptable” is meant a material that is not biologically or otherwise undesirable, i.e., the material may be incorporated into a pharmaceutical composition administered to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. Food and Drug administration. “Pharmacologically active” (or simply “active”) as in a “pharmacologically active” (or “active”) derivative or analog, refers to a derivative or analog having the same type of pharmacological activity as the parent compound and approximately equivalent in degree. The term “pharmaceutically acceptable salts” include acid addition salts which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

As used herein, “pharmaceutically acceptable carrier” includes any and all solvents, dispersion media, coatings, antibacterial and antifungal agents, isotonic and absorption delaying agents, and the like that are physiologically compatible. The composition can include a pharmaceutically acceptable salt, e.g., an acid addition salt or a base addition salt.

As used herein, “programmable” as in “programmable PEST” or “programmable PEST degron” or “programmable proteasome-targeting PEST motif” refers to a PEST degron capable of being modified or altered in such a way so as to introduce certain mutations (i.e., amino acid substitutions, described herein elsewhere) that may increase or decrease relative to an unmodified or unaltered version of the same PEST degron, the degradation of a protein (e.g., α-synuclein, tau, or huntingtin) that is the target of an antigen binding domain fused to the PEST degron. In some embodiments, a PEST motif can have different mutations that increase the level of degradation of a protein (e.g., α-synuclein, tau, or huntingtin) in the cell from a baseline level, e.g., from a low level (e.g., 5%) of reduction from baseline to a high level (e.g., 100%) of reduction from baseline. This increased degradation can be seen compared to controls, such as empty vector controls.

As used herein, “increased degradation” refers to an increased or enhanced targeting of a protein for transport or delivery to or into a proteasome for degradation of the protein, by virtue of the addition of a PEST sequence as described herein to the protein. Likewise, “decreased degradation” or “reduced degradation” refers to a reduction or decrease in the targeting of a protein for transport or delivery to or into a proteasome for degradation of the protein, by virtue of the addition of a PEST sequence as described herein to the protein. Mutations of the PEST consensus sequence that may be useful for achieving increased or decreased degradation of a protein, such as α-synuclein, tau, or huntingtin, are described herein. Degradation of a protein, such as huntingtin, are described herein.

When comparing levels of protein degradation as described herein, an increase or decrease in degradation of a target protein may be compared to an empty vector, wild-type hPEST, hPEST scramble, or irrelevant antigen control (B8-hPEST). As used herein, a “scrambled control PEST” or “Scr” refers to a randomized polypeptide having the same number of amino acids as the programmable PEST, but that will not target the protein to the proteasome. This experimental degradation control allows quantification of how efficient a particular PEST degron is at degrading a target protein, e.g., α-synuclein, tau, or huntingtin. For example, a PEST degron described herein may increase degradation of a protein by a certain percentage compared to a scrambled control PEST, or it may decrease degradation of a protein by a certain percentage compared to a scrambled control PEST. In some embodiments, a particular PEST degron may be compared to an empty vector control, referred to herein as a “EV-CON” or “EV.” An empty vector control as used herein refers to an experimental control for comparing or quantifying the level of protein degradation in which the vector used for transfection of a cell with a construct encoding a PEST degron, or an intrabody fused to a PEST degron, lacks the sequence(s) encoding the PEST degron or the intrabody. In some embodiments, a particular PEST degron may be compared to an unmodified or unaltered version of the same PEST degron (i.e., a wild-type PEST sequence).

As used herein, “reducing” refers to a lowering or lessening, such as reducing cellular toxicity after spinal cord injury (SCI), or reducing the amount or concentration of a protein as described herein, such as α-synuclein, tau, or huntingtin. In some embodiments, administration of a bi-functional polypeptide as described herein may result in “reduced” or lessened protein aggregation or associated symptoms in the patient compared to a patient not been administered such a bi-functional polypeptide. “Reducing” may also refer to a reduction in disease symptoms as a result of a treatment as described herein, either alone, or co-administered with another drug.

As used herein, “subject” or “individual” or “patient” refers to any patient for whom or which therapy is desired, and generally refers to the recipient of the therapy. A “subject” or “patient” refers to any animal classified as a mammal, e.g., human and non-human mammals. Examples of non-human animals include dogs, cats, cattle, horses, sheep, pigs, goats, rabbits, etc. Unless otherwise noted, the terms “patient” or “subject” are used herein interchangeably. In some embodiments, a subject amenable for therapeutic applications may be a primate, e.g., human and non-human primates.

As used herein, “tau” refers to human tau, and is a protein implicated in a number of neurological diseases. As described herein, tau is the target of an intrabody as described herein, which is degraded to a desired level due to the addition of a human programmable PEST sequence.

As used herein, administration of a polynucleotide or vector into a host cell or a subject refers to introduction into the cell or the subject via any routinely practiced methods. This includes “transduction,” “transfection,” “transformation,” or “transducing,” as well known in the art. These terms all refer to standard processes for the introduction of an exogenous polynucleotide into a host cell, leading to expression of the polynucleotide, e.g., the transgene in the cell, and includes the use of plasmids and/or recombinant viruses to introduce the exogenous polynucleotide to the host cell. Transduction, transfection, or transformation of a polynucleotide in a cell may be determined by methods well known to the art including, but not limited to, protein expression (including steady state levels), e.g., by ELISA, flow cytometry and western blot, measurement of DNA and RNA by assays, e.g., northern blots, Southern blots, reporter function (Luc) assays, and/or gel shift mobility assays. Methods used for the introduction of the exogenous polynucleotide include well-known techniques such as bacterial and/or viral infection or transfection, lipofection, transformation, and electroporation, as well as other non-viral gene delivery techniques such as the introduction of stabilized RNA molecules. The introduced polynucleotide may be stably or transiently maintained in the host cell.

“Transcriptional regulatory sequences” or “TRS” described herein generally include at least one transcriptional promoter and may also include one or more enhancers and/or terminators of transcription. “Operably linked” refers to an arrangement of two or more components, wherein the components so described are in a relationship permitting them to function in a coordinated manner. By way of illustration, a transcriptional regulatory sequence or a promoter is operably linked to a coding sequence if the TRS or promoter promotes transcription of the coding sequence. An operably linked TRS is generally joined in cis with the coding sequence, but it is not necessarily directly adjacent to it.

The terms “treating” and “treatment” or “alleviating” as used herein refer to reduction or lessening in severity and/or frequency of symptoms, elimination of symptoms and/or underlying cause, and improvement or remediation of damage. In certain aspects, the term “treating” and “treatment” as used herein refer to the prevention of the occurrence of symptoms. In other aspects, the term “treating” and “treatment” as used herein refer to the prevention of the underlying cause of symptoms associated with a disease or condition, such as spinal cord injury (SCI). The phrase “administering to a patient” refers to the process of introducing a composition or dosage form into the patient via an art-recognized means of introduction. “Treating” or “alleviating” also includes the administration of compounds or agents to a subject to prevent or delay the onset of the symptoms, complications, or biochemical indicia of a disease (e.g., SCI), alleviating the symptoms or arresting or inhibiting further development of the disease, condition, or disorder. Subjects in need of treatment include those already suffering from the disease or condition, as well as those being at risk of developing the disease or condition. Treatment may be prophylactic (to prevent or delay the onset of the disease or condition, or to prevent the manifestation of clinical or subclinical symptoms thereof) or therapeutic suppression, or alleviation of symptoms after the manifestation of the disease or condition.

A “vector” is a nucleic acid with or without a carrier that can be introduced into a cell. Vectors capable of directing the expression of genes encoding for one or more polypeptides are referred to as “expression vectors.” Examples of suitable vectors include, e.g., viral vectors, plasmid vectors, liposomes, and other gene delivery vehicles.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present embodiments and does not pose a limitation on the scope of the embodiments otherwise claimed. No language in the specification should be construed as indicating any non-claimed element essential to the practice of the present embodiments

Groupings of alternative elements or embodiments disclosed herein are not to be construed as limitations. Each group member can be referred to and claimed individually or in any combination with other members of the group or other elements found herein. One or more members of a group can be included in, or deleted from, a group for reasons of convenience or patentability.

Having described the present embodiments in detail, it will be apparent that modifications, variations, and equivalent embodiments are possible without departing the scope defined in the appended claims. Furthermore, it should be appreciated that all examples described herein are provided as non-limiting examples.

TABLE 2
Sequence Listings

| SEQ ID | | |
|---|---|---|
| NO: | DESCRIPTION | SEQUENCE |
| 1 | Consensus | PDFPPEVEEQDASTLPVSCAWESGMKRH |
| | sequence of PEST | |
| | degron | |
| 2 | Sequence of | SHGFPPEVEEQDDGTLPMSCAQESGMDRHPAACASARINV |
| | mPEST (ODC-_{PEST422-461}) | |
| 3 | Sequence of | NPDFPPEVEEQDASTLPVSCAWESGMKRHRAACASASINV |
| | hPEST (ODC-_{PEST422-461}) | |
| 4 | α-synuclein | MDVFMKGLSKAKEGVVAAAEKTKQGVAEAAGKTKEG |
| | protein; GenBank | VLYVGSKTKEGVVHGVATVAEKTKEQVTNVGGAVVTG |
| | Accession No. | VTAVAQKTVEGAGSIAAATGFVKKDQLGKNEEGAPQEG |
| | CR541653 or | ILEDMPVDPDNEAYEMPSEEGYQDYEPEA |
| | CAG46454.1 | |
| 5 | DB1, Camelid | MAEVQLQASGGGFVQPGGSLRLSCAASGFTSWEDTMG |
| | Single domain | WFRQAPGKEREFVSAISFDANDLSDTSVYYADSVKGRF |
| | antibody, capable | TISRDNSKNTVYLQMNSLRAEDTATYYCAVASFEILLY |
| | of binding to | GESLHIYWGQGTQVTVSS |
| | lamprey and | |
| | human α- | |
| | synuclein | |

SEQ ID NOs: 6-11-Sequences of anti-synuclein intrabodies-Antigen synuclein amino acid residues
53-95. Set#1: VH14 (Human Single domain) Antigen synuclein amino acids 53-95

| | | |
|---|---|---|
| 6 | Sequence of | MSSQVQLQQSGPGRVKPSQTLSLACVISGDTVSSKTVSWNWIRQ |
| | VH14-hPEST | SPSRGLEWLGRTYFWPKWYTEYGASVKGRITIHPDTSKNQFSLQL |
| | | SSVTPEDTAVYYCARSKTKRPPYYYAGMDVWGQGTTVTVSSAST |
| | | KGPSGILGSGGGGSGILAAAYPYDVPDYANPDFPPEVEEQDASTLP |
| | | VSCAWESGMKRHRAACASASINV |
| 7 | VH14-hPEST- | MSSQVQLQQSGPGRVKPSQTLSLACVISGDTVSSKTVSWNWIRQSP |
| | P426A/P427A | SRGLEWLGRTYFWPKWYTEYGASVKGRITIHPDTSKNQFSLQLSSV |
| | | TPEDTAVYYCARSKTKRPPYYYAGMDVWGQGTTVTVSSASTKGPS |
| | | GILGSGGGGSGILAAAYPYDVPDYANPDFAAEVEEQDASTLPVSCA |
| | | WESGMKRHRAACASASINV |
| 8 | VH14-hPEST- | MSSQVQLQQSGPGRVKPSQTLSLACVISGDTVSSKTVSWNWIRQSP |
| | D433A | SRGLEWLGRTYFWPKWYTEYGASVKGRITIHPDTSKNQFSLQLSSV |
| | | TPEDTAVYYCARSKTKRPPYYYAGMDVWGQGTTVTVSSASTKGPS |
| | | GILGSGGGGSGILAAAYPYDVPDYANPDFPPEVEEQAASTLPVSCAW |
| | | ESGMKRHRAACASASINV |
| 9 | VH14-hPEST- | MSSQVQLQQSGPGRVKPSQTLSLACVISGDTVSSKTVSWNWIRQSP |
| | C441A | SRGLEWLGRTYFWPKWYTEYGASVKGRITIHPDTSKNQFSLQLSSV |
| | | TPEDTAVYYCARSKTKRPPYYYAGMDVWGQGTTVTVSSASTKGP |
| | | SGILGSGGGGSGILAAAYPYDVPDYANPDFPPEVEEQDASTLPVSAA |
| | | WESGMKRHRAACASASINV |
| 10 | VH14-hPEST- | MSSQVQLQQSGPGRVKPSQTLSLACVISGDTVSSKTVSWNWIRQSP |
| | S445A | SRGLEWLGRTYFWPKWYTEYGASVKGRITIHPDTSKNQFSLQLSSV |
| | | TPEDTAVYYCARSKTKRPPYYYAGMDVWGQGTTVTVSSASTKGPS |
| | | GILGSGGGGSGILAAAYPYDVPDYANPDFPPEVEEQDASTLPVSCA |
| | | WEAGMKRHRAACASASINV |
| 11 | VH14-hPEST- | MSSQVQLQQSGPGRVKPSQTLSLACVISGDTVSSKTVSWNWIRQSP |
| | Scramble | SRGLEWLGRTYFWPKWYTEYGASVKGRITIHPDTSKNQFSLQLSSV |
| | | TPEDTAVYYCARSKTKRPPYYYAGMDVWGQGTTVTVSSASTKGPS |
| | | GILGSGGGGSGILAAAYPYDVPDYAACCGEHPIRPPVEDFEESRASS |
| | | TASAWLANMVQNKPVSDA |
| 12 | DB1-hPEST | MAEVQLQASGGGFVQPGGSLRLSCAASGFTSWEDTMGWFRQAPG |
| | (Camelid Single | KEREFVSAISFDANDLSDTSVYYADSVKGRFTISRDNSKNTVYLQM |
| | domain) Antigen | NSLRAEDTATYYCAVASFEILLYGESLHIYWGQGTQVTVSSAAAYP |
| | synuclein amino | YDVPDYANPDFPPEVEEQDASTLPVSCAWESGMKRHRAACASASINV |
| | acids 53-95, Set | |
| | #2. | |
| 13 | N77D-4C-hPEST | MSRQVQLVESGGGGLVQPGGSLRLSCVTPGGIFSDYAMGWYRQAP |
| | (Camelid Single | GKQRELVADITVSDTTRYSDPVKGRFTISRDNAKDMVYLQMNSLKP |
| | domain) Antigen | EDAAIYTCNAQKWEDRYGSSERYDYWGQGTQVTVSSAHHSEDPAA |
| | synuclein amino | AYPYDVPDYANPDFPPEVEEQDASTLPVSCAWESGMKRHRAACA |
| | acids 53-95, Set | SASINV |
| | #3 | |
| 14 | glycine-serine | GGGGSGGGGSGGGGS |
| | linker that | |
| | connects the VH | |
| | to the VL to allow | |
| | proper folding | |
| | between the VH | |
| | and VL in the | |
| | intracellular | |
| | compartment of | |
| | cells | |
| 15 | HA tag for | YPYDVPDYA |
| | identification of | |
| | expression of the | |
| | intrabody under | |
| | experimental | |
| | conditions | |
| 16 | anti-α-synuclein | VOLVESGGGGLVQPGGSLRLSCVTPGGIFSDYAMGWYRQAPGKQ |
| | VHH-4C-N77D | RELVADITVSDTTRYSDPVKGRFTISRDNAKDMVYLQMNSLKPED |
| | (FIG. 16 top line) | AAIYTCNAQKWEDRYGSSERYDYWGQGTQVTVSS |
| 17 | anti-α-synuclein | EVQLQASGGGFVQPGGSLRLSCAASGFTSWEDTMGWFRQAPGKE |
| | VHH-DB1 | REFVSAISFDANDLSDTSVYYADSVKGRFTISRDNSKNTVYLQMNS |
| | (FIG. 16 middle | LRAEDTATYYCAVASFEILLYGESLHIYWGQGTQVTVSS |
| | line) | |
| 18 | anti-α-synuclein | QVQLQQSGPGRVKPSQTLSLACVISGDTVSSKTVSWNWIRQSPSRG |
| | VH14 | LEWLGRTYFWPKWYTEYGASVKGRITIHPDTSKNQFSLQLSSVTPE |
| | (FIG. 16 bottom | DTAVYYCARSKTKRPPYYYAGMDVWGQGTTVTVSS |
| | line) | |
| 19 | pcDNA3.1(-) | GACGGATCGGGAGATCTCCCGATCCCCTATGGTGCACTCTCAGTA |
| | empty vector | CAATCTGCTCTGATGCCGCATAGTTAAGCCAGTATCTGCTCCCTG |
| | | CTTGTGTGTTGGAGGTCGCTGAGTAGTGCGCGAGCAAAATTTAAG |
| | | CTACAACAAGGCAAGGCTTGACCGACAATTGCATGAAGAATCTGC |
| | | TTAGGGTTAGGCGTTTTGCGCTGCTTCGCGATGTACGGGCCAGAT |
| | | ATACGCGTTGACATTGATTATTGACTAGTTATTAATAGTAATCAA |
| | | TTACGGGGTCATTAGTTCATAGCCCATATATGGAGTTCCGCGTTA |
| | | CATAACTTACGGTAAATGGCCCGCCTGGCTGACCGCCCAACGACC |
| | | CCCGCCCATTGACGTCAATAATGACGTATGTTCCCATAGTAACGC |
| | | CAATAGGGACTTTCCATTGACGTCAATGGGTGGAGTATTTACGGT |
| | | AAACTGCCCACTTGGCAGTACATCAAGTGTATCATATGCCAAGTA |
| | | CGCCCCCTATTGACGTCAATGACGGTAAATGGCCCGCCTGGCATT |
| | | ATGCCCAGTACATGACCTTATGGGACTTTCCTACTTGGCAGTACA |
| | | TCTACGTATTAGTCATCGCTATTACCATGGTGATGCGGTTTTGGC |
| | | AGTACATCAATGGGCGTGGATAGCGGTTTGACTCACGGGGATTTC |
| | | CAAGTCTCCACCCCATTGACGTCAATGGGAGTTTGTTTTGGCACC |
| | | AAAATCAACGGGACTTTCCAAAATGTCGTAACAACTCCGCCCCAT |
| | | TGACGCAAATGGGCGGTAGGCGTGTACGGTGGGAGGTCTATATAA |
| | | GCAGAGCTCTCTGGCTAACTAGAGAACCCACTGCTTACTGGCTTA |
| | | TCGAAATTAATACGACTCACTATAGGGAGACCCAAGCTGGCTAGC |
| | | GTTTAAACGGGCCCTCTAGACTCGAGCGGCCGCCACTGTGCTGGA |
| | | TATCTGCAGAATTCCACCACACTGGACTAGTGGATCCGAGCTCGG |
| | | TACCAAGCTTAAGTTTAAACCGCTGATCAGCCTCGACTGTGCCTT |
| | | CTAGTTGCCAGCCATCTGTTGTTTGCCCCTCCCCCGTGCCTTCCT |
| | | TGACCCTGGAAGGTGCCACTCCCACTGTCCTTTCCTAATAAAATG |
| | | AGGAAATTGCATCGCATTGTCTGAGTAGGTGTCATTCTATTCTGG |
| | | GGGGTGGGGTGGGGCAGGACAGCAAGGGGGAGGATTGGGAAGACA |
| | | ATAGCAGGCATGCTGGGGATGCGGTGGGCTCTATGGCTTCTGAGG |
| | | CGGAAAGAACCAGCTGGGGCTCTAGGGGGTATCCCCACGCGCCCT |
| | | GTAGCGGCGCATTAAGCGCGGCGGGTGTGGTGGTTACGCGCAGCG |
| | | TGACCGCTACACTTGCCAGCGCCCTAGCGCCCGCTCCTTTCGCTT |
| | | TCTTCCCTTCCTTTCTCGCCACGTTCGCCGGCTTTCCCCGTCAAG |
| | | CTCTAAATCGGGGGCTCCCTTTAGGGTTCCGATTTAGTGCTTTAC |
| | | GGCACCTCGACCCCAAAAAACTTGATTAGGGTGATGGTTCACGTA |
| | | GTGGGCCATCGCCCTGATAGACGGTTTTTCGCCCTTTGACGTTGG |
| | | AGTCCACGTTCTTTAATAGTGGACTCTTGTTCCAAACTGGAACAA |
| | | CACTCAACCCTATCTCGGTCTATTCTTTTGATTTATAAGGGATTT |
| | | TGCCGATTTCGGCCTATTGGTTAAAAAATGAGCTGATTTAACAAA |
| | | AATTTAACGCGAATTAATTCTGTGGAATGTGTGTCAGTTAGGGTG |
| | | TGGAAAGTCCCCAGGCTCCCCAGCAGGCAGAAGTATGCAAAGCAT |
| | | GCATCTCAATTAGTCAGCAACCAGGTGTGGAAAGTCCCCAGGCTC |
| | | CCCAGCAGGCAGAAGTATGCAAAGCATGCATCTCAATTAGTCAGC |
| | | AACCATAGTCCCGCCCCTAACTCCGCCCATCCCGCCCCTAACTCC |
| | | GCCCAGTTCCGCCCATTCTCCGCCCCATGGCTGACTAATTTTTTT |
| | | TATTTATGCAGAGGCCGAGGCCGCCTCTGCCTCTGAGCTATTCCA |
| | | GAAGTAGTGAGGAGGCTTTTTTGGAGGCCTAGGCTTTTGCAAAAA |
| | | GCTCCCGGGAGCTTGTATATCCATTTTCGGATCTGATCAAGAGAC |
| | | AGGATGAGGATCGTTTCGCATGATTGAACAAGATGGATTGCACGC |
| | | AGGTTCTCCGGCCGCTTGGGTGGAGAGGCTATTCGGCTATGACTG |
| | | GGCACAACAGACAATCGGCTGCTCTGATGCCGCCGTGTTCCGGCT |
| | | GTCAGCGCAGGGGCGCCCGGTTCTTTTTGTCAAGACCGACCTGTC |
| | | CGGTGCCCTGAATGAACTGCAGGACGAGGCAGCGCGGCTATCGTG |
| | | GCTGGCCACGACGGGCGTTCCTTGCGCAGCTGTGCTCGACGTTGT |
| | | CACTGAAGCGGGAAGGGACTGGCTGCTATTGGGCGAAGTGCCGGG |
| | | GCAGGATCTCCTGTCATCTCACCTTGCTCCTGCCGAGAAAGTATC |
| | | CATCATGGCTGATGCAATGCGGCGGCTGCATACGCTTGATCCGGC |
| | | TACCTGCCCATTCGACCACCAAGCGAAACATCGCATCGAGCGAGC |
| | | ACGTACTCGGATGGAAGCCGGTCTTGTCGATCAGGATGATCTGGA |
| | | CGAAGAGCATCAGGGGCTCGCGCCAGCCGAACTGTTCGCCAGGCT |
| | | CAAGGCGCGCATGCCCGACGGCGAGGATCTCGTCGTGACCCATGG |
| | | CGATGCCTGCTTGCCGAATATCATGGTGGAAAATGGCCGCTTTTC |
| | | TGGATTCATCGACTGTGGCCGGCTGGGTGTGGCGGACCGCTATCA |
| | | GGACATAGCGTTGGCTACCCGTGATATTGCTGAAGAGCTTGGCGG |
| | | CGAATGGGCTGACCGCTTCCTCGTGCTTTACGGTATCGCCGCTCC |
| | | CGATTCGCAGCGCATCGCCTTCTATCGCCTTCTTGACGAGTTCTT |
| | | CTGAGCGGGACTCTGGGGTTCGAAATGACCGACCAAGCGACGCCC |
| | | AACCTGCCATCACGAGATTTCGATTCCACCGCCGCCTTCTATGAA |
| | | AGGTTGGGCTTCGGAATCGTTTTCCGGGACGCCGGCTGGATGATC |
| | | CTCCAGCGCGGGGATCTCATGCTGGAGTTCTTCGCCCACCCCAAC |
| | | TTGTTTATTGCAGCTTATAATGGTTACAAATAAAGCAATAGCATC |
| | | ACAAATTTCACAAATAAAGCATTTTTTTCACTGCATTCTAGTTGT |
| | | GGTTTGTCCAAACTCATCAATGTATCTTATCATGTCTGTATACCG |
| | | TCGACCTCTAGCTAGAGCTTGGCGTAATCATGGTCATAGCTGTTT |
| | | CCTGTGTGAAATTGTTATCCGCTCACAATTCCACACAACATACGA |
| | | GCCGGAAGCATAAAGTGTAAAGCCTGGGGTGCCTAATGAGTGAGC |
| | | TAACTCACATTAATTGCGTTGCGCTCACTGCCCGCTTTCCAGTCG |
| | | GGAAACCTGTCGTGCCAGCTGCATTAATGAATCGGCCAACGCGCG |
| | | GGGAGAGGCGGTTTGCGTATTGGGCGCTCTTCCGCTTCCTCGCTC |
| | | ACTGACTCGCTGCGCTCGGTCGTTCGGCTGCGGCGAGCGGTATCA |
| | | GCTCACTCAAAGGCGGTAATACGGTTATCCACAGAATCAGGGGAT |
| | | AACGCAGGAAAGAACATGTGAGCAAAAGGCCAGCAAAAGGCCAGG |
| | | AACCGTAAAAAGGCCGCGTTGCTGGCGTTTTTCCATAGGCTCCGC |
| | | CCCCCTGACGAGCATCACAAAAATCGACGCTCAAGTCAGAGGTGG |
| | | CGAAACCCGACAGGACTATAAAGATACCAGGCGTTTCCCCCTGGA |
| | | AGCTCCCTCGTGCGCTCTCCTGTTCCGACCCTGCCGCTTACCGGA |
| | | TACCTGTCCGCCTTTCTCCCTTCGGGAAGCGTGGCGCTTTCTCAT |
| | | AGCTCACGCTGTAGGTATCTCAGTTCGGTGTAGGTCGTTCGCTCC |
| | | AAGCTGGGCTGTGTGCACGAACCCCCCGTTCAGCCCGACCGCTGC |
| | | GCCTTATCCGGTAACTATCGTCTTGAGTCCAACCCGGTAAGACAC |
| | | GACTTATCGCCACTGGCAGCAGCCACTGGTAACAGGATTAGCAGA |
| | | GCGAGGTATGTAGGCGGTGCTACAGAGTTCTTGAAGTGGTGGCCT |
| | | AACTACGGCTACACTAGAAGAACAGTATTTGGTATCTGCGCTCTG |
| | | CTGAAGCCAGTTACCTTCGGAAAAAGAGTTGGTAGCTCTTGATCC |
| | | GGCAAACAAACCACCGCTGGTAGCGGTTTTTTTGTTTGCAAGCAG |
| | | CAGATTACGCGCAGAAAAAAAGGATCTCAAGAAGATCCTTTGATC |
| | | TTTTCTACGGGGTCTGACGCTCAGTGGAACGAAAACTCACGTTAA |
| | | GGGATTTTGGTCATGAGATTATCAAAAAGGATCTTCACCTAGATC |
| | | CTTTTAAATTAAAAATGAAGTTTTAAATCAATCTAAAGTATATAT |
| | | GAGTAAACTTGGTCTGACAGTTACCAATGCTTAATCAGTGAGGCA |
| | | CCTATCTCAGCGATCTGTCTATTTCGTTCATCCATAGTTGCCTGA |
| | | CTCCCCGTCGTGTAGATAACTACGATACGGGAGGGCTTACCATCT |
| | | GGCCCCAGTGCTGCAATGATACCGCGAGACCCACGCTCACCGGCT |
| | | CCAGATTTATCAGCAATAAACCAGCCAGCCGGAAGGGCCGAGCGC |
| | | AGAAGTGGTCCTGCAACTTTATCCGCCTCCATCCAGTCTATTAAT |
| | | TGTTGCCGGGAAGCTAGAGTAAGTAGTTCGCCAGTTAATAGTTTG |
| | | CGCAACGTTGTTGCCATTGCTACAGGCATCGTGGTGTCACGCTCG |
| | | TCGTTTGGTATGGCTTCATTCAGCTCCGGTTCCCAACGATCAAGG |
| | | CGAGTTACATGATCCCCCATGTTGTGCAAAAAAGCGGTTAGCTCC |
| | | TTCGGTCCTCCGATCGTTGTCAGAAGTAAGTTGGCCGCAGTGTTA |
| | | TCACTCATGGTTATGGCAGCACTGCATAATTCTCTTACTGTCATG |
| | | CCATCCGTAAGATGCTTTTCTGTGACTGGTGAGTACTCAACCAAG |
| | | TCATTCTGAGAATAGTGTATGCGGCGACCGAGTTGCTCTTGCCCG |
| | | GCGTCAATACGGGATAATACCGCGCCACATAGCAGAACTTTAAAA |
| | | GTGCTCATCATTGGAAAACGTTCTTCGGGGCGAAAACTCTCAAGG |
| | | ATCTTACCGCTGTTGAGATCCAGTTCGATGTAACCCACTCGTGCA |
| | | CCCAACTGATCTTCAGCATCTTTTACTTTCACCAGCGTTTCTGGG |
| | | TGAGCAAAAACAGGAAGGCAAAATGCCGCAAAAAAGGGAATAAGG |
| | | GCGACACGGAAATGTTGAATACTCATACTCTTCCTTTTTCAATAT |
| | | TATTGAAGCATTTATCAGGGTTATTGTCTCATGAGCGGATACATA |
| | | TTTGAATGTATTTAGAAAAATAAACAAATAGGGGTTCCGCGCACA |
| | | TTTCCCCGAAAAGTGCCACCTGACGTC |
| 20 | pAAV-MCS | CCTGCAGGCAGCTGCGCGCTCGCTCGCTCACTGAGGCCGCCCGGG |
| | | CAAAGCCCGGGCGTCGGGCGACCTTTGGTCGCCCGGCCTCAGTGA |
| | | GCGAGCGAGCGCGCAGAGAGGGAGTGGCCAACTCCATCACTAGGG |
| | | GTTCCTGCGGCCGCACGCGTGGAGCTAGTTATTAATAGTAATCAA |
| | | TTACGGGGTCATTAGTTCATAGCCCATATATGGAGTTCCGCGTTA |
| | | CATAACTTACGGTAAATGGCCCGCCTGGCTGACCGCCCAACGACC |
| | | CCCGCCCATTGACGTCAATAATGACGTATGTTCCCATAGTAACGT |
| | | CAATAGGGACTTTCCATTGACGTCAATGGGTGGAGTATTTACGGT |
| | | AAACTGCCCACTTGGCAGTACATCAAGTGTATCATATGCCAAGTA |
| | | CGCCCCCTATTGACGTCAATGACGGTAAATGGCCCGCCTGGCATT |
| | | ATGCCCAGTACATGACCTTATGGGACTTTCCTACTTGGCAGTACA |
| | | TCTACGTATTAGTCATCGCTATTACCATGGTGATGCGGTTTTGGC |
| | | AGTACATCAATGGGCGTGGATAGCGGTTTGACTCACGGGGATTTC |
| | | CAAGTCTCCACCCCATTGACGTCAATGGGAGTTTGTTTTGCACCA |
| | | AAATCAACGGGACTTTCCAAAATGTCGTAACAACTCCGCCCCATT |
| | | GACGCAAATGGGCGGTAGGCGTGTACGGTGGGAGGTCTATATAAG |
| | | CAGAGCTCGTTTAGTGAACCGTCAGATCGCCTGGAGACGCCATCC |
| | | ACGCTGTTTTGACCTCCATAGAAGACACCGGGACCGATCCAGCCT |
| | | CCGCGGATTCGAATCCCGGCCGGGAACGGTGCATTGGAACGCGGA |
| | | TTCCCCGTGCCAAGAGTGACGTAAGTACCGCCTATAGAGTCTATA |
| | | GGCCCACAAAAAATGCTTTCTTCTTTTAATATACTTTTTTGTTTA |
| | | TCTTATTTCTAATACTTTCCCTAATCTCTTTCTTTCAGGGCAATA |
| | | ATGATACAATGTATCATGCCTCTTTGCACCATTCTAAAGAATAAC |
| | | AGTGATAATTTCTGGGTTAAGGCAATAGCAATATTTCTGCATATA |
| | | AATATTTCTGCATATAAATTGTAACTGATGTAAGAGGTTTCATAT |
| | | TGCTAATAGCAGCTACAATCCAGCTACCATTCTGCTTTTATTTTA |
| | | TGGTTGGGATAAGGCTGGATTATTCTGAGTCCAAGCTAGGCCCTT |
| | | TTGCTAATCATGTTCATACCTCTTATCTTCCTCCCACAGCTCCTG |
| | | GGCAACGTGCTGGTCTGTGTGCTGGCCCATCACTTTGGCAAAGAA |
| | | TTGGGATTCGAACATCGATTGAATTCCCCGGGGATCCTCTAGAGT |
| | | CGACCTGCAGAAGCTTGCCTCGAGCAGCGCTGCTCGAGAGATCTA |
| | | CGGGTGGCATCCCTGTGACCCCTCCCCAGTGCCTCTCCTGGCCCT |
| | | GGAAGTTGCCACTCCAGTGCCCACCAGCCTTGTCCTAATAAAATT |
| | | AAGTTGCATCATTTTGTCTGACTAGGTGTCCTTCTATAATATTAT |
| | | GGGGTGGAGGGGGGTGGTATGGAGCAAGGGGCAAGTTGGGAAGAC |
| | | AACCTGTAGGGCCTGCGGGGTCTATTGGGAACCAAGCTGGAGTGC |
| | | AGTGGCACAATCTTGGCTCACTGCAATCTCCGCCTCCTGGGTTCA |
| | | AGCGATTCTCCTGCCTCAGCCTCCCGAGTTGTTGGGATTCCAGGC |
| | | ATGCATGACCAGGCTCAGCTAATTTTTGTTTTTTTGGTAGAGACG |
| | | GGGTTTCACCATATTGGCCAGGCTGGTCTCCAACTCCTAATCTCA |
| | | GGTGATCTACCCACCTTGGCCTCCCAAATTGCTGGGATTACAGGC |
| | | GTGAACCACTGCTCCCTTCCCTGTCCTTCTGATTTTGTAGGTAAC |
| | | CACGTGCGGACCGAGCGGCCGCAGGAACCCCTAGTGATGGAGTTG |
| | | GCCACTCCCTCTCTGCGCGCTCGCTCGCTCACTGAGGCCGGGCGA |
| | | CCAAAGGTCGCCCGACGCCCGGGCTTTGCCCGGGCGGCCTCAGTG |
| | | AGCGAGCGAGCGCGCAGCTGCCTGCAGGGGCGCCTGATGCGGTAT |
| | | TTTCTCCTTACGCATCTGTGCGGTATTTCACACCGCATACGTCAA |
| | | AGCAACCATAGTACGCGCCCTGTAGCGGCGCATTAAGCGCGGCGG |
| | | GTGTGGTGGTTACGCGCAGCGTGACCGCTACACTTGCCAGCGCCC |
| | | TAGCGCCCGCTCCTTTCGCTTTCTTCCCTTCCTTTCTCGCCACGT |
| | | TCGCCGGCTTTCCCCGTCAAGCTCTAAATCGGGGGCTCCCTTTAG |
| | | GGTTCCGATTTAGTGCTTTACGGCACCTCGACCCCAAAAAACTTG |
| | | ATTTGGGTGATGGTTCACGTAGTGGGCCATCGCCCTGATAGACGG |
| | | TTTTTCGCCCTTTGACGTTGGAGTCCACGTTCTTTAATAGTGGAC |
| | | TCTTGTTCCAAACTGGAACAACACTCAACCCTATCTCGGGCTATT |
| | | CTTTTGATTTATAAGGGATTTTGCCGATTTCGGCCTATTGGTTAA |
| | | AAAATGAGCTGATTTAACAAAAATTTAACGCGAATTTTAACAAAA |
| | | TATTAACGTTTACAATTTTATGGTGCACTCTCAGTACAATCTGCT |
| | | CTGATGCCGCATAGTTAAGCCAGCCCCGACACCCGCCAACACCCG |
| | | CTGACGCGCCCTGACGGGCTTGTCTGCTCCCGGCATCCGCTTACA |
| | | GACAAGCTGTGACCGTCTCCGGGAGCTGCATGTGTCAGAGGTTTT |
| | | CACCGTCATCACCGAAACGCGCGAGACGAAAGGGCCTCGTGATAC |
| | | GCCTATTTTTATAGGTTAATGTCATGATAATAATGGTTTCTTAGA |
| | | CGTCAGGTGGCACTTTTCGGGGAAATGTGCGCGGAACCCCTATTT |
| | | GTTTATTTTTCTAAATACATTCAAATATGTATCCGCTCATGAGAC |
| | | AATAACCCTGATAAATGCTTCAATAATATTGAAAAAGGAAGAGTA |
| | | TGAGTATTCAACATTTCCGTGTCGCCCTTATTCCCTTTTTTGCGG |
| | | CATTTTGCCTTCCTGTTTTTGCTCACCCAGAAACGCTGGTGAAAG |
| | | TAAAAGATGCTGAAGATCAGTTGGGTGCACGAGTGGGTTACATCG |
| | | AACTGGATCTCAACAGCGGTAAGATCCTTGAGAGTTTTCGCCCCG |
| | | AAGAACGTTTTCCAATGATGAGCACTTTTAAAGTTCTGCTATGTG |
| | | GCGCGGTATTATCCCGTATTGACGCCGGGCAAGAGCAACTCGGTC |
| | | GCCGCATACACTATTCTCAGAATGACTTGGTTGAGTACTCACCAG |
| | | TCACAGAAAAGCATCTTACGGATGGCATGACAGTAAGAGAATTAT |
| | | GCAGTGCTGCCATAACCATGAGTGATAACACTGCGGCCAACTTAC |
| | | TTCTGACAACGATCGGAGGACCGAAGGAGCTAACCGCTTTTTTGC |
| | | ACAACATGGGGGATCATGTAACTCGCCTTGATCGTTGGGAACCGG |
| | | AGCTGAATGAAGCCATACCAAACGACGAGCGTGACACCACGATGC |
| | | CTGTAGCAATGGCAACAACGTTGCGCAAACTATTAACTGGCGAAC |
| | | TACTTACTCTAGCTTCCCGGCAACAATTAATAGACTGGATGGAGG |
| | | CGGATAAAGTTGCAGGACCACTTCTGCGCTCGGCCCTTCCGGCTG |
| | | GCTGGTTTATTGCTGATAAATCTGGAGCCGGTGAGCGTGGGTCTC |
| | | GCGGTATCATTGCAGCACTGGGGCCAGATGGTAAGCCCTCCCGTA |
| | | TCGTAGTTATCTACACGACGGGGAGTCAGGCAACTATGGATGAAC |
| | | GAAATAGACAGATCGCTGAGATAGGTGCCTCACTGATTAAGCATT |
| | | GGTAACTGTCAGACCAAGTTTACTCATATATACTTTAGATTGATT |
| | | TAAAACTTCATTTTTAATTTAAAAGGATCTAGGTGAAGATCCTTT |
| | | TTGATAATCTCATGACCAAAATCCCTTAACGTGAGTTTTCGTTCC |
| | | ACTGAGCGTCAGACCCCGTAGAAAAGATCAAAGGATCTTCTTGAG |
| | | ATCCTTTTTTTCTGCGCGTAATCTGCTGCTTGCAAACAAAAAAAC |
| | | CACCGCTACCAGCGGTGGTTTGTTTGCCGGATCAAGAGCTACCAA |
| | | CTCTTTTTCCGAAGGTAACTGGCTTCAGCAGAGCGCAGATACCAA |
| | | ATACTGTCCTTCTAGTGTAGCCGTAGTTAGGCCACCACTTCAAGA |
| | | ACTCTGTAGCACCGCCTACATACCTCGCTCTGCTAATCCTGTTAC |
| | | CAGTGGCTGCTGCCAGTGGCGATAAGTCGTGTCTTACCGGGTTGG |
| | | ACTCAAGACGATAGTTACCGGATAAGGCGCAGCGGTCGGGCTGAA |
| | | CGGGGGGTTCGTGCACACAGCCCAGCTTGGAGCGAACGACCTACA |
| | | CCGAACTGAGATACCTACAGCGTGAGCTATGAGAAAGCGCCACGC |
| | | TTCCCGAAGGGAGAAAGGCGGACAGGTATCCGGTAAGCGGCAGGG |
| | | TCGGAACAGGAGAGCGCACGAGGGAGCTTCCAGGGGGAAACGCCT |
| | | GGTATCTTTATAGTCCTGTCGGGTTTCGCCACCTCTGACTTGAGC |
| | | GTCGATTTTTGTGATGCTCGTCAGGGGGGCGGAGCCTATGGAAAA |
| | | ACGCCAGCAACGCGGCCTTTTTACGGTTCCTGGCCTTTTGCTGGC |
| | | CTTTTGCTCACATGT |
| 21 | pTetO FUW | CGATGTACGGGCCAGATATACGCGTTGACATTGATTATTGACTAG |
| | empty vector | TTATTAATAGTAATCAATTACGGGGTCATTAGTTCATAGCCCATA |
| | | TATGGAGTTCCGCGTTACATAACTTACGGTAAATGGCCCGCCTGG |
| | | CTGACCGCCCAACGACCCCCGCCCATTGACGTCAATAATGACGTA |
| | | TGTTCCCATAGTAACGCCAATAGGGACTTTCCATTGACGTCAATG |
| | | GGTGGAGTATTTACGGTAAACTGCCCACTTGGCAGTACATCAAGT |
| | | GTATCATATGCCAAGTACGCCCCCTATTGACGTCAATGACGGTAA |
| | | ATGGCCCGCCTGGCATTATGCCCAGTACATGACCTTATGGGACTT |
| | | TCCTACTTGGCAGTACATCTACGTATTAGTCATCGCTATTACCAT |
| | | GGTGATGCGGTTTTGGCAGTACATCAATGGGCGTGGATAGCGGTT |
| | | TGACTCACGGGGATTTCCAAGTCTCCACCCCATTGACGTCAATGG |
| | | GAGTTTGTTTTGGCACCAAAATCAACGGGACTTTCCAAAATGTCG |
| | | TAACAACTCCGCCCCATTGACGCAAATGGGCGGTAGGCGTGTACG |
| | | GTGGGAGGTCTATATAAGCAGCGCGTTTTGCCTGTACTGGGTCTC |
| | | TCTGGTTAGACCAGATCTGAGCCTGGGAGCTCTCTGGCTAACTAG |
| | | GGAACCCACTGCTTAAGCCTCAATAAAGCTTGCCTTGAGTGCTTC |
| | | AAGTAGTGTGTGCCCGTCTGTTGTGTGACTCTGGTAACTAGAGAT |
| | | CCCTCAGACCCTTTTAGTCAGTGTGGAAAATCTCTAGCAGTGGCG |
| | | CCCGAACAGGGACTTGAAAGCGAAAGGGAAACCAGAGGAGCTCTC |
| | | TCGACGCAGGACTCGGCTTGCTGAAGCGCGCACGGCAAGAGGCGA |
| | | GGGGCGGCGACTGGTGAGTACGCCAAAAATTTTGACTAGCGGAGG |
| | | CTAGAAGGAGAGAGATGGGTGCGAGAGCGTCAGTATTAAGCGGGG |
| | | GAGAATTAGATCGCGATGGGAAAAAATTCGGTTAAGGCCAGGGGG |
| | | AAAGAAAAAATATAAATTAAAACATATAGTATGGGCAAGCAGGGA |
| | | GCTAGAACGATTCGCAGTTAATCCTGGCCTGTTAGAAACATCAGA |
| | | AGGCTGTAGACAAATACTGGGACAGCTACAACCATCCCTTCAGAC |
| | | AGGATCAGAAGAACTTAGATCATTATATAATACAGTAGCAACCCT |
| | | CTATTGTGTGCATCAAAGGATAGAGATAAAAGACACCAAGGAAGC |
| | | TTTAGACAAGATAGAGGAAGAGCAAAACAAAAGTAAGACCACCGC |
| | | ACAGCAAGCGGCCGCTGATCTTCAGACCTGGAGGAGGAGATATGA |
| | | GGGACAATTGGAGAAGTGAATTATATAAATATAAAGTAGTAAAAA |
| | | TTGAACCATTAGGAGTAGCACCCACCAAGGCAAAGAGAAGAGTGG |
| | | TGCAGAGAGAAAAAAGAGCAGTGGGAATAGGAGCTTTGTTCCTTG |
| | | GGTTCTTGGGAGCAGCAGGAAGCACTATGGGCGCAGCGTCAATGA |
| | | CGCTGACGGTACAGGCCAGACAATTATTGTCTGGTATAGTGCAGC |
| | | AGCAGAACAATTTGCTGAGGGCTATTGAGGCGCAACAGCATCTGT |
| | | TGCAACTCACAGTCTGGGGCATCAAGCAGCTCCAGGCAAGAATCC |
| | | TGGCTGTGGAAAGATACCTAAAGGATCAACAGCTCCTGGGGATTT |
| | | GGGGTTGCTCTGGAAAACTCATTTGCACCACTGCTGTGCCTTGGA |
| | | ATGCTAGTTGGAGTAATAAATCTCTGGAACAGATTTGGAATCACA |
| | | CGACCTGGATGGAGTGGGACAGAGAAATTAACAATTACACAAGCT |
| | | TAATACACTCCTTAATTGAAGAATCGCAAAACCAGCAAGAAAAGA |
| | | ATGAACAAGAATTATTGGAATTAGATAAATGGGCAAGTTTGTGGA |
| | | ATTGGTTTAACATAACAAATTGGCTGTGGTATATAAAATTATTCA |
| | | TAATGATAGTAGGAGGCTTGGTAGGTTTAAGAATAGTTTTTGCTG |
| | | TACTTTCTATAGTGAATAGAGTTAGGCAGGGATATTCACCATTAT |
| | | CGTTTCAGACCCACCTCCCAACCCCGAGGGGACCCGACAGGCCCG |
| | | AAGGAATAGAAGAAGAAGGTGGAGAGAGAGACAGAGACAGATCCA |
| | | TTCGATTAGTGAACGGATCGGCACTGCGTGCGCCAATTCTGCAGA |
| | | CAAATGGCAGTATTCATCCACAATTTTAAAAGAAAAGGGGGGATT |
| | | GGGGGGTACAGTGCAGGGGAAAGAATAGTAGACATAATAGCAACA |
| | | GACATACAAACTAAAGAATTACAAAAACAAATTACAAAAATTCAA |
| | | AATTTTCGGGTTTATTACAGGGACAGCAGAGATCCAGTTTGGTTA |
| | | TCGAGTTTACCACTCCCTATCAGTGATAGAGAAAAGTGAAAGTCG |
| | | AGTTTACCACTCCCTATCAGTGATAGAGAAAAGTGAAAGTCGAGT |
| | | TTACCACTCCCTATCAGTGATAGAGAAAAGTGAAAGTCGAGTTTA |
| | | CCACTCCCTATCAGTGATAGAGAAAAGTGAAAGTCGAGTTTACCA |
| | | CTCCCTATCAGTGATAGAGAAAAGTGAAAGTCGAGTTTACCACTC |
| | | CCTATCAGTGATAGAGAAAAGTGAAAGTCGAGTTTACCACTCCCT |
| | | ATCAGTGATAGAGAAAAGTGAAAGTCGAGCTCGGTACCCGGGTCG |
| | | AGGTAGGCGTGTACGGTGGGAGGCCTATATAAGCAGAGCTCGTTT |
| | | AGTGAACCGTCAGATCGCCTGGAGACGCCATCCACGCTGTTTTGA |
| | | CCTCCATAGAAGACACCGGGACCGATCCAGCCTCCGCGGCCCCGA |
| | | ATTACAGGCTAGCTATCAGGATCCTACGAGGCGCGCCAGATAGAA |
| | | TTCTCTTGGGCCTCAAGGGCCATGAGTTAACCAGTTCTAGATGTG |
| | | ACTCATGTCAATTCGATATCAAGCTTATCGATAATCAACCTCTGG |
| | | ATTACAAAATTTGTGAAAGATTGACTGGTATTCTTAACTATGTTG |
| | | CTCCTTTTACGCTATGTGGATACGCTGCTTTAATGCCTTTGTATC |
| | | ATGCTATTGCTTCCCGTATGGCTTTCATTTTCTCCTCCTTGTATA |
| | | AATCCTGGTTGCTGTCTCTTTATGAGGAGTTGTGGCCCGTTGTCA |
| | | GGCAACGTGGCGTGGTGTGCACTGTGTTTGCTGACGCAACCCCCA |
| | | CTGGTTGGGGCATTGCCACCACCTGTCAGCTCCTTTCCGGGACTT |
| | | TCGCTTTCCCCCTCCCTATTGCCACGGCGGAACTCATCGCCGCCT |
| | | GCCTTGCCCGCTGCTGGACAGGGGCTCGGCTGTTGGGCACTGACA |
| | | ATTCCGTGGTGTTGTCGGGGAAATCATCGTCCTTTCCTTGGCTGC |
| | | TCGCCTGTGTTGCCACCTGGATTCTGCGCGGGACGTCCTTCTGCT |
| | | ACGTCCCTTCGGCCCTCAATCCAGCGGACCTTCCTTCCCGCGGCC |
| | | TGCTGCCGGCTCTGCGGCCTCTTCCGCGTCTTCGCCTTCGCCCTC |
| | | AGACGAGTCGGATCTCCCTTTGGGCCGCCTCCCCGCATCGATACC |
| | | GTCGACCTCGAGACCTAGAAAAACATGGAGCAATCACAAGTAGCA |
| | | ATACAGCAGCTACCAATGCTGATTGTGCCTGGCTAGAAGCACAAG |
| | | AGGAGGAGGAGGTGGGTTTTCCAGTCACACCTCAGGTACCTTTAA |
| | | GACCAATGACTTACAAGGCAGCTGTAGATCTTAGCCACTTTTTAA |
| | | AAGAAAAGGGGGGACTGGAAGGGCTAATTCACTCCCAACGAAGAC |
| | | AAGATATCCTTGATCTGTGGATCTACCACACACAAGGCTACTTCC |
| | | CTGATTGGCAGAACTACACACCAGGGCCAGGGATCAGATATCCAC |
| | | TGACCTTTGGATGGTGCTACAAGCTAGTACCAGTTGAGCAAGAGA |
| | | AGGTAGAAGAAGCCAATGAAGGAGAGAACACCCGCTTGTTACACC |
| | | CTGTGAGCCTGCATGGGATGGATGACCCGGAGAGAGAAGTATTAG |
| | | AGTGGAGGTTTGACAGCCGCCTAGCATTTCATCACATGGCCCGAG |
| | | AGCTGCATCCGGACTGTACTGGGTCTCTCTGGTTAGACCAGATCT |
| | | GAGCCTGGGAGCTCTCTGGCTAACTAGGGAACCCACTGCTTAAGC |
| | | CTCAATAAAGCTTGCCTTGAGTGCTTCAAGTAGTGTGTGCCCGTC |
| | | TGTTGTGTGACTCTGGTAACTAGAGATCCCTCAGACCCTTTTAGT |
| | | CAGTGTGGAAAATCTCTAGCAGGGCCCGTTTAAACCCGCTGATCA |
| | | GCCTCGACTGTGCCTTCTAGTTGCCAGCCATCTGTTGTTTGCCCC |
| | | TCCCCCGTGCCTTCCTTGACCCTGGAAGGTGCCACTCCCACTGTC |
| | | CTTTCCTAATAAAATGAGGAAATTGCATCGCATTGTCTGAGTAGG |
| | | TGTCATTCTATTCTGGGGGGTGGGGTGGGGCAGGACAGCAAGGGG |
| | | GAGGATTGGGAAGACAATAGCAGGCATGCTGGGGATGCGGTGGGC |
| | | TCTATGGCTTCTGAGGCGGAAAGAACCAGCTGGGGCTCTAGGGGG |
| | | TATCCCCACGCGCCCTGTAGCGGCGCATTAAGCGCGGCGGGTGTG |
| | | GTGGTTACGCGCAGCGTGACCGCTACACTTGCCAGCGCCCTAGCG |
| | | CCCGCTCCTTTCGCTTTCTTCCCTTCCTTTCTCGCCACGTTCGCC |
| | | GGCTTTCCCCGTCAAGCTCTAAATCGGGGGCTCCCTTTAGGGTTC |
| | | CGATTTAGTGCTTTACGGCACCTCGACCCCAAAAAACTTGATTAG |
| | | GGTGATGGTTCACGTAGTGGGCCATCGCCCTGATAGACGGTTTTT |
| | | CGCCCTTTGACGTTGGAGTCCACGTTCTTTAATAGTGGACTCTTG |
| | | TTCCAAACTGGAACAACACTCAACCCTATCTCGGTCTATTCTTTT |
| | | GATTTATAAGGGATTTTGCCGATTTCGGCCTATTGGTTAAAAAAT |
| | | GAGCTGATTTAACAAAAATTTAACGCGAATTAATTCTGTGGAATG |
| | | TGTGTCAGTTAGGGTGTGGAAAGTCCCCAGGCTCCCCAGCAGGCA |
| | | GAAGTATGCAAAGCATGCATCTCAATTAGTCAGCAACCAGGTGTG |
| | | GAAAGTCCCCAGGCTCCCCAGCAGGCAGAAGTATGCAAAGCATGC |
| | | ATCTCAATTAGTCAGCAACCATAGTCCCGCCCCTAACTCCGCCCA |
| | | TCCCGCCCCTAACTCCGCCCAGTTCCGCCCATTCTCCGCCCCATG |
| | | GCTGACTAATTTTTTTTATTTATGCAGAGGCCGAGGCCGCCTCTG |
| | | CCTCTGAGCTATTCCAGAAGTAGTGAGGAGGCTTTTTTGGAGGCC |
| | | TAGGCTTTTGCAAAAAGCTCCCGGGAGCTTGTATATCCATTTTCG |
| | | GATCTGATCAGCACGTGTTGACAATTAATCATCGGCATAGTATAT |
| | | CGGCATAGTATAATACGACAAGGTGAGGAACTAAACCATGGCCAA |
| | | GTTGACCAGTGCCGTTCCGGTGCTCACCGCGCGCGACGTCGCCGG |
| | | AGCGGTCGAGTTCTGGACCGACCGGCTCGGGTTCTCCCGGGACTT |
| | | CGTGGAGGACGACTTCGCCGGTGTGGTCCGGGACGACGTGACCCT |
| | | GTTCATCAGCGCGGTCCAGGACCAGGTGGTGCCGGACAACACCCT |
| | | GGCCTGGGTGTGGGTGCGCGGCCTGGACGAGCTGTACGCCGAGTG |
| | | GTCGGAGGTCGTGTCCACGAACTTCCGGGACGCCTCCGGGCCGGC |
| | | CATGACCGAGATCGGCGAGCAGCCGTGGGGGCGGGAGTTCGCCCT |
| | | GCGCGACCCGGCCGGCAACTGCGTGCACTTCGTGGCCGAGGAGCA |
| | | GGACTGACACGTGCTACGAGATTTCGATTCCACCGCCGCCTTCTA |
| | | TGAAAGGTTGGGCTTCGGAATCGTTTTCCGGGACGCCGGCTGGAT |
| | | GATCCTCCAGCGCGGGGATCTCATGCTGGAGTTCTTCGCCCACCC |
| | | CAACTTGTTTATTGCAGCTTATAATGGTTACAAATAAAGCAATAG |
| | | CATCACAAATTTCACAAATAAAGCATTTTTTTCACTGCATTCTAG |
| | | TTGTGGTTTGTCCAAACTCATCAATGTATCTTATCATGTCTGTAT |
| | | ACCGTCGACCTCTAGCTAGAGCTTGGCGTAATCATGGTCATAGCT |
| | | GTTTCCTGTGTGAAATTGTTATCCGCTCACAATTCCACACAACAT |
| | | ACGAGCCGGAAGCATAAAGTGTAAAGCCTGGGGTGCCTAATGAGT |
| | | GAGCTAACTCACATTAATTGCGTTGCGCTCACTGCCCGCTTTCCA |
| | | GTCGGGAAACCTGTCGTGCCAGCTGCATTAATGAATCGGCCAACG |
| | | CGCGGGGAGAGGCGGTTTGCGTATTGGGCGCTCTTCCGCTTCCTC |
| | | GCTCACTGACTCGCTGCGCTCGGTCGTTCGGCTGCGGCGAGCGGT |
| | | ATCAGCTCACTCAAAGGCGGTAATACGGTTATCCACAGAATCAGG |
| | | GGATAACGCAGGAAAGAACATGTGAGCAAAAGGCCAGCAAAAGGC |
| | | CAGGAACCGTAAAAAGGCCGCGTTGCTGGCGTTTTTCCATAGGCT |
| | | CCGCCCCCCTGACGAGCATCACAAAAATCGACGCTCAAGTCAGAG |
| | | GTGGCGAAACCCGACAGGACTATAAAGATACCAGGCGTTTCCCCC |
| | | TGGAAGCTCCCTCGTGCGCTCTCCTGTTCCGACCCTGCCGCTTAC |
| | | CGGATACCTGTCCGCCTTTCTCCCTTCGGGAAGCGTGGCGCTTTC |
| | | TCATAGCTCACGCTGTAGGTATCTCAGTTCGGTGTAGGTCGTTCG |
| | | CTCCAAGCTGGGCTGTGTGCACGAACCCCCCGTTCAGCCCGACCG |
| | | CTGCGCCTTATCCGGTAACTATCGTCTTGAGTCCAACCCGGTAAG |
| | | ACACGACTTATCGCCACTGGCAGCAGCCACTGGTAACAGGATTAG |
| | | CAGAGCGAGGTATGTAGGCGGTGCTACAGAGTTCTTGAAGTGGTG |
| | | GCCTAACTACGGCTACACTAGAAGAACAGTATTTGGTATCTGCGC |
| | | TCTGCTGAAGCCAGTTACCTTCGGAAAAAGAGTTGGTAGCTCTTG |
| | | ATCCGGCAAACAAACCACCGCTGGTAGCGGTGGTTTTTTTGTTTG |
| | | CAAGCAGCAGATTACGCGCAGAAAAAAAGGATCTCAAGAAGATCC |
| | | TTTGATCTTTTCTACGGGGTCTGACGCTCAGTGGAACGAAAACTC |
| | | ACGTTAAGGGATTTTGGTCATGAGATTATCAAAAAGGATCTTCAC |
| | | CTAGATCCTTTTAAATTAAAAATGAAGTTTTAAATCAATCTAAAG |
| | | TATATATGAGTAAACTTGGTCTGACAGTTACCAATGCTTAATCAG |
| | | TGAGGCACCTATCTCAGCGATCTGTCTATTTCGTTCATCCATAGT |
| | | TGCCTGACTCCCCGTCGTGTAGATAACTACGATACGGGAGGGCTT |
| | | ACCATCTGGCCCCAGTGCTGCAATGATACCGCGAGACCCACGCTC |
| | | ACCGGCTCCAGATTTATCAGCAATAAACCAGCCAGCCGGAAGGGC |
| | | CGAGCGCAGAAGTGGTCCTGCAACTTTATCCGCCTCCATCCAGTC |
| | | TATTAATTGTTGCCGGGAAGCTAGAGTAAGTAGTTCGCCAGTTAA |
| | | TAGTTTGCGCAACGTTGTTGCCATTGCTACAGGCATCGTGGTGTC |
| | | ACGCTCGTCGTTTGGTATGGCTTCATTCAGCTCCGGTTCCCAACG |
| | | ATCAAGGCGAGTTACATGATCCCCCATGTTGTGCAAAAAAGCGGT |
| | | TAGCTCCTTCGGTCCTCCGATCGTTGTCAGAAGTAAGTTGGCCGC |
| | | AGTGTTATCACTCATGGTTATGGCAGCACTGCATAATTCTCTTAC |
| | | TGTCATGCCATCCGTAAGATGCTTTTCTGTGACTGGTGAGTACTC |
| | | AACCAAGTCATTCTGAGAATAGTGTATGCGGCGACCGAGTTGCTC |
| | | TTGCCCGGCGTCAATACGGGATAATACCGCGCCACATAGCAGAAC |
| | | TTTAAAAGTGCTCATCATTGGAAAACGTTCTTCGGGGCGAAAACT |
| | | CTCAAGGATCTTACCGCTGTTGAGATCCAGTTCGATGTAACCCAC |
| | | TCGTGCACCCAACTGATCTTCAGCATCTTTTACTTTCACCAGCGT |
| | | TTCTGGGTGAGCAAAAACAGGAAGGCAAAATGCCGCAAAAAAGGG |
| | | AATAAGGGCGACACGGAAATGTTGAATACTCATACTCTTCCTTTT |
| | | TCAATATTATTGAAGCATTTATCAGGGTTATTGTCTCATGAGCGG |
| | | ATACATATTTGAATGTATTTAGAAAAATAAACAAATAGGGGTTCC |
| | | GCGCACATTTCCCCGAAAAGTGCCACCTGACGTCGACGGATCGGG |
| | | AGATCTCCCGATCCCCTATGGTGCACTCTCAGTACAATCTGCTCT |
| | | GATGCCGCATAGTTAAGCCAGTATCTGCTCCCTGCTTGTGTGTTG |
| | | GAGGTCGCTGAGTAGTGCGCGAGCAAAATTTAAGCTACAACAAGG |
| | | CAAGGCTTGACCGACAATTGCATGAAGAATCTGCTTAGGGTTAGG |
| | | CGTTTTGCGCTGCTTCG |
| 22 | tau protein amino | MAEPRQEFEVMEDHAGTYGLGDRKDQGGYTMHQDQEGDTDAGLKE |
| | acid 1-441 | SPLQTPTEDGSEEPGSETSDAKSTPTAEDVTAPLVDEGAPGKQAA |
| | (GenBank | AQPHTEIPEGTTAEEAGIGDTPSLEDEAAGHVTQARMVSKSKDGT |
| | Accession No. | GSDDKKAKGADGKTKIATPRGAAPPGQKGQANATRIPAKTPPAPK |
| | NP_005901) | TPPSSGEPPKSGDRSGYSSPGSPGTPGSRSRTPSLPTPPTREPKK |
| | | VAVVRTPPKSPSSAKSRLQTAPVPMPDLKNVKSKIGSTENLKHQP |
| | | GGGKVQIINKKLDLSNVQSKCGSKDNIKHVPGGGSVQIVYKPVDL |
| | | SKVTSKCGSLGNIHHKPGGGQVEVKSEKLDFKDRVQSKIGSLDNI |
| | | THVPGGGNKKIETHKLTFRENAKAKTDHGAEIVYKSPVVSGDTSP |
| | | RHLSNVSSTGSIDMVDSPQLATLADEVSASLAKQGL |

SEQ ID NOs: 23-34-Sequences of anti-tau-scFv-V (Set #1) epitope currently identified within
the region of tau amino acids 151-441

| | | |
|---|---|---|
| 23 | scFv-V-hPEST | MQVQLVESGGGLVKPGGSLRLSCAASGFTFSSYAMSWVRQAPGKG |
| | | LEWVAAISGSGDNTYYADSVKGRFTISRDNSENTVHLQMAGLRAED |
| | | TALYFCAKDGPEVGNPGGYFDFWGRGTLVTVSSGGGGSGGGGSGG |
| | | GGSDIVMTKSPDSLAVSLGERATINCKSSQSLLYSSKNKDYLAWYQK |
| | | KPGQSPRLLISWASTRESGVPDRFSGSGSGTDFTLTINRLQAEDVAVY |
| | | YCQHYYSYPLTFGQGTKAAAYPYDVPDYANPDFPPEVEEQDASTLP |
| | | VSCAWESGMKRHRAACASASINV |
| 24 | scFv-V-hPEST- | MQVQLVESGGGLVKPGGSLRLSCAASGFTFSSYAMSWVRQAPGKG |
| | C441A | LEWVAAISGSGDNTYYADSVKGRFTISRDNSENTVHLQMAGLRAE |
| | | DTALYFCAKDGPEVGNPGGYFDFWGRGTLVTVSSGGGGSGGGGS |
| | | GGGGSDIVMTKSPDSLAVSLGERATINCKSSQSLLYSSKNKDYLAW |
| | | YQKKPGQSPRLLISWASTRESGVPDRFSGSGSGTDFTLTINRLQAED |
| | | VAVYYCQHYYSYPLTFGQGTKAAAYPYDVPDYANPDFPPEVEEQD |
| | | ASTLPVSAAWESGMKRHRAACASASINV |
| 25 | scFv-V-hPEST- | MQVQLVESGGGLVKPGGSLRLSCAASGFTFSSYAMSWVRQAPGK |
| | D433A | GLEWVAAISGSGDNTYYADSVKGRFTISRDNSENTVHLQMAGLR |
| | | AEDTALYFCAKDGPEVGNPGGYFDFWGRGTLVTVSSGGGGSGG |
| | | GGSGGGGSDIVMTKSPDSLAVSLGERATINCKSSQSLLYSSKNKD |
| | | YLAWYQKKPGQSPRLLISWASTRESGVPDRFSGSGSGTDFTLTIN |
| | | RLQAEDVAVYYCQHYYSYPLTFGQGTKAAAYPYDVPDYANPDF |
| | | PPEVEEQAASTLPVSCAWESGMKRHRAACASASINV |
| 26 | scFv-V-hPEST- | MQVQLVESGGGLVKPGGSLRLSCAASGFTFSSYAMSWVRQAPGK |
| | E428A-E430A- | GLEWVAAISGSGDNTYYADSVKGRFTISRDNSENTVHLQMAGLR |
| | E431A | AEDTALYFCAKDGPEVGNPGGYFDFWGRGTLVTVSSGGGGSGGG |
| | | GSGGGGSDIVMTKSPDSLAVSLGERATINCKSSQSLLYSSKNKDYL |
| | | AWYQKKPGQSPRLLISWASTRESGVPDRFSGSGSGTDFTLTINRLQ |
| | | AEDVAVYYCQHYYSYPLTFGQGTKAAAYPYDVPDYANPDFPPAV |
| | | AAQDASTLPVSCAWESGMKRHRAACASASINV |
| 27 | scFv-V-hPEST- | MQVQLVESGGGLVKPGGSLRLSCAASGFTFSSYAMSWVRQAPGK |
| | E444A | GLEWVAAISGSGDNTYYADSVKGRFTISRDNSENTVHLQMAGLR |
| | | AEDTALYFCAKDGPEVGNPGGYFDFWGRGTLVTVSSGGGGSGGG |
| | | GSGGGGSDIVMTKSPDSLAVSLGERATINCKSSQSLLYSSKNKDYL |
| | | AWYQKKPGQSPRLLISWASTRESGVPDRFSGSGSGTDFTLTINRLQ |
| | | AEDVAVYYCQHYYSYPLTFGQGTKAAAYPYDVPDYANPDFPPEV |
| | | EEQDASTLPVSCAWASGMKRHRAACASASINV |
| 28 | scFv-V-hPEST- | MQVQLVESGGGLVKPGGSLRLSCAASGFTFSSYAMSWVRQAPGK |
| | K448A | GLEWVAAISGSGDNTYYADSVKGRFTISRDNSENTVHLQMAGLR |
| | | AEDTALYFCAKDGPEVGNPGGYFDFWGRGTLVTVSSGGGGSGGG |
| | | GSGGGGSDIVMTKSPDSLAVSLGERATINCKSSQSLLYSSKNKDYL |
| | | AWYQKKPGQSPRLLISWASTRESGVPDRFSGSGSGTDFTLTINRLQ |
| | | AEDVAVYYCQHYYSYPLTFGQGTKAAAYPYDVPDYANPDFPPEV |
| | | EEQDASTLPVSCAWESGMARHRAACASASINV |
| 29 | scFv-V-hPEST- | MQVQLVESGGGLVKPGGSLRLSCAASGFTFSSYAMSWVRQAPGK |
| | P426A/P427A | GLEWVAAISGSGDNTYYADSVKGRFTISRDNSENTVHLQMAGLR |
| | | AEDTALYFCAKDGPEVGNPGGYFDFWGRGTLVTVSSGGGGSGG |
| | | GGSGGGGSDIVMTKSPDSLAVSLGERATINCKSSQSLLYSSKNKD |
| | | YLAWYQKKPGQSPRLLISWASTRESGVPDRFSGSGSGTDFTLTIN |
| | | RLQAEDVAVYYCQHYYSYPLTFGQGTKAAAYPYDVPDYANPDF |
| | | AAEVEEQDASTLPVSCAWESGMKRHRAACASASINV |
| 30 | scFv-V-hPEST- | MQVQLVESGGGLVKPGGSLRLSCAASGFTFSSYAMSWVRQAPGK |
| | P438A | GLEWVAAISGSGDNTYYADSVKGRFTISRDNSENTVHLQMAGLR |
| | | AEDTALYFCAKDGPEVGNPGGYFDFWGRGTLVTVSSGGGGSGGG |
| | | GSGGGGSDIVMTKSPDSLAVSLGERATINCKSSQSLLYSSKNKDYL |
| | | AWYQKKPGQSPRLLISWASTRESGVPDRFSGSGSGTDFTLTINRLQ |
| | | AEDVAVYYCQHYYSYPLTFGQGTKAAAYPYDVPDYANPDFPPEV |
| | | EEQDASTLAVSCAWESGMKRHRAACASASINV |
| 31 | scFv-V-hPEST- | MQVQLVESGGGLVKPGGSLRLSCAASGFTFSSYAMSWVRQAPGK |
| | S435A | GLEWVAAISGSGDNTYYADSVKGRFTISRDNSENTVHLQMAGLR |
| | | AEDTALYFCAKDGPEVGNPGGYFDFWGRGTLVTVSSGGGGSGGG |
| | | GSGGGGSDIVMTKSPDSLAVSLGERATINCKSSQSLLYSSKNKDYL |
| | | AWYQKKPGQSPRLLISWASTRESGVPDRFSGSGSGTDFTLTINRLQ |
| | | AEDVAVYYCQHYYSYPLTFGQGTKAAAYPYDVPDYANPDFPPEV |
| | | EEQDAATLPVSCAWESGMKRHRAACASASINV |
| 32 | scFv-V-hPEST- | MQVQLVESGGGLVKPGGSLRLSCAASGFTFSSYAMSWVRQAPGK |
| | S440A | GLEWVAAISGSGDNTYYADSVKGRFTISRDNSENTVHLQMAGLR |
| | | AEDTALYFCAKDGPEVGNPGGYFDFWGRGTLVTVSSGGGGSGGG |
| | | GSGGGGSDIVMTKSPDSLAVSLGERATINCKSSQSLLYSSKNKDYL |
| | | AWYQKKPGQSPRLLISWASTRESGVPDRFSGSGSGTDFTLTINRLQ |
| | | AEDVAVYYCQHYYSYPLTFGQGTKAAAYPYDVPDYANPDFPPEV |
| | | EEQDASTLPVACAWESGMKRHRAACASASINV |
| 33 | scFv-V-hPEST- | MQVQLVESGGGLVKPGGSLRLSCAASGFTFSSYAMSWVRQAPGKG |
| | S445A | LEWVAAISGSGDNTYYADSVKGRFTISRDNSENTVHLQMAGLRAED |
| | | TALYFCAKDGPEVGNPGGYFDFWGRGTLVTVSSGGGGSGGGGSGG |
| | | GGSDIVMTKSPDSLAVSLGERATINCKSSQSLLYSSKNKDYLAWYQ |
| | | KKPGQSPRLLISWASTRESGVPDRFSGSGSGTDFTLTINRLQAEDVAV |
| | | YYCQHYYSYPLTFGQGTKAAAYPYDVPDYANPDFPPEVEEQDASTL |
| | | PVSCAWEAGMKRHRAACASASINV |
| 34 | scFv-V-hPEST- | MQVQLVESGGGLVKPGGSLRLSCAASGFTFSSYAMSWVRQAPGKG |
| | Scramble | LEWVAAISGSGDNTYYADSVKGRFTISRDNSENTVHLQMAGLRAED |
| | | TALYFCAKDGPEVGNPGGYFDFWGRGTLVTVSSGGGGSGGGGSGG |
| | | GGSDIVMTKSPDSLAVSLGERATINCKSSQSLLYSSKNKDYLAWYQ |
| | | KKPGQSPRLLISWASTRESGVPDRFSGSGSGTDFTLTINRLQAEDVAV |
| | | YYCQHYYSYPLTFGQGTKAAAYPYDVPDYAACCGEHPIRPPVEDFE |
| | | ESRASSTASAWLANMVQNKPVSDA |

SEQ ID NOs: 35-46-Sequences of anti-tau-scFv-N (Set #2) epitope currently identified within
the region of tau amino acids 151-441

| | | |
|---|---|---|
| 35 | scFv-N-hPEST | QVQLVESGGGLVKPGGSLRLSCAASGFTFSSYAMSWVRQAPGKGLE |
| | | WVAAISGSGDNTYYADSVKGRFTISRDNSENTVHLQMAGLRAEDTA |
| | | LYFCAKDGPAVGNPGGYFDFWGRGTLVTVSSGGGGSGGGGSGGGG |
| | | SDIVMTQSPDSLAVSLGERATINCKSSQSLLYSSNNKDYLAWYQQKP |
| | | GQSPRLLIPWASTRESGVPDRFSGSGSGTDFTLTINRLQAEDVAVYYC |
| | | QHYYSYPLTFGQGTKAAAYPYDVPDYANPDFPPEVEEQDASTLPVS |
| | | CAWESGMKRHRAACASASINV |
| 36 | scFv-N-hPEST- | QVQLVESGGGLVKPGGSLRLSCAASGFTFSSYAMSWVRQAPGKGL |
| | C441A | EWVAAISGSGDNTYYADSVKGRFTISRDNSENTVHLQMAGLRAED |
| | | TALYFCAKDGPAVGNPGGYFDFWGRGTLVTVSSGGGGSGGGGSG |
| | | GGGSDIVMTQSPDSLAVSLGERATINCKSSQSLLYSSNNKDYLAWY |
| | | QQKPGQSPRLLIPWASTRESGVPDRFSGSGSGTDFTLTINRLQAEDV |
| | | AVYYCQHYYSYPLTFGQGTKAAAYPYDVPDYANPDFPPEVEEQDA |
| | | STLPVSAAWESGMKRHRAACASASINV |
| 37 | scFv-N-hPEST- | QVQLVESGGGLVKPGGSLRLSCAASGFTFSSYAMSWVRQAPGKGL |
| | D433A | EWVAAISGSGDNTYYADSVKGRFTISRDNSENTVHLQMAGLRAED |
| | | TALYFCAKDGPAVGNPGGYFDFWGRGTLVTVSSGGGGSGGGGSG |
| | | GGGSDIVMTQSPDSLAVSLGERATINCKSSQSLLYSSNNKDYLAWY |
| | | QQKPGQSPRLLIPWASTRESGVPDRFSGSGSGTDFTLTINRLQAEDV |
| | | AVYYCQHYYSYPLTFGQGTKAAAYPYDVPDYANPDFPPEVEEQAA |
| | | STLPVSCAWESGMKRHRAACASASINV |
| 38 | scFv-N-hPEST- | MQVQLVESGGGLVKPGGSLRLSCAASGFTFSSYAMSWVRQAPGK |
| | 431A | GLEWVAAISGSGDNTYYADSVKGRFTISRDNSENTVHLQMAGLRA |
| | | EDTALYFCAKDGPAVGNPGGYFDFWGRGTLVTVSSGGGGSGGGG |
| | | SGGGGSDIVMTQSPDSLAVSLGERATINCKSSQSLLYSSNNKDYLA |
| | | WYQQKPGQSPRLLIPWASTRESGVPDRFSGSGSGTDFTLTINRLQA |
| | | EDVAVYYCQHYYSYPLTFGQGTKAAAYPYDVPDYANPDFPPAV |
| | | AAQDASTLPVSCAWESGMKRHRAACASASINV |
| 39 | scFv-N-hPEST- | MQVQLVESGGGLVKPGGSLRLSCAASGFTFSSYAMSWVRQAPGK |
| | E444A | GLEWVAAISGSGDNTYYADSVKGRFTISRDNSENTVHLQMAGLR |
| | | AEDTALYFCAKDGPAVGNPGGYFDFWGRGTLVTVSSGGGGSGGG |
| | | GSGGGGSDIVMTQSPDSLAVSLGERATINCKSSQSLLYSSNNKDYL |
| | | AWYQQKPGQSPRLLIPWASTRESGVPDRFSGSGSGTDFTLTINRLQ |
| | | AEDVAVYYCQHYYSYPLTFGQGTKAAAYPYDVPDYANPDFPPEV |
| | | EEQDASTLPVSCAWASGMKRHRAACASASINV |
| 40 | scFv-N-hPEST- | MQVQLVESGGGLVKPGGSLRLSCAASGFTFSSYAMSWVRQAPGK |
| | K448A | GLEWVAAISGSGDNTYYADSVKGRFTISRDNSENTVHLQMAGLR |
| | | AEDTALYFCAKDGPAVGNPGGYFDFWGRGTLVTVSSGGGGSGG |
| | | GGSGGGGSDIVMTQSPDSLAVSLGERATINCKSSQSLLYSSNNKD |
| | | YLAWYQQKPGQSPRLLIPWASTRESGVPDRFSGSGSGTDFTLTIN |
| | | RLQAEDVAVYYCQHYYSYPLTFGQGTKAAAYPYDVPDYANPDF |
| | | PPEVEEQDASTLPVSCAWESGMARHRAACASASINV |
| 41 | scFv-N-hPEST- | QVQLVESGGGLVKPGGSLRLSCAASGFTFSSYAMSWVRQAPGKG |
| | P426A/P427A | LEWVAAISGSGDNTYYADSVKGRFTISRDNSENTVHLQMAGLRA |
| | | EDTALYFCAKDGPAVGNPGGYFDFWGRGTLVTVSSGGGGSGGG |
| | | GSGGGGSDIVMTQSPDSLAVSLGERATINCKSSQSLLYSSNNKDYL |
| | | AWYQQKPGQSPRLLIPWASTRESGVPDRFSGSGSGTDFTLTINRLQ |
| | | AEDVAVYYCQHYYSYPLTFGQGTKAAAYPYDVPDYANPDFAAE |
| | | VEEQDASTLPVSCAWESGMKRHRAACASASINV |
| 42 | scFv-N-hPEST- | MQVQLVESGGGLVKPGGSLRLSCAASGFTFSSYAMSWVRQAPGK |
| | P438A | GLEWVAAISGSGDNTYYADSVKGRFTISRDNSENTVHLQMAGLR |
| | | AEDTALYFCAKDGPAVGNPGGYFDFWGRGTLVTVSSGGGGSGG |
| | | GGSGGGGSDIVMTQSPDSLAVSLGERATINCKSSQSLLYSSNNKD |
| | | YLAWYQQKPGQSPRLLIPWASTRESGVPDRFSGSGSGTDFTLTINR |
| | | LQAEDVAVYYCQHYYSYPLTFGQGTKAAAYPYDVPDYANPDFPP |
| | | EVEEQDASTLAVSCAWESGMKRHRAACASASINV |
| 43 | scFv-N-hPEST- | MQVQLVESGGGLVKPGGSLRLSCAASGFTFSSYAMSWVRQAPGK |
| | S435A | GLEWVAAISGSGDNTYYADSVKGRFTISRDNSENTVHLQMAGLR |
| | | AEDTALYFCAKDGPAVGNPGGYFDFWGRGTLVTVSSGGGGSGGG |
| | | GSGGGGSDIVMTQSPDSLAVSLGERATINCKSSQSLLYSSNNKDYL |
| | | AWYQQKPGQSPRLLIPWASTRESGVPDRFSGSGSGTDFTLTINRLQ |
| | | AEDVAVYYCQHYYSYPLTFGQGTKAAAYPYDVPDYANPDFPPEV |
| | | EEQDAATLPVSCAWESGMKRHRAACASASINV |
| 44 | scFv-N-hPEST- | MQVQLVESGGGLVKPGGSLRLSCAASGFTFSSYAMSWVRQAPGK |
| | S440A | GLEWVAAISGSGDNTYYADSVKGRFTISRDNSENTVHLQMAGLRA |
| | | EDTALYFCAKDGPAVGNPGGYFDFWGRGTLVTVSSGGGGSGGGG |
| | | SGGGGSDIVMTQSPDSLAVSLGERATINCKSSQSLLYSSNNKDYLA |
| | | WYQQKPGQSPRLLIPWASTRESGVPDRFSGSGSGTDFTLTINRLQ |
| | | AEDVAVYYCQHYYSYPLTFGQGTKAAAYPYDVPDYANPDFPPE |
| | | VEEQDASTLPVACAWESGMKRHRAACASASINV |
| 45 | scFv-N-hPEST- | QVQLVESGGGLVKPGGSLRLSCAASGFTFSSYAMSWVRQAPGKG |
| | S445A | LEWVAAISGSGDNTYYADSVKGRFTISRDNSENTVHLQMAGLRA |
| | | EDTALYFCAKDGPAVGNPGGYFDFWGRGTLVTVSSGGGGSGGG |
| | | GSGGGGSDIVMTQSPDSLAVSLGERATINCKSSQSLLYSSNNKDY |
| | | LAWYQQKPGQSPRLLIPWASTRESGVPDRFSGSGSGTDFTLTINRL |
| | | QAEDVAVYYCQHYYSYPLTFGQGTKAAAYPYDVPDYANPDFPPE |
| | | VEEQDASTLPVSCAWEAGMKRHRAACASASINV |
| 46 | scFv-N-hPEST- | QVQLVESGGGLVKPGGSLRLSCAASGFTFSSYAMSWVRQAPGKG |
| | Scramble | LEWVAAISGSGDNTYYADSVKGRFTISRDNSENTVHLQMAGLRA |
| | | EDTALYFCAKDGPAVGNPGGYFDFWGRGTLVTVSSGGGGSGGGG |
| | | SGGGGSDIVMTQSPDSLAVSLGERATINCKSSQSLLYSSNNKDYLA |
| | | WYQQKPGQSPRLLIPWASTRESGVPDRFSGSGSGTDFTLTINRLQA |
| | | EDVAVYYCQHYYSYPLTFGQGTKAAAYPYDVPDYAACCGEHPIR |
| | | PPVEDFEESRASSTASAWLANMVQNKPVSDA |

SEQ ID NOs: 47-58-Sequences of anti-tau-scFv-F (Set #3) epitope currently identified within
the regions of tau amino acids 151-276 and 368-391

| | | |
|---|---|---|
| 47 | scFv-F-hPEST | MQVQLVESGGGLVKPGGSLRLSCAASGFTFSDYYMSWIRQAPGKG |
| | | LEWVSSISSSSSYIYYADSVKGRFTISRDNAKNSLYLQMNSLRAEDT |
| | | AVYYCARDGIAARSGYYGMDVWGQGTLVTVSSGGGGSGGGGSG |
| | | GGGSEIVLTQSPSTLSASVGDRVTITCRASQSISSWLAWYQQKPGK |
| | | APKLLIYAASILQSGVPSRFSGSGSGTDFTLTISSLQPEDFATYYCLQ |
| | | DSNPYPLLTFGGGTKAAAYPYDVPDYANPDFPPEVEEQDASTLPVS |
| | | CAWESGMKRHRAACASASINV |
| 48 | scFv-F-hPEST- | MQVQLVESGGGLVKPGGSLRLSCAASGFTFSDYYMSWIRQAPGK |
| | C441A | GLEWVSSISSSSSYIYYADSVKGRFTISRDNAKNSLYLQMNSLRAE |
| | | DTAVYYCARDGIAARSGYYGMDVWGQGTLVTVSSGGGGSGGGG |
| | | SGGGGSEIVLTQSPSTLSASVGDRVTITCRASQSISSWLAWYQQKP |
| | | GKAPKLLIYAASILQSGVPSRFSGSGSGTDFTLTISSLQPEDFATYY |
| | | CLQDSNPYPLLTFGGGTKAAAYPYDVPDYANPDFPPEVEEQDAS |
| | | TLPVSAAWESGMKRHRAACASASINV |
| 49 | scFv-F-hPEST- | MQVQLVESGGGLVKPGGSLRLSCAASGFTFSDYYMSWIRQAPGK |
| | D433A | GLEWVSSISSSSSYIYYADSVKGRFTISRDNAKNSLYLQMNSLRAE |
| | | DTAVYYCARDGIAARSGYYGMDVWGQGTLVTVSSGGGGSGGGG |
| | | SGGGGSEIVLTQSPSTLSASVGDRVTITCRASQSISSWLAWYQQKP |
| | | GKAPKLLIYAASILQSGVPSRFSGSGSGTDFTLTISSLQPEDFATYY |
| | | CLQDSNPYPLLTFGGGTKAAAYPYDVPDYANPDFPPEVEEQAAST |
| | | LPVSCAWESGMKRHRAACASASINV |
| 50 | scFv-F-hPEST- | MQVQLVESGGGLVKPGGSLRLSCAASGFTFSDYYMSWIRQAPGK |
| | E428A-E430A- | GLEWVSSISSSSSYIYYADSVKGRFTISRDNAKNSLYLQMNSLRAE |
| | E431A | DTAVYYCARDGIAARSGYYGMDVWGQGTLVTVSSGGGGSGGGG |
| | | SGGGGSEIVLTQSPSTLSASVGDRVTITCRASQSISSWLAWYQQKP |
| | | GKAPKLLIYAASILQSGVPSRFSGSGSGTDFTLTISSLQPEDFATYYC |
| | | LQDSNPYPLLTFGGGTKAAAYPYDVPDYANPDFPPAVAAQDASTL |
| | | PVSCAWESGMKRHRAACASASINV |
| 51 | scFv-F-hPEST- | MQVQLVESGGGLVKPGGSLRLSCAASGFTFSDYYMSWIRQAPGKG |
| | E444A | LEWVSSISSSSSYIYYADSVKGRFTISRDNAKNSLYLQMNSLRAEDT |
| | | AVYYCARDGIAARSGYYGMDVWGQGTLVTVSSGGGGSGGGGSGG |
| | | GGSEIVLTQSPSTLSASVGDRVTITCRASQSISSWLAWYQQKPGKAP |
| | | KLLIYAASILQSGVPSRFSGSGSGTDFTLTISSLQPEDFATYYCLQDS |
| | | NPYPLLTFGGGTKAAAYPYDVPDYANPDFPPEVEEQDASTLPVSCA |
| | | WASGMKRHRAACASASINV |
| 52 | scFv-F-hPEST- | MQVQLVESGGGLVKPGGSLRLSCAASGFTFSDYYMSWIRQAPGKG |
| | K448A | LEWVSSISSSSSYIYYADSVKGRFTISRDNAKNSLYLQMNSLRAEDT |
| | | AVYYCARDGIAARSGYYGMDVWGQGTLVTVSSGGGGSGGGGSG |
| | | GGGSEIVLTQSPSTLSASVGDRVTITCRASQSISSWLAWYQQKPGK |
| | | APKLLIYAASILQSGVPSRFSGSGSGTDFTLTISSLQPEDFATYYCLQ |
| | | DSNPYPLLTFGGGTKAAAYPYDVPDYANPDFPPEVEEQDASTLPV |
| | | SCAWESGMARHRAACASASINV |
| 53 | scFv-F-hPEST- | MQVQLVESGGGLVKPGGSLRLSCAASGFTFSDYYMSWIRQAPGKGL |
| | P426A/P427A | EWVSSISSSSSYIYYADSVKGRFTISRDNAKNSLYLQMNSLRAEDTA |
| | | VYYCARDGIAARSGYYGMDVWGQGTLVTVSSGGGGSGGGGSGGG |
| | | GSEIVLTQSPSTLSASVGDRVTITCRASQSISSWLAWYQQKPGKAPKL |
| | | LIYAASILQSGVPSRFSGSGSGTDFTLTISSLQPEDFATYYCLQDSNPY |
| | | PLLTFGGGTKAAAYPYDVPDYANPDFAAEVEEQDASTLPVSCAWES |
| | | GMKRHRAACASASINV |
| 54 | scFv-F-hPEST- | MQVQLVESGGGLVKPGGSLRLSCAASGFTFSDYYMSWIRQAPGKGL |
| | P438A | EWVSSISSSSSYIYYADSVKGRFTISRDNAKNSLYLQMNSLRAEDTA |
| | | VYYCARDGIAARSGYYGMDVWGQGTLVTVSSGGGGSGGGGSGGG |
| | | GSEIVLTQSPSTLSASVGDRVTITCRASQSISSWLAWYQQKPGKAPKL |
| | | LIYAASILQSGVPSRFSGSGSGTDFTLTISSLQPEDFATYYCLQDSNPY |
| | | PLLTFGGGTKAAAYPYDVPDYANPDFPPEVEEQDASTLAVSCAWES |
| | | GMKRHRAACASASINV |
| 55 | scFv-F-hPEST- | MQVQLVESGGGLVKPGGSLRLSCAASGFTFSDYYMSWIRQAPGKG |
| | S435A | LEWVSSISSSSSYIYYADSVKGRFTISRDNAKNSLYLQMNSLRAEDT |
| | | AVYYCARDGIAARSGYYGMDVWGQGTLVTVSSGGGGSGGGGSGG |
| | | GGSEIVLTQSPSTLSASVGDRVTITCRASQSISSWLAWYQQKPGKAP |
| | | KLLIYAASILQSGVPSRFSGSGSGTDFTLTISSLQPEDFATYYCLQDSN |
| | | PYPLLTFGGGTKAAAYPYDVPDYANPDFPPEVEEQDAATLPVSCAW |
| | | ESGMKRHRAACASASINV |
| 56 | scFv-F-hPEST- | MQVQLVESGGGLVKPGGSLRLSCAASGFTFSDYYMSWIRQAPGKG |
| | S440A | LEWVSSISSSSSYIYYADSVKGRFTISRDNAKNSLYLQMNSLRAED |
| | | TAVYYCARDGIAARSGYYGMDVWGQGTLVTVSSGGGGSGGGGS |
| | | GGGGSEIVLTQSPSTLSASVGDRVTITCRASQSISSWLAWYQQKPG |
| | | KAPKLLIYAASILQSGVPSRFSGSGSGTDFTLTISSLQPEDFATYYCL |
| | | QDSNPYPLLTFGGGTKAAAYPYDVPDYANPDFPPEVEEQDASTLP |
| | | VACAWESGMKRHRAACASASINV |
| 57 | scFv-F-hPEST- | MQVQLVESGGGLVKPGGSLRLSCAASGFTFSDYYMSWIRQAPGKG |
| | S445A | LEWVSSISSSSSYIYYADSVKGRFTISRDNAKNSLYLQMNSLRAEDT |
| | | AVYYCARDGIAARSGYYGMDVWGQGTLVTVSSGGGGSGGGGSG |
| | | GGGSEIVLTQSPSTLSASVGDRVTITCRASQSISSWLAWYQQKPGKA |
| | | PKLLIYAASILQSGVPSRFSGSGSGTDFTLTISSLQPEDFATYYCLQDS |
| | | NPYPLLTFGGGTKAAAYPYDVPDYANPDFPPEVEEQDASTLPVSCA |
| | | WEAGMKRHRAACASASINV |
| 58 | scFv-F-hPEST- | MQVQLVESGGGLVKPGGSLRLSCAASGFTFSDYYMSWIRQAPGKGL |
| | Scramble | EWVSSISSSSSYIYYADSVKGRFTISRDNAKNSLYLQMNSLRAEDTA |
| | | VYYCARDGIAARSGYYGMDVWGQGTLVTVSSGGGGSGGGGSGGG |
| | | GSEIVLTQSPSTLSASVGDRVTITCRASQSISSWLAWYQQKPGKAPK |
| | | LLIYAASILQSGVPSRFSGSGSGTDFTLTISSLQPEDFATYYCLQDSNP |
| | | YPLLTFGGGTKAAAYPYDVPDYAACCGEHPIRPPVEDFEESRASSTA |
| | | SAWLANMVQNKPVSDA |

SEQ ID NOs: 59-64-Sequences of anti-tau-scFv-A (Set #4) epitope currently identified within
the regions of tau amino acids 151-276 and 368-391

| | | |
|---|---|---|
| 59 | scFv-A-hPEST | MQVQLQESGGGLVQPGRSLRLSCAASGFTFDDYAMHWVRQAPGK |
| | | GLEWVAVISYDGSNKYYADSVKGRFTISRDNSKNTLYLQMNSLRAE |
| | | DTAVYYCARDFAGAIAYWGQGTLVTVSSGGGGSGGGGSGGGGSEI |
| | | VLTQSPSFLSASVGDRVTITCRASHGINNYLAWYQQKPGKAPKLLIY |
| | | AASSLQSGVPSRFSGSGSGTDFTLTISSLQPEDFATYYCQQANSFPLT |
| | | FGGGTKAAAYPYDVPDYANPDFPPEVEEQDASTLPVSCAWESGMK |
| | | RHRAACASASINV |
| 60 | scFv-A-hPEST- | MQVQLQESGGGLVQPGRSLRLSCAASGFTFDDYAMHWVRQAPGK |
| | P426A/P427A | GLEWVAVISYDGSNKYYADSVKGRFTISRDNSKNTLYLQMNSLRA |
| | | EDTAVYYCARDFAGAIAYWGQGTLVTVSSGGGGSGGGGSGGGGS |
| | | EIVLTQSPSFLSASVGDRVTITCRASHGINNYLAWYQQKPGKAPKL |
| | | LIYAASSLQSGVPSRFSGSGSGTDFTLTISSLOPEDFATYYCQQANSF |
| | | PLTFGGGTKAAAYPYDVPDYANPDFAAEVEEQDASTLPVSCAWES |
| | | GMKRHRAACASASINV |
| 61 | scFv-A-hPEST- | MQVQLQESGGGLVQPGRSLRLSCAASGFTFDDYAMHWVRQAPGK |
| | D433A | GLEWVAVISYDGSNKYYADSVKGRFTISRDNSKNTLYLQMNSLRA |
| | | EDTAVYYCARDFAGAIAYWGQGTLVTVSSGGGGSGGGGSGGGGS |
| | | EIVLTQSPSFLSASVGDRVTITCRASHGINNYLAWYQQKPGKAPKL |
| | | LIYAASSLQSGVPSRFSGSGSGTDFTLTISSLQPEDFATYYCQQANS |
| | | FPLTFGGGTKAAAYPYDVPDYANPDFPPEVEEQAASTLPVSCAWE |
| | | SGMKRHRAACASASINV |
| 62 | scFv-A-hPEST- | MQVQLQESGGGLVQPGRSLRLSCAASGFTFDDYAMHWVRQAPGK |
| | C441A | GLEWVAVISYDGSNKYYADSVKGRFTISRDNSKNTLYLQMNSLRA |
| | | EDTAVYYCARDFAGAIAYWGQGTLVTVSSGGGGSGGGGSGGGGS |
| | | EIVLTQSPSFLSASVGDRVTITCRASHGINNYLAWYQQKPGKAPKL |
| | | LIYAASSLQSGVPSRFSGSGSGTDFTLTISSLQPEDFATYYCQQANSF |
| | | PLTFGGGTKAAAYPYDVPDYANPDFPPEVEEQDASTLPVSAAWES |
| | | GMKRHRAACASASINV |
| 63 | scFv-A-hPEST- | MQVQLQESGGGLVQPGRSLRLSCAASGFTFDDYAMHWVRQAPGK |
| | S445A | GLEWVAVISYDGSNKYYADSVKGRFTISRDNSKNTLYLQMNSLRA |
| | | EDTAVYYCARDFAGAIAYWGQGTLVTVSSGGGGSGGGGSGGGG |
| | | SEIVLTQSPSFLSASVGDRVTITCRASHGINNYLAWYQQKPGKAPK |
| | | LLIYAASSLQSGVPSRFSGSGSGTDFTLTISSLQPEDFATYYCQQAN |
| | | SFPLTFGGGTKAAAYPYDVPDYANPDFPPEVEEQDASTLPVSCAW |
| | | EAGMKRHRAACASASINV |
| 64 | scFv-A-hPEST- | MQVQLQESGGGLVQPGRSLRLSCAASGFTFDDYAMHWVRQAPG |
| | Scramble | KGLEWVAVISYDGSNKYYADSVKGRFTISRDNSKNTLYLQMNSLR |
| | | AEDTAVYYCARDFAGAIAYWGQGTLVTVSSGGGGSGGGGSGGGG |
| | | SEIVLTQSPSFLSASVGDRVTITCRASHGINNYLAWYQQKPGKAPK |
| | | LLIYAASSLQSGVPSRFSGSGSGTDFTLTISSLQPEDFATYYCQQAN |
| | | SFPLTFGGGTKAAAYPYDVPDYAACCGEHPIRPPVEDFEESRASST |
| | | ASAWLANMVQNKPVSDA |

SEQ ID NOs: 65-81-Sequences of anti-tau-scFv (VH)

| | | |
|---|---|---|
| 65 | anti-tau scFv (VH) | QVQLQESGGGLVQPGRSLRLSCAASGFTFDDYAMHWVRQAPGK |
| | A | GLEWVAVISYDGSNKYYADSVKGRFTISRDNSKNTLYLQMNSLR |
| | | AEDTAVYYCARDFAGAIAYWGQGTLVTVSS |
| 66 | anti-tau scFv (VH) | QVQLQQSGGGVVQPGRSLRLSCAASGFTFSSYGMHWVRQAPGK |
| | B | GLEWVAVISYDGSNKYYADSVKGRFTISRDNSKNTLYLQMNSLR |
| | | AEDTAVYYCAKDLVGAKGNWGQGTLVTVSS |
| 67 | anti-tau scFv (VH) | QVQLQESGGGLVQPGRSLRLSCAASGFTFDDYAMHWVRQAPGK |
| | C | GLEWVAVISYDGSNKYYADSVKGRFTISRDNSKNTLYLQMNSLR |
| | | AEDTAVYYCARDFAGAIAYWGQGTLVTVSS |
| 68 | anti-tau scFv (VH) | QVQLVESGGGLVKPGGSLRLSCAASGFTFSSYAMSWVRQAPGKG |
| | D | LEWVAAISGSGDNTYYADSVKGRFTISRDNSENTVHLQMAGLRA |
| | | EDTALYFCAKDGPAVGNPQGYFDFWGRGTLVTVSS |
| 69 | anti-tau scFv (VH) | QVQLVQSGGGVVQPGRSLRLSCAASGFTFSSYGMHWVRQAPGK |
| | E | GLEWVASMSYDGNNKYYADSVKGRFTISRDNSKNTLYLQMNSL |
| | | RAEDTAVYYCARDLRGALDYWGQGTLVTVSS |
| 70 | anti-tau scFv (VH) | QVQLVESGGGLVKPGGSLRLSCAASGFTFSDYYMSWIRQAPGKG |
| | F | LEWVSSISSSSSYIYYADSVKGRFTISRDNAKNSLYLQMNSLRAED |
| | | TAVYYCARDGIAARSGYYGMDVWGQGTLVTVSS |
| 71 | anti-tau scFv (VH) | QVQLQESGGGLVQPGRSLRLSCAASGFTFDDYAMHWVRQAPG |
| | G | KGLEWVAVISYDGSNKYYADSVKGRFTISRDNSKNTLYLQMNS |
| | | LRAEDTAVYYCARDFAGAIAYWGQGTLVTVSS |
| 72 | anti-tau scFv (VH) | QVQLVESGGGLVKPGGSLRLSCAASGFTFSSYAMSWVRQAPGK |
| | K | GLEWVAAISGSGDNTYYADSVKGRFTISRDNSENTVHLQMAGL |
| | | RAEDTALYFCAKDGPAVGNPQGYFDFWGRGTLVTVSS |
| 73 | anti-tau scFv (VH) | QVQLVQSGGGVVQPGRSLRLSCAASGFTFSSYGMHWVRQAPGK |
| | M | GLEWVAVISYDGSNKYYADSVKGRFTISRDNSKNTLYLQMNSLR |
| | | AEDTAVYYCAKDLPDSNGYWGQGTLVTVSS |
| 74 | anti-tau scFv (VH) | QVQLVESGGGLVKPGGSLRLSCAASGFTFSSYAMSWVRQAPGK |
| | N | GLEWVAAISGSGDNTYYADSVKGRFTISRDNSENTVHLQMAGL |
| | | RAEDTALYFCAKDGPAVGNPGGYFDFWGRGTLVTVSS |
| 75 | anti-tau scFv (VH) | QVQLVQSGGGVVHPGRSLRLSCAASGFTFSSYGMHWVRQAPGK |
| | O | GLEWVASMSYDGNNKYYADSVKGRFTTPRDNSKNTLYLQMNS |
| | | LRAEDTAVYYCARDLRGALDYWGQGTLVTVSS |
| 76 | anti-tau scFv (VH) | QVQLQESGGGLVQPGRSLRLSCAASGFTFDDYAMHWVRQAPGK |
| | Q | GLEWVAVISYDGSNKYYADSVKGRFTISRDNSKNTLYLQMNSL |
| | | RAEDTAVYYCARDFAGAIAYWGQGTLVTVSS |
| 77 | anti-tau scFv (VH) | QVQLQQSGGGVVQPGRSLRLSCAASGFTFSSYGMHWARQAPGK |
| | S | GLEWVAVISYDGSNKYYADSVKGRFTISRDNSKNTLYLQMNSL |
| | | RAEDTAVYYCAKDLVGAKGNWAQGTLVTVSS |
| 78 | anti-tau scFv (VH) | QVQLQQSGGGVVQPGRSLRLSCAASGFTFSSYGMHWVRQAPG |
| | T | KGLEWVAVISYDGSNKYYADSVKGRFTISRDNSKNTLYLQMNS |
| | | LRAEDTAVYYCAKDLVGAKGNWGQGTLVTVSS |
| 79 | anti-tau scFv (VH) | QVQLVESGGGLVKPGGSLRLSCAASGFTFSSYAMSWVRQAPGK |
| | V | GLEWVAAISGSGDNTYYADSVKGRFTISRDNSENTVHLQMAGL |
| | | RAEDTALYFCAKDGPEVGNPGGYFDFWGRGTLVTVSS |
| 80 | anti-tau scFv (VH) | QVQLQQSGEGVVQPGRSLRLSCAASGFTFSSYGMHWVRQAP |
| | X | GKGLEWVAVISYDGSNKYYADSVKGRFTISRDNSKNTLYLQ |
| | | MNSLRAEDTAVYYCAKDLVGAKGNWGQGTLVTVSS |
| 81 | anti-tau scFv (VH) | QVQLVQSGGGVVQPGRSLRLSCAASGFTFSSYGMHWVRQAPG |
| | Y | KGLEWVASMSYDGDNKYYADSVKGRFTISRDNSKNTLYLQMN |
| | | SLRAEDTAVYYCARDLRGALDYWGQGTLVTVSS |

SEQ ID NOs: 82-98-Sequences of anti-tau-scFv (VL)

| | | |
|---|---|---|
| 82 | anti-tau scFv (VL) | EIVLTQSPSFLSASVGDRVTITCRASHGINNYLAWYQQKPGKAP |
| | A | KLLIYAASSLQSGVPSRFSGSGSGTDFTLTISSLQPEDFATYYCQQ |
| | | ANSFPLTFGGGTK |
| 83 | anti-tau scFv (VL) | EIVLTQSPSTLSASVGERVTITCRASQSISSWLAWYQQKPGKAPK |
| | B | VLIYKASSLESGVPSRFSGSGSGTEFTLTISSLQPDDFATYYCQQYS |
| | | TYLWTFGQGTK |
| 84 | anti-tau scFv (VL) | EIVLTQSPSILSASVGDRVTITCRASHGINNYLAWYQQKPGKAPK |
| | C | LLIYAASSLQSGVPSRFSGSGSGTDFTLTISSLQPEDFATYYCQQA |
| | | NSFPWTFGQGTK |
| 85 | anti-tau scFv (VL) | DIVMTQSPDSLAVSLGERATINCKSSQSLLYSSNNKDYLAWYQQ |
| | D | KPGQSPRLLISWASTRESGVPDRFSGSGSGTDFTLTINRLQAEDVA |
| | | VYYCQHYYSYPLTFGQGTK |
| 86 | anti-tau scFv (VL) | EIVLTQSPSTLSASIGDRVTITCRASQGISNYLAWYQQKPGKAPK |
| | E | LLIYAASTLQSGVPSRFSGSGSGTEFTLTISGLLPEDFASYFCQQASV |
| | | FPVTFGGGTK |
| 87 | anti-tau scFv (VL) | EIVLTQSPSTLSASVGDRVTITCRASQSISSWLAWYQQKPGKAPKLL |
| | F | IYAASILQSGVPSRFSGSGSGTDFTLTISSLQPEDFATYYCLQDSNPYP |
| | | LLTFGGGTK |
| 88 | anti-tau scFv (VL) | EIVLTQSPSFLSASVGDRVTITCRASHGINNYLAWYQQKPGKAPK |
| | G | LLIYAASSLQSGVPSRFSGSGSGTDFTLTISSLQPEDFATYYCQQANS |
| | | FPLTFARTK |
| 89 | anti-tau scFv (VL) | DIVMTQSPDSLAVSLGERATINCKSSQSLLYSSNNKDYLAWYQQK |
| | K | PGQSPRLLISWASTRESGVPSRFSGSGSGTDFTLTINRLQAEDVAVY |
| | | YCQHYYSYPLTFGQGTK |
| 90 | anti-tau scFv (VL) | DVVMTQSPSTLSASVGDRVTITCRASENINRWLAWYQQKPGKAP |
| | M | KLLIYKASSLESGVPSRCSGSGSGTEFTLTISSLQPDDFATYYCHQ |
| | | YTTYLWTFGQGTK |
| 91 | anti-tau scFv (VL) | DIVMTQSPDSLAVSLGERATINCKSSQSLLYSSNNKDYLAWYQQK |
| | N | PGQSPRLLIPWASTRESGVPDRFSGSGSGTDFTLTINRLQAEDVAV |
| | | YYCQHYYSYPLTFGQGTK |
| 92 | anti-tau scFv (VL) | EIVLTQSPSTLSASIGDRVTITCRASQGISNYLAWYQQKPGKAPKL |
| | O | LIYAASTLQSGVPSRFSGSGSGTEFTLTISGLLPEDFASYFCQQASV |
| | | FPVTFARTK |
| 93 | anti-tau scFv (VL) | EICVTQSPSFLSASVGDRVTITCRASHGINNYLAWYQQKPGKAP |
| | Q | KLLIYAASSLQSGVPSRFSGSGSGTDFTLTISSLQPEDFATYYCQQ |
| | | ANSFPLTFGGGTK |
| 94 | anti-tau scFv (VL) | EIVLTQSPSTLSASVGERVTITCRASQSISSWLAWYQQKPGKAPK |
| | S | VLIYKASSLESGVPSRFSGSGSGTEFTLTISSLQPDDFATYYCQQYS |
| | | TYLWTFGQGTK |
| 95 | anti-tau scFv (VL) | EIVLTQSPSTLSASVGERVTITCRASQSISSWLAWYQQKPGKAPK |
| | T | VLIYKASSLESGVPDRFSGSGSGTEFTLTISSLQPDDFATYYCQQYS |
| | | TYLWTFGQGTK |
| 96 | anti-tau scFv (VL) | DIVMTKSPDSLAVSLGERATINCKSSQSLLYSSKNKDYLAWYQK |
| | V | KPGQSPRLLISWASTRESGVPDRFSGSGSGTDFTLTINRLQAEDVA |
| | | VYYCQHYYSYPLTFGQGTK |
| 97 | anti-tau scFv (VL) | EIVLTQSPSTLSASVGERVTITCRASQSISSWLAWYQQKPGKAPKV |
| | X | LIYKASSLESGVPSFRSGSGSGTEFTLTISSLQPDDFATYYCQQYST |
| | | YLWTFGQGTK |
| 98 | anti-tau scFv (VL) | EIVLTQSPSTLSASIGDRVTITCRASQGISNYLAWYQQKPGKAP |
| | Y | KLLIYAASTLQSGVPSRFSGSGSGTEFTLTISGLLPEDFASYFCLQA |
| | | SVFPVTFGGGTK |
| 99 | huntingtin protein | MATLEKLMKAFESLKSFQQQQQQQQQQQQQQQQQQQQQQQPPPPP |
| | (GenBank | PPPPPPQLPQPPPQAQPLLPQPQPPPPPPPPPPGPAVAEEPLHRP |
| | Accession No. | KKELSATKKDRVNHCLTICENIVAQSVRNSPEFQKLLGIAMELFL |
| | NM_002111 or | LCSDDAESDVRMVADECLNKVIKALMDSNLPRLQLELYKEIKKNG |
| | NP_002102 | APRSLRAALWRFAELAHLVRPQKCRPYLVNLLPCLTRTSKRPEES |
| | | VQETLAAAVPKIMASFGNFANDNEIKVLLKAFIANLKSSSPTIRR |
| | | TAAGSAVSICQHSRRTQYFYSWLLNVLLGLLVPVEDEHSTLLILG |
| | | VLLTLRYLVPLLQQQVKDTSLKGSFGVTRKEMEVSPSAEQLVQVY |
| | | ELTLHHTQHQDHNVVTGALELLQQLFRTPPPELLQTLTAVGGIGQ |
| | | LTAAKEESGGRSRSGSIVELIAGGGSSCSPVLSRKQKGKVLLGEE |
| | | EALEDDSESRSDVSSSALTASVKDEISGELAASSGVSTPGSAGHD |
| | | IITEQPRSQHTLQADSVDLASCDLTSSATDGDEEDILSHSSSQVS |
| | | AVPSDPAMDLNDGTQASSPISDSSQTTTEGPDSAVTPSDSSEIVL |
| | | DGTDNQYLGLQIGQPQDEDEEATGILPDEASEAFRNSSMALQQAH |
| | | LLKNMSHCRQPSDSSVDKFVLRDEATEPGDQENKPCRIKGDIGQS |
| | | TDDDSAPLVHCVRLLSASFLLTGGKNVLVPDRDVRVSVKALALSC |
| | | VGAAVALHPESFFSKLYKVPLDTTEYPEEQYVSDILNYIDHGDPQ |
| | | VRGATAILCGTLICSILSRSRFHVGDWMGTIRTLTGNTFSLADCI |
| | | PLLRKTLKDESSVTCKLACTAVRNCVMSLCSSSYSELGLQLIIDV |
| | | LTLRNSSYWLVRTELLETLAEIDFRLVSFLEAKAENLHRGAHHYT |
| | | GLLKLQERVLNNVVIHLLGDEDPRVRHVAAASLIRLVPKLFYKCD |
| | | QGQADPVVAVARDQSSVYLKLLMHETQPPSHFSVSTITRIYRGYN |
| | | LLPSITDVTMENNLSRVIAAVSHELITSTTRALTFGCCEALCLLS |
| | | TAFPVCIWSLGWHCGVPPLSASDESRKSCTVGMATMILTLLSSAW |
| | | FPLDLSAHQDALILAGNLLAASAPKSLRSSWASEEEANPAATKQE |
| | | EVWPALGDRALVPMVEQLFSHLLKVINICAHVLDDVAPGPAIKAA |
| | | LPSLTNPPSLSPIRRKGKEKEPGEQASVPLSPKKGSEASAASRQS |
| | | DTSGPVTTSKSSSLGSFYHLPSYLKLHDVLKATHANYKVTLDLQN |
| | | STEKFGGFLRSALDVLSQILELATLQDIGKCVEEILGYLKSCFSR |
| | | EPMMATVCVQQLLKTLFGTNLASQFDGLSSNPSKSQGRAQRLGSS |
| | | SVRPGLYHYCFMAPYTHFTQALADASLRNMVQAEQENDTSGWFDV |
| | | LQKVSTQLKTNLTSVTKNRADKNAIHNHIRLFEPLVIKALKQYTT |
| | | TTCVQLQKQVLDLLAQLVQLRVNYCLLDSDQVFIGFVLKQFEYIE |
| | | VGQFRESEAIIPNIFFFLVLLSYERYHSKQIIGIPKIIQLCDGIM |
| | | ASGRKAVTHAIPALQPIVHDLFVLRGTNKADAGKELETQKEVVVS |
| | | MLLRLIQYHQVLEMFILVLQQCHKENEDKWKRLSRQIADIILPML |
| | | AKQQMHIDSHEALGVLNTLFEILAPSSLRPVDMLLRSMFVTPNTM |
| | | ASVSTVQLWISGILAILRVLISQSTEDIVLSRIQELSFSPYLISC |
| | | TVINRLRDGDSTSTLEEHSEGKQIKNLPEETFSRFLLQLVGILLE |
| | | DIVTKQLKVEMSEQQHTFYCQELGTLLMCLIHIFKSGMFRRITAA |
| | | ATRLFRSDGCGGSFYTLDSLNLRARSMITTHPALVLLWCQILLLV |
| | | NHTDYRWWAEVQQTPKRHSLSSTKLLSPQMSGEEEDSDLAAKLGM |
| | | CNREIVRRGALILFCDYVCQNLHDSEHLTWLIVNHIQDLISLSHE |
| | | PPVQDFISAVHRNSAASGLFIQAIQSRCENLSTPTMLKKTLQCLE |
| | | GIHLSQSGAVLTLYVDRLLCTPFRVLARMVDILACRRVEMLLAAN |
| | | LOSSMAQLPMEELNRIQEYLQSSGLAQRHQRLYSLLDRFRLSTMQ |
| | | DSLSPSPPVSSHPLDGDGHVSLETVSPDKDWYVHLVKSQCWTRSD |
| | | SALLEGAELVNRIPAEDMNAFMMNSEFNLSLLAPCLSLGMSEISG |
| | | GQKSALFEAAREVTLARVSGTVQQLPAVHHVFQPELPAEPAAYWS |
| | | KLNDLFGDAALYQSLPTLARALAQYLVVVSKLPSHLHLPPEKEKD |
| | | IVKFVVATLEALSWHLIHEQIPLSLDLQAGLDCCCLALQLPGLWS |
| | | VVSSTEFVTHACSLIYCVHFILEAVAVQPGEQLLSPERRTNTPKA |
| | | ISEEEEEVDPNTQNPKYITAACEMVAEMVESLQSVLALGHKRNSG |
| | | VPAFLTPLLRNIIISLARLPLVNSYTRVPPLVWKLGWSPKPGGDF |
| | | GTAFPEIPVEFLQEKEVFKEFIYRINTLGWTSRTQFEETWATLLG |
| | | VLVTQPLVMEQEESPPEEDTERTQINVLAVQAITSLVLSAMTVPV |
| | | AGNPAVSCLEQQPRNKPLKALDTRFGRKLSIIRGIVEQEIQAMVS |
| | | KRENIATHHLYQAWDPVPSLSPATTGALISHEKLLLQINPERELG |
| | | SMSYKLGQVSIHSVWLGNSITPLREEEWDEEEEEEADAPAPSSPP |
| | | TSPVNSRKHRAGVDIHSCSQFLLELYSRWILPSSSARRTPAILIS |
| | | EVVRSLLVVSDLFTERNQFELMYVTLTELRRVHPSEDEILAQYLV |
| | | PATCKAAAVLGMDKAVAEPVSRLLESTLRSSHLPSRVGALHGVLY |
| | | VLECDLLDDTAKQLIPVISDYLLSNLKGIAHCVNIHSQQHVLVMC |
| | | ATAFYLIENYPLDVGPEFSASIIQMCGVMLSGSEESTPSIIYHCA |
| | | LRGLERLLLSEQLSRLDAESLVKLSVDRVNVHSPHRAMAALGLML |
| | | TCMYTGKEKVSPGRTSDPNPAAPDSESVIVAMERVSVLFDRIRKG |
| | | FPCEARVVARILPQFLDDFFPPQDIMNKVIGEFLSNQQPYPQFMA |
| | | TVVYKVFQTLHSTGQSSMVRDWVMLSLSNFTQRAPVAMATWSLSC |
| | | FFVSASTSPWVAAILPHVISRMGKLEQVDVNLFCLVATDFYRHQI |
| | | EEELDRRAFQSVLEVVAAPGSPYHRLLTCLRNVHKVTTC |
| 100 | scFv-C4-hPEST | MAQVQLQESGGGLVQPGGSLRLSCAASGFTFSSYSMSWVRQAPGK |
| | | GLEWVAVISYDGSNKYYADSVKGRFTISRDNSKNTLYLQMNSLRAE |
| | | DTAVYYCARDRYFDLWGRGTLVTVSSGGGGSGGGGSGGGGSQSAL |
| | | TQPASVSGSPGQSITISCTGTSSDIGAYNYVSWYQQYPGKAPKLLIYD |
| | | VSNRPSGISNRFSGSKSGDTASLTISGLQAEDEADYYCSSFANSGPLF |
| | | GGGTKVTVLGAAAYPYDVPDYANPDFPPEVEEQDASTLPVSCAWE |
| | | SGMKRHRAACASASINV |
| 101 | scFv-C4-hPEST- | MAQVQLQESGGGLVQPGGSLRLSCAASGFTFSSYSMSWVRQAPG |
| | S440A | KGLEWVAVISYDGSNKYYADSVKGRFTISRDNSKNTLYLQMNSL |
| | | RAEDTAVYYCARDRYFDLWGRGTLVTVSSGGGGSGGGGSGGGG |
| | | SQSALTQPASVSGSPGQSITISCTGTSSDIGAYNYVSWYQQYPGKA |
| | | PKLLIYDVSNRPSGISNRFSGSKSGDTASLTISGLQAEDEADYYCSS |
| | | FANSGPLFGGGTKVTVLGAAAYPYDVPDYANPDFPPEVEEQDAS |
| | | TLPVACAWESGMKRHRAACASASINV |
| 102 | scFv-C4-hPEST- | MAQVQLQESGGGLVQPGGSLRLSCAASGFTFSSYSMSWVRQAP |
| | E428A-E430A- | GKGLEWVAVISYDGSNKYYADSVKGRFTISRDNSKNTLYLQMN |
| | E431A | SLRAEDTAVYYCARDRYFDLWGRGTLVTVSSGGGGSGGGGSGG |
| | | GGSQSALTQPASVSGSPGQSITISCTGTSSDIGAYNYVSWYQQYP |
| | | GKAPKLLIYDVSNRPSGISNRFSGSKSGDTASLTISGLQAEDEADY |
| | | YCSSFANSGPLFGGGTKVTVLGAAAYPYDVPDYANPDFPPAVA |
| | | AQDASTLPVSCAWESGMKRHRAACASASINV |
| 103 | scFv-C4-hPEST- | MAQVQLQESGGGLVQPGGSLRLSCAASGFTFSSYSMSWVRQAPG |
| | E444A | KGLEWVAVISYDGSNKYYADSVKGRFTISRDNSKNTLYLQMNSL |
| | | RAEDTAVYYCARDRYFDLWGRGTLVTVSSGGGGSGGGGSGGGG |
| | | SQSALTQPASVSGSPGQSITISCTGTSSDIGAYNYVSWYQQYPGKA |
| | | PKLLIYDVSNRPSGISNRFSGSKSGDTASLTISGLQAEDEADYYCS |
| | | SFANSGPLFGGGTKVTVLGAAAYPYDVPDYANPDFPPEVEEQDAS |
| | | TLPVSCAWASGMKRHRAACASASINV |
| 104 | scFv-C4-hPEST- | MAQVQLQESGGGLVQPGGSLRLSCAASGFTFSSYSMSWVRQAPGK |
| | P438A | GLEWVAVISYDGSNKYYADSVKGRFTISRDNSKNTLYLQMNSLRA |
| | | EDTAVYYCARDRYFDLWGRGTLVTVSSGGGGSGGGGSGGGGSQS |
| | | ALTQPASVSGSPGQSITISCTGTSSDIGAYNYVSWYQQYPGKAPKLL |
| | | IYDVSNRPSGISNRFSGSKSGDTASLTISGLQAEDEADYYCSSFANS |
| | | GPLFGGGTKVTVLGAAAYPYDVPDYANPDFPPEVEEQDASTLAVS |
| | | CAWESGMKRHRAACASASINV |
| 105 | scFv-C4-hPEST- | MAQVQLQESGGGLVQPGGSLRLSCAASGFTFSSYSMSWVRQAPG |
| | T436A | KGLEWVAVISYDGSNKYYADSVKGRFTISRDNSKNTLYLQMNSL |
| | | RAEDTAVYYCARDRYFDLWGRGTLVTVSSGGGGSGGGGSGGGG |
| | | SQSALTQPASVSGSPGQSITISCTGTSSDIGAYNYVSWYQQYPGK |
| | | APKLLIYDVSNRPSGISNRFSGSKSGDTASLTISGLQAEDEADYY |
| | | CSSFANSGPLFGGGTKVTVLGAAAYPYDVPDYANPDFPPEVEE |
| | | QDASALPVSCAWESGMKRHRAACASASINV |

SEQ ID NOs: 106-107-Anti-Huntingtin scFv sequences

| | | |
|---|---|---|
| 106 | C4 VH | QVQLQESGGGLVQPGGSLRLSCAASGFTFSSYSMSWVRQAP |
| | | GKGLEWVAVISYDGSNKYYADSVK.GRFTISRDNSKNTLYLQM |
| | | NSLRAEDTAVYYCARDRYFDLWGRGTLV |
| 107 | C4 VL | QSALTQPAS.VSGSPGQSITISCTGTSSDIGAYNYVSWYQQYPGK |
| | | APKLLIYDVSNRPSGIS.NRFSGSKSGDTASLTISGLQAEDEADYY |
| | | CSSFANSGPLFGGGTKVTVL |
| 108 | Anti-Huntingtin | QPVLTQSPSVSAAPRQRVTISVSGSNSNIGSNTVNWIQQLPGRAP |
| | VL12.3 | ELLMYDDLLAPGVSDRFSGSRSGTSASLTISGLQSEDEADYYAAT |
| | | WDDSLNGWVFGGGTKVTVL |
| 109 | Programmable | NPDFX₁X₂X₃VX₄X₅QX₆AX₇X₈ |
| | proteasome- | LX₉VX₁₀X₁₁AWX₁₂X₁₃GMX₁₄RHRAACASASINV |
| | targeting human | |
| | PEST domain | |
| | sequence | |
| 110- | PEST degron | see FIGS. 15, 29, and 35 |
| 132 | variant sequences | |
| 133 | (Gly4Ser)4 Linker | See FIG. 1 |
| 134 | 6x His tag | |
| 135- | CAG repeats | |
| 138 | | |
| 139 | Linker | Gly Gly Gly Ser |
| 140 | Linker | Ser Gly Gly Gly |
| 141 | Linker | Gly Gly Gly Gly Ser |
| 142 | Linker | Ser Gly Gly Gly Gly |
| 143 | Linker | Gly Gly Gly Gly Gly Ser |
| 144 | Linker | Ser Gly Gly Gly Gly Gly |
| 145 | Linker | Gly Gly Gly Gly Gly Gly Ser |
| 146 | Linker | Ser Gly Gly Gly Gly Gly Gly |
| 147 | Linker | (Gly Gly Gly Gly Ser)n, wherein n is an integer of one |
| | | or more |
| 148 | Linker | (Ser Gly Gly Gly Gly)n, wherein n is an integer of one |
| | | or more |

### EXAMPLES

Examples of embodiments described herein are provided in the following examples. The following examples are presented only by way of illustration and to assist one of ordinary skill. The examples are not intended in any way to otherwise limit the scope of the present embodiments.

### Example 1—Identification of Anti-α-Synuclein Intrabodies that Most Efficiently Target Synuclein to the Proteasome for Degradation, Study Overview

As described herein, bi-functional proteasome-targeting intrabodies that prevent protein misfolding while targeting their bound cargo to the proteasome for degradation via a fusion to the PEST degron from mouse ornithine decarboxylase (mPEST) have been developed. As described herein, the level of α-synuclein reduction can be controlled with a human PEST degron. It is demonstrated herein that this works in multiple systems. Without being bound by any theory, the targeted degradation of α-synuclein protein using the cell's normal protein clearing process may reduce the amount of α-synuclein available to misfold and thus reduce the physiological effects of synucleninopathies.

The targeted degradation of synuclein protein using the cell's normal protein clearing process will reduce the amount of synuclein available to misfold and thus reduce cellular toxicity due to synuclein-related neurodegenerative disease or after SCI.

### Example 2—Humanization of Bi-Functional Anti-Synuclein-PEST Intrabodies

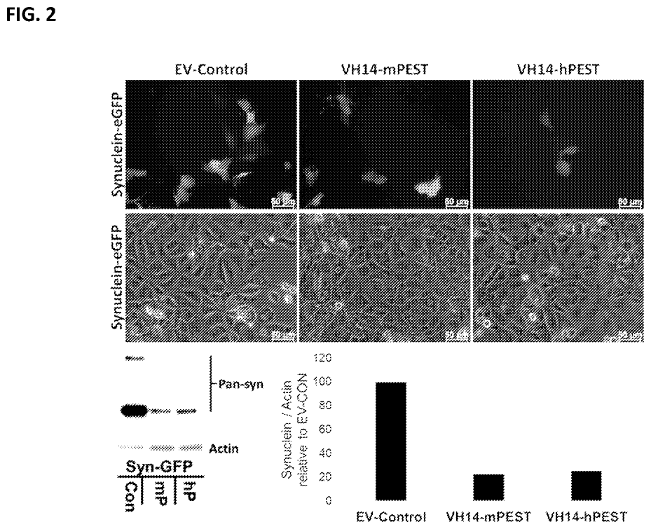

The PEST degron was optimized for human use. To accomplish this goal, the mouse PEST degron (mPEST) was substituted with the human PEST (hPEST) degron. A comparison of the mouse and human PEST degron from the ornithine decarboxylase (ODC) gene is shown in Table 3. The hPEST degron can be transferred to GFP transcriptional reporters and reduce their half-life to similar levels as GFP-mPEST reporters. Fusion of the hPEST degron to the anti-synuclein intrabodies directs synuclein to the proteasome for degradation as efficiently as mPEST degron. Therefore, the hPEST degron was cloned from human ornithine decarboxylase onto the anti-α-synuclein intrabody, VH14. VH14-hPEST reduced synuclein-GFP fluorescence to similar levels as VH14-mPEST (FIG. 2) in murine ST14A neural precursor cells. α-synuclein-GFP was co-transfected with either VH14-mPEST or VH14-hPEST. 72 h after transfection, cells were live imaged.

Western blot analysis confirmed the live cell imaging result, as VH14-hPEST reduced the steady state protein levels of human α-synuclein-GFP to same extent as VH14-mPEST. As shown in FIG. 2, α-synuclein-GFP monomer in VH14-mPEST and VH14-hPEST transfected cells were reduced compared to empty vector control. VH14-mPEST and VH14-hPEST also reduced the presence of high molecular species of α-synuclein-GFP observed in empty vector control samples. Actin was probed as a loading control. Graphs show densitometric analysis of western blot signals. Each bar represents pan-synuclein/actin loading control expressed as a percentage of empty vector control.

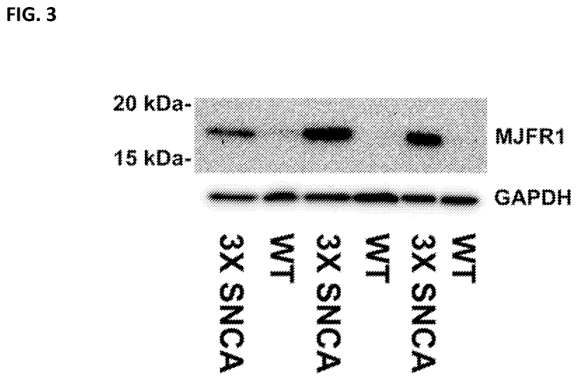

Patient-derived induced pluripotent stem cells (iPSCs) from Parkinson's disease patients were established with an increased copy number mutation in the Synuclein Alpha (SNCA) gene (SNCA Triplication (RUCDR; ND50040) encoding α-synuclein. Patients with this mutation develop autosomal dominant Parkinson's disease. Optimization was then done for a human iPSC 3-Dimensional (3D) cerebral organoid protocol developed by Sergiu Pasca and known in the art. Cultures utilizing this protocol result in cerebral organoids which are approximately 1-3 mm diameter balls of human neural cells that consist predominately of neurons but also include macroglia. This protocol was selected for its high reproducibility and ability to produce all the major cerebral cortical cell types, including CTIP2-positive neurons, which are present in cortical layer V, and send their axons to deep brain structures, such as the thalamus (corticothalamic neurons), the striatum (corticostriatal neurons), pons (corticopontine neurons), tectum (corticotectal neurons), and spinal cord (corticospinal motor neurons, CSMN). Endogenous synuclein overexpression was verified in human iPSC-derived 3D organoid 60-day-old cortical neurons with the SNCA gene triplication (3×SNCA) compared to wild type (WT) healthy control by western blotting (FIG. 3). As shown in the figure, 3×-SNCA and WT iPSCs were differentiated into 3D forebrain organoids. Following 60 days in vitro (DIV), organoids were harvested for western blotting. 20 ug (left 4 lanes) or 10 μg (lanes 5 and 6) of total protein was separated by gel electrophoresis and transferred onto PVDF membranes. Endogenous synuclein was detected using an MJFR1 anti-synuclein antibody (1:1000; Abcam #ab138501). GAPDH was used as a loading control (1:10,000; Abcam, #ab181602).

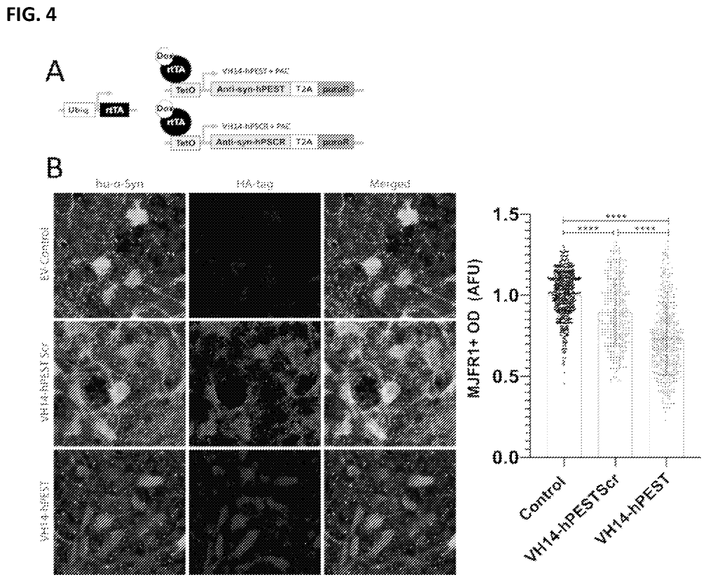

To evaluate the targeted degradation of α-synuclein in human neurons, WT and 3×-SNCA 3D cortical organoids were transduced with inducible lentivirus expressing either anti-α-synuclein VH14 with the human PEST degron (VH14-hPEST), or VH14-hPEST with a scrambled PEST degron control (VH14-hPEST-Scr). FIG. 4A shows design of Tet-On inducible anti-synuclein lentiviral constructs. Briefly, anti-synuclein mPEST and hPEST intrabodies were subcloned into pTet-O-Ngn2-puro (Addgene plasmid #52047). The Ngn2 insert was replaced with VH14-mPEST, VH14-hPEST, VH14-mPEST-Scramble-control, VH14-hPEST-Scramble-control, VHH-B8-mPEST, and VHH-B8-hPEST. The 5′ cloning site was EcoRI and the 3′ cloning site was XbaI. Following 30 days of treatment, VH14-hPEST significantly reduced endogenous α-synuclein levels compared to empty virus control and VH14-hPEST-Scr control (FIG. 4B). Briefly, VH14-hPEST reduced levels of endogenous human α-synuclein in 3×SNCA forebrain organoids. Immunofluorescence shows reduction in α-synuclein (MJFR1; Abcam, Green) levels in organoids transduced (n=3) with lentivirus carrying VH14-hPEST compared to empty vector (EV)-treated organoids and organoids treated with VH14 fused to a scrambled (Scr) PEST. Densitometric quantification of α-synuclein signal in organoids confirm statistically significant reduction of target protein in both VH14-treated cohorts. (all ****p<0.0001). These experiments are a significant improvement upon previous intrabody drug screening models relying on immortalized tumor-derived cell lines, which may contain genetic and metabolic abnormalities due to their mode of derivation. Additionally, experiments on such lines are typically limited to short time frames (2-4 days) due to their continuous proliferation. Human iPSC-derived cultures overcome these limitations by more faithfully simulating human neural cells and disease phenotypes.

### Example 3—Development of Control Intrabodies for In Vivo Studies

To control for the effects of both protein overexpression and proteasome degradation of PEST-tagged intrabodies, a control single-domain intrabody was generated against an antigen not expressed in any of the test systems. VHH-B8 is a well-characterized camelid nanobody that binds to Botulinum Neurotoxin, and has demonstrated excellent intracellular solubility in test systems.

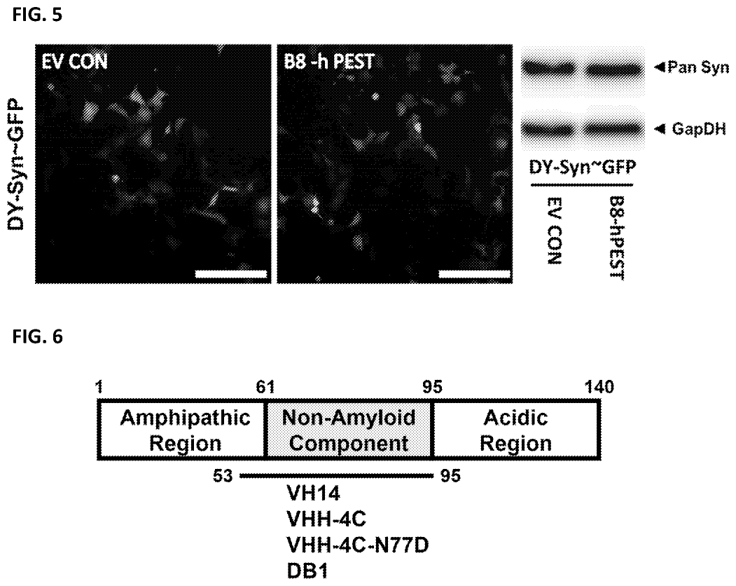

As expected, a bi-functional intrabody targeting botulinum toxin (B8) with hPEST (B8-hPEST) does not alter the clearance of the steady-state protein levels of lamprey DY-synuclein˜GFP (DY-syn˜GFP) (FIG. 5). Briefly, ST14A neuronal cells were co-transfected with DY-syn˜GFP and either B8-hPEST or Empty Vector control. 48 hours after transfection, cells were live cell imaged and then harvested for western blotting. Live Cell Imaging for DY-syn˜GFP (Scale bar 200 μm). Representative western blotting is as described above. In order to determine target engagement of the intrabody for endogenous human α-synuclein and the hPEST degron for the proteasome, N77D-mPEST, N77D-hPEST, N77D-mPEST-Scramble-control, and N77D-hPEST-Scramble-control intrabodies were cloned into a tetracycline-inducible lentiviral vector with puromycin resistance to allow for stable gene selection (Table 3).

TABLE 3
Intrabody Constructs Generated for Testing

| | | | | Vector Backbone | | |
|---|---|---|---|---|---|---|
| | | | | pcDNA3.1 | | pTetO-Fuw |
| | Intrabody | Type | Antigen | (—) | pAAV-MCS | (I.U./mL) |
| 1 | VH14-mPEST | Single Domain, | Human α-synuclein | 1000 ng/μL | 1000 ng/μL | 1.14 × 10⁸ |
| 2 | VH14-mPEST-Scramble | VH | synuclein | 1000 ng/μL | 1000 ng/μL | 1.10 × 10⁸ |
| 3 | VH14-hPEST | | | 1000 ng/μL | 1000 ng/μL | 1.34 × 10⁸ |
| 4 | VH14-hPEST-Scramble | | | 1000 ng/μL | 1000 ng/μL | 4.14 × 10⁸ |
| 5 | VH14-hPEST-P426/427 A | | | 1000 ng/μL | 1000 ng/μL | 1.34 × 10⁸ |
| 6 | VH14-hPEST-D433A | | | 1000 ng/μL | 1000 ng/μL | 1.34 × 10⁸ |
| 7 | VH14-hPEST-S445A | | | 1000 ng/μL | 1000 ng/μL | 3.97 × 10⁸ |
| 8 | VH14-hPEST-C441A | | | 1000 ng/μL | 1000 ng/μL | 1.34 × 10⁸ |
| | (inactive variant) | | | | | |
| 9 | N77D-mPEST | Camelid Single | Human α-synuclein | 1000 ng/μL | 1000 ng/μL | — |
| 10 | N77D-mPEST-Scramble | Domain, VHH | Lamprey DY-synuclein | 1000 ng/μL | 1000 ng/μL | — |
| 11 | N77D-hPEST | | | 1000 ng/μL | 1000 ng/μL | 9.02 × 10⁷ |
| 12 | N77D-hPEST-Scramble | | | 1000 ng/μL | 1000 ng/μL | 6.07 × 10⁷ |
| 13 | DB1-pmCherry | | Human α-synuclein | 1000 ng/μL | — | — |
| 14 | DB1-hPEST | | Lamprey DY-synuclein | 1000 ng/μL | — | — |
| 15 | B8-mPEST | | Botulinum Neurotoxin | 1000 ng/μL | 1000 ng/μL | 1.08 × 10⁹ |
| 16 | B8-hPEST | | (Irrelevant Antigen) | 1000 ng/μL | 1000 ng/μL | 2.22 × 10⁸ |

### Example 4—Identification of Candidate Intrabodies Targeting Human and Model Organism Synuclein

Lampreys express three synuclein isoforms (DY-synuclein˜GFP, FD-synuclein˜GFP, and syn3˜GFP), which have significant homology to human synucleins. The most abundantly expressed synuclein isoform in RS neurons is a γ-synuclein, DY-synuclein, which is ˜70% identical and ˜90% similar to the first 90 amino acids of human α-synuclein. Human α-synuclein and lamprey synuclein are highly conserved in their N-terminal domains (see diagram of human α-synuclein protein in FIG. 6), and published studies confirmed they have similar biochemical properties (e.g., lipid membrane binding) and functional effects in neurons (e.g., synaptic vesicle trafficking). To identify the optimal therapeutic target to reduce human α-synuclein toxicity following SCI, a series of bi-functional anti-α-synuclein intrabodies directed to each of the major regions of α-synuclein were generated and fused to a proteasomal targeting motif (FIG. 6). The proposed intrabody binding site locations of VH14, VHH-4C, VHH-4C-N77D, and DB1 to the non-amyloid component (NAC) hydrophobic domain of α-synuclein that is prone to aggregation and has been shown to be critical for misfolding

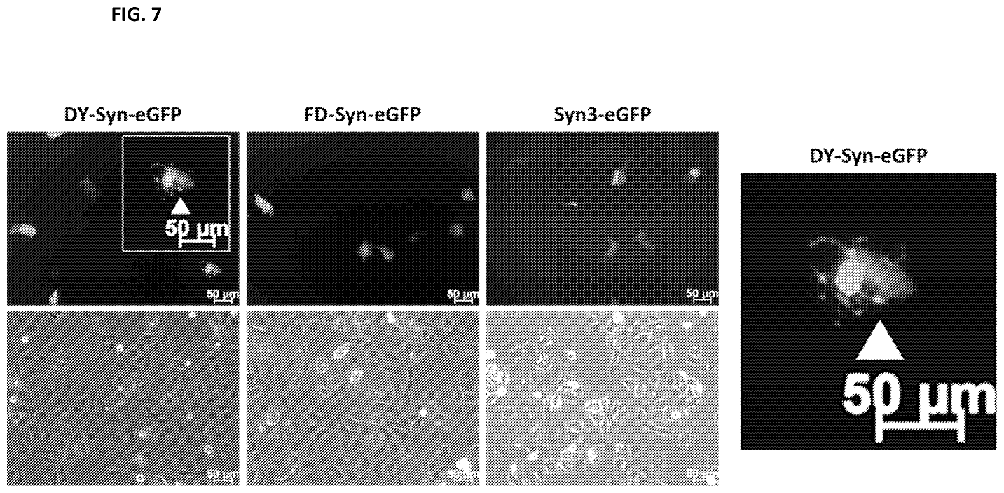

Lamprey DY-synuclein˜GFP, FD-synuclein˜GFP, and syn3-synuclein˜GFP were cloned into a mammalian pcDNA3.1 expression plasmid. After sequence verification of these plasmids, their expression in ST14A neuronal cell line was verified (FIG. 7). The figure shows expression of lamprey synuclein. DY-syn˜GFP, FD-syn˜GFP, and Syn3˜GFP were separately transfected into ST14A neuronal cells. 48 hours after transfection, cells were live cell imaged. In a subset of cells, DY-synuclein forms puncta indicative of α-synuclein aggregation (FIG. 7 Inset). Because DY-synuclein is the predominant variant of lamprey synuclein, initial focus was placed on this in preliminary studies. The established intrabodies to human α-synuclein d5PEST, VH14PEST, NAC32PEST, syn2PEST, and syn87PEST were screened against lamprey DY-synuclein, but significant turnover of lamprey DY-synuclein˜GFP was not observed in the culture system. Antibodies to antigen proteins from different species that share 75% sequence homology are generally predicted to cross-react. The lack of DY-synuclein degradation is likely due to sequence homology differences between human α-synuclein and lamprey DY synuclein, since they only share 67% sequence homology with each other.

### Example 5—Development of Novel Anti-Synuclein Nanobodies

To identify an intrabody that can cross-react to human α-synuclein and lamprey DY-synuclein, a VHH nanobody was produced by Hybribody services.

Camelid single-domain nanobodies were screened against DY-synuclein. Camelids produce a unique class of immunoglobulins, which are devoid of light chains and are therefore termed heavy-chain antibodies (HCAbs). Camelid HCAbs demonstrate binding affinities similar to conventional antibodies for many antigens. Unlike conventional antibodies, however, HCAbs use a single variable heavy chain (VHH) to bind an epitope, eliminating the need for the hinged structure that characterizes the single-chain antibody Fv fragments comprised of both variable heavy and light chains. Camelid VHH nanobodies were chosen because they have an extensive antigen-binding repertoire, and exhibit highly favorable properties for therapeutic research applications such as their small size, high solubility, thermal stability, refolding capacity, good tissue penetration in vivo, and ability to bind unique epitopes. This approach identified antibody named DB1 (SEQ ID NO:5):

MAEVQLQASGGGFVQPGGSLRLSCAASGFTSWEDTMGWF
RQAPGKEREFVSAISFDANDLSDTSVYY ADSVKGRFTI
SRDNSKNTVYLQMNSLRAEDTATYYCAVASFEILLYGES
LHIYWGQGTQVTVSS.

### Example 6—Intrabody Screening

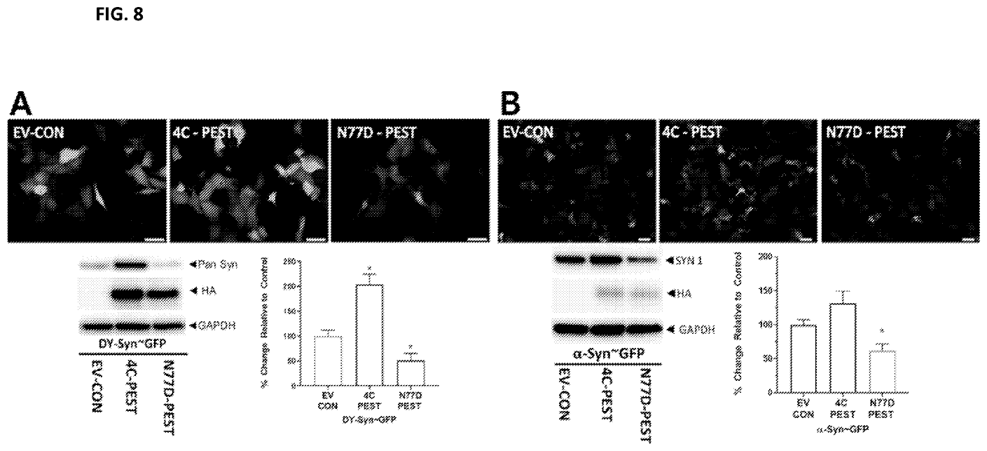

Two additional anti-synuclein-VHH single domain intrabodies were screened, VHH-4C and VHH-4C-N77D (referred to herein as N77D). These intrabodies were derived from the immunized phagemid synuclein alpaca VHH immune library (Addgene #1000000071) as used above but were isolated via functional ligand-binding identification by Tat-based recognition of associating proteins. N77D was developed through computational affinity maturation and differs from its parental by one amino acid (N77D). N77D displayed enhanced nanomolar affinity to α-synuclein compared to the micromolar affinity of VHH-4C through an increased association rate verified by surface plasmon resonance (SPR) experiments. N77D-mPEST reduced the steady state levels of lamprey DY-synuclein˜GFP (FIG. 8A) and human α-synuclein˜GFP (FIG. 8B) by approximately 40%.

In this synuclein overexpression system, VHH-4C-PEST increased the soluble monomeric levels of DY-synuclein relative to empty vector control (FIG. 8A). VHH-4C-PEST also increased the levels of α-synuclein relative to empty vector control (FIG. 8B). Without wishing to being bound by any theory, the enhanced levels may be due to a decreased ability to pull synuclein into the proteasome. Based on the cell free experiments, and again without wishing to being bound by any theory, VHH-4C may bind to the DY-synuclein but not have sufficient affinity to re-direct DY-synuclein into the proteasome. This binding reaction could potentially reduce the normal turnover of the protein. However, no signs of overt cell death were observed, which would be indicative of toxicity, and no signs of DY-synuclein˜GFP puncta were observed which are reflective of aggregated synuclein as shown in FIG. 7. The enhanced affinity of N77D to synuclein may be responsible for increased functional activity within cells compared to VHH-4C.

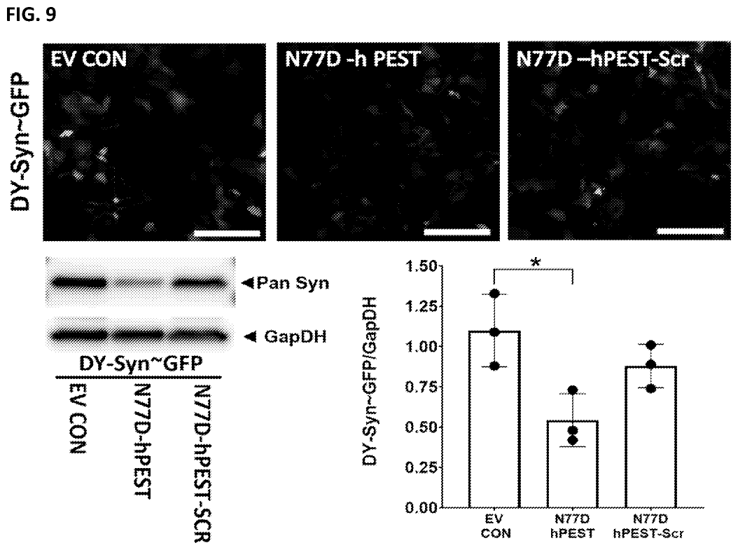

After verifying that N77D-PEST significantly increased the degradation of DY-synuclein˜GFP (FIG. 8A) and α-synuclein˜GFP (FIG. 8B), the PEST degron from human ornithine decarboxylase (hPEST) was cloned onto VHH-N77D and it was determined that N77D-hPEST can redirect lamprey DY-synuclein˜GFP into the proteasome for degradation in ST14A rat medium spiny neuron precursor cells (FIG. 9).

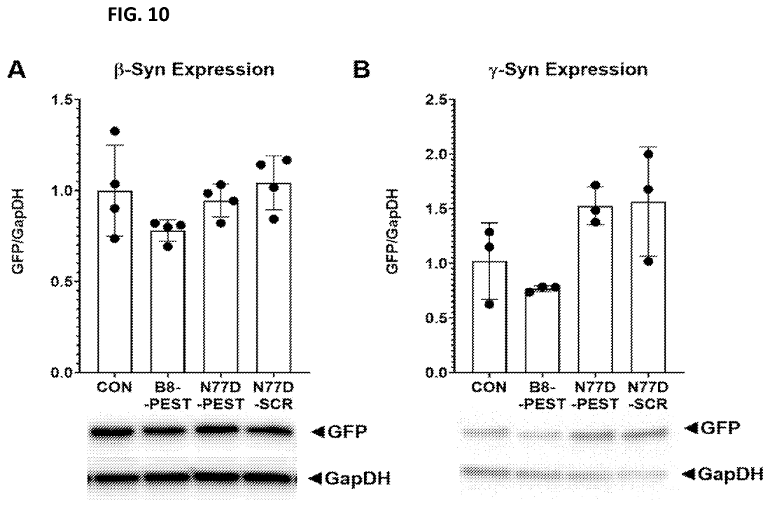

The specificity vs off-target binding of N77D-hPEST was examined. β-synuclein and γ-synuclein were cloned into GFP-pcDNA3.1(−) plasmid to generate β-synuclein˜GFP and γ-synuclein˜GFP fusion proteins. ST14A cells were then co-transfected with either β-synuclein˜GFP or γ-synuclein˜GFP and either empty vector control, N77D-hPEST, N77D-hPEST-Scr control, or B8-hPEST control. N77D-hPEST did not significantly alter the degradation of either β-synuclein˜GFP (FIG. or γ-synuclein˜GFP (FIG. 10B).

### Example 7—Controlled Degradation of Intracellular Proteins Using a Human PEST

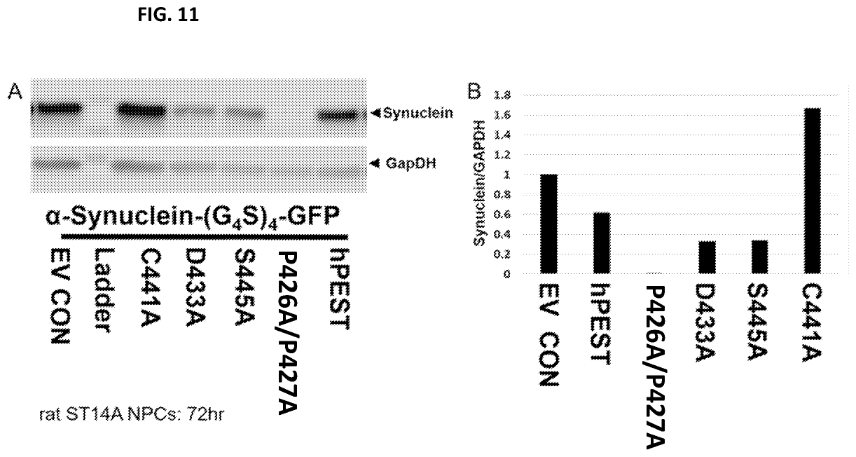

The present method uses human ODC and controls the level of degradation by creating mutations at specific locations (FIG. 15). We have shown that by altering the PEST sequence at designated sites, different levels of degradation of human α-synuclein are achieved. C441A renders the PEST sequence inactive and causes an apparent increase in observed α-synuclein compared to empty vector control. A significant increase in degradation relative to empty vector control was observed with VH14-hPEST modifications S445A and D433A (FIG. 11). A significant increase in degradation compared to S445A and D433A was observed with the VH14-hPEST modifications P426A and P427A (a compound mutation) (FIG. 11). This shows different levels of degradation are achievable with different PEST modifications.

As shown in FIG. 15, a mutation that may be present in a PEST degron relative to SEQ ID NO:1 for targeting a protein to the proteasome to achieve increased degradation of the protein may be a mutation of one or more proline (P) residue to an alanine (A) residue, such as P426A/P427A (i.e., mutation of 2 consecutive P residues to 2 consecutive A residues), a mutation of one or more aspartic acid (D) residue to an A residue, such as D433A, a mutation of one or more serine (S) residue to an A residue, such as S445A, and/or a mutation of one or more lysine (K) residue to an A residue, such as K448A. In some embodiments, additional mutations may be made in one or more amino acid residues of the human ODC PEST degron to enhance the degradation of a protein, such as α-synuclein.

In other embodiments, a mutation that may be present in a PEST degron relative to SEQ ID NO:1 as described herein for targeting a protein to the proteasome to achieve reduced degradation of the protein may be a mutation of a P residue to an A residue, such as P438A, a mutation of a glutamic acid (E) residue to an A residue, such as E444A, a mutation of an S residue to an A residue, such as S440A, and/or a mutation of a threonine (T) residue to an A residue, such as T436A.

### Example 8—Human Ornithine Decarboxylase (ODC) PEST (hPEST) Degron Variants

A panel of intrabodies that target α-synuclein to the proteasome for degradation via a human proline (P), glutamic acid (E), aspartic acid (D), serine (S) and threonine (T) (PEST) degron fusion has been developed. FIG. 15 shows the PEST degron variants identified in this study. Certain mutations within the hPEST degron (highlighted in grey) alter the targeted degradation of the intrabody and its bound antigen. The PEST degron is shaded on the top row in grey (ODC amino acids 423-450).

### Example 9—Efficacy Testing for Mutations that Increase or Decrease Degradation of α-Synuclein Proteins

Testing of the efficacy of a mutation for increasing degradation of α-synuclein is done using any methods for protein binding, translocation, and the like. For example, a mutation, such as including, but not limited to, P426A/P427A, D433A, S445A, and/or K448A is introduced into a human PEST degron sequence as described herein (SEQ ID NO:1). For visualization, a GFP marker or other appropriate screenable marker is used to be able to visualize translocation of the protein to the proteasome. Appropriate controls are used for comparison and to determine success of the mutation in increasing transport of the protein to the proteasome. A cysteine (C)-to-A mutation at residue 441 (C441A or C20A) can be used as a control, as this mutation does not have a therapeutic effect.

In addition, a subset of mutations using glycine (G) instead of alanine (A) are made and tested using the same methods.

### Example 10—Validated Target Engagement of Intrabodies to α-Synuclein and hPEST Degron to the Proteasome in iPSC Derived Cortical Neurons

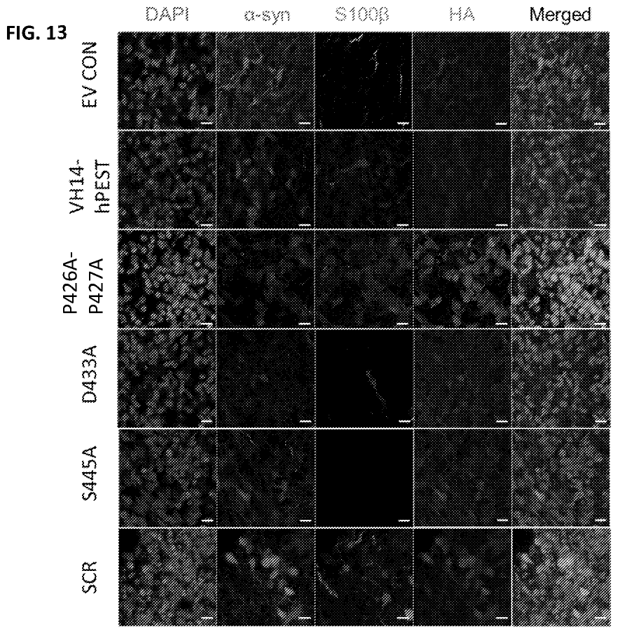

PEST degrons as described herein will be used in an iPSC-derived cortical and midbrain organoid system as shown in FIG. 13. Patient-derived induced pluripotent stem cells (iPSCs) from Parkinson's disease patients with an increased copy number mutation in the SNCA gene (SNCA Triplication (RUCDR; ND50040) encoding α-synuclein and iPSCs from healthy donors will be used as a control. Patients with this mutation develop autosomal dominant Parkinson's disease. The test system used for this optimization screening will be lentiviral transduction of wild type iPSC-derived cortical forebrain and midbrain organoids with bi-functional anti-α-synuclein-hPEST intrabodies. Candidate intrabodies described herein will be subcloned into a tetracycline inducible pTetO-puromycin resistant lentiviral vector, and then transduced into cortical or midbrain organoids at 30 days. Endogenous bi-functional α-synuclein degradation will be verified by immunofluorescent staining and by quantitative western blotting with anti-synuclein monoclonal antibody MJFR1 (1:1,000). HA-tagged intrabodies will be probed with monoclonal anti-HA (1:5,000, Covance). Samples will be normalized to either actin or GAPDH housekeeping proteins with monoclonal (anti-actin; 1:1000, Sigma or anti-GAPDH; 1:10,000 Abcam) antibodies. Densitometry will be quantified with Image J software. An n of 3 samples per treatment group will be analyzed.

### Example 11—Controlled Degradation of Intracellular Synculein Protein Using Bifunctional Anti-Synculein Intrabody with a Human PEST Degron in Rat ST14A Neural Precursor Cells

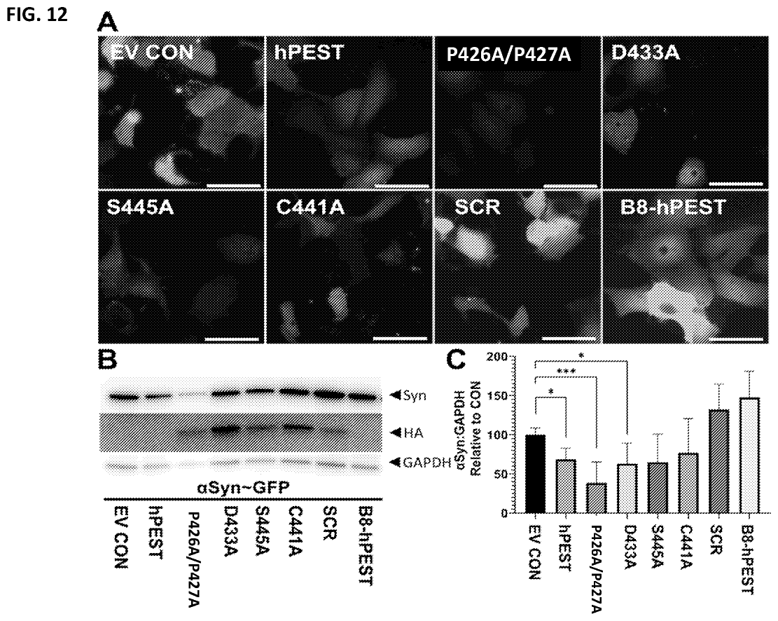

To identify hPEST degron variants that alter the degradation of tau to desired levels, ST14A neuronal cells were transfected with α-synuclein˜GFP and either empty vector control (EV CON), VH14-hPEST, VH14-hPEST degron variants P426A/P427A, D433A, C441A, S445A, inactive scrambled PEST degron control (SCR), or irrelevant antigen control (B8-hPEST). 72 h after transfection, samples were collected for: (A) Live cell imaging. (B) Western blot. (C) The relative protein expression was determined by the ratio of α-synuclein to an internal standard control (GAPDH). Samples were then normalized to EV-control. Human PEST degron variants resulted in altered protein degradation levels compared empty vector control (EV CON) (FIG. 12). Compound mutation variant P426A/P427A resulted in altered synuclein expression compared to VH14-hPEST and a significant (p<0.001) protein degradation level compared to empty vector control (EV CON). VH14-hPEST and VH14-hPEST variant D433A significantly reduced (P<0.01) synuclein compared to EV CON.

### Example 12—Controlled Degradation of Intracellular Proteins Using Anti-Synculein Intrabody with a Human PEST Degron in iPSC Derived Midbrain Neurons

The present method uses human ODC and controls the level of degradation by creating mutations at specific locations (FIG. 15). We have shown that by altering the PEST sequence at designated sites, different levels of degradation of human □-synuclein are achieved. PEST degrons as described herein were used in an iPSC-derived midbrain organoid system as shown in FIG. 13. Patient-derived induced pluripotent stem cells (iPSCs) from Parkinson's disease patients with an increased copy number mutation in the SNCA gene (SNCA Triplication (RUCDR; ND50040) encoding α-synuclein and iPSCs from healthy donors were used as a control. Patients with this mutation develop autosomal dominant Parkinson's disease. The test system used for this optimization screening was lentiviral transduction of wild type iPSC-derived midbrain organoids with bi-functional anti-α-synuclein-hPEST intrabodies. Candidate intrabodies described herein were subcloned into a tetracycline inducible pTetO-puromycin resistant lentiviral vector, and then transduced into midbrain organoids at 30 days. Endogenous bi-functional α-synuclein mediated degradation was verified by immunofluorescent staining at 60 days. An n of 3 samples per treatment group was analyzed. A significant increase in degradation relative to empty vector control and VH14-PEST-SCR (inactive PEST degron control) was observed with VH14-hPEST. VH14-hPEST variants S445A, D433A, and P426A and P427A (a compound mutation) displayed increased degradation compared to VH14-PEST (FIG. 13). This shows different levels of degradation are achievable with different PEST modifications following prolonged expression in disease relevant cell types.

For Examples 11 and 12, Transfection-Rat Progenitor cells (ST14A), which display neuronal characteristics were utilized for transfection. ST14A cells were cultured using standard protocols. Cells were cultured into 12 well plates for transfection. Co-transfection was performed using 0.75 μg prk5-GFP-α-synuclein per well and 2.25 μg Anti-α-synuclein-hPEST or Anti-α-synuclein-hPEST variants expressed in pcDNA3.1(−) expression intrabody expression vectors per well. PEI DNA transfection reagent was utilized in order to transiently transfect cells. All cultures were imaged 72 hours after transfection, after which cultures were harvested for western blot analysis.

Western Blotting—72 hours after transfection, ST14A cells were imaged for GFP expression. After imaging, samples were collected from 6 well plates by trypsinization. Cell samples were washed with 1×PBS followed by cell lysis using RIPA buffer plus 1× protease inhibiter cocktail (50 mM Tris pH 7.5, 150 mM NaCl, 1% NP40, 0.25% sodium deoxycholate, 2% SDS). Samples were then sonicated for minutes. DC protein assays were performed on samples to generate protein concentration data. From protein assays, sample concentrations were normalized to 1 ng/mL in 2× denaturing sample buffer (125 mM Tris, 4% SDS, 20% glycerol, 10% 2-mercaptoethanol, 0.02% bromophenol blue, pH 6.8) and heated in order to ensure denaturation of proteins. 10 μg of each lysate sample were separated though sodium dodecyl sulfate polyacrylamide gel electrophoresis (SDS-PAGE) using 4-20% Criterion precast gels (Bio-Rad #456-1095). Proteins were blotted onto PDVF membranes (Millipore) using a transblot semi-dry (SD) electroblotter (Bio-Rad) at 24-27V for 30 min. The PDVF membranes were probed for Total α-synuclein (MJFRI or synI; 1:1,000) and GAPDH (as a loading control, Abcam; 1:5,000).

### Example 13—Bi-Functional VH14-PEST Enhance Viability in iPSC Derived Midbrain Cultures with 3×SNCA Mutation

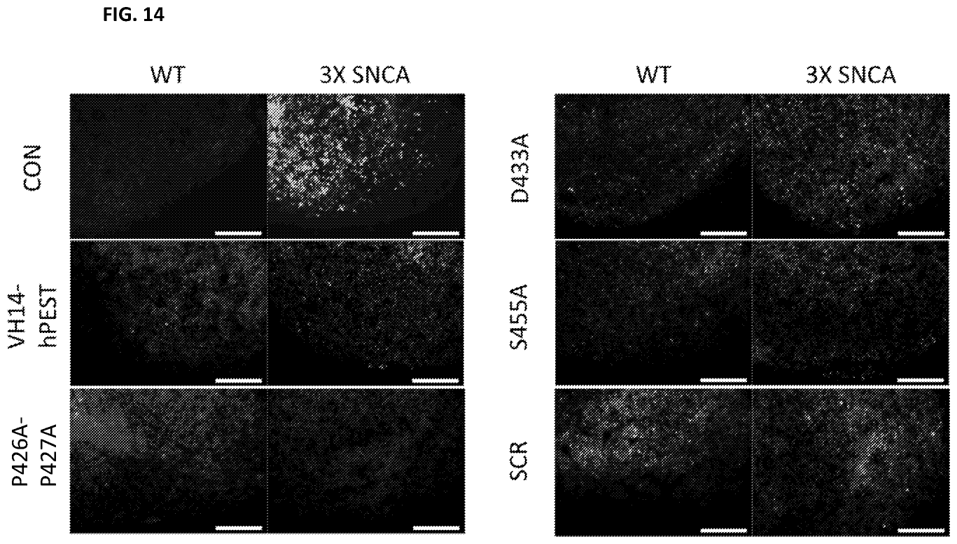

To test the safety and efficacy of anti-synuclein bi-functional intrabodies, patient derived iPSC with the 3×SNCA gene triplication and iPSCs from a healthy control (WT) were differentiated into 3D midbrain organoids, an area of the brain affected in Parkinson's Disease. At 30 days, organoid was transduced with either empty vector control (EV), VH14-hPEST, VH14-hPEST degron variants P426A/P427A, D433A, S445A, or scrambled PEST degron control (SCR). At 60 days, cell death was evaluated using a terminal deoxynucleotidyl transferase dUTP nick end labeling staining, also called the TUNEL assay (DeadEnd Fluorometric TUNEL system; Promega #G3250). As expected, mutant 3×SNCA midbrain organoids displayed increased TUNEL reactivity compared to WT midbrain organoids (FIG. 14). There was minimal TUNEL reactivity in WT or 3×SNCA midbrain organoids treated with VH14-hPEST and VH14-hPEST degron variants P426A/P427A, D433A, S445A (FIG. 14). VH14-hPEST, which displayed the lowest level of synuclein reduction in immortalized cells (FIG. 11 and FIG. 12) and midbrain organoids (FIG. 13) was as effective as strongest synuclein reduced VH14-hPEST degron variant P426A/P427A at preserving cell viability following prolonged treatment. Thus, the minimal level of synuclein reduction to provide a therapeutic effect can be used with this technology.

### Example 14—Identification of Anti-Tau Intrabodies that Most Efficiently Target Tau to the Proteasome for Degradation, Study Overview

Tau is a protein that is involved in a number of neurodegenerative diseases, such as tauopathies including Alzheimer's Disease (AD) and Frontotemporal dementia (FTD), as well as traumatic brain injury (TBI) and spinal cord injury (SCI). Tauopathies result when tau protein accumulates into aggregates, resulting in neurological symptoms as a result of neuronal and glial cell dysfunction and death. Targeted degradation of abnormal tau protein is therefore an important therapeutic target. Intrabodies can be designed and selected to bind to various protein conformations and epitopes on their targets. In addition, they can be further engineered to relocate target proteins to different cellular compartments such as the nucleus, endoplasmic reticulum, and proteasome (FIG. 1).

As described herein, bi-functional proteasome-targeting intrabodies that prevent protein misfolding while targeting their bound cargo to the proteasome for degradation via a fusion to the PEST degron from human ornithine decarboxylase (hPEST) has been developed. As described herein, the level of tau reduction can be controlled with a human PEST degron. It is demonstrated herein that this works in multiple systems. Without being bound by any theory, the targeted degradation of tau protein using the cell's normal protein clearing process may reduce the amount of tau available to misfold and thus reduce the physiological effects of tauopathies.

### Example 15—Humanization of Bi-Functional Anti-Tau-PEST Intrabodies

The PEST degron may be optimized for human use by substituting the mouse PEST degron (mPEST) with the human PEST (hPEST) degron. A comparison of the mouse and human PEST degron from the ornithine decarboxylase (ODC) gene is shown in FIG. 17. The hPEST degron can be transferred to GFP transcriptional reporters and reduce their half-life to similar levels as GFP-mPEST reporters. Fusion of the hPEST degron to the anti-tau intrabodies may direct tau to the proteasome for degradation as efficiently as the mPEST degron. Therefore, the hPEST degron is cloned from human ornithine decarboxylase onto an anti-tau intrabody.

While previous experiments utilizing immortalized tumor-derived cell lines have generated valuable information, they do present significant limitations due to their derivation. Patient-derived induced pluripotent stem cells (iPSCs) can overcome these limitations by more faithfully simulating human disease phenotypes observed in the CNS. iPSC lines from patients with tauopathies are established with a mutation in the gene encoding tau. Optimization is then done using a human iPSC 3-Dimensional (3D) cerebral organoid protocol developed by Sergiu Pasca and known in the art. Cultures utilizing this protocol result in cerebral organoids which are approximately 1-3 mm diameter balls of human neural cells that consist predominately of neurons but also include macroglia. This protocol was selected for its high reproducibility and ability to produce all the major cortical cell types, including CTIP2-positive neurons, which are present in cortical layer V, and send their axons to deep brain structures, such as the thalamus (corticothalamic neurons), the striatum (corticostriatal neurons), pons (corticopontine neurons), tectum (corticotectal neurons), and spinal cord (corticospinal motor neurons, CSMN). Expression of endogenous tau having a mutation as described herein is verified in 3-Dimensional (3D) 60-day-old cortical neurons with the mutation compared to wild type (WT) healthy control by western blotting.

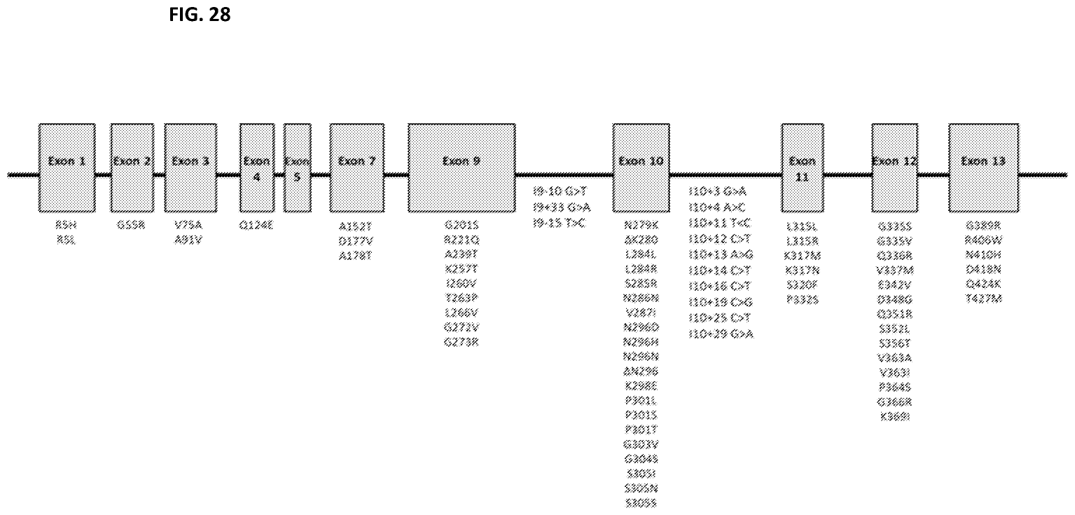

FIG. 28 shows a schematic of the MAPT (tau) gene, along with mutations in particular exons and introns. These mutations include, but are not limited to, the following mutations:
Exon 1: R5H and R5L;
Exon 2: G55R;
Exon 3: V75A and A91V;
Exon 4: Q124E,
Exon 7: A152T, D177V, and A178T;
Exon 9: G201S, R221Q, A239T, K257T, 1260V, T263P, L266V, G272V, and G273R;
Intron 9: I9-10 G>T, I9+33 G>A, and I9-15 T>C;
Exon 10: N279K, ΔK280, L284L, L284R, S285R, N286N, V2871, N296D, N296H, N296N, ΔN296, K298E, P301L, P301S, P301T, G303V, G304S, S305I, S305N, and S305S;
Intron 10: I10+3 G>A, I10+4 A>C, I10+11 T<C, I10+12 C>T, I10+13 A>G, I10+14 C>T, I10+16 C>T, I10+19 C>G, I10+25 C>T, and I10+29 G>A;
Exon 11: L315L, L315R, K317M, K317N, S320F, and P332S;
Exon 12: G335S, G335V, Q336R, V337M, E342V, D348G, Q351R, S352L, S356T, V363A, V363I, P364S, G366R, and K369I;
Exon 13: G389R, R406W, N410H, D418N, Q424K, and T427M. iPSCs have been previously prepared (Karch PMID:31631020) for a number of the mutations listed above, including A152T, N279K, P301L, S305N, IVS10+16, G335S, G335V, V337M, G389R, and R406W.

### Example 16—Controlled Degradation of Intracellular Proteins Using a Human PEST Variant

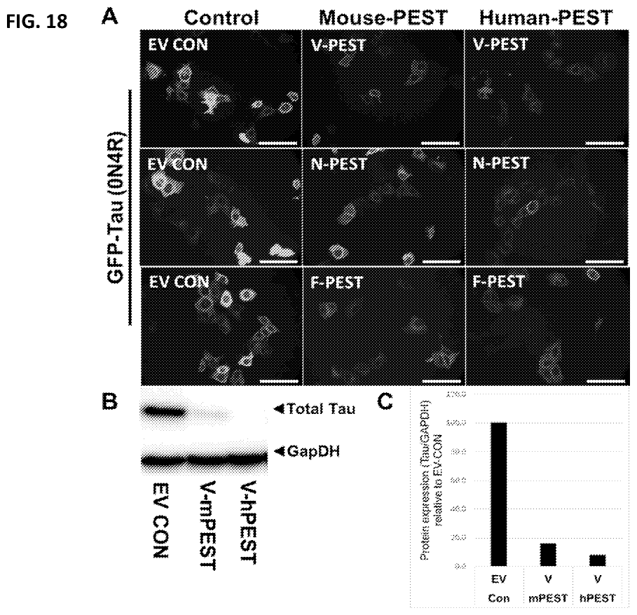

As described herein, the level of tau reduction can be controlled with a human PEST degron by altering the PEST sequence at designated sites. As shown in FIG. 18, the hPEST degron reduced tau to a greater extent than the mPEST degron (92% vs 84% reduction) in immortalized murine ST14A neural precursor cells. Briefly, ST14A cells were transfected with GFP-Tau and either empty vector control (EV CON), V-mPEST, or V-hPEST. 72 h after transfection, samples were collected for: (A) Live cell imaging. (B) Western blot. (C) The relative protein expression was determined by the ratio of total tau to an internal standard control (GAPDH). Samples were then normalized to EV-control.

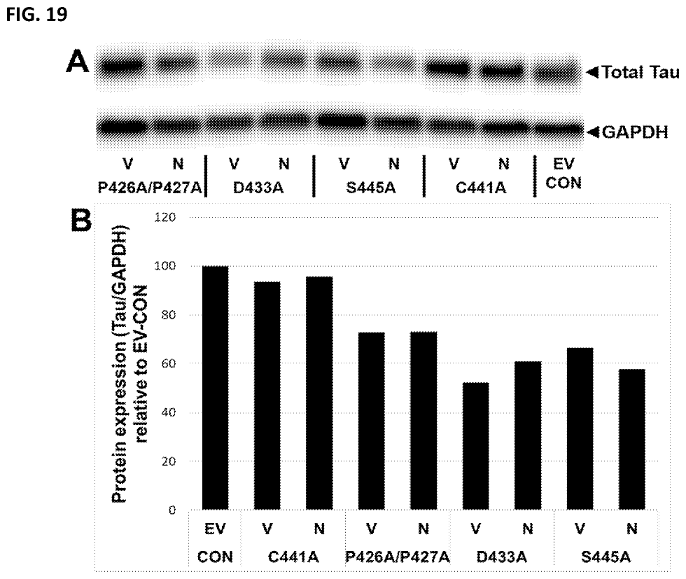

To optimize the level of tau protein reduction within cells, we generated a series of anti-tau-hPEST intrabodies, V-hPEST, N-hPEST, F-hPEST, and A-hPEST that were selected against tau amino acids 151-441. We then generated hPEST degron variants P426A/P427A, E428A-E430A-E431A, D433A, P438A, S435A, S440A, E444A, K448A, S445A, C441A and hPEST-Scramble anti-tau-hPEST intrabodies (V, N, and F). Additionally, we made hPEST variants P426A/P427A, D433A, S445A, C441A and hPEST-Scramble for A-hPEST. The intrabodies were then subcloned into pcDNA3.1(−) expression vector. FIG. 19 shows-controlled degradation of tau by varying the hPEST construct sequence of V-hPEST and N-hPEST intrabodies. ST14A cells were co-transfected with either V-hPEST or N-hPEST or their respective hPEST degron variants P426A/P427A, D433A, S445A, and C441A. Control constructs include empty vector (EV) and an inactive human PEST degron we made by mutating C441 to A, a residue previously identified as critical for proteasome recognition in the mouse PEST degron, and which has been shown to render the human degron inactive. V-hPEST and N-hPEST degron variants D433A, S445A, and P426A/P427A, a compound variant, were then compared. 72-hours after co-transfection with GFP-Tau, the compound V-hPEST-P426A/P427A and N-hPEST-P426A/P427A variants reduced tau by ˜25% compared to EV-CON. The V-hPEST-D433A, N-hPEST-D433A, N-hPEST-S445A and N-hPEST-S445A-hPEST variants resulted in ˜40% reduction of total tau compared to EV-CON. This shows different levels of degradation are achievable with different PEST modifications across multiple intrabodies.

A mutation that may be present in a PEST degron relative to SEQ ID NO:1 as described herein for targeting a protein to the proteasome to achieve reduced degradation of the protein may be a mutation of one or more proline (P) residue to an alanine (A) residue, such as P426A/P427A (i.e., mutation of 2 consecutive P residues to 2 consecutive A residues), a mutation of one or more aspartic acid (D) residue to an A residue, such as D433A, a mutation of one or more serine (S) residue to an A residue, such as S445A.

In other embodiments, a mutation that may be present in a PEST degron relative to SEQ ID NO:1 as described herein for targeting a protein to the proteasome to achieve reduced degradation of the protein may be a mutation of a P residue to an A residue, such as P438A, a mutation of a glutamic acid (E) residue to an A residue, such as E444A, a mutation of an S residue to an A residue, such as S440A, and/or a mutation of a threonine (T) residue to an A residue, such as T436A. Transfection-Rat Progenitor cells (ST14A), which display neuronal characteristics, were utilized for transfection. ST14A cells were cultured using standard protocols.

Cells were cultured into 6 well plates for transfection. Co-transfection was performed using prk5-GFP-tau per well and Anti-tau-hPEST or Anti-tau-hPEST variants expressed in pcDNA3.1(−) expression intrabody expression vectors per well. PEI DNA transfection reagent was utilized to transiently transfect cells. All cultures were imaged 72 hours after transfection, after which cultures were harvested for western blot analysis.

Western Blotting—72 hours after transfection, ST14A cells were imaged for GFP expression. After imaging, samples were collected from 6-well plates by trypsinization. Cell samples were washed with 1×PBS followed by cell lysis using RIPA buffer plus 1× protease inhibiter cocktail (50 mM Tris pH 7.5, 150 mM NaCl, 1% NP40, 0.25% sodium deoxycholate, 2% SDS). Samples were then sonicated for minutes. DC protein assays were performed on samples to generate protein concentration data. From protein assays, sample concentrations were normalized to 1 ng/mL in 2× denaturing sample buffer (125 mM Tris, 4% SDS, 20% glycerol, 10% 2-mercaptoethanol, 0.02% bromophenol blue, pH 6.8) and heated in order to ensure denaturation of proteins. 10 μg of each lysate sample were separated though sodium dodecyl sulfate polyacrylamide gel electrophoresis (SDS-PAGE) using 4-20% Criterion precast gels (Bio-Rad #456-1095). Proteins were blotted onto PDVF membranes (Millipore) using a transblot semi-dry (SD) electroblotter (Bio-Rad) at 24-27V for 30 min. The PDVF membranes were probed for Total tau (DA9; 1:1,000) and GAPDH (as a loading control, Abcam; 1:5,000). Tau degradation readouts from immunofluorescent staining were then verified by western blotting with pan-specific anti-tau monoclonal antibody (1:1,000); HA-tagged intrabodies were probed with monoclonal anti-HA (1:5,000, Covance). Samples were normalized to either actin or GAPDH housekeeping proteins with monoclonal (anti-actin; 1:1000, Sigma or anti-GAPDH; 1:10,000 Abcam) antibodies. Densitometry is quantified with Image J software.

### Example 17—Human Ornithine Decarboxylase (ODC) PEST (hPEST) Degron Variants

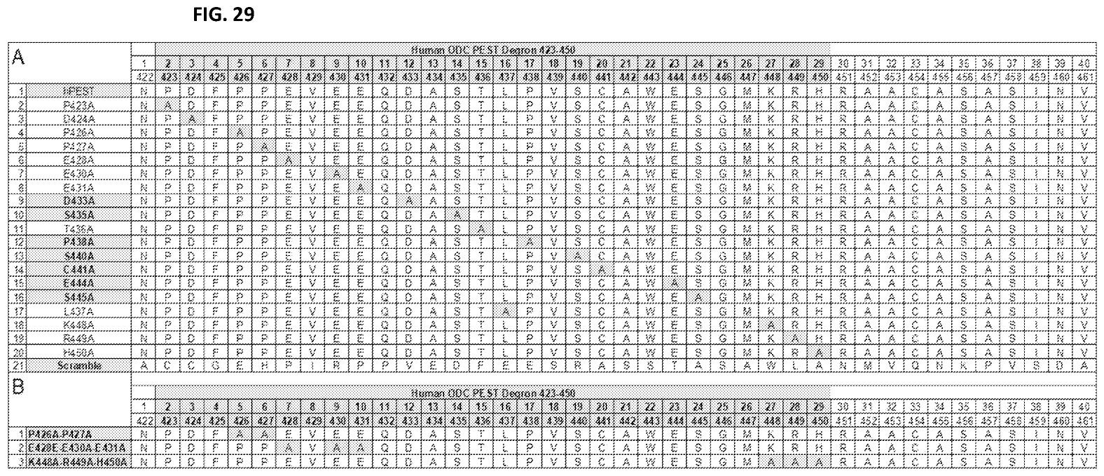

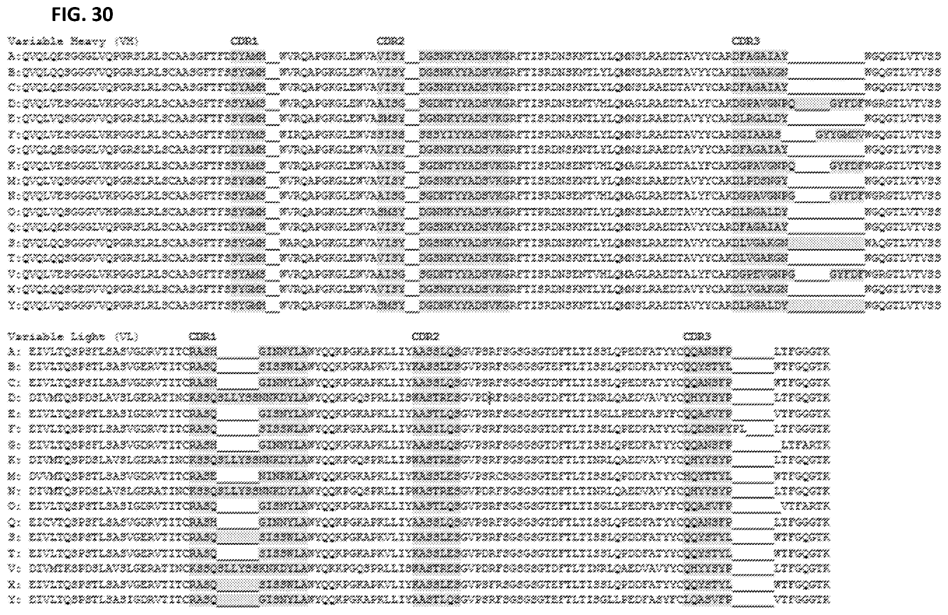

A panel of intrabodies that target tau to the proteasome for degradation via a human proline (P), glutamic acid (E), aspartic acid (D), serine (S) and threonine (T) (PEST) degron fusion has been developed. FIG. 29 shows the PEST degron variants identified in this study. The PEST degron is shaded on the top row (ODC amino acids 423-450). Critical single (A) and compound (B) mutations within the hPEST degron (highlighted in yellow) are predicted to alter the targeted degradation of the intrabody and its bound antigen. This has been demonstrated by ones highlighted in green in FIG. 29.

### Example 18—Efficacy Testing for Mutations that Increase or Decrease Degradation of Tau Protein

Testing of the efficacy of a mutation as described herein for increasing degradation of tau is done using any methods for protein binding, translocation, and the like. For example, a mutation, such as including, but not limited to, P426A/P427A, D433A, S445A is introduced into a human PEST degron sequence as described herein (SEQ ID NO:1). For visualization, a GFP marker or other appropriate screenable marker is used to be able to visualize translocation of the protein to the proteasome. Appropriate controls are used for comparison and to determine success of the mutation in increasing transport of the protein to the proteasome. A cysteine (C)-to-A mutation at residue 441 (C441A) can be used as a control, as this mutation does not have a therapeutic effect. In addition, a subset of mutations using glycine (G) instead of alanine (A) are made and tested using the same methods.

### Example 19—Validate Target Engagement of the Bifunctional Anti-Tau-PEST Intrabodies to Endogenous Human Tau in iPSC-Derived Cortical Neurons

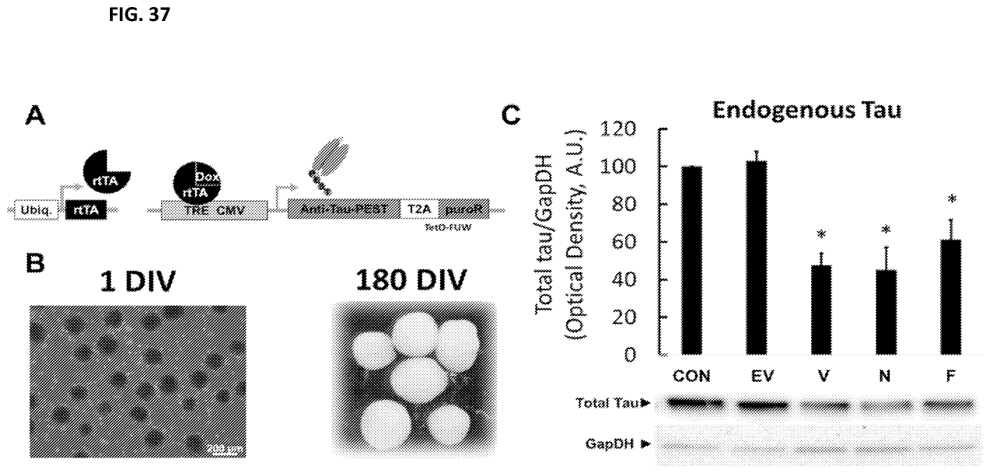

Organoid Differentiation—FTD Patient-derived induced pluripotent stem cells (iPSCs) will be used for validation of bi-functional anti-tau-hPEST and hPEST variants. A human iPSC 3-Dimensional (3D) cerebral organoid protocol developed by Sergiu Pasca and known in the art. Cultures utilizing this protocol result in cerebral organoids which are approximately 1 mm diameter balls of human neural cells that consist predominately of neurons but also include macroglia (FIG. 37). This protocol was selected for its high reproducibility and ability to produce all the major cerebral cortical cell types, including CTIP2-positive neurons, which are present in cortical layer V, and send their axons to deep brain structures, such as the thalamus (corticothalamic neurons), the striatum (corticostriatal neurons), pons (corticopontine neurons), tectum (corticotectal neurons), and spinal cord (corticospinal motor neurons, CSMN).

To evaluate the target engagement of the bifunctional anti-tau-PEST intrabodies to endogenous human tau produced by iPSCs, 3D cortical organoids from a healthy donor (WT) were transduced with inducible lentivirus expressing anti-tau with the mouse PEST degron (tau-mPEST) intrabodies V-mPEST, N-mPEST, and F-mPEST. Following 21 days of treatment, tau levels were compared to empty virus control and untreated controls by western blotting (see methods below). As shown in FIG. 37, V-PEST, N-PEST, and F-PEST significantly (p<0.05) reduced endogenous tau protein levels compared to untreated control (CON) and empty vector (EV) control. Moreover, this result demonstrates that bifunctional anti-tau-PEST intrabodies work in disease-relevant cells. These experiments are a significant improvement upon previous intrabody drug screening models relying on immortalized tumor-derived cell lines, which may contain genetic and metabolic abnormalities due to their mode of derivation. Furthermore, experiments on such lines tend to be limited to shorter time frames (2-4 days) because of their continuous proliferation. By more accurately simulating human disease phenotypes, human iPSC-derived cultures overcome these limitations, serving as a test platform for therapeutic drugs over long periods of time.

Western Blotting—21 days after lentiviral transduction, organoid samples were washed with 1×PBS followed by cell lysis using RIPA buffer plus 1× protease inhibiter cocktail (50 mM Tris pH 7.5, 150 mM NaCl, 1% NP40, 0.25% sodium deoxycholate, 2% SDS). Samples were sonicated for 10 minutes. DC protein assays were performed on samples to generate protein concentration data. From protein assays, sample concentrations were normalized to 1 ng/mL in 2× denaturing sample buffer (125 mM Tris, 4% SDS, 20% glycerol, 10% 2-mercaptoethanol, 0.02% bromophenol blue, pH 6.8) and heated in order to ensure denaturation of proteins. 10 μg of each lysate sample were separated though sodium dodecyl sulfate polyacrylamide gel electrophoresis (SDS-PAGE) using 4-20% Criterion precast gels (Bio-Rad #456-1095). Proteins were blotted onto PDVF membranes (Millipore) using a transblot semi-dry (SD) electroblotter (Bio-Rad) at 24-27V for 30 min. The PDVF membranes were probed for Total tau (DA9; 1:1,000) and GAPDH (as a loading control, Abcam; 1:5,000). Tau degradation readouts from immunofluorescent staining will be verified by western blotting with an anti-tau monoclonal antibody DA9 that recognizes all tau isoforms (1:1,000); HA-tagged intrabodies were probed with monoclonal anti-HA (1:5,000, Covance). Samples were normalized to GAPDH housekeeping proteins with monoclonal (anti-GAPDH; 1:10,000 Abcam) antibodies. Densitometry will be quantified with Image J as previously described.

### Example 20—Controlled Degradation of Tau Using Bifunctional Anti-Tau Intrabody V with a Human PEST Degron in ST14A Neural Precursor Cells

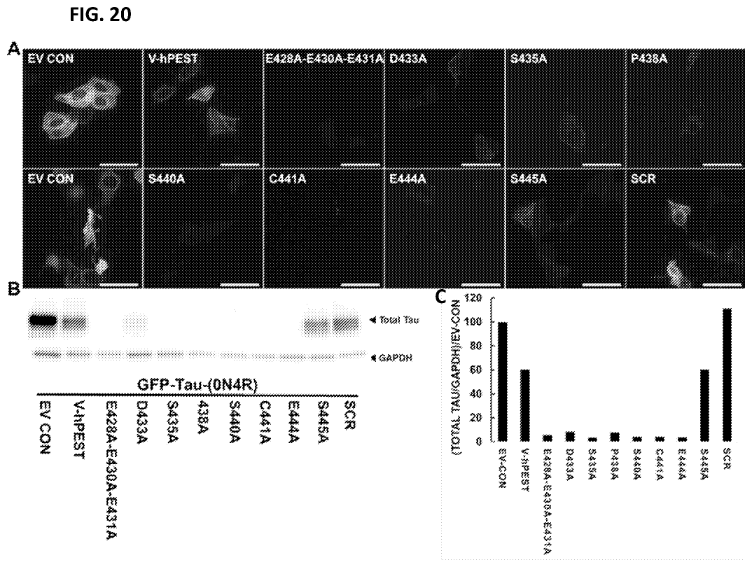

To identify hPEST degron variants that alter the degradation of tau to desired levels, ST14A neuronal cells were transfected with GFP-Tau-(0N4R) and either empty vector control (EV CON), V-hPEST, V-hPEST degron variants E428A/E430A/E431A, D433A, S435A, P438A, S440A, C441A, E444A, S445A, or inactive scrambled PEST degron control (SCR). 72 h after transfection, samples were collected for: (A) Live cell imaging. (B) Western blot. (C) The relative protein expression was determined by the ratio of total tau to an internal standard control (GAPDH). Samples were then normalized to EV-control. Human PEST degron variants resulted in altered protein degradation levels compared empty vector control (EV CON) (FIG. 20). V-PEST variants E428A/E430A/E431A, D433A, S435A, P438A, S440A, C441A and E444A reduced GFP-Tau to 75-100% relative to control. V-PEST and V-PEST variant S445A reduced GFP-Tau to 50-75% of control.

### Example 21—Controlled Degradation of Tau Using Bifunctional Anti-Tau Intrabody N with a Human PEST Degron in ST14A Neural Precursor Cells

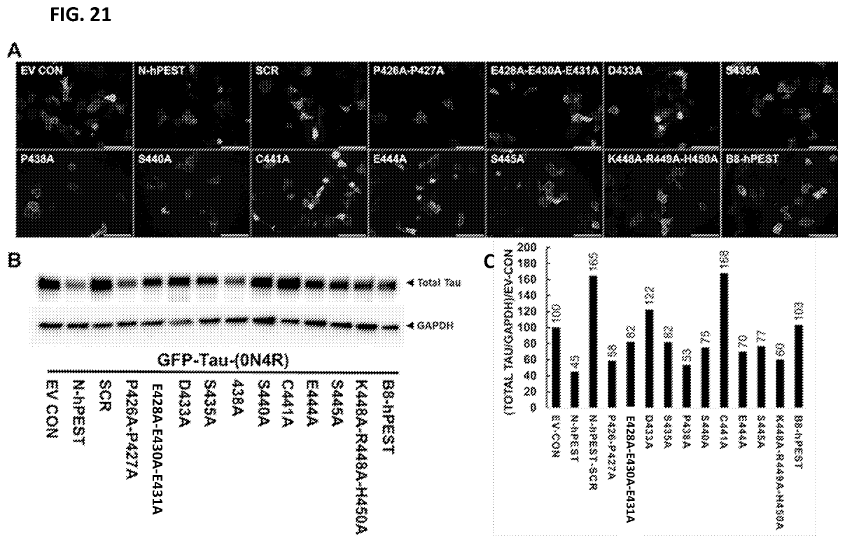

To identify hPEST degron variants that alter the degradation of tau to desired levels, ST14A neuronal cells were transfected with GFP-Tau-(0N4R) and either empty vector control (EV CON), N-hPEST, N-hPEST degron variants E428A/E430A/E431A, D433A, S435A, P438A, S440A, C441A, E444A, S445A, or inactive scrambled PEST degron control (SCR). 72 h after transfection, samples were collected for: (A) Live cell imaging. (B) Western blot. (C) The relative protein expression was determined by the ratio of total tau to an internal standard control (GAPDH). Samples were then normalized to EV-control. Human PEST degron variants resulted in altered protein degradation levels compared empty vector control (EV CON) (FIG. 21). N-PEST reduced GFP-Tau 50-75% relative to control and N-PEST variants P426A/P427A, P438A, E444A, and K448A/R449A/H450A reduced GFP-Tau 25-50% relative to control. N-PEST variants E428A/E430A/E431A, S435A, S440A, and S445A reduced GFP-Tau 0-25% relative to control. B8-PEST, N-PEST-SCR, and N-PEST variants D433A and C441A increased the level of GFP-Tau compared to EV CON.

### Example 22—Controlled Degradation of Tau Using Bifunctional Anti-Tau Intrabody F with a Human PEST Degron in ST14A Neural Precursor Cells

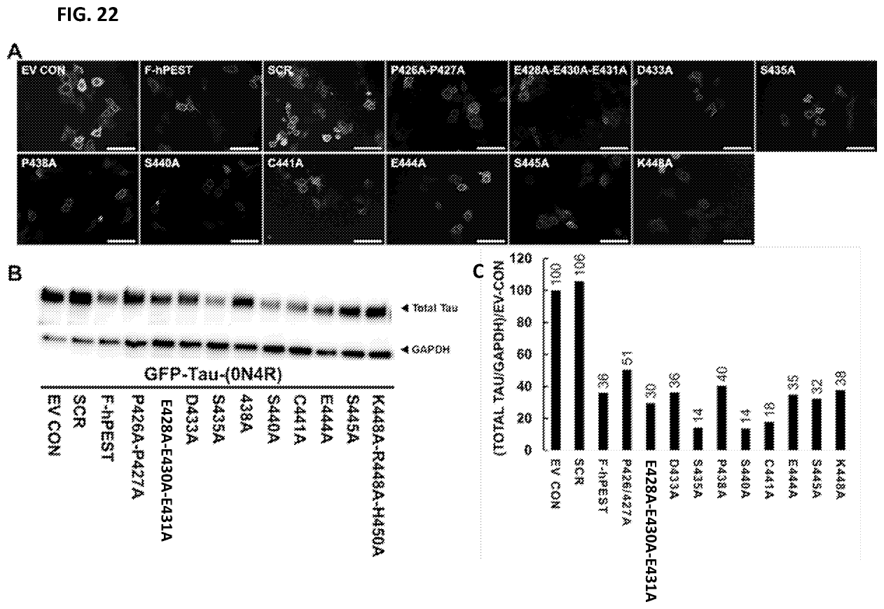

To identify hPEST degron variants that alter the degradation of tau to desired levels, ST14A neuronal cells were transfected with GFP-Tau-(0N4R) and either empty vector control (EV CON), F-hPEST, F-hPEST degron variants P246A/P427A, E428A/E430A/E431A, D433A, S435A, P438A, S440A, C441A, E444A, S445A, K448A/R449A/H450A, or inactive scrambled PEST degron control (SCR). 72 h after transfection, samples were collected for: (A) Live cell imaging. Scale bar=50 μm. (B) Western blot. (C) The relative protein expression was determined by the ratio of total tau to an internal standard control (GAPDH). Samples were then normalized to EV-control. Human PEST degron variants resulted in altered protein degradation levels compared empty vector control (EV CON) (FIG. 22). F-PEST and F-PEST variants E428A/E430A/E431A, D433A, P438A, E444A, S445A, and K448A/R449A/H450A reduced GFP-Tau 50-75% relative to control. F-PEST variant P426A/P427A reduced GFP-Tau 25-50% relative to control. F-PEST-SCR expressed similar levels of GFP-Tau compared to EV CON.

### Example 23—Establishment of a Rigorous Cell Death Assay Using a Series of Neuronal Cells Derived from Human iPSC Lines from Donors Carrying the Disease-Causing MAPT V337M Mutation and their Corresponding Gene-Corrected Controls V337V

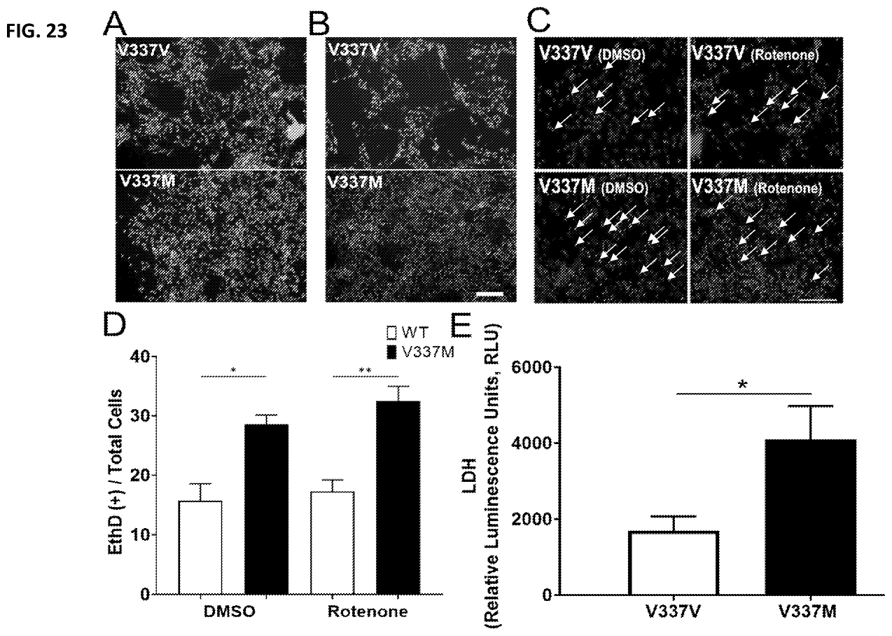

This cell death assay is useful for both modeling FTD and the screening of therapeutic molecules for tauopathies. V337M and control V337V iPSCs were generated into neural progenitor cells (NPCs) that displayed signature features of forebrain identity at 20 days (FIG. 23). Using a protocol developed by the Temple lab, these NPCs and were differentiated into highly enriched cortical neurons of all major cortical cell subtypes by 45 days. Next, the survival of the V337M mutant versus control neurons was performed. Previously, work has shown that A152T MAPT mutation neurons are more susceptible to stressors such as rotenone, demonstrating elevated cell death (Silva, Cheng et al. (2016) Stem Cell Reports 7(3): 325-340). Notably, this vulnerability to stressors only appeared after cultures were ˜100 days old. Therefore, we compared the level of cell death in 110-day V337M vs V337V control cortical neurons with and without rotenone treatment. Cell death was quantified using an ethidium homodimer assay (Thermo Fisher); in this assay, dying cells are indicated by nuclear dye incorporation into damaged DNA. There was significantly increased cell death in the 110-day V337M mutant neurons versus isogenic V337V control (FIG. 23). The presence of dead cells that have lost membrane integrity can be detected by measuring markers that leak from the cytoplasm such as lactate dehydrogenase (LDH) into the culture medium (Riss, Niles et al. (2004) Assay Guidance Manual. S. Markossian, G. S. Sittampalam, A. Grossman et al. Bethesda (MD). Cell death was significantly increased in V337M mutant cultures by the LDH-Glo™ Cytotoxicity Assay (Promega) kit (FIG. 23). Ehrlich, et al. recently showed patient-derived iPSC lines with a MAPT V337M mutation make neurons with enhanced tau fragmentation and phosphorylation, decreased neurite extension, and enhanced susceptibility to oxidative stress (Ehrlich, Hallmann et al. (2015) Stem Cell Reports 5(1): 83-96). Surprisingly, as shown, we did not observe a significant increase in cell death when stressing the cells with rotenone, an environmental toxin that inhibits the mitochondrial electron transport chain (ETC) complex I. The main difference between the studies were that we generated forebrain neurons compared to midbrain neurons that were produced by Ehrlich. This is relevant because a subset of patients with the V337M mutation have severe frontal lobe atrophy with a high density of NFTs, pretangles, and neuropil threads (Spina, Schonhaut et al. 2017) while the substantia nigra (located in the midbrain region) in the same patients displayed mild atrophy and NFT pathology (Spina, Schonhaut et al. (2017) Neurology 88(8): 758-766). These results, combined with clinical data, suggests that there is selective vulnerability in forebrain neurons to the V337M mutation alone without additional stressors.

### Example 24—the Proteasome is Impaired in iPSC Derived Cortical Neurons with a MAPT V337M Mutation Compared to Isogenic V337V Controls

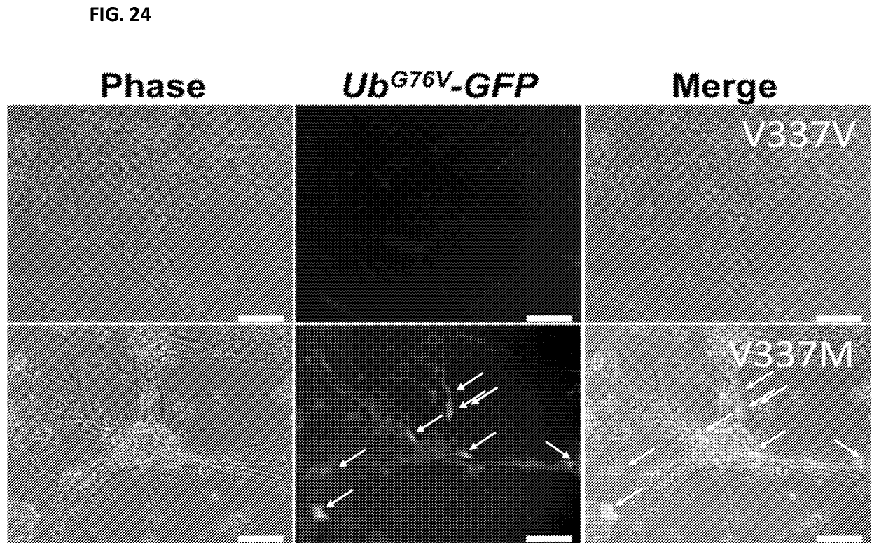

To monitor proteasome function, mutant (V337M) and isogenic control (V337V) cortical cultures were transduced with a Ubiquitin^{G76V}GFP (Ub^{Gb76V}GFP) reporter. This reporter is widely used for monitoring the role of ubiquitin/proteasome-dependent proteolysis in diverse disorders, and for efficacy trials testing the effect of compounds on the ubiquitin/proteasome system. V337V and V337M cortical cultures were transduced at 90 days. At 110 days in culture, a timepoint where cell death is increased in MAPT V377M mutant cultures compared to isogenic V337V control (FIG. 23), cells were live imaged to measure expression levels of Ub^{G76V}GFP reporter. In healthy, V337V control cultures, the Ub^{G76V}GFP (arrows) reporter is rapidly degraded; however, in MAPT V337M mutant cortical neurons, there is accumulation of UB G76V GFP reporter which implies that ubiquitin proteasome system is impaired (FIG. 24) and potentially contributing to cell death observed in FIG. 23.

### Example 25—Bi-Functional Anti-Tau-PEST Intrabodies Alleviate Proteasome Impairment in Induced Pluripotent Stem Cell (iPSC) Derived Cortical Neurons with a MAPT V337M Mutation Compared to Isogenic V337V Controls

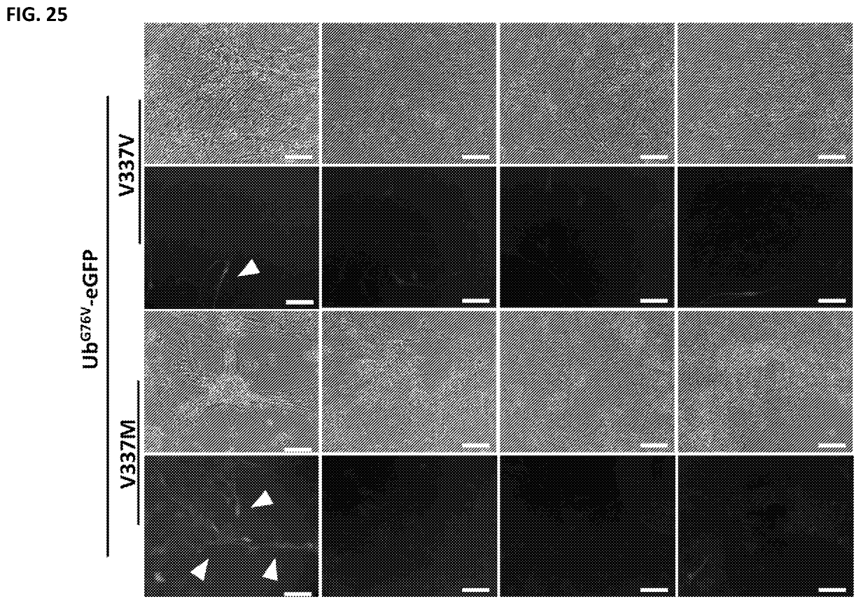

To determine if anti-tau-PEST intrabodies can counteract proteasome impairment caused by mutant V337M tau, Mutant (V337M) and isogenic control (V337V) cortical cultures were transduced with Ubiquitin^{G76V}GFP (Ub^{G76V}GFP) reporter as described in (FIG. 24) and either empty vector control (EV), V-hPEST, N-hPEST, or F-hPEST. Following 20 days of treatment, the cells were live imaged to measure the fluorescent expression levels of Ub^{G76V}GFP reporter. In V337M mutant cultures treated with anti-tau-hPEST intrabodies, the Ub G76 VGFP reporter is rapidly degraded (FIG. 25). This demonstrates that bifunctional anti-tau-PEST intrabodies, which undergo ubiquitin independent proteolysis, can counteract proteasome impairment due to V337M tau toxicity.

### Example 26—Anti-Tau-hPEST Intrabodies, V-hPEST and N-hPEST, Reduced Cell Death in Human iPSC Derived Cortical Neurons with a MAPT V337M Mutation

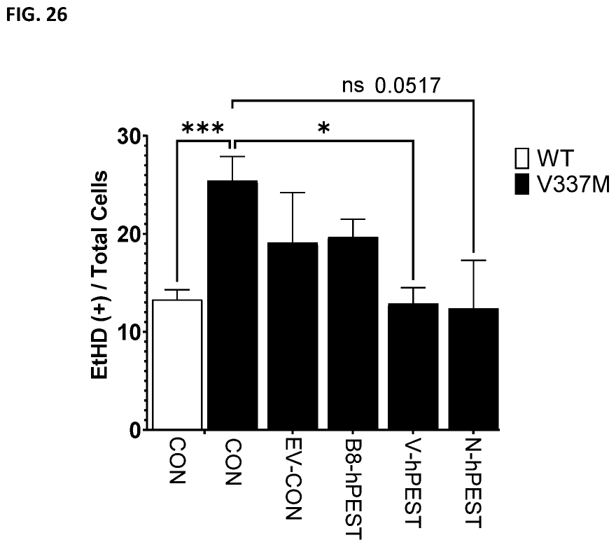

After showing that V337M mutant cortical cultures display elevated cell death (FIG. 23) and proteasome impairment (FIG. 24), which can be counteracted by anti-tau-PEST intrabodies (FIG. 25) cell death, was evaluated following treatment with bifunctial anti-tau-PEST intrabodies. V337M cortical cultures were transduced at 90 days with either empty vector control (EV-CON), V-hPEST, N-hPEST, or B8-hPEST control intrabody to an irrelevant antigen, botulinum toxin. At 110 days, an ethidium homodimer (EtHD) assay was used to detect dead and/or dying cells. As expected, cell death levels were reduced to control levels by V-hPEST and N-hPEST intrabodies (FIG. 26). Cell death was significantly reduced by V-hPEST (*, p=while N-hPEST approached significance (p=0.0517) in V337M cultures compared to isogenic V337V control.

### Example 27—with Programmable Target Antigen Proteolysis (P-TAP) Technology, Using Anti-Tau-PEST Intrabodies, the Lowest Effective Level of Tau Degradation to Achieve Neuroprotection can be Determined in Disease Relevant Human iPSC Derived Cortical Neurons

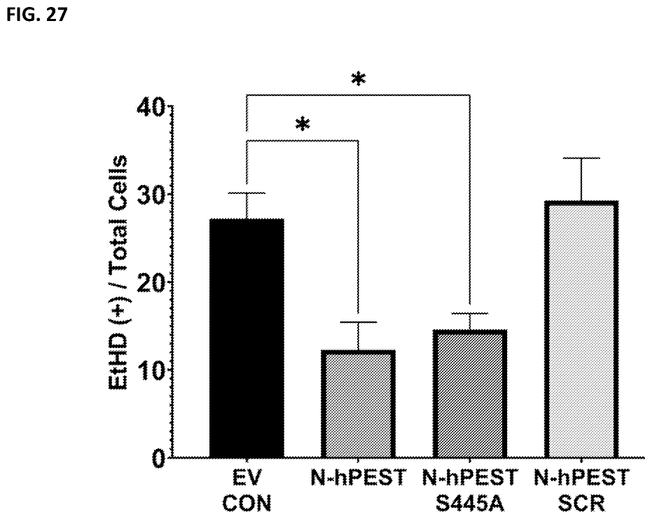

To determine the level of tau reduction necessary to achieve neuroprotection in human cells mutant MAPT V337M cortical cultures were transduced with N-hPEST, a strong reducer of Tau (50-75%; FIG. 21) or N-hPEST degron variant S445A, a low reducer of Tau (0-25%; FIG. 21) and compared to either empty vector control or N-hPEST with a scrambled inactive PEST degron. In this experiment, cultures were transduced at 60 days and then cell death was evaluated using the ethidium homodimer (EtHD) assay at 90 days as described in (FIG. 23 and FIG. 26). In agreement with FIG. 26, cell death levels were significantly (*, p<0.05) reduced by N-hPEST, a strong reducer of Tau (50-75%) compared to EV CON (FIG. 27). N-hPEST degron variant S445A, a low reducer of Tau (0-25%), significantly (*, p<0.05) reduced cell death in V337M cortical cultures compared to EV CON (FIG. 27). This novel finding demonstrates that following prolonged expression with only ˜25% tau reduction is sufficient to achieve neuroprotection.

### Example 28—Identification of Bi-Functional Anti-Huntingtin-Human-PEST Variants that Control the Degradation of Huntingtin

Huntingtin is a protein that is causative of Huntington's Disease. Expansion of a CAG repeat in exon 1 of the HTT gene results in a protein with an abnormal polyglutamine (polyQ) stretch at the N-terminus. This polyQ stretch adopts a number of conformations including an α-helix, random coil, and extended loop. Huntington's Disease results when mutant huntingtin protein aggregates, resulting in neurological symptoms as a result of neuronal cell death. Targeted degradation of abnormal huntingtin protein is therefore an important therapeutic target. Intrabodies can be designed and selected to bind to various protein conformations and epitopes on their targets. In addition, they can be further engineered to relocate target proteins to different cellular compartments such as the nucleus, endoplasmic reticulum, and proteasome (FIG. 1).

As described herein, the Inventors have developed bi-functional proteasome-targeting intrabodies that prevent protein misfolding while targeting their bound cargo to the proteasome for degradation via a fusion to the PEST degron from human ornithine decarboxylase (hPEST). The overarching hypothesis of this study is that targeted degradation of huntingtin protein using the cell's normal protein clearing process will reduce the amount of huntingtin available to misfold and thus reduce the physiological effects of Huntington's Disease or other diseases associated with mutation and/or aggregation of huntingtin.

### Example 29—Controlled Degradation of Intracellular Proteins Using a Human PEST

As described herein, the level of huntingtin reduction can be controlled with a human PEST degron. It is demonstrated herein that this works in multiple systems. Without being bound by any theory, the targeted degradation of huntingtin protein using the cell's normal protein clearing process may reduce the amount of huntingtin available to misfold and thus reduce the physiological effects of Huntington's Disease.

For example, it is shown that modification of the degron allows control of the level of huntingtin degradation in cells in culture (e.g., ST14A rat neural precursor cells) are planned.

Assumptions made are that (1) Antigen-intrabody association is a given, and (2) Degradation of antigen-intrabody-degron complex is proportional to degradation of unbound intrabody-degron. The huntingtin protein was targeted herein, which is a naturally occurring protein, with intrabodies having a PEST degron from human ODC.

As shown in FIG. 35, a mutation that may be present in a PEST degron relative to SEQ ID NO:1 as described herein for targeting a protein to the proteasome to achieve altered degradation of the protein may be a mutation of a proline (P) residue to an alanine (A) residue, such as P426A/P427A (i.e., a compound mutation of 2 consecutive P residues to 2 consecutive A residues), a single mutation of an aspartic acid (D) residue to an A residue, such as D433A, a single mutation of a serine (S) residue to an A residue, such as S445A, and/or a mutation of a lysine (K) residue to an A residue, such as K448A.

Testing of the efficacy of a mutation as described herein for increasing degradation of huntingtin is done using any methods for protein binding, translocation, and the like. For example, a mutation, such as including, but not limited to, P426A/P427A, D433A, S445A, and/or K448A is introduced into a human PEST degron sequence as described herein (SEQ ID NO:1). For visualization, a GFP marker or other appropriate screenable marker is used to be able to visualize translocation of the protein to the proteasome. Appropriate controls are used for comparison and to determine success of the mutation in increasing transport of the protein to the proteasome. A cysteine (C)-to-A mutation at residue 441 (C441A) can be used as a control, as this mutation does not have a therapeutic effect.

Cloning—Anti-huntingtin-PEST and their respective PEST variants (SEQ ID NO: 100-105) have been subcloned into pAAV-MCS. To identify expression of the intrabodies, a hemagglutinin (HA) epitope tag (amino acid sequence YPYDVPDYA) is fused to the C-terminal end of the intrabodies. To direct the intrabodies and their cargo to the proteasome, a standard PEST motif corresponding to amino acids 422-461 from human ODC (GenBank accession number AH002917.2) is added C-terminal of the HA-tag. The scFv intrabodies are arranged as 5′-VH-(G4S)3-VL-HA-PEST-3′. The intrabodies are subcloned with standard cloning techniques into pAAV-MCS according to the following cloning strategy: XbaI-intrabody-NotI-HA-PEST degron-HindIII. All expression plasmids are verified by Sanger DNA sequencing (Genewiz, NJ) and prepared with Nucleobind Xtra Midi Endotoxin free (Takara #740420.5) prep kits according to the manufacturer's protocol.

Transfection-Rat Progenitor cells (ST14A), which display neuronal characteristics, will be utilized for transfection. ST14A cells will be cultured using standard protocols. Cells will be cultured into 6 well plates for transfection. Co-transfection is performed using 0.75 μg mHTTex1-72-eGFP-pcDNA3.1 per well and 2.25 μg Anti-huntingtin-hPEST or Anti-huntingtin-hPEST variants expressed in pAAV expression vectors per well. PEI DNA transfection reagent is utilized to transiently transfect cells. All cultures will be imaged 72 hours after transfection, after which cultures will be harvested for western blot analysis.

Western Blotting—72 hours after transfection, ST14A cells will be imaged for GFP expression. After imaging, samples will be collected from 6-well plates by trypsinization. Cell samples will be washed with 1×PBS followed by cell lysis using RIPA buffer plus 1× protease inhibiter cocktail (50 mM Tris pH 7.5, 150 mM NaCl, 1% NP40, 0.25% sodium deoxycholate, 2% SDS). Samples will be sonicated for 10 minutes. DC protein assays will be performed on samples to generate protein concentration data. From protein assays, sample concentrations will be normalized to 1 ng/mL in 2× denaturing sample buffer (125 mM Tris, 4% SDS, 20% glycerol, 10% 2-mercaptoethanol, 0.02% bromophenol blue, pH 6.8) and heated in order to ensure denaturation of proteins. 10 μg of each lysate sample will be separated though sodium dodecyl sulfate polyacrylamide gel electrophoresis (SDS-PAGE) using 4-20% Criterion precast gels (Bio-Rad #456-1095). Proteins will be blotted onto PDVF membranes (Millipore) using a transblot semi-dry (SD) electroblotter (Bio-Rad) at 24-27V for 30 min. The PDVF membranes will be probed for mutant huntingtin (EM48; 1:1,000) and GAPDH (as a loading control, Abcam; 1:5,000). Densitometry will be quantified with Image J software.

### Example 30—Humanization of Bi-Functional Anti-HTT-PEST Intrabodies

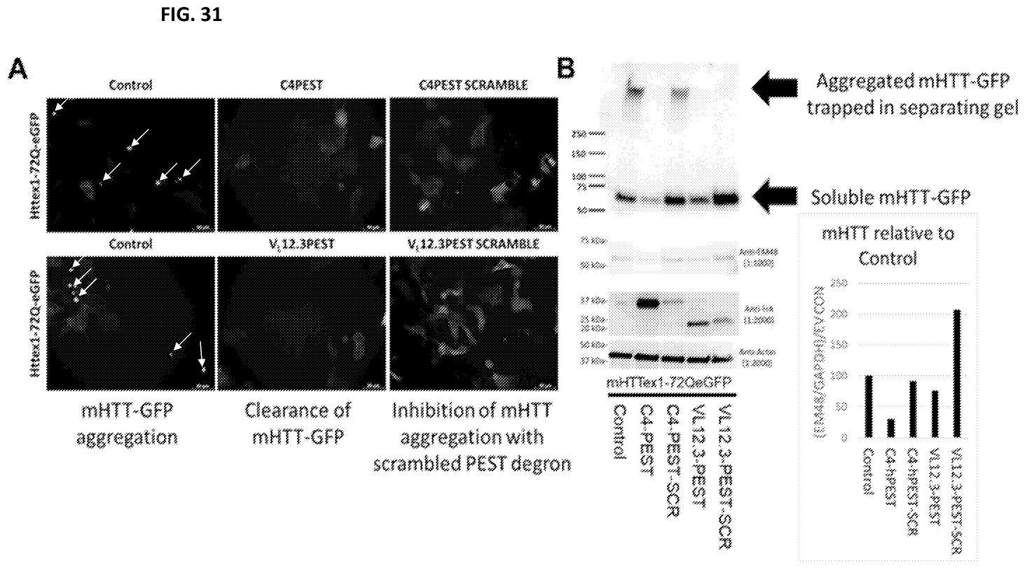

The PEST degron was optimized for human use. To accomplish this goal, the mouse PEST degron (mPEST) was substituted with the human PEST (hPEST) degron. A comparison of the mouse and human PEST degron from the ornithine decarboxylase (ODC) gene is shown in Table 3. Fusion of the hPEST degron to the anti-HTT intrabodies directs mutant HTT exon1 protein fragments to the proteasome for degradation as efficiently as mPEST degron. Therefore, the hPEST degron was cloned from human ornithine decarboxylase onto the anti-HTT C4 scFv intrabody and VL12.3 single domain intrabody. mHTTex1-72Q-eGFP was co-transfected with either Empty Vector Control (Control), C4-PEST, VL12.3-PEST, or C4 and VL12.3 with an inactive scrambled PEST degron that does not promote protein degradation (C4-PEST-SCR), or (VL12.3-PEST-SCR). 72 h after transfection, cells were live imaged and harvested for western blotting. As shown in FIG. 31, live cell imaging revealed that mHTTex1-72Q-GFP readily formed aggregates (puncta) in the EV CON cells. In C4-hPEST and VL12.3-hPEST treated cells, the florescent signal for mHTTex1-72Q-GFP was barely detectible. C4-PEST and VL12.3-PEST prevent mHTTex1-72Q-GFP aggregation compared to control. Western blot analysis confirmed the live cell imaging result. Soluble and insoluble (high molecular weight species) mHTTex1-72Q-GFP, detected with monoclonal antibody EM48 (Millipore, Cat #MAB5374), was reduced in C4-PEST and VL12.3-PEST treated cells compared to empty vector control and C4-PEST-SCR and VL12.3-PEST-SCR controls (FIG. 31).

### Example 31—Mutant HTT Exon 1 Protein Fragments with Either 46Q or 72Q Repeats Impair the Ubiquitin Proteasome System

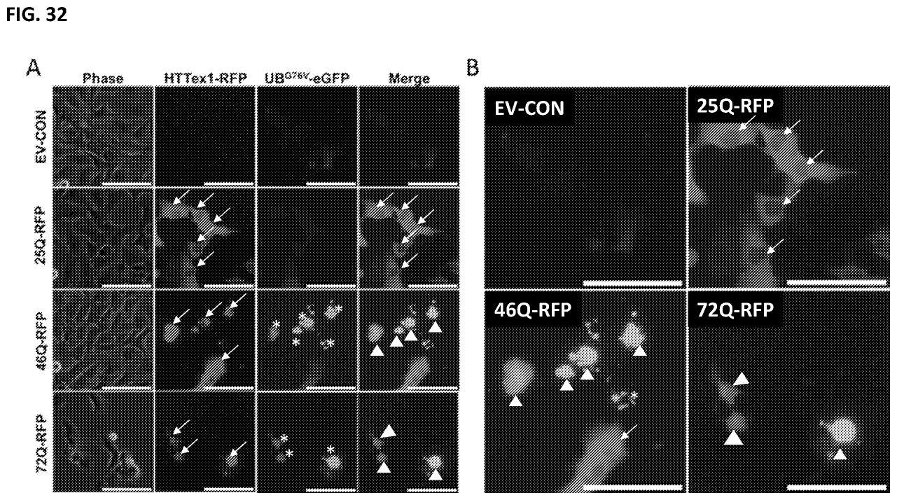

To determine if the ubiquitin-proteasome is impaired by toxic mutant HTT protein fragments, ST14A cells were co-transfected with a Ubiquitin^{G76V}GFP (Ubiquitin^{G76V}GFP) reporter and either empty vector control (EV CON), mHTTex1-25Q-RFP (46Q-RFP), mHTTex1-46Q-RFP (46Q-RFP), or mHTTex1-72Q-RFP (72Q-RFP). 72 hours after transfection, the cells were live imaged for mHTT aggregation and the accumulation of UB G76v GFP (FIG. 32). In healthy cells, the Ubiquitin^{G76V}GFP is quickly degraded by the proteasome and when the proteasome is impaired, the Ubiquitin^{G76V}GFP reporter accumulates. As expected, in healthy EV CON cells, the Ubiquitin^{G76V}GFP is quickly degraded by the proteasome. In cells transfected with a non-pathogenic polyglutamine repeat length, 25Q-RFP, the UB G76v GFP reported was also efficiently degraded. However, in cells treated with pathogenic polyglutamine repeat lengths, 46Q-RFP and 72Q-RFP, the UB G76v GFP reporter accumulated and was also incorporated into the mHTT aggregates. Collectively, this implies that ubiquitin proteasome system is impaired by mHTT aggregation.

### Example 32—Bi-Functional Intrabody, C4-PEST, Counteracts Proteasome Impairment Caused by mHTT Exon 1 Protein Fragments

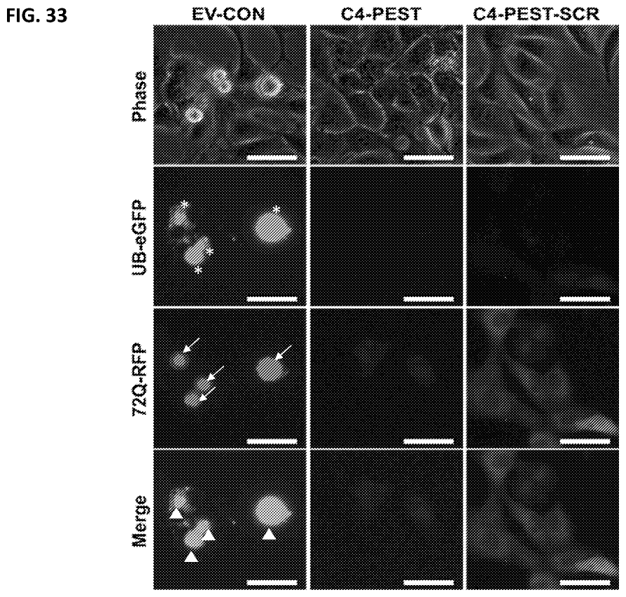

To determine if the inhibition of mHTTex1-72Q-RFP aggregation and its clearance through the proteasome through ubiquitin independent proteolysis via C4-hPEST, ST14A cells were co-transfected with Ubiquitin^{G76V}GFP (Ubiquitin^{G76V}GFP) reporter, mHTTex1-72Q-RFP (72Q-RFP), and either Empty Vector Control (EV CON), C4 with a human PEST degron (C4-PEST), or C4 with an inactive scrambled human PEST degron (C4-PEST-SCR). 72 hours after transfection, the cells were live imaged for mHTT aggregation and the accumulation of UB G76v GFP. As shown in FIG. 33, the proteasome was impaired by Q72-RFP aggregation. As also shown in FIG. 32, the UB G76v GFP reporter accumulated in these cells and was incorporated into 72Q-RFP aggregates; however, in cells treated with C4-PEST-SCR, which only inhibits 72Q-RFP aggregation, the UB G76v GFP reporter quickly was degraded by the proteasome. Furthermore, in cells Q72-RFP treated with C4-PEST, which inhibits Q72-RFP aggregation and directs Q72-RFP to the proteasome via ubiquitin independent proteolysis, both Q72-RFP and the UB G76v GFP reporter are effectively cleared by the cell (FIG. 33).

### Example 33—Controlled Degradation of Toxic Intracellular mHTT Fragments Using Bifunctional Anti-HTT Intrabody C4 with a Human PEST Degron in Rat ST14A Neural Precursor Cells

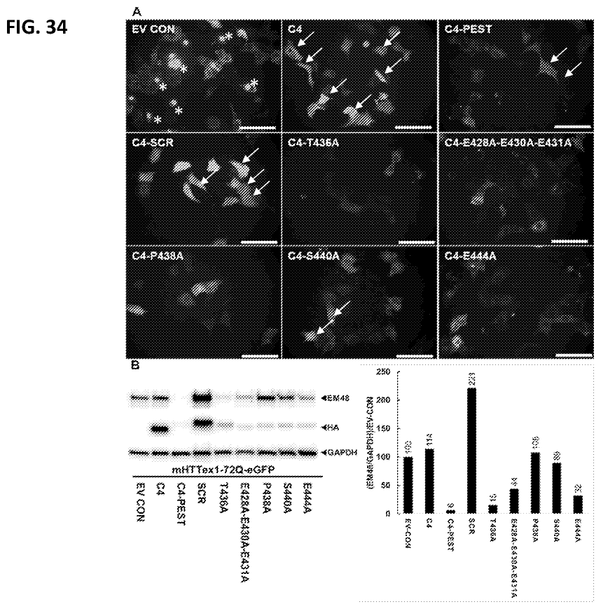

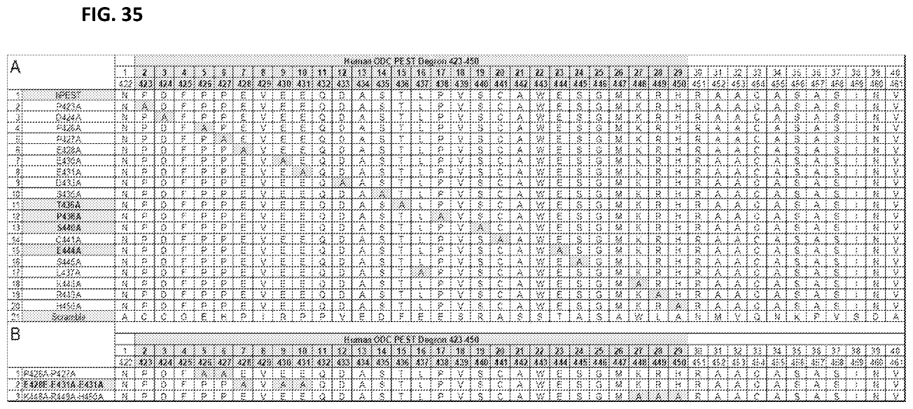

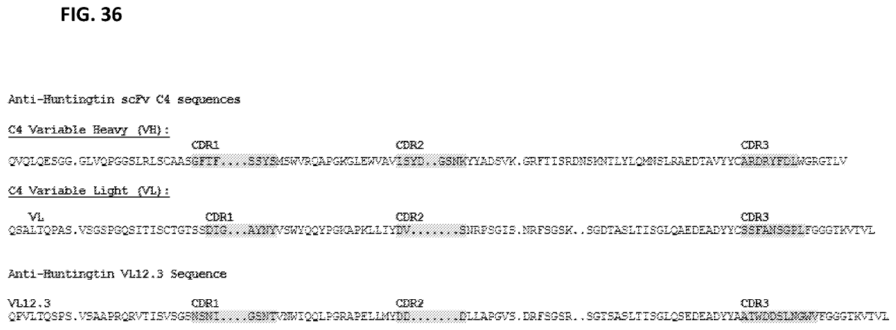

To identify hPEST degron variants that alter the degradation of mHTT to desired levels, ST14A neuronal cells were transfected with human mHTTex1-72Q-GFP and either empty vector control (EV CON), C4, C4-PEST, and C4-PEST variants (T436A, P438A, S440A, E444A), compound mutation variant (E428A/E430A/E431A), or inactive scrambled PEST degron control (SCR). 72 h after transfection, cells were live imaged and harvested for western blotting. As shown in FIG. 34, live cell imaging revealed that mHTTex1-72Q-GFP readily formed aggregates (puncta) in the EV CON cells. In agreement with FIGS. 31, C4 and C4-PEST-SCR prevented mHTTex1-72Q-GFP aggregation. In C4-PEST and C4-PEST variant T436A treated cells, the presence of soluble mHTT was barely detectible. Cells treated with C4-PEsST variants E428A/E430/E431A, E444A, and S440A displayed increased levels of soluble mHTTex1-72Q-GFP compared to C4-PEST. Western blotting was used confirm the live cell imaging results (FIG. 31). For western blotting: mHTTex1-72Q-GFP was detected using a monoclonal antibody EM48 (Millipore, Cat #MAB5374). The intrabodies were detected by probing for their HA-tag with anti-HA, and GAPDH was probed as a loading control. The relative protein expression was determined by the ratio of soluble mHTTex1-72Q-GFP (EM48) to an internal standard control (GAPDH). Samples were then normalized to EV-CON. Human PEST degron variants T436A, S440A, E444A, and compound mutation variant E428/430/431A result in altered protein degradation levels compared to EV CON. C4-PEST and C4-PEST variant T436A reduced mHTT to 75-100% relative to control. C4-PEST variants E428A/E430/E431A and E444A reduced mHTT to 50-75% of control. C4-PEST variant S440A reduced mHTT to 25-0% of control, with C4, C4-PEST-SCR, and C4-PEST variant P438A all increasing the percentage of mHTT relative to control. These results show different levels of mHTT degradation are achievable with different PEST modifications.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A programmable proteasome-targeting human PEST domain comprising a sequence that is at least 85% identical, at least 90% identical, or at least 95% identical to an amino acid sequence as set forth in SEQ ID NO: 1 and having at least one amino acid substitution, wherein, when the PEST domain is fused to an antigen binding domain that binds to a protein, the at least one amino acid substitution increases degradation of the protein relative to an empty vector (EV) control.

2. The programmable proteasome-targeting human PEST domain of claim 1, wherein at least one amino acid substitution determines the relative increase in degradation.

3. The programmable proteasome-targeting human PEST domain of claim 2, wherein at least one amino acid substitution is selected from alanine, glycine, valine, leucine, or isoleucine.

4-5. (canceled)

6. The programmable proteasome-targeting human PEST domain of claim 1, wherein the programmable proteasome-targeting PEST domain increases degradation of the protein relative to an empty vector control in an amount of between (a) about 10% to about 30%, (b) about 30% to about 50%, (c) about 50% to about 70% or (d) about 70% to about 90%.

7-11. (canceled)

12. A recombinant polypeptide comprising:
an antigen-binding domain that binds a protein; and
the programmable proteasome-targeting human PEST domain of claim 1.

13. The recombinant polypeptide of claim 12, wherein the antigen-binding domain is an intrabody.

14. The recombinant polypeptide of claim 13, wherein the intrabody is a single-chain variable fragment (scFv) or a single-domain antibody.

15.-30. (canceled)

31. A method for the treatment of a protein aggregation disease in a patient in need thereof, the method comprising administering to the patient a therapeutically effective amount of a recombinant polypeptide including an antigen-binding domain that binds a protein; and the programmable proteasome-targeting human PEST domain of claim 1.

32. The method of claim 31, wherein the protein is α-synuclein and the protein aggregation disease is selected from the group consisting of Parkinson's Disease (PD), multiple system atrophy (MSA), Lewy Body dementia, Alzheimer's Disease (AD), frontotemporal dementia (FTD), progressive supranuclear palsy (PSP), chronic traumatic encephalopathy (CTE), spinal cord injury (SCI), traumatic brain injury (TBI), and other synucleinopathies.

33-102. (canceled)

103. A polypeptide that binds huntingtin, comprising an amino acid sequence that is at least 80%, at least 85%, at least 90%, at least 95%, at least 97%, or 100% identical to any one of SEQ ID NOs: 100-108.

104. A polypeptide that binds α-synuclein, comprising an amino acid sequence that is at least 80%, at least 85%, at least 90%, at least 95%, at least 97%, or 100% identical to any one of SEQ ID NOs: 6-10, 12 and 13.

105. A polypeptide that binds tau, comprising an amino acid sequence that is at least 80%, at least 85%, at least 90%, at least 95%, at least 97%, or 100% identical to any one of SEQ ID NOs: 23-33, 35-45, 47-57 and 59-63.

106. The recombinant polypeptide according to claim 12, wherein the antigen-binding domain binds tau.

107. The recombinant polypeptide according to claim 12, wherein the antigen-binding domain binds α-synuclein.

108. The recombinant polypeptide according to claim 12, wherein the antigen-binding domain binds huntingtin.

109. A programmable proteasome-targeting human PEST domain comprising the sequence NPDFX₁X₂X₃VX₄X₅QX₆AX₇X₈LX₉VX₁₀X₁₁AWX₁₂X₁₃GMXHRHRAACASASINV (SEQ ID NO: 109),
wherein X₁ is (P/A), X₂ is (P/A), X₃ (E/A), X₄ is (E/A), X₅ is (E/A), X₆ is (D/A), X₇ is (S/A), X₈ is (T/A), X₉ is (P/A), X₁₀ is (S/A), X₁₁ is (C/A), X₁₂ is (E/A), X₁₃ is (S/A), and X₁₄ is (K/A),
wherein the sequence is not NPDFPPEVEEQDASTLPVSCAWESGMKRHR AACASASINV (SEQ ID NO:3).

110. The programmable proteasome-targeting human PEST domain of claim 109, wherein the domain comprises the sequence:
X₁ is (P), X₂ is (A), X₃ is (E), X₄ is (E), X₅ is (E), X₆ is (D), X₇ is (5), X₈ is (T), X₉ is (P), X₁₀ is (S), X₁₁ is (C), X₁₂ is (E), X₁₃ is (S), and X₁₄ is (K) (amino acids 164-191 in SEQ ID NO:7) or
X₁ is (P), X₂ is (P), X₃ is (E), X₄ is (E), X₅ is (E), X₆ is (A), X₇ is (S), X₈ is (T), X₉ is (P), X₁₀ is (S), X₁₁ is (C), X₁₂ is (E), X₁₃ is (S), and X₁₄ is (K) (amino acids 164-191 in SEQ ID NO:8) or
X₁ is (P), X₂ is (P), X₃ is (E), X₄ is (E), X₅ is (E), X₆ is (D), X₇ is (5), X₈ is (T), X₉ is (P), X₁₀ is (S), X₁₁ is (C), X₁₂ is (E), X₁₃ is (A), and X₁₄ is (K) (amino acids 164-191 in SEQ ID NO:10).

111. The method of claim 31, wherein the protein is tau and the protein aggregation disease is selected from Frontotemporal dementia (FTD), Alzheimer's Disease (AD) progressive supranuclear palsy (PSP), frontotemporal dementia with Parkinsonism on chromosome-17 (FTDP-17), frontotemporal lobar degeneration (FTLD-TAU), corticobasal degeneration (CBD), primary age-related tauopathy, Pick's disease, chronic traumatic encephalopathy (CTE), Lewy Body dementia, Vascular dementia, tuberous sclerosis, spinal cord injury (SCI), traumatic brain injury (TBI) or other tauopathies.

112. The method of claim 31, wherein the protein is huntingtin and the protein aggregation disease is selected from Huntington's disease, or other protein aggregation neurodegeneration diseases including Parkinson's Disease (PD), multiple system atrophy (MSA), and Lewy Body dementia, Alzheimer's Disease (AD), frontotemporal dementia (FTD), progressive supranuclear palsy (PSP), chronic traumatic encephalopathy (CTE), and spinal cord injury (SCI), and traumatic brain injury (TBI).
